SEQUENCE LISTING (58 sequences)

SEQ ID NO: 1 - DNA, 10 bp, Artificial Sequence
  ISRE canonical motif
  misc_feature (5)..(6): n is a, c, g, or t
gaaanngaaa

SEQ ID NO: 2 - DNA, 10 bp, Artificial Sequence
  EICE-type canonical motif
  misc_feature (5)..(6): n is a, c, g, or t
ggaanngaaa

SEQ ID NO: 3 - DNA, 20 bp, Artificial Sequence
  CpG DNA oligonucleotide
tccatgacgttcctgacgtt

SEQ ID NO: 4 - protein, 1284 aa, Mus musculus
MEGAKGPRLRGFLSGSLATWALGLAGLVGEAEESAGGTEEEEEEEEEEGALCTEKRFLRLIDGALLLRVLGIIAPSSRGGLRMVRGHDGPAACRMWNLCHLWGRLRDFYQEELQLLILSPPPDLQTMGCDPFSEEAVDELESILRLLLGASVQCEHRELFIRHIRGLSLDVQSELAGAIQEVTQPGAGVVLALAGPESGELVAEELEMQLRSLTGMMSRLARERDLGAQRLAELLLEREPAHLLLPEAPANASAEGVSHHLALQLTNAKAQLRRLRQEVEEKAEQLLDSQAEVQGLEAEIRRLRQETQALSAQAKRAELYREEAEALRERAGRLPRLQEELRRCREKLQAAEVFKGQLEEERVLSEALEASKVLLEEQLEVARERSARLHETQRENLLLRTRLGEAHADLDSLRHQLEQLVEENVELELELQRSLEPPPGSPGEASLPGAAPSLQDEVREAEAGRLRAVERENRELRGQLQMLQAQLGSQHPLLEEQRENSRQPPVPNRDPATPSALHHSPQSPACQIGGEGSESLDLPSPASYSDITRSPKCSQAPDSHPELESPLQMVSQDPQTSDQALQESDPTVETHQCLEKSGHRVPLQSPIVWDPPQGPEVRIEVQELLGETGSREAPQGELVHKAQVLKQESPKCRPRSAELTLREPLKDQKALDRELELSKQQKETGRHEQRPKGLESKLGPQKPQQTSEGVPDAWSREEPTPGETLVSAIPEEQALRDEVAQLRREVAGLEVKLQAQAQRLEARSAEALCLSEELAQARRTEAEAHQEAEAQAREQARLREAVDTASLELEAASREREALAEALAAAGRERRQWERDGPRLRAQVEAAEQQVQALESQVRCHLEEAEREHAEKQALREELEKAVLRGQELGDRLEHLQEELEQAALERQKFLQEQENQHQRYRHLEQRLEAELQAASTSKEEALMELKARALQLEEELIQLRQYPVDLATGARAGPRTVETQNGRLIEVERNNATLVAEKAALQGQLQHLEGQLGSLQGRAQELLLQSQRAQEHSSRLQAEKSMMEMQGQELHRKLGVLEEEVRAARRAQEETRGQQQALLRDHEALVQLQRRQETELEGLLVRHRDLKANMRALELAHRELQGRHEQLQAQRANVEAQEVALLAERERLMQDGHRQRGLEEELRRLQNEHERAQMLLAEVSRERGELQGERGELRSRLARLELERAQLEIQSQQLRESNQQLDLSACRLTTQSSDPASQRAGGGEQAAVSRGPGPEPGEQGAAGAQPGEPRPSASRAARVPGPA

SEQ ID NO: 5 - DNA, 53 bp, Artificial Sequence
  Sense oligonucloetide for ISH
gcgctataatacgactcactatagggagatccgaatctttggacctgccttct

SEQ ID NO: 6 - DNA, 53 bp, Artificial Sequence
  Antisense oligonucleotide for ISH
gcattaatttaggtgacactatagaagcgaagctagccgtatccactgcttca

SEQ ID NO: 7 - DNA, 21 bp, Artificial Sequence
  Oligonucleotide
gatctgggggcacagcggttg

SEQ ID NO: 8 - DNA, 21 bp, Artificial Sequence
  Oligonucleotide
gcgtctcagctgggccttggc

SEQ ID NO: 9 - DNA, 21 bp, Artificial Sequence
  Oligonucleotide
tccagcaggtcagcaaagaac

SEQ ID NO: 10 - DNA, 22 bp, Artificial Sequence
  Oligonucleotide
ggactgattatggacaggactg

SEQ ID NO: 11 - DNA, 21 bp, Artificial Sequence
  Oligonucleotide
aatgaatgccttcaacagtgg

SEQ ID NO: 12 - DNA, 21 bp, Artificial Sequence
  Oligonucleotide
acaatgccaactttcagaagc

SEQ ID NO: 13 - DNA, 20 bp, Artificial Sequence
  Oligonucleotide
gatgaggttcgcctgctatt

SEQ ID NO: 14 - DNA, 21 bp, Artificial Sequence
  Oligonucleotide
gacttgggtgatcttggactc

SEQ ID NO: 15 - DNA, 24 bp, Artificial Sequence
  Oligonucleotide
tcttccttgctcttggtgtatatc

SEQ ID NO: 16 - DNA, 23 bp, Artificial Sequence
  Oligonucleotide
gagatggctgtactggtcatatt

SEQ ID NO: 17 - DNA, 22 bp, Artificial Sequence
  Oligonucleotide
tcgacttcagcgcctacattga

SEQ ID NO: 18 - DNA, 22 bp, Artificial Sequence
  Oligonucleotide
ccgctttgtggttgctgttgaa

SEQ ID NO: 19 - DNA, 22 bp, Artificial Sequence
  Oligonucleotide
cgggagtgtgagtcctacttta

SEQ ID NO: 20 - DNA, 24 bp, Artificial Sequence
  21
gtggaggtgaaccatccttatatc

SEQ ID NO: 21 - DNA, 21 bp, Artificial Sequence
  Oligonucleotide
tcagtcaacgggggacataaa

SEQ ID NO: 22 - DNA, 21 bp, Artificial Sequence
  Oligonucleotide
ggggctgtactgcttaaccag

SEQ ID NO: 23 - DNA, 21 bp, Artificial Sequence
  Oligonucleotide
gatccagaaagccgagatcaa

SEQ ID NO: 24 - DNA, 22 bp, Artificial Sequence
  Oligonucleotide
ctggaagtggatctcaaggatg

SEQ ID NO: 25 - DNA, 20 bp, Artificial Sequence
  Oligonucleotide
ctgaactgctcagcccacac

SEQ ID NO: 26 - DNA, 21 bp, Artificial Sequence
  Oligonucleotide
tggacatacttccttccctga

SEQ ID NO: 27 - DNA, 24 bp, Artificial Sequence
  Oligonucleotide
cgacttcagcactttcttccgaga

SEQ ID NO: 28 - DNA, 24 bp, Artificial Sequence
  Oligonucleotide
agatggtgtagtgtggtgaccctt

SEQ ID NO: 29 - DNA, 22 bp, Artificial Sequence
  Oligonucleotide
aaatgggaggaccaatggcgtt

SEQ ID NO: 30 - DNA, 24 bp, Artificial Sequence
  Oligonucleotide
atagatgaaggtgagcagcagcga

SEQ ID NO: 31 - DNA, 23 bp, Artificial Sequence
  Oligonucleotide
gaaagtagcaaggagtgtgtttg

SEQ ID NO: 32 - DNA, 22 bp, Artificial Sequence
  Oligonucleotide
gggtctaaagccaggtcataag

SEQ ID NO: 33 - DNA, 22 bp, Artificial Sequence
  Oligonucleotide
ggatgaagacgactatcccaac

SEQ ID NO: 34 - DNA, 22 bp, Artificial Sequence
  Oligonucleotide
cctcactctcaggaacattcac

SEQ ID NO: 35 - DNA, 20 bp, Artificial Sequence
  Oligonucleotide
ggacccgttccccgacctgt

SEQ ID NO: 36 - DNA, 20 bp, Artificial Sequence
  Oligonucleotide
cgacctcccggtttctcgcc

SEQ ID NO: 37 - DNA, 22 bp, Artificial Sequence
  Oligonucleotide
catcatgtcagcttcaggagat

SEQ ID NO: 38 - DNA, 21 bp, Artificial Sequence
  Oligonucleotide
gggtctgttgctgtttgtaag

SEQ ID NO: 39 - DNA, 20 bp, Artificial Sequence
  Oligonucleotide
aactgaaggcagaggttgag

SEQ ID NO: 40 - DNA, 23 bp, Artificial Sequence
  Oligonucleotide
cccttggtagattcccattatca

SEQ ID NO: 41 - DNA, 21 bp, Artificial Sequence
  Oligonucleotide
cgtgcttgagagggtcatttg

SEQ ID NO: 42 - DNA, 20 bp, Artificial Sequence
  Oligonucleotide
ggtcgggagtccacaacttc

SEQ ID NO: 43 - protein, 42 aa, Mus musculus
DDTSHIRFDDRQLRLDERSFLALDWDPDLKKRYFDENAAEDF

SEQ ID NO: 44 - protein, 42 aa, Rattus norvegicus
DDTRHIRFDDRQLRLDERSFLALDWDPDLKKRYFDENAAEDF

SEQ ID NO: 45 - protein, 42 aa, Homo sapiens; identical to SEQ ID NO: 44

SEQ ID NO: 46 - protein, 42 aa, Pongo pygmaeus; identical to SEQ ID NO: 44

SEQ ID NO: 47 - protein, 42 aa, Equus caballus; identical to SEQ ID NO: 44

SEQ ID NO: 48 - protein, 42 aa, Sus scrofa; identical to SEQ ID NO: 44

SEQ ID NO: 49 - protein, 42 aa, Oryctolagus cuniculus
DDTRHIRFDDRQLRLDERSFLALDWDPELKKRYFDENAAEDF

SEQ ID NO: 50 - protein, 42 aa, Gallus gallus
DDTRHIRFDDRQPRLDERSFLALDWDPELKKRYFDDSAAEDF

SEQ ID NO: 51 - protein, 42 aa, Xenopus laevis
DDTKYIRFDERQLRLDERSYLALDWDPKLKKKFFDENAAEDF

SEQ ID NO: 52 - protein, 41 aa, Danio rerio
TGEPHAQIHFQEERLSDRCYLSLVWEPEMKRSFFNEAAVDF

SEQ ID NO: 53 - protein, 981 aa, Mus musculus
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFNIPDEKEARLWNKYMSNTFEPLNKPDSTIQDAGLYQGQVLVIEQKNEDGTWPRGPSTPKSPGASNFSTLPKISPSSLSNNYNNINNRNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERSLEVYLVRMDPLAKPMQYKVIVPKIGNILDLCTALSALSGVPADKMIVTDIYNHRFHRIFAVDENLSSIMERDDIYVFEININRAEDTEHVVIPVCLREKFRHSSYTHHTGSSLFGQPFLMAIPRNNTEDKLYNLLLLRMCRYVKMSTETEETDGHLRCCEDQNINGNGPNGLHEEGSPSEMETDEPDDESSQDQELPSENENSQSEDSVGGDNDSENGLCTEETCKGQLTGHKKRLFTFQFNNLGNNDINYIKDDTSHIRFDDRQLRLDERSFLALDWDPDLKKRYFDENAAEDFEKHESVEYKPPKRPFVKLKDCIELFTTKEKLGAEDPWYCPNCKEHQQATKKLDLWSLPPVLVVHLKRFSYSRYMRDKLDTLVDFPISDLDMSEFLINPNAGPCRYNLIAVSNHYGGMGGGHYTAFAKNKDDGKWYYFDDSSVSTASEDQIVSKAAYVLFYQRQDTFSGTGFFPLDRETKGASAATGIPLESDEDSNDNDNDLENENCMHTN

SEQ ID NO: 54 - protein, 981 aa, Mus musculus
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFNIPDEKEARLWNKYMSNTFEPLNKPDSTIQDAGLYQGQVLVIEQKNEDGTWPRGPSTPKSPGASNFSTLPKISPSSLSNNYNNINNRNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERSLEVYLVRMDPLAKPMQYKVIVPKIGNILDLCTALSALSGVPADKMIVTDIYNHRFHRIFAVDENLSSIMERDDIYVFEININRAEDTEHVVIPVCLREKFRHSSYTHHTGSSLFGQPFLMAIPRNNTEDKLYNLLLLRMCRYVKMSTETEETDGHLRCCEDQNINGNGPNGLHEEGSPSEMETDEPDDESSQDQELPSENENSQSEDSVGGDNDSENGLCTEETCKGQLTGHKKRLFTFQFNNLGNNDINYIKDDTSHIRFDDRQLRLDERSFRALDWDPDLKKRYFDENAAEDFEKHESVEYKPPKRPFVKLKDCIELFTTKEKLGAEDPWYCPNCKEHQQATKKLDLWSLPPVLVVHLKRFSYSRYMRDKLDTLVDFPISDLDMSEFLINPNAGPCRYNLIAVSNHYGGMGGGHYTAFAKNKDDGKWYYFDDSSVSTASEDQIVSKAAYVLFYQRQDTFSGTGFFPLDRETKGASAATGIPLESDEDSNDNDNDLENENCMHTN

SEQ ID NO: 55 - protein, 981 aa, Homo sapiens
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFSIPDEKETRLWNKYMSNTFEPLNKPDSTIQDAGLYQGQVLVIEQKNEDGTWPRGPSTPKSPGASNFSTLPKISPSSLSNNYNNMNNRNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERTLEVYLVRMDPLTKPMQYKVVVPKIGNILDLCTALSALSGIPADKMIVTDIYNHRFHRIFAMDENLSSIMERDDIYVFEININRTEDTEHVIIPVCLREKFRHSSYTHHTGSSLFGQPFLMAVPRNNTEDKLYNLLLLRMCRYVKISTETEETEGSLHCCKDQNINGNGPNGIHEEGSPSEMETDEPDDESSQDQELPSENENSQSEDSVGGDNDSENGLCTEDTCKGQLTGHKKRLFTFQFNNLGNTDINYIKDDTRHIRFDDRQLRLDERSFLALDWDPDLKKRYFDENAAEDFEKHESVEYKPPKKPFVKLKDCIELFTTKEKLGAEDPWYCPNCKEHQQATKKLDLWSLPPVLVVHLKRFSYSRYMRDKLDTLVDFPINDLDMSEFLINPNAGPCRYNLIAVSNHYGGMGGGHYTAFAKNKDDGKWYYFDDSSVSTASEDQIVSKAAYVLFYQRQDTFSGTGFFPLDRETKGASAATGIPLESDEDSNDNDNDIENENCMHTN

SEQ ID NO: 56 - protein, 981 aa, Artificial Sequence
  Predicted L749R substitution of SEQ ID NO 55
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFSIPDEKETRLWNKYMSNTFEPLNKPDSTIQDAGLYQGQVLVIEQKNEDGTWPRGPSTPKSPGASNFSTLPKISPSSLSNNYNNMNNRNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERTLEVYLVRMDPLTKPMQYKVVVPKIGNILDLCTALSALSGIPADKMIVTDIYNHRFHRIFAMDENLSSIMERDDIYVFEININRTEDTEHVIIPVCLREKFRHSSYTHHTGSSLFGQPFLMAVPRNNTEDKLYNLLLLRMCRYVKISTETEETEGSLHCCKDQNINGNGPNGIHEEGSPSEMETDEPDDESSQDQELPSENENSQSEDSVGGDNDSENGLCTEDTCKGQLTGHKKRLFTFQFNNLGNTDINYIKDDTRHIRFDDRQLRLDERSFRALDWDPDLKKRYFDENAAEDFEKHESVEYKPPKKPFVKLKDCIELFTTKEKLGAEDPWYCPNCKEHQQATKKLDLWSLPPVLVVHLKRFSYSRYMRDKLDTLVDFPINDLDMSEFLINPNAGPCRYNLIAVSNHYGGMGGGHYTAFAKNKDDGKWYYFDDSSVSTASEDQIVSKAAYVLFYQRQDTFSGTGFFPLDRETKGASAATGIPLESDEDSNDNDNDIENENCMHTN

SEQ ID NO: 57 - protein, 952 aa, Homo sapiens
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFSIPDEKETRLWNKYMSNTFEPLNKPDSTIQDAGLYQGQVLVIEQKNEDGTWPRGPSTPNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERTLEVYLVRMDPLTKPMQYKVVVPKIGNILDLCTALSALSGIPADKMIVTDIYNHRFHRIFAMDENLSSIMERDDIYVFEININRTEDTEHVIIPVCLREKFRHSSYTHHTGSSLFGQPFLMAVPRNNTEDKLYNLLLLRMCRYVKISTETEETEGSLHCCKDQNINGNGPNGIHEEGSPSEMETDEPDDESSQDQELPSENENSQSEDSVGGDNDSENGLCTEDTCKGQLTGHKKRLFTFQFNNLGNTDINYIKDDTRHIRFDDRQLRLDERSFLALDWDPDLKKRYFDENAAEDFEKHESVEYKPPKKPFVKLKDCIELFTTKEKLGAEDPWYCPNCKEHQQATKKLDLWSLPPVLVVHLKRFSYSRYMRDKLDTLVDFPINDLDMSEFLINPNAGPCRYNLIAVSNHYGGMGGGHYTAFAKNKDDGKWYYFDDSSVSTASEDQIVSKAAYVLFYQRQDTFSGTGFFPLDRETKGASAATGIPLESDEDSNDNDNDIENENCMHTN

SEQ ID NO: 58 - protein, 952 aa, Artificial Sequence
  Predicted L720R substition of SEQ ID NO 56
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFSIPDEKETRLWNKYMSNTFEPLNKPDSTIQDAGLYQGQVLVIEQKNEDGTWPRGPSTPNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERTLEVYLVRMDPLTKPMQYKVVVPKIGNILDLCTALSALSGIPADKMIVTDIYNHRFHRIFAMDENLSSIMERDDIYVFEININRTEDTEHVIIPVCLREKFRHSSYTHHTGSSLFGQPFLMAVPRNNTEDKLYNLLLLRMCRYVKISTETEETEGSLHCCKDQNINGNGPNGIHEEGSPSEMETDEPDDESSQDQELPSENENSQSEDSVGGDNDSENGLCTEDTCKGQLTGHKKRLFTFQFNNLGNTDINYIKDDTRHIRFDDRQLRLDERSFRALDWDPDLKKRYFDENAAEDFEKHESVEYKPPKKPFVKLKDCIELFTTKEKLGAEDPWYCPNCKEHQQATKKLDLWSLPPVLVVHLKRFSYSRYMRDKLDTLVDFPINDLDMSEFLINPNAGPCRYNLIAVSNHYGGMGGGHYTAFAKNKDDGKWYYFDDSSVSTASEDQIVSKAAYVLFYQRQDTFSGTGFFPLDRETKGASAATGIPLESDEDSNDNDNDIENENCMHTN

(19) United States
(12) Patent Application Publication
(10) Pub. No.: US 2018/0258484 A1
(43) Pub. Date: Sep. 13, 2018

(54) INFLAMMATION-ENABLING POLYPEPTIDES AND USES THEREOF

(71) Applicant: THE ROYAL INSTITUTION FOR THE ADVANCEMENT OF LEARNING/MCGILL UNIVERSITY, Montréal (CA)

(72) Inventor: Philippe Gros, Saint-Lambert (CA)

(21) Appl. No.: 15/907,406

(22) Filed: Feb. 28, 2018

Related U.S. Application Data
(63) Continuation-in-part of application No. 15/281,666, filed on Sep. 30, 2016, now abandoned.
Application No. 15/281,666 is a continuation-in-part of application No. 15/049,491, filed on Feb. 22, 2016, now abandoned.
Application No. 15/049,491 is a continuation of application No. 14/404,209, filed on Nov. 26, 2014, now abandoned.
(60) Provisional application No. 61/652,271, filed on May 28, 2012.

(51) Int. Cl. C12Q 1/6876; C12Q 1/6883; A61K 31/7088; G01N 33/68; C07K 14/47

(52) U.S. Cl. CPC C12Q 1/6876; C12Q 2600/156; C12Q 1/6883; C12Q 2600/158; C12Q 2600/136; G01N 2800/7095; G01N 33/68; C07K 14/47; A61K 31/7088

(57) ABSTRACT

This present technology relates to the use of inflammation-enabling polypeptides (or their coding sequences) to screen for agents useful for the prevention, treatment and/or alleviations of symptoms associated with an inflammatory disorder, to identify individuals susceptible of developing an exacerbated inflammatory response as well as to determine if a therapeutic regimen is capable of preventing, treating or alleviating the symptoms associated to an inflammatory disorder in an individual. The present technology also provides methods for preventing, treating and/or alleviating the symptoms associated to an inflammatory condition based on the inhibition of expression or activity of the inflammation-enabling targets.

### CROSS-REFERENCE TO RELATED APPLICATIONS AND DOCUMENT

This application is a continuation-in-part of Ser. No. 15/281,666 which is a continuation-in-part of U.S. patent application Ser. No. 15/049,491 which is a continuation-in-part of U.S. patent application Ser. No. 14/404,209 which corresponding to the national phase entry in the United States of PCT/CA2013/050403 filed on May 27, 2013 and claims priority from U.S. provisional patent application 61/652,271 filed on May 28, 2012. The content of these applications is herewith incorporated herewith in its entirety.

This application is concurrently filed with a sequence listing in an electronic format. The content of the sequence listing is also incorporated herewith in its entirety.

### TECHNOLOGICAL FIELD

The present disclosure relates to the use of polypeptides (or their coding sequences) to screen for agents useful for the prevention, treatment and/or alleviations of symptoms associated with an inflammatory disorder, to identify individuals susceptible of developing an exacerbated inflammatory response as well as to determine if a therapeutic regimen is capable of preventing, treating or alleviating the symptoms associated to an inflammatory disorder in an individual. The present application also provides methods for preventing, treating and/or alleviating the symptoms associated to an inflammatory condition based on the inhibition of expression or activity of the inflammation-enabling targets.

### BACKGROUND

Inflammation is a normal physiological response to tissue injury caused by infections, burns, trauma and other insults. Tight regulation of this response is important for initial recognition of the associated danger signals, elimination of the causative lesion and restoration of homeostasis. This process involves a complex interplay between hematopoietic and stromal cells including the crosstalk between fibroblasts, endothelial and epithelial cells with cells of the innate and adaptive immune systems. The early production of pro-inflammatory soluble mediators and remodeling enzymes, and the timely synthesis of anti-inflammatory molecules that dampen and terminate the process are characteristic features of such a regulated inflammatory response. However, dysregulation of this process results in acute or chronic inflammatory conditions. Although many classes and types of anti-inflammatory drugs exist, their efficacy is limited and often transient, and their long-term use causes significant adverse side effects. Hence, inflammatory diseases represent an unmet pharmacological need in a large market.

The inflammatory response involves a complex cascade of events including the initial activation of pattern recognition receptors (PRRs) and inflammasomes by danger signals in epithelial, endothelial and tissue resident immune cells. This is rapidly followed by recruitment of immune cells such as neutrophils, basophils, monocytes, macrophages, CD4+ and CD8+T lymphocytes from distant sites to the site of injury. These infiltrates release a number of soluble mediators (histamine, leukotrienes, nitric oxide), cytokines (TNFα, IFNγ, IL-1), chemokines (IL-8, MCP1, KC) and enzymes (lysosomal proteases), which along with certain plasma proteins (complement, bradykinin, plasmin) establish and amplify the inflammatory response. Timely production of anti-inflammatory molecules (PGE2, IL-10, TGFβ, IL-1Ra) dampens and ultimately terminates this response. In the presence of persistent tissue injury or of an unusual infectious/environmental insult, overexpression of pro-inflammatory mediators or insufficient production of anti-inflammatory signals results in acute or chronic debilitating conditions. Acute inflammatory conditions, including sepsis and encephalitis, are difficult to manage clinically and have high mortality rates. Chronic inflammatory diseases show a high incidence in North America, and include rheumatoid arthritis (RA, incidence 75-1000/105), inflammatory bowel disease (IBD, ˜0.5-25/105), systemic lupus erythematosus (SLE, 40-200/105), psoriasis (PA, 2/100), multiple sclerosis (MS, 18-350/105), type 1 diabetes (T1D, 8-17/105), and celiac disease (CeD, 1/100). It has also been proposed that chronic obstructive pulmonary disease, coronary atherosclerosis, diabetes, cancer and neurodegenerative disorders may have a contributing inflammatory component. These global “inflammatory” diseases are a significant burden on human health, and represent a large pharmaceutical market for anti-inflammatory drugs. Several classes of such drugs have been developed, including steroids (cortisone), nonsteroidal anti-inflammatory drugs including COX2 inhibitors (colecoxib), and derivatives of proprionic acid (ibuprofen), acetic acid (indomethacin), enolic and fenamic acid. However, the benefit of these drugs is limited, and they show significant person-to-person variability in efficacy and their long-term use has severe side effects, raising the need for specific targeted therapies. Recently, biologicals targeting the TNF (infliximab, etanercept, adalimumab), IL-12p40 (monoclonal antibodies) and IL-1 (anakinra) pathways, as well as B cells (rituximab) and T cells (abatacept) have shown clinical efficacy in inflammatory conditions, supporting the relevance of our host-based approach to identify novel anti-inflammatory targets.

Chronic inflammatory conditions share several clinical features, including persistent activation of the innate and acquired immune systems. Rupture of normal epithelial barriers, tissue damage, or persistent infections may lead to chronic exposure to inert environmental or host-derived products (food, monosodium urate crystals, asbestos, silica, etc.), microbial products (including commensal bacteria, viruses, parasites and fungi), and/or exposure to enticing self-antigens (nucleic acids, damaged proteins). These are recognized by intracellular or cell surface sensors of the innate immune system or by receptors of the acquired immune system. Engagement of these receptors and activation of associated signaling pathways in myeloid cells leads to the production of pro-inflammatory cytokines (IL-1, IL-18, IL-12, IL-23) and mediators (leukotrienes), and to the release of toxic species (reactive oxygen radicals) and proteases (lysosomal enzymes) in situ that exacerbate the local inflammatory environment by recruiting and activating other myeloid and lymphoid cells from systemic sites, including the engagement of CD8+ and CD4+T lymphocytes (Th1, Th2 and Th17 cells). Persistence of pro-inflammatory T helper programs in these cells (Th1, Th2, Th17) and/or defects in suppressive T regulatory (Treg) responses lead to unrelenting tissue damage and is a major common feature of these inflammatory diseases.

Familial aggregation and twin studies have long established that chronic inflammatory conditions have a strong genetic component. Genome-wide association studies (GWAS) have shown that the genetic component of these diseases is complex with >250 loci detected, and notably several of these risk loci are shared in rheumatoid arthritis (RA), psoriasis (PA), systemic lupus erythematosus (SLE), multiple sclerosis (MS), type 1 diabetes (T1D), celiac disease (CeD), and inflammatory bowel disease (IBD), with additive small genetic effects combining to cause disease by a threshold mechanism. Genetic risks common to these conditions are found in immune pathways including: a) pattern recognition receptors of the innate immune system (NLRs, TLRs) and associated signaling cascades (inflammasomes); b) antigen processing and presentation, production of cytocidal species (ROS, iNOS), and secretion of pro-inflammatory mediators by myeloid cells (IL-18, IL-12); c) T and B lymphocyte maturation (e.g. by IL-2), including control of auto-reactive T and B cells; d) antigen receptors of T and B cells for recognition in the context of Class I or class II MHC (HLA); e) production of pro-inflammatory cytokines (IL-12, IL-18, IL-23), and associated regulation of Th1, Th17, Treg polarization of the immune response; f) activation of ubiquitous cellular responses such as autophagy, ER stress, and others. The proteins and pathways defined by these shared genetic variants may be excellent candidate targets for drug development and therapeutic intervention. However, the complexity of the genetic control renders the identification and prioritization of key non-redundant targets in these pathways difficult.

It would be highly desirable to be provided with additional polypeptide targets which enable the mounting and/or persistence of an inflammatory response. These targets should preferably be host proteins which are involved in inflammation, regardless of the etiology of the disease.

### BRIEF SUMMARY

One aim of the present disclosure is to provide host-derived inflammation-enabling polypeptides responsible for mounting and maintaining a pathological inflammatory response, independent of the etiology of the inflammatory disease.

In accordance with the present disclosure, there is provided a method for assessing the ability of an agent to prevent, treat and/or alleviate the symptoms associated with an inflammatory condition in an individual. Broadly, the method comprises (a) combining the agent with a reagent to obtain a parameter associated with the biological activity of at least one inflammation-enabling polypeptide (IEP); (b) measuring the parameter of the reagent of step (a) to obtain a test level; (c) comparing the test level to a control level, wherein the control level is associated with the biological activity of the at least one inflammation-enabling polypeptide observed during the onset or maintenance of the inflammatory condition; and (d) characterizing the agent based on the comparison. Since the biological activity IEP is increased during onset or maintenance of inflammation, the agent is considered useful for the prevention, treatment and/or alleviation of the symptoms associated with the inflammatory condition when it is capable to reduce the biological activity of the IEP. Alternatively, the agent is considered not to be useful for (e.g. lacking utility) for the prevention, treatment and/or alleviation of the symptoms associated with the inflammatory condition if it cannot reduce the biological activity of the IEP (e.g. if the biological activity in the presence of the agent is equal to or higher than the control biological activity). In an embodiment, the inflammation-enabling polypeptide is at least one of LYST, ZBTB7B, BPGM1, RASAL3 CCDC88B, USP15, IRF8, IRF1, IRGM1, THEMIS and/or FOXN1. In an embodiment, the reagent is a nucleic acid molecule. In another embodiment, the nucleic acid molecule comprises a promoter of a gene encoding the at least one inflammation-enabling polypeptide and the parameter is a measure of the expression driven by the promoter. Alternatively, the nucleic acid molecule can be a transcript encoding the at least one inflammation-enabling polypeptide and the parameter can be a measure of the amount of the transcript. In other embodiment, the nucleic acid molecule is a transcript of at least one gene whose expression can be modified by the inflammation-enabling polypeptide and the parameter is the amount of the transcript. For example, when the inflammation-enabling polypeptide is IRF8, the at least one gene whose expression can be modified by the inflammation-associated polypeptide can be selected from the group consisting of the genes presented in Table 1, 2 and 3. In yet another embodiment, the reagent can be a polypeptide. In still a further embodiment, the polypeptide is the at least one inflammation-enabling polypeptide. In such embodiment, the parameter is the amount of the at least one inflammation-enabling polypeptide. Alternatively, the parameter can be at least one biological activity of the at least one inflammation-enabling polypeptide. For example, when the at least one inflammation-enabling polypeptide is selected from the group consisting of FOXN1, IRF8 and IRF1, the at least one biological activity can be a transcription factor activity. As another example, when the at least one inflammation-enabling polypeptide is CCDC88B, the at least one biological activity can be protein folding. As yet another example, when the at least one inflammation-enabling polypeptide is USP15, the at least one biological activity can be a de-ubiquitinating activity. As still another example, when the at least one inflammation-enabling polypeptide is THEMIS, the at least one biological activity can be a protein signaling activity. In another embodiment, the polypeptide is a binding partner of the at least one inflammation-enabling polypeptide. For example, when the at least one inflammation-enabling polypeptide is USP15, the binding partner can be a polypeptide that can be de-ubiquinated by USP15. In another embodiment, the polypeptide is associated with the signaling pathway of the inflammation-enabling polypeptide. In an embodiment, step (i) is conducted in vitro orin vivo. In yet another embodiment, step (i) is conducted in a non-human animal, such as, for example, in an animal bearing at least one gene copy of a non-functional inflammation-enabling polypeptide. In still another embodiment, step (i) is conducted in a human.

In accordance with the present disclosure, there is provided a method for assessing the ability of an agent to treat and/or alleviate the symptoms associated with an inflammatory condition in an individual in need thereof. Broadly, the method comprises (a) administering a trigger for inducing an inflammatory response in an animal heterozygous for the gene encoding an inflammation-enabling polypeptide; (b) administering the agent to the animal experiencing the inflammatory response 9 e.g. being in an inflammatory state); (c) measuring a parameter of the inflammatory response in the animal to provide a test level; (d) comparing the test level to a control level, wherein the control level is associated with the biological activity of the inflammation-enabling polypeptide observed during the onset or maintenance of the inflammatory condition; and (e) characterizing the agent based on the comparison. Since the biological activity IEP is increased during onset or maintenance of inflammation, the agent is considered useful for the treatment and/or alleviation of the symptoms associated with the inflammatory condition when it is shown to reduce the biological activity of the IEP in the treated animal. Alternatively, the agent is considered as lacking utility for the treatment and/or alleviation of the symptoms associated with the inflammatory condition when the biological activity of the IEP, in the presence of the agent, is equal to or higher than the control level in the treated animal. In an embodiment, the inflammation-enabling polypeptide is at least one of LYST, ZBTB7B, BPGM1, RASAL3 CCDC88B, USP15, IRF8, IRF1, IRGM1, THEMIS and/or FOXN1. In still another embodiment, the trigger is an infectious agent. In yet another embodiment, the parameter is a survival rate. In yet another embodiment, the parameter is a neurological symptom selected from the group consisting to fever, tremors, lethargy, hind limb paralysis and coma. In still yet another embodiment, the parameter is an inflammation-associated parameters selected from the group consisting of immune cells number, immune cell type, cytokine profile, chemokine profile, immunoglobulin profile, edema and blood-brain-barrier permeability.

In accordance with the present disclosure, there is provided a method for assessing the ability of an agent to prevent an inflammatory condition in an individual. Broadly, the method comprises (a) administering an agent to an animal heterozygous for the gene encoding an inflammation-enabling polypeptide; followed by (b) administering a trigger capable of inducing an inflammatory response in the animal in the absence of the agent; (c) measuring a parameter of the inflammatory response in the animal to provide a test level; (d) comparing the test level to a control level, wherein the control level is associated with the biological activity of the at least one inflammation-enabling polypeptide observed during the onset or maintenance of the inflammatory condition; and (e) characterizing the utility of the agent in the individual based on the comparison. Since the biological activity IEP is increased during onset or maintenance of inflammation, the agent is considered useful for the prevention of the inflammatory condition when it is shown to reduce the biological activity of the IEP in the treated animal. Alternatively, the agent is considered as lacking utility for the prevention of the inflammatory condition in the individual when the biological activity of the IEP, in the presence of the agent, is equal to or higher than the control level in the treated animal. Embodiments described herein with respect to the inflammation-enabling polypeptide, the trigger or the parameter described herein can also be used in this method.

In accordance with the present disclosure, there is provided a method for determining if a therapeutic agent is useful for the prevention, treatment and/or alleviation of symptoms associated with an inflammatory condition in an individual. Broadly, the method comprises (a) providing a biological sample of the individual having received at least one dose of the therapeutic agent; (b) measuring a parameter of a reagent associated to at least one inflammation-enabling polypeptide to provide a test level; (c) comparing the test level to a control level, wherein the control level is associated with the biological activity of the at least one inflammation-enabling polypeptide observed during the onset or maintenance of the inflammatory condition; and (d) characterizing the usefulness of the therapeutic agent based on the comparison. Since the biological activity IEP is increased during onset or maintenance of inflammation, the agent is considered useful for the prevention, treatment and/or alleviation of the symptoms associated with the inflammatory condition in the individual when it is shown to reduce the biological activity of the IEP. Alternatively, the agent is considered as lacking utility for the prevention, treatment and/or alleviation of the symptoms associated with the inflammatory condition in the individual when the biological activity of the IEP, in the presence of the agent, is equal to or higher than the control level. Embodiments described herein with respect to the reagent, the parameter, the biological activity, the inflammation-enabling polypeptide, the polypeptide can also be used in this method. In yet a further embodiment, the characterization is performed in at least two biological samples (and in some embodiments, maybe more), preferably at two distinct time periods to evaluate the usefulness of the therapeutic agent in the individual in function of time.

In accordance with the present disclosure, there is provided a method for determining the predisposition to or presence of an inflammatory condition in an individual. Broadly, the method comprises (a) providing a biological sample from the individual; (b) measuring a parameter of a reagent associated to at least one inflammation-enabling polypeptide to provide a test level; (c) comparing the test level to a control level, wherein the control level is associated with the biological activity of the at least one inflammation-enabling polypeptide observed during the absence of the inflammatory condition; and (d) characterizing the individual based on the comparison. Embodiments described herein with respect to the reagent, the parameter, the biological activity, the inflammation-enabling polypeptide, the polypeptide can also be used in this method.

In accordance with the present disclosure, there is provided a method for preventing, treating and/or alleviating the symptoms associated to an inflammatory condition in an individual in need thereof. Broadly, the method comprises administering an agent capable of inhibiting at least one parameter of an inflammation-enabling polypeptide so as to prevent, treat and/or alleviation the symptoms associated to the inflammatory condition in the individual. Also provided herein, is the use of an agent capable of inhibiting at least one parameter of an inflammation-enabling polypeptide for the prevention, treatment and/or alleviation the symptoms associated to the inflammatory condition in the individual; the use of an agent capable of inhibiting at least one parameter of an inflammation-enabling polypeptide for the manufacture of a medicament for the prevention, treatment and/or alleviation the symptoms associated to the inflammatory condition in the individual; as well as an agent capable of inhibiting at least one parameter of an inflammation-enabling polypeptide for the prevention, treatment and/or alleviation the symptoms associated to the inflammatory condition in the individual. In an embodiment, the agent is a nucleic acid molecule capable of limiting the expression of the inflammation-enabling polypeptide. Embodiments with respect to the inflammation-enabling polypeptides described do apply to these therapeutic uses. In another embodiment, the agent is an antibody capable of limiting the biological activity of the inflammation-enabling polypeptide. In still another embodiment, the inflammatory condition is selected from the group consisting of neuroinflammation, rheumatoid arthritis, systemic lupus erythematosus, multiple sclerosis, type 1 diabetes and celiac disease.

In accordance with the present disclosure, there are provided various tools associated with inflammatory enabling polypeptides. In an embodiment, an isolated polypeptide is provided. The isolated polypeptide is a mutant of an inflammatory enabling polypeptide (IEP). The mutant IEP has, when expressed in a subject, the ability to prevent the onset and/or maintenance of an inflammatory condition in the subject. Embodiments associated with the different types of IEP described above can be applied herein. In an embodiment, the isolated polypeptide has the amino acid sequence of SEQ ID NO: 4. In yet another embodiment, a nucleic acid vector is provided and encodes the mutant IEP. In still another embodiment, a cell (or a cell line) is provided. The cell can be heterozygous for a gene encoding an inflammatory enabling polypeptide (IEP). In such embodiment, the heterozygous cell has a first gene copy encoding IEP and a second gene copy encoding a mutant of the IEP. Alternatively, the cell can be homozygous for a gene encoding for a mutant of an inflammatory enabling polypeptide (IEP). The cell can be, in an embodiment, a transgenic cell and can have, for example, the nucleic acid vector described herein. In yet another embodiment, an animal is provided. The animal can be heterozygous for a gene encoding an inflammatory enabling polypeptide (IEP). In such embodiment, the heterozygous animal has a first gene copy encoding IEP and a second gene copy encoding a mutant of the IEP. In another embodiment, the animal can be homozygous for a gene encoding for a mutant of an inflammatory enabling polypeptide (IEP). In some embodiments, the animal can be a transgenic animal, for example, those manipulated to have the nucleic acid vector described herein.

In accordance with the present disclosure, there is provided a method for assessing the ability of an agent to prevent, treat and/or alleviate the symptoms associated with an inflammatory condition in an individual. Broadly, the method comprises (a) combining the agent with a USP15 polypeptide; (b) measuring a biological activity of the USP15 polypeptide of step (a) to obtain a test level; (c) comparing the test level to a control level, wherein the control level is associated with the biological activity of the USP15 polypeptide observed during the onset or maintenance of the inflammatory condition; and (d) characterizing the agent as (i) useful for the prevention, treatment and/or alleviation of the symptoms associated with the inflammatory condition when the at least one biological activity associated with the test level is lower than the biological activity associated with the control level or (ii) lacking utility for the prevention, treatment and/or alleviation of the symptoms associated with the inflammatory condition when the at least one biological activity associated with the test level is equal to or higher than the biological activity associated with the control level. In an embodiment, the biological activity of the USP15 polypeptide is a de-ubiquitinating activity. In another embodiment, the method further comprises, in step (a), combining the agent and the USP15 polypeptide with a ubiquinated (fully or partially) TRIM25 polypeptide. In an embodiment, the method can further comprising measuring the biological activity of USP15 by determining the level of expression of at least one of the following genes Gzmb, Gzma, Fcgr4, Plaur, Ms4a6d, Cebpd, Maff, Socs3, Arrdc2, Mt1, Mt2, Cdknla, Srgn, Zfp36, Map3k6, Fkbp5, Itgb7, Rhoj, Hmgb2, Ucp2, Entpd4 or Rbm3. For example, the biological activity of USP15 can be measured by determining the level of expression of: (i) at least one of the following genes Gzmb, Gzma, Fcgr4, Plaur, Ms4a6d, Cebpd, Maff, Socs3, Mt1, Mt2, Cdknla, Zfp36, Fkbp5 or Itgb7; and (ii) at least one of the following genes Arrdc2, Srgn, Map3k6, Rhoj, Hmgb2, Ucp2, Entpd4 or Rbm3. In still another embodiment, the method further comprises, in step (b), measuring the level of the ubiquinated, partially ubiquinated or de-ubiquinated TRIM25 to measure the biological activity of the USP15 polypeptide. In an embodiment, step (a) is conducted or the control level of step (b) is obtained in vitro. In still another embodiment, step (a) is conducted in or the control level of step (b) is obtained from a cell. In still another embodiment, the cell bears one gene copy coding for a non-functional USP15 polypeptide (such as, for example, the non-functional USP15 polypeptide having the amino acid sequence of 54, 56 or 58 as well as fragments or variants thereof). In an embodiment, step (a) is conducted or the control of step (b) is obtained in vivo. In still another embodiment, step (a) is conducted in or the control level of step (b) is obtained from a non-human animal. In yet another embodiment, the non-human animal bears one gene copy coding for a non-functional USP15 polypeptide (such as, for example, the non-functional USP15 polypeptide having the amino acid sequence of 54, 56 or 58 as well as fragments or variants thereof). In still another embodiment, the inflammatory condition is neuroinflammation.

According to the present disclosure, there is also provided a method for determining the predisposition to or presence of an inflammatory condition in an individual (such as, for example, a human). Broadly, the method comprises (a) providing a biological sample from the individual; (b) measuring a biological activity of a USP15 polypeptide in the biological sample to provide a test level; (c) comparing the test level to a control level, wherein the control level is associated with the biological activity of the USP15 polypeptide observed during the absence of the inflammatory condition; and (d) characterizing the individual as (i) predisposed to the inflammatory condition when the at least one biological activity associated with the test level is equal to or higher than the biological activity associated with the control level or (ii) lacking a predisposition for the inflammatory condition when the at least one biological activity associated with the test level is lower than the biological activity associated with the control level. In an embodiment, the biological activity of the USP15 polypeptide is a de-ubiquitinating activity. In still another embodiment, the method further comprises, in step (b), determining the biological activity of the USP15 polypeptide by measuring the level of a ubiquinated, a partially ubiquinated or a de-ubiquinated TRIM25 in the biological sample. In yet another embodiment, the inflammatory condition is neuroinflammation.

According to another aspect, the present disclosure provides an isolated non-functional USP15 polypeptide having a lysine to arginine substitution at a residue corresponding to position 720 or 749 of the wild-type USP15 polypeptide. In an embodiment, the isolated non-functional USP15 polypeptide has the amino acid sequence of SEQ ID NO: 54, 56 or 58.

According to a further aspect, the present disclosure provide a cell or a non-human animal being homozygous or heterozygous for a gene encoding a USP15 polypeptide, wherein the cell has at least one gene copy encoding a non-functional USP15 polypeptide, wherein the non-functional USP15 polypeptide is defined herein.

Throughout this text, various terms are used according to their plain definition in the art. However, for purposes of clarity, some specific terms are defined below.

Biological sample. A biological sample is a sample of an individual's bodily fluid, cells or tissues. In this present disclosure, the biological sample can be derived from the individual's blood. The biological sample can be used without prior modification in the various methods described herein. Optionally, the biological sample can be treated (mechanically, enzymatically, etc.) prior to the assays described herein.

Heterozygote. Zygosity refers to the similarities of alleles for a genetic trait in an individual organism. If both alleles are the same, the individual (an homozygote) is considered homozygous for the trait. If both alleles are different, the individual (an heterozygote) is heterozygous for that trait. If one allele is missing, the individual (an hemizygote) is considered hemizygous, and, if both alleles are missing, the individual (a nullizygote) is considered nullizygous. An heterozygote of an inflammation-enabling polypeptide bears a first allele coding for a functional inflammation-enabling polypeptide and a second allele coding for a non-functional inflammation-enabling polypeptide.

Inflammation and inflammatory response. Inflammation is a response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. As used herein, the terms “inflammation” and “inflammatory response” refer to the non-pathological aspect of this response and may even be considered benefic to the individual experiencing it.

Inflammatory condition, disease or disorder. As used herein, these terms collectively refer to a dysregulated inflammatory response which causes a pathological cellular destruction of tissues in an afflicted individual. The inflammation can either be acute or chronic. Acute inflammatory conditions include, but are not limited to sepsis and encephalitis. Chronic inflammatory conditions share several clinical features, including persistent activation of the innate and acquired immune systems. The chronic inflammatory conditions can include the production of pro-inflammatory cytokines (IL-1, IL-18, IL-12, IL-23) and mediators (leukotrienes), the release of toxic species (reactive oxygen radicals) and proteases (lysosomal enzymes). In some embodiments, the chronic inflammatory condition also includes recruiting and activating other myeloid and lymphoid cells from systemic sites, such as, for example, CD8+ and CD4+T lymphocytes (Th1, Th2 and Th17 cells). Persistence of pro-inflammatory T helper programs in these cells (Th1, Th2, Th17) and/or defects in suppressive T regulatory (Treg) responses can lead to unrelenting tissue damage. Chronic inflammatory conditions includes, but are not limited to, rheumatoid arthritis (RA), inflammatory bowel disease (IBD), systemic lupus erythematosus (SLE), psoriasis (PA), multiple sclerosis (MS), type 1 diabetes (T1D), and celiac disease (CeD). Other conditions associated with chronic inflammation include, but are not limited to chronic obstructive pulmonary disease, coronary atherosclerosis, diabetes, metabolic syndrome X, cancer and neurodegenerative disorders.

Inflammation-enabling polypeptide (IEP). As used herein, this term refers to polypeptides which, when their expression and/or is limited or inhibited, prevent the onset and/or maintenance of an inflammatory condition (either acute or chronic). These polypeptides are preferentially identified by the genetic screen described below and in Bongfen et al. (P. berghei challenge of ENU-mutated animals). These polypeptides are preferentially selected from the group consisting of LYST, ZBTB7B, BPGM1, RASAL3, IRF8, IRF1, IRGM1, CCDC88B, THEMIS, FOXN1 and USP15, more preferably from the group consisting of IRF8, CCDC88B, FOXN1 and USP15, even more preferably from the group consisting of CCDC88B, FOXN1 and USP15 and, in the most preferred embodiment, from the group consisting of CCDC88B and USP15. In an embodiment, the IEP do not include JAK-3. As it will be shown below, it may be necessary to provide heterozygous animals for these inflammatory-enabling polypeptides. Such heterozygous animals bear a first functional gene copy coding for a functional IEP and a second gene copy coding for a non-functional IEP. A “functional” IEP (also referred to as the wild-type IEP) refers to a polypeptide capable of being expressed and providing its biological activity for mounting and/or maintaining an inflammatory response. By contrast, a “non-functional” IEP (also referred to as a mutant IEP) refers to a polypeptide that is not being expressed from its corresponding gene copy, expressed at lower level (when compared to the functional IEP) and/or bearing a mutation in its coding sequence which ultimately lead to a decrease (and even in the absence) of the inflammation-associated biological activity of the IEP.

Pharmaceutically effective amount or therapeutically effective amount. These expressions refer to an amount (dose) effective in mediating a therapeutic benefit to a patient (for example prevention, treatment and/or alleviation of symptoms of an inflammatory condition). It is also to be understood herein that a “pharmaceutically effective amount” may be interpreted as an amount giving a desired therapeutic effect, either taken in one dose or in any dosage or route, taken alone or in combination with other therapeutic agents.

Pharmaceutically acceptable salt. This expression refers to conventional acid-addition salts or base-addition salts that retain the biological effectiveness and properties of the therapeutic agent described herein. They are formed from suitable non-toxic organic or inorganic acids or organic or inorganic bases. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid, and the like. Sample base-addition salts include those derived from ammonium, potassium, sodium and, quaternary ammonium hydroxides, such as e.g., tetramethylammonium hydroxide. The chemical modification of an agent into a salt is a well known technique which is used in attempting to improve properties involving physical or chemical stability, e.g., hygroscopicity, flowability or solubility of compounds.

Prevention, treatment and/or alleviation of symptoms. These expressions refer to the ability of a method or an agent to limit the development, progression and/or symptomology of an inflammatory condition. The expressions include the prevent, treatment and/or alleviation of at least one symptoms associated to an inflammatory condition.

Acute inflammation can be observed in cerebral malaria, and/or encephalitis. Acute inflammation is usually characterized by the following symptoms: dolor (pain), calor (heat), rubor (redness), tumor (swelling) and/or functio laesa (loss of function). Redness and heat are considered to be due to increased blood flow at body core temperature to the inflamed site; swelling is considered to be caused by accumulation of fluid; pain is considered to be due to release of chemicals that stimulate nerve endings. Chronic inflammation can be characterized by the following symptoms: vasodilation; organ dysfunction; increased presence of acute-phase proteins (e.g. C-reactive protein, serum amyloid A, and serum amyloid P) that can cause fever, increased blood pressure, decreased sweating, malaise, loss of appetite and/or somnolence; modulation in leukocyte numbers (e.g. neutrophilia, eosinophiliam, leucopenia, etc.); modulation in interleukin, cytokine, hormone or growth factor concentration (e.g. IL-6, IL-8, IL-18, TNF-α, CRP, insulin, leptin); hyperglycemia; and/or heat.

Granulomatous inflammation is characterized by the formation of granulomas often observed in tuberculosis, leprosy, sarcoidosis, and syphilis. Fibrinous inflammation results in a large increase in vascular permeability and allows fibrin to pass through the blood vessels. If an appropriate procoagulative stimulus is present (e.g. cancer cells) a fibrinous exudate is deposited. This is commonly seen in serous cavities, where the conversion of fibrinous exudate into a scar can occur between serous membranes, limiting their function. Purulent inflammation results in large amount of pus (e.g. neutrophils, dead cells, and fluid). Serous inflammation is characterized by the copious effusion of non-viscous serous fluid, commonly produced by mesothelial cells of serous membranes, but may be derived from blood plasma. Ulcerative inflammation occurs near an epithelium and can result in the necrotic loss of tissue from the surface, exposing lower layers.

Trigger. As used herein, the term “trigger” (also referred to as an inflammatory response trigger) refers to agents capable of inducing and/or maintaining an inflammatory response in an individual. In some embodiment, the “trigger” can even lead to the onset of an inflammatory condition in the individual. Triggers includes, but are not limited to, bacterial infection, bacterial-derived components (such as bacteria or components derived therefrom), viral infection, viral-derived components, foreign antigens, and self antigens. In a preferred embodiment, the trigger is P. berghei.

### BRIEF DESCRIPTION OF THE DRAWINGS

Having thus generally described the nature of the invention, reference will now be made to the accompanying drawings, showing by way of illustration, a preferred embodiment thereof, and in which:

FIG. 1 illustrates the percent survival rate of various mice upon challenge with P. berghei. Jak3^{+/-} mice are heterozygote for one normal copy of the jak3 gene and one non functional copy of the gene that carries an internal deletion. Jak3^{W81R/+}mice are heterozygote for one normal copy of the jak3 gene and one non functional copy of the gene that codes for a protein bearing the W81R variant. B6 mice are homozygote for the jak3 gene and bear two functional copies of the gene. Jak^{-/-} mice are homozygote non functional copy of the gene that carries an internal deletion. Jak^{W81R} mice are homozygote for the jak3 gene and both gene copies bear non-functional mutations. “Treated” refers to animal who have received a Jak3 kinase inhibitor (tasocitinib). “Untreated” refers to animal who have note received a Jak3 kinase inhibitor.

FIGS. 2A to D illustrate the expression of CCDC88B. (FIG. 2A) qPCR was performed to investigate Ccdc88b's mRNA expression across a panel of murine organs, in adult mice. Results are shown as relative expression (as a ratio to the house-keeping gene Hprt) in function of organ. (FIG. 2B) qPCR was performed to investigate Ccdc88b's mRNA expression across a panel of murine cells, in adult mice. Results are shown as relative expression expression (as a ratio to the house-keeping gene Hprt) in function of cell type. (FIG. 2C) Immunophenotyping of spleen cells of wild type animals (grey bars) and of hemyzygote animals (Ccdc88b^{+/-}, white bars). Results are shown as number of spleen cells (×10⁶) per cell type. (FIG. 2D) qPCR was performed to investigate Ccdc88b's mRNA expression in human astrocyte, microglia or blood-brain barrier endothelial cells. The relative Ccdc88b's mRNA expression level is provided for astrocytes either left unstimulated or stimulated with IFNγ and TNFα (D1) or with IFNγ and IL1b (D2); for microglia either left unstimulated or stimulated with IFNγ and LPS (D3); for BBB endothelial cells either left unstimulated or simulated with IFNγ, TNFα or with IFNγ and TNFα (D4).

FIGS. 3A to D illustrate that BXH2 mice are resistant to cerebral malaria following P. berghei infection. (FIG. 3A) Survival curve of BXH2 mice, heterozygous (BXH2×B6)F1 offspring, and susceptible B6 and C3H parental controls. All curves are statistically different from one another. (FIG. 3B) Quantification of Evans blue dye accumulation in perfused brains from uninfected and infected B6 (white bars) and BXH2 (grey bars) mice. Data are averaged from three mice per condition. (FIG. 3C) Qualitative comparison of representative Evans blue dyed brains from uninfected and infected B6 and BXH2 mice at different points in time (either 7 days post infection or d7; sixteen days post infection or d16) which clearly indicate breakdown of the blood-brain barrier in infected B6 (d7 PbA), but not BXH2 (d7 PbA or d16 PbA) mice. (FIG. 3D) Blood parasitemia levels (percentage of parasitemia) following infection with P. berghei of susceptible B6 and C3H parental controls, BXH2 mice and heterozygous (BXH2×B6)F1 offspring.

FIGS. 4A to F illustrate that BXH2 mice have a dampened serum cytokine and chemokine response compared to CM susceptible parental controls. Serum from P. berghei infected mice was assayed via multiplex ELISA (n=5 mice/strain) and average cytokine levels were determined. The levels of (FIG. 3A) IFN-γ (pg/mL; *=p=0.07), (FIG. 3B) IL-10 (pg/mL), (FIG. 3C) MIP-1β (pg/mL), (FIG. 3D) CCL2 (pg/mL), (FIG. 3E) TNF-α (pg/mL; *=p<0.001) and (FIG. 3F) RANTES (pg/mL) are provided in function of mouse type (susceptible B6 and C3H parental controls and BXH2 mice).

FIGS. 5A to D show that transcript profiling of B6 and BXH2 brains reveals strain and infection specific differences in gene expression. (FIG. 5A) Unsupervised principal components analysis clusters samples according to strain and infection. Results are shown for component 2 (24.2%) in function of component 1 (39.4%) for B6 (d0=∘; d7=●) and BXH2 mouse (d0=Δ; d7=▴). (FIG. 5B) Intersection of gene lists generated by pairwise comparisons between infected and uninfected B6 and BXH2 transcript profiles. (FIG. 5C) Euclidean clustered heat map of transcripts regulated in both a strain and infection specific manner (two-factor ANOVA, padj-interaction<0.05) illustrated as infection-induced fold change in each strain (d7/d0). Each row represents a unique gene, and in cases where two or more transcript probes for a gene were significant, the average fold change was used. Differential expression patterns clustered into three groups with group 1 genes being upregulated by infection in both strains, group 2 genes upregulated by infection in B6 mice and unresponsive in BXH2 and group 3 genes downregulated by infection, typically more so in B6 than BXH2. See Table 1 for details. Shaded heat map indicates the presence of one or more direct IRF8 binding sites within 20 kb of the gene transcription start site. (FIG. 5D) Gene ontology for transcripts differentially regulated by infection during CM pathology in B6 mice. Upregulated genes are predominantly involved in innate and adaptive immunity processes, while downregulated genes do not form a clear message, but include a variety of homeostatic biological and metabolic processes.

FIGS. 6A to C illustrate that genes upregulated during CM pathology in B6 mice are significantly enriched for IRF8 binding sites defined by chromatin immunoprecipitation and DNA sequencing (ChIP-Seq). (FIG. 6A) Known binding targets of IRF8 (e.g. TIr4, H2-Q4, TIr9, Cd74 and Socs1) were used to validate the success of the ChIP by quantitative RT-PCR. These targets were highly enriched in IRF8-immunoprecipitated DNA when compared to control IgG preparations. Representative data shown for n=5 experiments. (FIG. 6B) ChIP-Seq peaks were aligned along the mouse reference genome. Light blue (top) track indicates non-specific (IgG) binding sites and dark blue track (below) displays direct IRF8 binding sites. Genes were considered to have an IRF8 binding site if a peak was found within 20 kb of the transcription start site. (FIG. 6C) The list of genes regulated by infection in B6 mice (d7/d0 pairwise) was interrogated for IRF8 binding sites within 20 kb of the transcription start site. Transcripts which were upregulated in response to infection were significantly enriched for IRF8 binding sites, while downregulated transcripts showed no enrichment.

FIGS. 7A to I illustrate the survival curves for targeted gene knockout mice infected with 10⁶ P. berghei parasites i.p. Cerebral malaria susceptible mice succumbed with neurological symptoms between d5 and d10 post-infection, while mice who survived longer than 13 days never developed CM symptoms and were categorized as resistant. Infection specific B6 and BXH2 controls are plotted alongside each knockout strain. Results are shown as percentage of survivals in function of days post-infection for knock-outs of (FIG. 6A) IFN-γ, (FIG. 6B) STAT1, (FIG. 6C) JAK3, (FIG. 6D) IRF1, (FIG. 6E) IRGM1, (FIG. 6F) IL12p40, (FIG. 6G) IFIT1, (FIG. 6H) ISG15, and (FIG. 6I) NLRC4.

FIGS. 8A to E illustrate that an ENU-induced mutation in USP15 protected mice against development of experimental cerebral malaria. (FIG. 8A) Breeding scheme for the production of ENU-induced mutant mice. (FIG. 8B) G2 females, Doshia and Kala, were backcrossed to their G1 father Corbin, and their G3 offspring were infected with 10⁶ PbA-parasitized red blood cells and monitored for appearance of neurological symptoms and for survival. (FIG. 8C) Whole-exome sequencing identified a T-to-G transversion in exon 17 of the Usp15 gene in pedigree Corbin. The transversion causes a leucine (L) to arginine (R) amino acid substitution at position 749 of the protein. (FIG. 8D) Survival plots of PbA-infected Usp15 homozygote mutants (Usp15^{L749R}), heterozygotes (Usp15^{L749R/+}), Usp15 knockout (Usp15^{KO}), double heterozygotes (Usp15^{L749R/+}:Usp15^{KO/+}) and wild type Usp15 or B6 controls (data is from 5-8 PbA-infections). Statistical significance for survival between Usp15-related mutants and B6 wild type was determined by the Log-Rank test. **** p<0.0001. (E) Blood stage parasitemia during PbA infection of B6 and Usp15^{L749R} homozygotes. Data represents a single experiment (5 mice per group) and expressed as a mean±SD. Statistical significance was calculated using a two-tailed unpaired student t-test.

FIGS. 9A to D illustrate the reduced protein expression and reduced stability of the USP15 variant in vivo and in vitro. (FIG. 9A) Major predicted structural features of USP15, including the approximate positions of the deubiquitinase specific (DUSP), the ubiquitin-like (UBL) domain, and the L749 residue in the carboxy-terminal portion of the protein; the leucine (L) to arginine (R) amino acid substitution at position 749 in the catalytic domain of USP15 is highly conserved across species (indicated to the left). Mouse (SEQ ID NO: 43), rat (SEQ ID NO: 44), human (SEQ ID NO: 45), orangutan (SEQ ID NO: 46), horse (SEQ ID NO: 47), pig (SEQ ID NO: 48), rabbit (SEQ ID NO: 49), chicken (SEQ ID NO: 50), xenopus (SEQ ID NO: 51) and zebrafish (SEQ ID NO: 52) USP15 amino acid sequences are shown. (FIG. 9B) Immunoblotting analysis of USP15 protein expression in lymphoid and myeloid cells. Protein extracts were prepared from wild type B6 cells following cell sorting of total spleen and thymus cells, and from in vitro derived bone marrow (macrophages (mac) and dendritic cells (DC)), and from mouse embryonic fibroblasts (MEF). Splenic CD4 T cells (CD4⁺CD8⁻), CD8 T cells (CD4⁻CD8⁺), NK cells (TCRb⁻CD49b⁺) and B cells (TCRb⁻CD19⁺); Thymic double negative T cells (DN: CD4⁻CD8⁻), double positive T cells (DP: CD4⁺CD8⁺), single positive CD4 T cells (CD4⁺CD8⁻) and single positive CD8 T cells (CD4⁻CD8⁺). (FIG. 9C) Cell extracts from spleen and thymus from control B6, 129S1, and from Usp15^{L749R} homozygote mutants were analyzed for USP15 protein expression. Data are representative of two independent experiments. (FIG. 9D) HEK293 cells stably expressing HA-tagged VVT or USP15^{L749R} proteins were treated with cycloheximide (CHX, 20 μg/ml) for 10, 15, 20, and 25 hours, and equal amounts of protein (25 μg) were analyzed by immunoblotting. Data is a representation of two independent experiments, assessing two different clones per construct, and expressed as a mean±SD.

FIGS. 10A to G illustrate a reduced ECM and EAE cerebral pathogenesis in Usp15^{L749R} homozygotes. (FIG. 10A) Representative FACS plots of brain cellular infiltrates in PbA-infected control B6 and Usp15^{L749R} mutant mice at day 5 post-infection. Data are expressed as the total number of viable cells in the brain. Data is a representation of two independent experiments. (FIG. 10B) Serum cytokines from PbA-infected mice at day 5 post-infection were analyzed by Luminex. Data is from a single representative experiment. EAE was induced in C57Bl/6J (n=8), Usp15^{L749R} (n=10), and Jak3^{-/-} (n=5) 8 week old female mice using 50 μg MOG₃₅₋₅₅ (d0) plus pertussis (d0, d2). Animals were monitored daily for (FIG. 10C) weight, (FIG. 10D) clinical score, and (FIG. 10E) survival. (FIG. 10F) Scores for individual B6 and Usp15^{L749R} animals. Data is representative of three independent experiments. (FIG. 10G) Serum cytokines were analyzed at day 2 and day 7 post-EAE induction by Luminex. (FIG. 10A-FIG. 10G) All data are expressed as a mean±SD for each group, and all statistical analyses were performed using the two-tailed unpaired student t-test.

FIGS. 11A to D illustrate the effect of USP15 on global gene expression during neuroinflammation of the brain, and of the spinal cord. Genome-wide gene expression was measured using RNA-seq on whole brain RNA extracts from controls and from PbA-infected (day 5) WT and Usp15 mutants mice, as well as from spinal cord of WT and Usp15 mutants undergoing EAE (day 7). (FIG. 11A) Dimension reduction analysis using partial least square method performed on normalized gene expression values for all RNA-seq datasets. The first three principal components are shown in a three-dimensional graph. (FIG. 11B) Dendogram presenting unbiased clustering of genes significantly dys-regulated (>1.5 fold change and adjusted p value <0.01) in Usp15 mutant mice during ECM and/or EAE. The 244 genes with lower reduced expression in Usp15 mice are identified as USP15-dependent genes. (FIG. 11C) Histogram showing gene ontology enrichment analysis of the USP15-dependent genes; the degree of statistical significance is shown. (FIG. 11D) The differential gene expression profiles of Usp15 mutant mice compared with WT mice in either the PbA or the EAE neuroinflammation models were subjected to GSEA analyses to identify immune cell signatures altered by the loss of USP15. GSEA graphs illustrate the cumulative enrichment score for each specific immunological gene signature comparison; the occurrence of the signature genes is reported as individual black lines over the distribution of brain or spinal cord gene profiles. Normalized enrichment scores (NES) and false-discovery rate (FDR) are shown for each displayed analysis. Representative GSEA graphs are shown for the most highly enriched signatures in the PbA and EAE conditions.

FIGS. 12A to F illustrates that cell populations and associated molecular pathways were differentially regulated in USP15-dependent fashion. (FIG. 12A) Dendogram showing unbiased clustering of genes driving (leading edge analysis; LEA) the significant enrichment of immunological signatures in GSEA analysis. Individual lists of leading genes and enriched signatures for PbA and EAE datasets were clustered (the separate PbA and EAE datasets are shown see FIG. 17). Clusters of enriched immunological signatures and functions are highlighted by color boxes: red=signatures of IFN activation, green=myeloid signatures and responses, and purple=T cell signatures. (FIG. 12B) Representative examples of Type I IFN response genes activated during PbA infection and differentially expressed in brains from WT and USP15^{L749R} mutants. The normalized sequence reads profile is shown over the gene structure (biological triplicates). (C) RT-qPCR analysis of Type I IFN stimulated genes activation over the course of PbA infection (days 0, 1, 3, and 5) in brains of WT (B6) and Usp15^{L749R} mutants. Gene expression was assessed for 4-5 mice per group, normalized relative to Hprt expression, and expressed as fold induction relative to t=0. Data is shown as mean±SD; p values were calculated for Usp15^{L749R} vs. B6 comparison using unpaired student's t-test (*<0.05, **<0.01, ***<0.001). (FIG. 12D-FIG. 12F) Relative gene expression was assessed by RT-qPCR as described in (FIG. 12C) for lymphoid-specific (FIG. 12D), and myeloid-specific markers (FIG. 12E) and for Plin4 (FIG. 12F).

FIGS. 13A to D illustrate that USP15 modulated the type-I IFN response through function on Trim25. (FIG. 13A) HEK293T cells transiently expressing Xpress (Xpr)-tagged human USP15 wild type (VVT) or mutant (L720R) USP15, with or without co-transfection with FLAG-tagged TRIM25, were lysed in 1% NP40 lysis buffer, followed by immunoprecipitation with either anti-Xpr or anti-FLAG antibodies. Proteins in whole cell lysates (WCL) and immunoprecipitate samples (IP) were separated by SDS-PAGE and analyzed by immunoblotting (IB) using the indicated antibodies. (FIG. 13B) HEK293T cells expressing Xpr-tagged human USP15 (hUSP15) or HA-tagged mouse USP15 (mUSP15) VVT or mutant variants, with or without FLAG-tagged TRIM25 were lysed in 1% NP40 RIPA buffer, followed by immunoprecipitation with anti-FLAG antibody. Proteins in WCL and IP samples were analyzed by western blot sequentially for ubiquitin and for TRIM25 expression. Top 2 panels represent different exposure of the same blot; immediately below, mono-ubiquitinated TRIM25 (upper band) and non-ubiquitinated TRIM25 (<75 kDa) band are detected. The presence of the upper band is consistent with the reduced ability of the indicated USP15 constructs to deubiquitinate TRIM25. (FIG. 13C-FIG. 13D) Survival plots of PbA-infected Trim25 mutants (Trim25^{-/-}), Trim25 heterozygotes (Trim25^{+/-)}, Usp15^{L749R} heterozygotes (Usp15^{L749R/+}), Usp15^{L749R/+}: Trim25^{-/-} double heterozygotes and B6 controls. Data are from 3 independent PbA-infections. Statistical significance for survival between groups of mice was determined by the Log-Rank test.

FIGS. 14A and B illustrate that mouse mutants bearing loss of function mutations in Socs1/Socs3 and Irf3 were protected against neuroinflammation. Survival plots of PbA-infected (FIG. 14A) Socs1^{fl/fl}/Socs3^{fl/fl} knockouts (Socs1^{fl/fl}/Socs3^{fl/fl}x Lck-Cre), (FIG. 14B) Irf3 knockouts (Irf3^{-/-}) and B6 controls. Statistical significance for survival between groups of mice was determined by the Log-Rank test (*<0.05, ****<0.0001).

FIGS. 15A to C illustrate the ubiquitous pattern of Usp15 mRNA expression in embryonic, post-natal and adult mice. (FIG. 15A) Mouse sections were stained with cresyl violet to localize Usp15 RNA to specific organs and structures. In situ hybridization was carried out using radiolabelled antisense (as) (FIG. 15B) and sense (s) (FIG. 15C) probes. The results shown are from X-ray film autoradiography obtained following 5-days exposure. The results show low-level ubiquitous pattern of Usp15 mRNA expression in most tissues, with high-level expression in the testis and in the ovary. Non-specific localized signals (visible with sense and anti-sense probes) are indicated with an asterisk (*); in the teeth (p10) and the large intestine lumen (p10 and adult). (Magnification: Embryonic x2.4, Post-natal x3, Adult x2.4). Abbreviations: Adr—adrenal gland; At—heart atrium; Br—brain; Bro—bronchcus; Car—cartilage; Cb—cerebellum; Co—colon; Cx—cerebral cortex; Du—duodenum; E—eye; Ep—epididymis; Es—esophagus; GB—gallbladder; HV—heart ventricle; II—ileum; Je—jejunum; Ki—kidney; Li—liver; LI—large intestine; Lu—lung; OL—olfactory lobe; Ov—ovary; Ovi—oviducts; PB—pelvis bone; Pc—pancreas; PG—pituitary gland; Pr—prostate; PTh—parathyroid gland; R—ribs; Sk—skin; Spl—spleen; St—stomach; SV—seminal vesicle; Te—testis; Th—thyroid gland; UB—urinary bladder; Ut—uterus; CA—central artery; GC—germinal center; LN—lymphatic nodule; RP—red pulp; Tr—trabeculum; V—vein; LF—lymphoid follicle; Me—medulla; MG—mammary glands; Cx—cortex.

FIGS. 16A to D illustrate the immunophenotyping of Usp15^{L749R} mutants at steady-state and following P. berghei ANKA infection. (FIG. 16A) The number and proportions of different types of spleen cells were analyzed by FACS, using standard markers of T cells (CD4, CD8), B cells (B220), NK cells (NK1.1), monocytes and neutrophils (CD11b, Ly6G), and tested at steady state, and 5 days following PbA infection (results are pooled from 5 experiments). (FIG. 16B) Splenocytes from naive and PbA-infected mice were cultured in vitro for 4 hours with either media (unstimulated), or with anti-CD3/anti-CD28 (TCR engagement) or with PMA/lonomycin, and the capacity of T cells to produce cytokines were assessed by intracellular staining and flow cytometry. (FIG. 16C) The activation state of CD4⁺ and CD8⁺ T cells was assessed by analysis of CD69 cell surface expression in response to TCR engagement (anti-CD3/anti-CD28). (FIG. 16D) The percentage of splenic CD4+ and CD8⁺ naive T cells (CD62L⁺CD44⁻) and memory effector T cells (CD62L⁻CD44⁺) were assessed by flow cytometry both at steady-state and at day 5 post-infection. The data show that Th1 response is not affected by the Usp15^{L749R} mutation during PbA infection. Data is from one representative experiment.

FIGS. 17A and B illustrate that the cell populations and associated molecular pathways differentially were regulated in USP15-dependent fashion. (FIG. 17A) LEA dendogram for genes with reduced expression in Usp15^{L749R} mutant mice compared to WT B6 (day 5 post-PbA infection) and that drive significant enrichment (FDR<0.01) of immunological expression signatures (GSEA). Enriched immunological signatures and functions are highlighted by color boxes: red=signatures of IFN activation, green=myeloid signatures and responses, and purple=T cell signatures. Refer to Materials and Methods for details on LEA analysis. (FIG. 17B) LEA clustering analysis as described in (FIG. 17A) for immunological signatures depleted in Usp15^{L749R} mutant mice during EAE neuroinflammation progression.

FIGS. 18A to E illustrate that USP15 negatively regulated CD4+ T cell activation in the Listeria monocytogenes model. Wild type B6 mice and Usp15^{L749R} mutants infected with 1×10⁴ CFU of Listeria monocytogenes (strain 10403s) were sacrificed on day 7 post-infection, and phenotyped for the activation of the T cell response in spleen cells populations. (FIG. 18A, FIG. 18B) CD44 expression (T cell activation) on CD4⁺ T cells (FIG. 18A), or CD8⁺ T cells (FIG. 18B), expressed as percentage and total cell numbers. (FIG. 18C, FIG. 18D) Cells were re-stimulated in vitro with Listeria-specific antigens, LLO or OVA, and IFNγ production was assessed by flow cytometry (FIG. 18C, intracellular staining), or by ELISA (FIG. 18D, culture supernatants) for CD4⁺ and CD8⁺ T cells. (FIG. 18E) Serum IFNγ levels were measured by ELISA, and plotted as optical density absorbance (OD) at 450 nm. (FIG. 18A-FIG. 18E) Data is a combination of two independent experiments. All data are expressed as a mean±SD for each group, and all statistical analyses were performed using the two-tailed unpaired student t-test.

FIGS. 19A to D illustrate that USP15 was expressed in resident cells of the brain, and is up-regulated in response to pro-inflammatory signals. Usp15 mRNA expression was monitored (qRT-PCR amplification) in primary human astrocytes (FIG. 19A, FIG. 19B), primary microglia (FIG. 19C), and in primary endothelial cells of the blood brain barrier (BBB) (FIG. 19D) from 3 individuals, either prior to or following stimulation with the indicated cocktails of pro-inflammatory molecules. Expression is relative to Gapdh which was used as an internal control.

### DETAILED DESCRIPTION

In accordance with the present disclosure, there is provided host polypeptides (also refer to as targets) which are herein shown to enable the induction and/or persistence of a pathological inflammatory response. These polypeptides are considered to be involved in any pathological inflammation response, regardless of the etiology of the disease. As also shown herein, reduction in the biological activity of these polypeptides is shown useful for the treatment, prevention and/or alleviation of symptoms associated with an inflammatory condition.

Common inflammatory diseases such as psoriasis, rheumatoid arthritis, multiple sclerosis, lupus, celiac disease and IBD show deregulation of common pathways and associated signaling networks. Therefore, pharmacological modulators interfering with such shared pathways associated with pathogenesis may be of clinical benefit for several inflammatory diseases. Large-scale genetic studies show that the genetic component of these diseases is diverse and highly complex and, importantly, that they do share several of the same genetic risk alleles. This suggests that genes discovered as playing a role in one inflammatory condition may prove valuable to better understand the pathophysiology of related inflammatory conditions and may represent valuable targets for drug discovery in these disorders. Studies in experimental models have established that the principal cell types, physiological responses, and associated pathways and soluble mediators underlying normal and aberrant inflammatory responses are conserved in the mouse. In addition, germ-line modification in mouse provides an ideal model to study in isolation the contribution of individual genes and proteins to pathological inflammation, while carefully controlling triggering environmental stimuli.

Building on these findings, a random mutagenesis in mice was performed followed by screening of the resultant mutants for resistance to acute neuroinflammation provides a systematic approach to identifying functionally validated factors that a) play crucial roles in a spectrum of inflammatory diseases, and b) constitute novel targets for drug discovery and pharmacological intervention in inflammatory conditions. As it will be provided below, such strategy has resulted in the identification of Jak3 (a known pharmacological target in RA) and Themis (a known genetic risk factor in SLE and celiac disease), providing convincing proof-of-principle of the robustness and power of this genetic approach. Thus, this approach provides the missing functional link absent in GWAS-derived information as well as an in vivo-validated pre-clinical system in which pharmacological inhibition would sufficiently blunt the inflammatory response to be clinically valuable.

IBD affect about 1.4 million individuals in North America and 2.2 million in Europe. Since the 1940s, the incidence of IBD has dramatically increased in countries with a more westernized lifestyle, suggesting the influence of environmental factors, including lifestyle, hygiene, diet and use of antibiotics, all of which may alter the microbiota in favor of disease onset and/or progression. A large body of evidence also points to genetic factors in the etiology of IBD, with more than 100 loci identified in GWAS. However, these loci account for only 20% of the estimated genetic risk for IBD. Furthermore, although the loci identified by GWAS point to specific pathways with implications in inflammation, epithelial barrier function, innate and adaptive immunity, autophagy, ER stress and others, it is currently impossible to rationally select a candidate druggable molecule/pathway from these studies due to lack of functional studies evaluating the non-redundant in vivo contribution of each of these loci to disease. The neuroinflammation model induced by Plasmodium berghei infection was chosen because it harbors several features common to inflammatory conditions, including recruitment of inflammatory myeloid and lymphoid cells to the site of infection, secretion of IL12, TNFα, IFNγ and subsequent disruption of endothelial cells integrity leading to a rapid and lethal encephalitis. Further, as it will be shown below, a number of genes which role in inflammation was previously unsuspected, establishing the proven potential of discovery of novel druggable targets.

### Genetic Screening and Characterization of Putative Inflammation-Enabling Targets

Mutagenesis with the alkylator N-ethyl N-nitrosourea is an efficient method to create random single point mutations in the mouse genome. A set of validated conditions was developed to successfully mutagenize the C57BL/6J (B6) mouse strain (Bongfen et al.). Briefly, founder B6 male mice (identified as G0) receive 1 intraperitoneal injection of 90 mg/Kg of ENU weekly for 3 weeks. Efficiently mutagenized males (G0) transiently lose fertility, which is regained after 11 weeks. Mutagenized G0 males are then crossed with wild-type B6 female mice to produce generation 1 (G1) offspring: these F1 hybrids carry one full set of mutagenized chromosomes and one full set of wild-type B6 chromosomes. Based on an average substitution rate of 1 nucleotide per 1 Mb, each G1 is expected to carry ˜3000 nucleotide variants in heterozygous state. Using an average frequency of reduction-of-function mutations of 1 per locus per 700 G1 mice, determined for visible loci, each G1 mouse is likely to be a heterozygous carrier for about 40 to 50 functional variants. Individual G1 males are bred as founders of separate B6 pedigrees aimed at bringing the ENU sequence variants (from the G0 male) to homozygosity, by first crossing the G1 male with a wild-type B6 female (to produce G2 offsprings) and then backcrossing two of the ensuing G2 daughters back with their G1 father. 50% of the sequence variants present in the G1 are inherited by each G2 daughter, and 25% percent of these (12.5% of the total) are expected to come to homozygosity in any one N3 offspring. Overall, each G3 offspring is thus expected to be homozygous for an estimated 375 sequence variants and 4 functional variants. During systematic phenotyping campaigns of these N3 pedigrees, the consistent appearance (at a frequency of about 25%) of individuals harboring a discordant or “phenodeviant” trait (e.g. resistance to lethal neuroinflammation) in successive G3 pedigrees from the same G1 grand-father, indicates segregation of a fully penetrant recessive mutation modifying response to a stimulus. Thus, by screening a minimum of 24-32 N3 offspring (six litters) per pedigree, one can expect to identify a cluster of 6-8 individuals with the same phenodeviant trait.

Plasmodium berghei ANKA (PbA) induces cerebral malaria, a condition characterized by acute neuroinflammation and rapidly fatal encephalitis. It is caused by trapping of PbA-parasitized erythrocytes at the blood-brain barrier (BBB), infiltration of inflammatory cells and production of IFNγ, TNFα, IL10, MCP-1, loss of endothelial integrity at the BBB, and precise onset of irreversible neurological symptoms that appear by day 5 post-infection and that are ultimately fatal by day 9-11. The infectious P. berghei ANKA (PbA) isolate was obtained from the Malaria Reference and Research Reagent Resource Centre (MR4), and stocks of parasitized erythrocytes (pRBCs) were prepared and stored frozen at −80° C. Prior to infection, the parasite is passaged intravenously, and the blood is diluted to prepare a large inoculum (titrated at 10⁷ pRBC/mL) to infect groups of 100-150 G3 mice (10⁶ pRBC, i.v). Starting at d3 post-infection, animals are monitored several times for the appearance of neurological symptoms which may range from shivering, tremors, hunched-back, lack of responsiveness to touch, and may progress to paralysis and coma. At first sight of such symptoms (usually by days 5 to 7), mice are euthanized and a tissue sample is collected by isolation of genomic DNA. Mice surviving d12 post-infection are flagged as resistant to neuroinflammation, and the corresponding G3 pedigree as phenodeviant. Additional G3 pedigrees from the same G1 founder are then phenotyped to ascertain stable segregation of the protective trait, and establish its frequency of transmission (expected about 25% G3 individuals in a positive pedigree) amongst a minimum of 24-32 G3 offspring (6 litters from the same G2 female). Resistant animals surviving the cerebral phase are tested for blood parasitemia (to ascertain productive infection), and are then drug-cured (Artemisin, s.c, 150 mg/kg, 3× per week for 3 weeks) to preserve the protective mutation in live animals for future propagation of the mutant line.

The sequence of all exons and exon-intron boundaries (exome sequencing) from 3 G3 mice from the same phenodeviant pedigree is determined using the Agilent SureSelectXT Mouse All Exon™ system. This system uses nested oligonucleotide primers to efficiently construct libraries that capture >221,784 exons within 24,306 annotated mouse genes (annotated using UCSC mm9/NCBI build 37 from reference C57BL/6J genome) for a total 49.6 Mb captured genomic DNA. The captured exons are then subjected to deep sequencing using the Illumina HiSeq™ sequencing platform. These two components have been optimized for efficient and reliable whole genome coverage and for detection of ENU-mutations: 200-300×10⁶ sequence reads of an average read length of 180-200 nucleotides being sufficient to provide 20× coverage for >90% of the genome and 10× coverage for >95%. A read alignment and variant-calling pipeline based on the freely available tools BWA, SAM tools, Picard, and GATK have been implemented. This list is curated to eliminate duplicate reads and to flag sequencing errors (ratios of SNP to reference allele <50%, multiple allele systems, end sequences) using the IGV Viewer™ software package (Broad Institute, MIT), and to identify other non-ENU irrelevant variants (previously seen in other exome files) or silent variants that do not affect the amino acid sequence. SNP lists from the 3 G3 mice are compared to identify variants common to the 3 mice (homozygote or heterozygote) states.

The causative nature of the ENU variant(s) are validated by genotyping 20 additional G3 mice from the same G1 grandfather, and by looking for co-segregation of homozygosity for the ENU-specific variant(s), and exclusion of homozygosity for wild type alleles in the “resistant” mice while ascertaining the reverse situation in “susceptible” mice. The cause for the loss-of-function in the protective variants are then examined. Although mis-sense variants causing either premature termination of the polypeptide, or intronic mutations in key donor or acceptor splice sites likely to affect RNA processing/splicing readily identify obvious loss-of-function, non-synonymous variants can be prioritized with respect to the type of substitution (non-conservative >conservative) and for residues showing cross-species conservation (BLAST or BLOSSOM searches). Genetic complementation in F1 mice double heterozygote for the ENU variant and for a null allele at the same gene (looking for recapitulation of protection against P. berghei) can be used as an ultimate validation test. Such null mutants may be obtained as live animals from independent laboratories or from public resources (Jackson Laboratories) or may be obtained as gene traps from the International Gene Trap Consortium (www.igtc.org.uk). To gain further insight into the functional role of poorly annotated proteins, the expression of their RNA by semi-quantitative reverse transcriptase coupled amplification (RT-PCR) in organs (thymus, spleen, bone marrow, blood leukocytes) and cells (myeloid, lymphoid, epithelial) associated with inflammatory responses in vivo can be performed. The promoter region of these genes can be studied for the presence of sequence elements associated with regulation by transcription factors known to regulate “pro-inflammatory” pathways (such as STAT1, STAT3, IRF1, and IRF8). Finally, mutant mouse lines homozygote for prioritized ENU-variants are expanded to generate sufficient numbers of mice for downstream characterization.

Mutant phenodeviant pedigrees are subjected to a streamlined and stratified immunophenotyping analysis to determine the integrity, composition and activation status of their central and peripheral immune systems. Specifically, the different immune cell populations are enumerated (hematopoeitic stem cells [HSC], granulocytes, myeloid cells, NK cells, T and B lymphocytes) in central and peripheral immune organs (bone marrow, thymus, spleen, lymphnodes and peripheral blood) as well as in the gut, using population-specific cell surface markers and intracellular cytokine staining in a high throughput multivariate flow cytometry analysis. The following antibody cocktails can be used: for T cells (anti-CD3, CD4, CD8, CD44, CD62L, CD25, TCRVβ, γδTCR), B cells (anti-B220, CD22, CD138, IgM, IgD, CD24), HSC (anti-CD117, Sca-1), erythroid cells (anti-Ter119), granulocytes (anti-Gr-1), NK cells (anti-NK1.1), macrophages, DCs, inflammatory monocytes (anti-CD11b, CD11c, F4/80), and eosinophils, mast cells, basophils (anti-SiglecF, CD117, FcERI). Immune cell functions are interrogated by analyzing different immunological responses both ex vivo and in vivo.

The genetic screen provides decisive proof-of-principle and support these conclusions: a) the ENU screen is a robust and effective platform to identify mutations (and ultimately genes) that blunt acute neuroinflammation; b) some of the mutations detected so far are in genes are already known to be critical for the inflammatory response (e.g. JAK3) and/or c) other mutations detected so far are in genes are already known to be important in the genetic etiology of human chronic inflammatory diseases (e.g. THEMIS, IRF8) indicating that genes discovered in an acute inflammation screen in mice are relevant to other types of human inflammatory conditions; d) likewise, the neuroinflammation protective effect of a genetic mutation in the screen (e.g. JAK3) can be mimicked by pharmacological inhibition of the corresponding target with compounds in clinical use for chronic human inflammatory diseases (refer to Example I); e) the acute neuroinflammation model can be used as a primary screen in vivo for the rapid and effective pre-clinical evaluation of novel anti-inflammatory drug candidates; f) novel genes/proteins with unknown function are also identified and represent novel windows of opportunity for anti-inflammatory drug discovery; g) the screen is very sensitive and can detect gene-dosage dependent pathways since the mutations show partial resistance to neuroinflammation suggesting that the screen can detect targets of which partial inhibition in vivo may be of clinical value. The genetic screen herewith presented is also advantageous because it provide inflammation-enabling polypeptides derived from the host.

To further characterize the targets of the genetic screen, for unknown genes/proteins or proteins with unknown function in inflammation, different cell populations can be sorted using the AutoMACS™ system or more precise cell sorting strategies when necessary (e.g. BD FACSAria™) and can be challenged in vitro with appropriate triggers including PRR agonists (LPS, PGN, CpG, polyI:C, MDP, DAP, B-DNA), recombinant cytokines or mitogens (IL-2, IL-7, anti-CD3/CD28, PMA+ionomycin). Cell-based assays can be conducted to monitor cell survival (by Annexin V/propidium iodide staining and enumeration by FACS), proliferation (by following the dilution of the CFSE stain by FACS) and activation (intracellular staining for IFNγ [Th1], IL-17 [Th17], IL-4 [Th2], FoxP3 [Treg], and cell surface staining of MHC Class II and the co-stimulatory molecules CD80/CD86 [DCs]). These assays can be complemented with in vivo challenge and immunization experiments to determine the activation, survival and migratory capacities of the granulocytes, myeloid and lymphoid cells under investigation. Immunohistochemistry, bead-based immunoassays (BioPlex) and ELISA assays for different serum cytokines, chemokines and immunoglobulins can be used to assess the in vivo innate and adaptive (cellular and humoral) immune responses. Mice can be injected intraperitoneally (IP) with PAMPs/DAMPs (e.g. LPS, PGN, MSU, Alum) and flow cytometry is used to quantify the frequencies and numbers of infiltrating granulocytes and myeloid cells into the peritoneal cavity (Gr-1+, CD11b+, F4/80+, SiglecF+, FcERI+). Cytokines and chemokines in the peritoneal lavage and the serum can be quantified by BioPlex and ELISA assays. Classical immunization experiments can involve the immunization of animals by IP injection of DNP-Ficoll or DNP-KLH in complete or incomplete Freund's adjuvant followed by an antigenic rechallenge/boost on day 28. The production of different immunoglobulins (IgA, IgG1, IgG2a/2b, IgG3, IgM) and cytokines (IFNγ, IL-4, IL-5, IL-13, IL-17) can be quantified by BioPlex and ELISA. Acquired cellular immune response can be interrogated in delayed-type hypersensitivity assays (intraperitoneal immunization with TNBS and rechallenge in the foot pad and measurement of foot pad swelling as a readout).

A more in-depth analysis of the impact of the mutation on the inflammatory response can be undertaken by identifying transcriptional signatures (using RNAseq™) in the relevant immune cell type distinguishing inflammation-resistant versus inflammation-susceptible mice. This comprehensive analysis not only defines the immunological mechanism(s) by which the identified targets impact the inflammatory response but can also provide an integral view of the cell types and pathways involved in pathologic inflammation, and might provide additional drug targets that function either upstream or downstream of the identified genes/proteins with equivalent consequence on immune cell function and physiopathology of inflammatory disease.

The relevance of neuroinflammation-protective mutations previously identified can be assessed in other inflammatory conditions, such as an animal models of IBD. Experimental animal models of IBD individually recapitulate aspects of the problem, and have provided important insights into the role of the pathways, cells and molecules required for intestinal homeostasis. This is best illustrated by the remarkably convergent mouse and human studies on the role of the IL-23-Th17 cell pathway in IBD. IL-23p19 was first cloned in the mouse and has been shown to be required for colitis to develop in multiple models. Neutralization of IL-23 completely reversed active colitis in one model, which is consistent with data showing that the dominant IL23R SNP, which is protective in IBD, encodes for an alternative splicing of the gene resulting in a soluble receptor antagonist of IL-23. Similarly, the Nlrp3 inflammasome-IL-18 axis has been linked to protection from colitis, which is consistent with human genetic studies identifying SNPs in the NLRP3 and IL-18 receptor accessory protein (IL18RAP) as risk alleles for IBD. The response of the mutant mice identified in the primary screen can be examined in a number of complimentary models of acute and chronic inflammation of the bowel that each interrogates a specific aspect of the immune response. Such models include, but are not limited to, the acute and chronic dextran sulfate sodium (DSS)-induced injury/colitis models to probe for innate immunity mechanisms; the CD4+CD45RB^{Hi} T cell transfer colitis model to probe for Th1/Th17 mechanisms as well as deregulated Treg functions; the oxazolone colitis model to examine Th2 mechanisms and infection with C. rodentium to probe for epithelial and innate immunity anti-microbial defense mechanisms. Various parameters can be systemically measured to determine the severity of colitis, including body weight loss, colon histology to assess crypt architecture, loss of goblet cells, erosion of surface epithelia, infiltration of inflammatory cells and edema, quantitative real time PCR and BioPlex/ELISA assays to measure the expression of various antimicrobial peptides (including defensins), chemokines and cytokines in the colon tissue and in the serum, qPCR of 16S rRNA to determine extent of bacterial invasion in the colon, and to systemic sites (mesenteric lymphnodes and spleen), and flow cytometry analysis to enumerate the frequencies and absolute numbers of the different populations of immune cells in the colitic gut (neutrophils, macrophages, T and B cells).

It can also be investigated whether common polymorphic variants (SNPs) within or near the human counterparts of the targets have been associated as genetic risks of common inflammatory diseases in genome wide association studies (GWAS). Published GWAS studies have identified a very complex genetic component to common inflammatory conditions including rheumatoid arthritis (RA), psoriasis (PA), celiac disease (CeD), multiple sclerosis (MS), type 1 diabetes (T1D), lupus (SLE), and inflammatory bowel disease (IBD). Although these GWAS studies have defined genetic signatures and associated immune/biochemical pathways that are either shared in common or that are specific for one disease, translating GWAS hit(s) into potential novel target(s) for drug discovery and therapeutic intervention is complicated by many factors, the most important being the lack of direct experimental validation of the biological role of the individual genes in onset/progression of disease. A positive correlation between the genes which inhibition in mice protects against acute inflammation, and genetic risks for either RA, PA, CeD, MS, T1D, SLE or IBD in published GWAS studies can bring biological validation on the role of this gene in human inflammatory diseases, a key criterium for prioritization of this gene and protein in our discovery pipeline.

It can also be determined if the expression of the inflammation-enabling target is modulated in tissue samples from patients suffering from chronic inflammatory diseases. One approach is based on reports showing that an important proportion (up to 30%) of allelic variants linked to disease (linked SNPs) are also associated with allele-specific differential expression of the associated genes (cis-acting eSNP, eQTL). These published datasets can be searched for presence of eSNPs in human counterparts of the inflammation-enabling mouse targets, and which differential expression may be associated with disease. In another approach, the expression of the inflammation-enabling target in normal intestinal mucosa can be compared to the expression of the inflammation-enabling target in inflammed mucosa from IBD patients (via, for example, RNA sequencing technology to capture in whole tissue RNA or following microdissection) whole transcriptome expressed in normal and inflamed tissues and PBLs. Finally, it can be tested whether rare sequence variants in human relatives of mouse targets are associated with specific clinical features of IBD, or with IBD onset and progression in certain genetically unique groups of patients such as familial cases, very early onset pediatric cases, and sporadic cases from genetically homogeneous isolated populations. In an embodiment, individual exons and exon-intron junctions of each gene can be PCR-amplified can subjected to DNA sequencing, looking for obvious loss of function alleles or for non-conservative and possibly pathological mis-sense mutations, using standard methodologies. In a complementary or alternative embodiment, intronic sequences can also be determined using standard methodologies.

If inflammation-enabling protein targets require further degrees of experimental characterization, for protein with a known biochemical function or ligand binding, tractable modification of substrate can be conducted. Bioinformatics tools can also be used to scrutinize the predicted amino acid sequence to a) deduce biochemical function by relatedness to other proteins which function is known, and b) sequence motifs and signatures, and associated structural folds associated with specific biochemical activity (nucleotide binding, protease site, DNA binding, kinase, phsophatase, etc.), and c) presence of secondary structure motifs indicative of protein localization (hydrophobic transmembrane domains, signal sequences, nuclear localization sequences), prioritizing for pharmacological access. Candidate biochemical activities for such proteins can be validated following transfection and overexpression in cultured cells and looking for appearance of candidate activity and/or changes in cellular components related to functional markers of inflammation, or conversely, looking for their disappearance following silencing by RNAi or ShRNA molecules.

In instances where an inflammation-enabling protein may not be an attractive pharmacological target, but may belong to a pathway that harbors other “druggable” sites, the pathways of the inflammation-enabling protein can be identified (using, for example, bioinformatic tools, or published results from large scale transcriptomics or proteomics analyses). In addition, direct experimentation in transfected cells may also be undertaken, for example chromatin immunoprecipitation/sequencing to identify targets of a DNA binding protein, and proteomic analyses to identify substrates of a modifying enzyme (protease, kinase, phosphatase, ubiquitin ligase). Such pathways may also harbor proteins for which small molecule modulators are already available.

### Screening Methods for Therapeutic Agents

The screening methods described herein are designed to capture the relationship between the inflammation-enabling polypeptides' expression and/or activity (collectively referred as the IEP's biological activity) and inflammation (such as, for example, neuroinflammation) to generate valuable information about the agent that is being screened.

Since the expression/activity of the inflammation-enabling polypeptides is shown to be up-regulated during inflammation, the agents identified by the screening methods provided herewith also likely to have the advantage of limiting inflammation and providing therapeutic benefits in conditions associated with exacerbated inflammation if they are shown to reduced the IEP's biological activity.

In screening applications, an agent to be screened is placed in contact with a reagent. A reagent suitable for this type of application has a parameter which is associated (directly or indirectly) to the biological activity of the inflammation-enabling polypeptide. If the biological activity of the inflammation-enabling polypeptide is limited, impeded or event inhibited by contacting with the screened agent, it is expected that such limitation, impediment or inhibition be also reflected in the reagent's parameter. In one embodiment, the parameter's level or measure is lowered (with respect to a control level) when the biological activity of the inflammation-enabling polypeptide is lowered by the presence and/or contact with the screened agent.

In a first embodiment, the reagent is a nucleic acid molecule. The nucleic acid molecule can be a promoter or a fragment thereof derived from one of the inflammation-enabling polypeptides and being capable of modulating the expression of its downstream operably-linked open reading frame (such as, for example, the gene encoding for the inflammation-enabling polypeptide or another suitable reporter gene). When the nucleic acid molecule is a promoter (or a functional fragment associated thereto), the parameter that is being measured is the level of expression of the downstream operably-linked reporter gene, which is associated with the ability (or lack thereof) of the screened agent to limit, impede or inhibit the genetic expression driven from the promoter (or functional fragment). If an agent is capable of limiting, impeding or inhibiting the expression of the reporter gene, it is assumed that the expression from the promoter is also limited, impeded or inhibited by the agent. Alternatively, if the agent is not capable of limiting, impeding or inhibiting the genetic expression of the reporter gene from the promoter, it is assumed that the expression from the promoter is not limited, impeded or inhibited by the agent (and that therefore the agent can lack the therapeutic utility).

In another embodiment, the nucleic acid molecule is a transcript, such as, for example a mRNA or its corresponding cDNA. As used herein, a transcript is a nucleic acid molecule copy of at least one section of a coding region of a gene. The transcript can be, for example, coding for the inflammation-enabling polypeptide. In this particular embodiment, the parameter is the stability and/or amount of the transcript. If the agent is capable of limiting, impeding or inhibiting the stability and/or amount of the transcript encoding the inflammation-associated polypeptide, it is assumed that the level of the transcript is limited, impeded or inhibited by the agent. Alternatively, if the agent is not capable of limiting, impeding or inhibiting the stability and/or amount of the transcript, it is assumed that the level of the transcript is not limited, impeded or inhibited by the agent. In still another embodiment, the nucleic acid molecule is a transcript expressed from a gene whose expression is modulated by the inflammation-enabling polypeptide. This embodiment is particularly advantageous when the inflammation-enabling polypeptide is a transcription factor modulating the expression of other gene (also referred to as downstream genes) or interacts with a transcription factor. In this embodiment, the parameter is the stability and/or amount of the transcript. Even though the transcript of a single gene whose expression is modulated by the inflammation-associated polypeptide can used in this method, it is also contemplated that the level of transcripts associated with at least 5, at least 10, at least 15, at least 20, at least 25, at least 50, at least 100, at least 200 or at least 250 genes whose expression is modulated by the inflammation-enabling polypeptide be used to perform the method. If the agent is capable of modulating the stability and/or amount of at least one, at least 5, at least 10, at least 15, at least 20, at least 25, at least 50, at least 100, at least 200 or at least 250 transcript(s) of gene whose expression is modulated by the inflammation-associated polypeptide, it is assumed that the level of the transcript(s) is modulated by the agent. Alternatively, if the agent is not capable of modulating the stability and/or amount of at least one, at least 5, at least 10, at least 15, at least 20 at least 25, at least 50, at least 100, at least 200 or at least 250 transcript(s) of gene whose expression is modulated by the inflammation-associated polypeptide, it is assumed that the level of the transcript is not modulated by the agent.

If the agent is capable of modulating (in an embodiment, reducing) the amount of the transcript whose expression is modulated (in an embodiment, increased) by the inflammation-associated polypeptide, it is determined that the agent limits, impedes or inhibits the biological activity of the inflammatory enabling polypeptide. Alternatively, if the agent is not capable of modulating (in an embodiment, reducing) the amount of the transcript whose expression is modulated (in an embodiment, increased) by the inflammatory enabling polypeptide, it is determined that the agent cannot limit, impede or inhibit the biological activity of the inflammatory enabling polypeptide.

As shown in Table 6 below, various genes have been listed and their expression is modulated by USP15 and TRIM25. The level of expressions of the genes listed in Table 6 are downregulated in the absence of a functional USP15 and in the absence of functional TRIM25, and their down-regulation is associated with protection from inflammation. By determining if an agent is capable of dowregulating the level of expression of the genes listed in Table 6, it can be determined if the agent will also exhibit anti-inflammatory properties. In an embodiment in which the inflammatory enabling polypeptide is USP15 or TRIM25, the level of expression of at least one or any of the combination of the following genes (presented in Table 6) can be used to determine the biological activity of USP15 and/or TRIM25: Gzmb, Gzma, Fcgr4, Plaur, Ms4a6d, Cebpd, Maff, Socs3, Arrdc2, Mt1, Mt2, Cdknla, Srgn, Zfp36, Map3k6, Fkbp5, Itgb7, Rhoj, Hmgb2, Ucp2, Entpd4 and/or Rbm3. In somes embodiments, the method described herein can use the level of expression of any combination of at least two, three, four, five, six, seven, eight, nine, ten, 11, 12, 13, 14, 15, 16, 17, 18, 19, 20 or 21 of the following genes: Gzmb, Gzma, Fcgr4, Plaur, Ms4a6d, Cebpd, Maff, Socs3, Arrdc2, Mt1, Mt2, Cdknla, Srgn, Zfp36, Map3k6, Fkbp5, Itgb7, Rhoj, Hmgb2, Ucp2, Entpd4 and/or Rbm3. In still another embodiment, the method can use the level of expression of all of the following genes: Gzmb, Gzma, Fcgr4, Plaur, Ms4a6d, Cebpd, Maff, Socs3, Arrdc2, Mt1, Mt2, Cdknla, Srgn, Zfp36, Map3k6, Fkbp5, Itgb7, Rhoj, Hmgb2, Ucp2, Entpd4 and/or Rbm3. In some embodiments, the method can use the level of expression of at least one or any combinations of genes from the IFN-stimulated family (Gzmb, Gzma, Fcgr4, Plaur, Ms4a6d, Cebpd, Maff, Socs3, Mt1, Mt2, Cdknla, Zfp36, Fkbp5 and/or Itgb7) and at least one or any combinations of genes that do not belong to the IFN-stimulated family (Arrdc2, Srgn, Map3k6, Rhoj, Hmgb2, Ucp2, Entpd4 and/or Rbm3).

In a further embodiment, the reagent is a polypeptide. The polypeptide can be, for example, the inflammation-enabling polypeptide or a functional fragment thereof. In this particular embodiment, the parameter can be the stability, amount and/or biological activity of the polypeptide. If the agent is capable of limiting, impeding or inhibiting the stability, amount and/or the biological activity of the inflammation-associated polypeptide, it is assumed that the polypeptide is limited, impeded or inhibited by the agent. Alternatively, if the agent is not capable of limiting, impeding or inhibiting the stability, amount and/or biological activity of the polypeptide, it is assumed that the polypeptide is not limited, impeded or inhibited by the agent.

In still a further embodiment, the reagent is a polypeptide (or a functional fragment thereof) in the same biological pathway as the inflammation-enabling polypeptide. In this particular embodiment, the parameter can be the stability, amount, chemical structure/modification (e.g. phorphorylation state or ubiquitination state) and/or biological activity of the polypeptide. If the agent is capable of limiting, impeding or inhibiting the stability, amount and/or the biological activity of the inflammation-associated polypeptide, it is assumed that the polypeptide is limited, impeded or inhibited by the agent. Alternatively, if the agent is not capable of limiting, impeding or inhibiting the stability, amount and/or biological activity of the polypeptide, it is assumed that the polypeptide is not limited, impeded or inhibited by the agent.

In yet a further embodiment, the reagent is a polypeptide (or a functional fragment thereof) capable of physically interacting (either directly or indirectly) with the inflammation-enabling polypeptide. In this particular embodiment, the parameter can be a measure of association between the inflammation-enabling polypeptide and its binding partner. In instances where the binding of the IEP with its partner can cause a modification of the partner's chemical structure (phosphorylation state or ubiquitination state), the parameter can be the stability, amount, chemical structure (e.g. phorphorylation state) and/or biological activity of the partner. If the agent is capable of limiting, impeding or inhibiting the association of the two polypeptides or the chemical modification of the partner, it is assumed that the inflammation-enabling polypeptide's biological activity is limited, impeded or inhibited by the agent. Alternatively, if the agent is not capable of limiting, impeding or inhibiting the association of the two polypeptides, it is assumed that the inflammation-enabling polypeptide is not limited, impeded or inhibited by the agent.

Even though a single parameter is required to enable the characterization of the agent, it is also provided that more than one parameters of the reagent may be measured and even that more than one reagents may be used in the screening applications provided herewith.

As it will be further discussed below, the contact between the agent and the reagent can be made in vitro (in a reaction vessel that can accommodate the measurement of a reagent's parameter for example) or in vivo (in an animal for example). For screening applications, a suitable in vitro environment for the screening assay described herewith can be an isolated reagent (such as an isolated nucleic acid molecule or an isolated polypeptide) in an appropriate buffer with the necessary other reagents. Another suitable in vitro environment can be a cell (such as a cultured cell) or a cellular extract. When a cultured cell is used, in some embodiment, it is advisable to maintain viability its viability in culture. The cultured cell(s) should be able to provide the reagent. The cell is preferably derived from a myeloid or lymphoid tissue (primary cell culture or cell line). If a primary cell culture is used, the cell may be isolated or in a tissue-like structure.

For in vivo screening applications, a further suitable environment is a non-human model, such as an animal model. If the characterization of the agent occurs in a non-human model, then the model (such as a rodent) is administered with the agent. Various dosage and modes of administration maybe used to fully characterize the agent's ability to prevent, treat and/or alleviate the symptoms of an inflammatory condition.

Once the agent has been contacted with the reagent, a measurement of the parameter of the reagent is made. This assessment may be made directly in the reaction vessel where the contact took place (by using a probe for example) or on a sample of such reaction vessel (by obtaining a biological sample of the cultured cells or non-human animals). The measurement of the parameter of the reagent can be made either at the DNA level, the RNA level and/or the polypeptide level.

The measuring step can rely on the addition of a quantifier specific to the parameter to be assessed to the reaction vessel or a sample thereof. The quantifier can specifically bind to a parameter of the reagent that is being assessed. In those instances, the amount of the quantifier that specifically bound (or that did not bind) to the reagent can be determined to provide a measurement of the parameter of the reagent. In another embodiment, the quantifier can be modified by a parameter of the reagent. In this specific instance, the amount of modified (or unmodified) quantifier will be used to provide a measurement of the parameter of the reagent. In an embodiment, the signal of the quantifier can be provided by a label that is either directly or indirectly attached to a quantifier. For example, the quantifier can be an antibody (monoclonal or polyclonal) that is specific for an epitope generated during the biological activity of the IEP. As such, the complexation of the antibody with the IEP or its target can be used as a surrogate to determine the biological activity of the IEP. For example, in the embodiment in which the IEP is USP15, it is possible to determine its biological activity by measuring the ubiquination state of its targets. One of USP15's targets is TRAM15 which is de-ubiquinated upon USP15's biological activity. As such, by using an antibody specific for ubiquitin, it is possible to determine the presence or absence (and in some embodiments the relative concentration of ubiquinated/deubiquinated) of ubiquination on USP15's targets (such as TRAM15) by using an antibody or a combination of antibodies.

To assess the transcription activity from a promoter of a gene of interest (either associated with the gene encoding the inflammation-enabling polypeptide or associated with target genes whose expression is modulated by the inflammation-enabling polypeptide), a reporter assay can be used. In reporter assays, a reporter vector is placed in contact with an agent and the level of expression (via the amount of the transcript) of the reporter gene is measured to provide for transcription activity from the promoter. The reporter vectors can include, but are not limited to, the promoter region (or a functional fragment thereof) of the gene of interest operably linked to a nucleotide encoding a reporter polypeptide (such as, for example, luciferase, 3-galactosidase, green-fluorescent protein, yellow-fluorescent protein, etc.). Upon the contact of the agent with the reagent, the promotion of transcription from the promoter is measured indirectly by measuring the transcription of the reporter polypeptide. In this particular embodiment, the quantifier is the reporter polypeptide and the signal associated to this quantifier that is being measured will vary upon the reporter polypeptide used.

Alternatively or complementarily, the stability and/or the expression level of the nucleic acid molecules can be assessed by quantifying the amount of the nucleic acid molecule (for example using qPCR or real-time PCR) or the stability of such molecule (for example by providing at least two measurements in function of time). In one assay format, the expression of a nucleic acid molecule in a cell or tissue sample is monitored via nucleic acid-hybridization techniques (in situ hybridization for example). In another assay format, cell lines or tissues can be exposed to the agent to be tested under appropriate conditions and time, and total RNA or mRNA isolated, optionally amplified, and quantified.

In some embodiments, the nucleic acid identity of a nucleic acid molecule or transcripts can be performed. Various methods of determining the nucleic acid sequence of a nucleic acid molecule are known to those skilled in the art and include, but are not limited to, chemical sequencing (e.g. Maxam-Gilbert sequencing), chain termination methods (e.g. Sanger sequencing, and dye-terminator sequencing), restriction digestion-based sequencing (e.g. RFLP), hybridization-based sequencing (e.g. DNA micro-array, RNA micro array, Molecular Beacon probes, TaqMan probes), mass spectrometry-based sequencing, next generation sequencing (e.g. whole exome sequencing, Massively Parallel Signature Sequencing or MPSS, Polony sequencing, pyrosequencing, Illumina™ (Solexa) sequencing, SOLiD™ sequencing, ion semiconductor sequencing, DNA nanoball sequencing, Helioscope™ single molecule sequencing, Single Molecule SMRT™ sequencing, Single Molecule real time (RNAP) sequencing, and Nanopore DNA sequencing).

If the measurement of the parameter is performed at the polypeptide level, an assessment of the polypeptide level of expression can be performed. In an embodiment, specifically the level of expression of the polypeptide is measured for example, through an antibody-based technique (such as an immunohistochemitry, BioPlex, ELISA, flow cytometry, protein micro-array, immunodetection), spectrometry, etc. In one embodiment, this assay is performed utilizing antibodies (or derivatives therefrom) specific to IEPs, binding partners of IEPs or targets of IEPs.

As shown below, some of the inflammation-enabling polypeptide (such as FOXN1) are transcription factors which modulates the expression of downstream target genes involved in the inflammation process. As such, one of the biological activity of these inflammation-enabling polypeptide is to bind to other transcription regulators (also referred to as binding partners) as well as to bind to its target nucleotide sequences to modulate gene expression and, ultimately facilitate the inflammatory response. A reduction of the transcription factor activity of an inflammation-enabling polypeptide, either by limiting the IEP to bind to its binding partner and/or to its target nucleic acid sequence, will be considered useful for preventing, treating and/or alleviating the symptoms associated to an inflammatory disorder.

Evaluation of biological activity can be made by determining the ability of the inflammation-enabling to form a multimeric complex with at least one of its binding partners. In vitro, the reaction mixture can include, e.g. a co-factor, a substrate or other binding partner or potentially interacting fragment thereof. Exemplary binding partners of IRF8 include, but are not limited to, members of the IRF (IRF1, IRF4) or ETS (PU.1). Exemplary binding partners of USP15 include, but are not limited to, E3 ubiquitin/ISG15 ligase (TRIM25), the COP9 signalosome and the receptor-activated SMAD transcription factors. Preferably, the binding partner is a direct binding partner. This type of assay can be accomplished, for example, by coupling one of the components (either the inflammation-enabling polypeptide or its binding partner), with a label such that binding of the labeled component to the other can be determined by detecting the labeled compound in a complex. A component can be labeled with ¹²⁵I, ³⁵S, ¹⁴C, or ³H, either directly or indirectly, and the radioisotope detected by direct counting of radioemmission or by scintillation counting. Alternatively, a component can be enzymatically labeled with, for example, horseradish peroxidase, alkaline phosphatase, or luciferase, and the enzymatic label detected by determination of conversion of an appropriate substrate to product. Competition assays can also be used to evaluate a physical interaction between a test compound and a target.

In another assay format, when the inflammation-enabling polypeptide is a transcription factor, its activity can be measured by quantifying (or semi-quantifying) the expression levels of its target genes whose expression is modulated by the presence and activity of the inflammation-enabling polypeptide. Such measurements can be made, for example, by PCR (such as qPCR). For example, the target genes of IRF8 include, but are not limited to, the genes listed in Tables 1, 2 and/or 3.

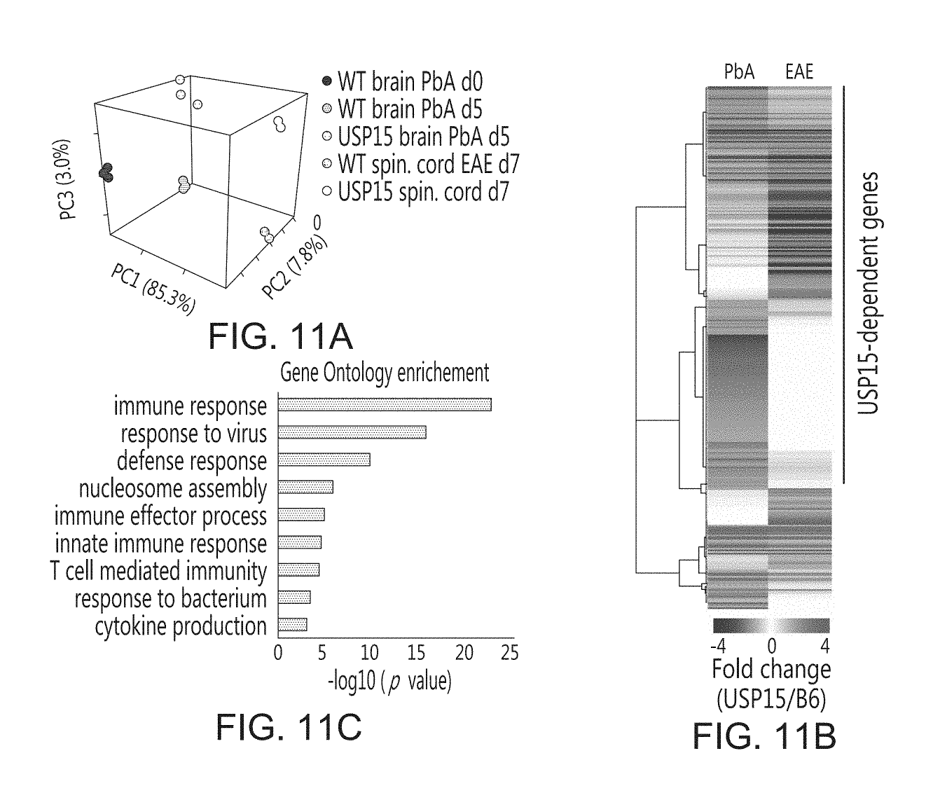

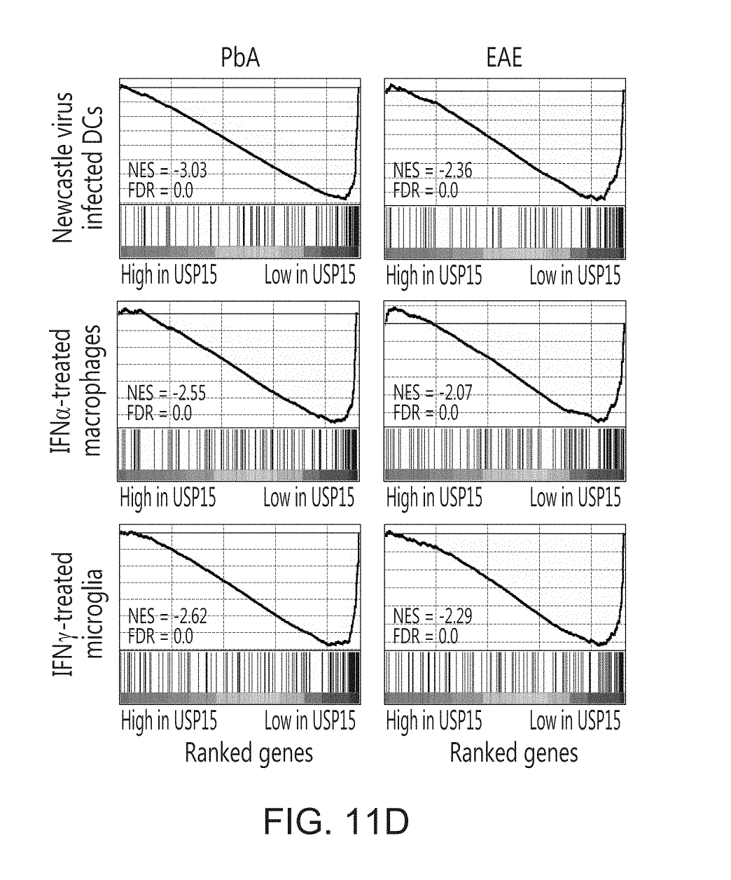

In still another assay format, the inflammaroty-enabling polypeptide is not a transcription factor but its activity can be measured by quantifying (or semi-quantifying) the expression levels of other genes whose expression is modulated indirectly by the presence and activity of the inflammatory polypeptide. Such measurements can be made, for example, by PCR (such as qPCR). For example, the target genes of USP15 include, but are not limited to, the genes listes in FIGS. 11, 12, 14, 17 as well as Table 4. In some embodiments, the target gens of USP15 include one or more of the following genes: OASL1/2, ISG15, IF141, IFIT1/3, IRF7/9, USP18, MT1/2, MX1 and/or PLIN4.

In yet another assay format, when the inflammation-enabling polypeptide is a transcription factor, its activity can be measured by determining the affinity of the transcription to at least one of its nucleic acid recognition motifs. Such measure can be made, for example, through gel-retardation shift assay. For example, the nucleic acid motifs recognized IRF8 include, but are not limited to, GAAAnnGAAA (SEQ ID NO: 1) and GGAAAnnGAAA (SEQ ID NO: 2).

As shown herewith, some inflammation-enabling polypeptides participate in various signaling pathways and interact with other polypeptides (e.g. CCDC88B, ZBTB7B and USP15). An agent capable of limiting the inflammation-enabling polypeptide to participate in the signaling cascade will be considered useful for preventing, treating and/or alleviating the symptoms associated to an inflammatory conditions. To identify agents that modulate with the interaction between the inflammation-enabling polypeptide and its binding partner(s), for example, a reaction mixture containing the reagent (e.g. the inflammation-enabling polypeptide) and the binding partner is prepared, under conditions and for a time sufficient, to allow the two polypeptides to form complex. In order to test if an agent modulates the interaction between the inflammation-enabling polypeptide and its binding partner, the reaction mixture can be provided in the presence and absence of the test agent. The test agent can be initially included in the reaction mixture, or can be added at a time subsequent to the addition of the target and its cellular or extracellular binding partner. Control reaction mixtures are incubated without the test agent or with vehicle. The formation of any complexes between the target product and the cellular or extracellular binding partner is then detected. The formation of a complex in the reaction mixture containing the test compound, but not in the control reaction, indicates that the test agent facilitates the interaction of the inflammation-enabling polypeptide and the interactive binding partner.

In an embodiment, it is possible to detect the formation of the inflammation-enabling polypeptide complex indirectly by measuring the level of expression of a reporter gene whose expression is modulated by the presence (or absence) of the complex.

In still another assay format, the direct interaction between two molecules (especially two polypeptides) can also be detected. Signal generation or detection systems that may be used in the methods include, but are not limited to, fluorescence methods such as fluorescence resonance energy transfer (FRET), bioluminescence resonance energy transfer (BRET), protein fragment complementation assay (PCA), Biomolecular Interaction Analysis (BIA), fluorescence quenching, fluorescence polarization as well as other chemiluminescence, electrochemiluminescence, Raman, radioactivity, colometric methods, hybridization protection assays and mass spectrometry methods.

An additional example for determining the biological activity of an inflammation-enabling polypeptide is the determination of the ability of the inflammation-enabling polypeptide to add and/or remove ubiquitin moieties to proteins. As shown herein, USP15 (an inflammation-enabling polypeptide) is a cysteine hydrolase which cleaves both the isopeptide bonds between ubiquitin (Ub) units, as well as the peptide bond between Ub (C-terminal Glycine) and the bound protein. When USP15 is the inflammation-enabling polypeptide, agent capable of limiting (even inhibiting) its de-ubiquitinase activity are considered useful for preventing, treating and/or alleviating the symptoms associated to the inflammatory condition. For example, a cell-free assay can be used. This assay can be based on the polypeptide-dependent removal of Ub moieties from a ubiquinated immobilized target (for example a ubiquinated SMAD3 and/or a ubiquinated TRIM25), optionally coupled to ELISA-based immunological detection of appearance of free Ub in the reaction. Alternately, if the inflammation-enabling polypeptide shows thiol-dependent hydrolysis of ester, thioester, as well as amide and isopeptide bonds, screens using fluorogenic peptides could also be used as primary assays. Indirectly, the modulation of expression of the inflammation-enabling polypeptide-dependent target (such as, for example, for USP15, TGFβ or BMP or reporter genes (pCAGA12-lux; ID1-BRE-lux)) can be detected.

Cell-free screening assays usually involve preparing a reaction mixture of the reagent and the test compound under conditions and for a time sufficient to allow the two components to interact and bind, thus forming a complex that can be removed and/or detected. In one embodiment, the reagent is anchored onto a solid phase. The reagent-related complexes anchored on the solid phase can be detected at the end of the reaction, e.g. the binding reaction. For example, the reagent can be anchored onto a solid surface, and the test compound, (which is not anchored), can be labeled, either directly or indirectly, with detectable labels discussed herein. Examples of such solid phase include microtiter plates, test tubes, array slides, beads and micro-centrifuge tubes. In one embodiment, an inflammation-enabling protein can be provided which adds a domain that allows one or both of the proteins to be bound to a matrix. Following incubation, the vessels are washed to remove any unbound components, the matrix immobilized in the case of beads, complex determined either directly or indirectly, for example, as described above. Alternatively, the complexes can be dissociated from the matrix, and the level of protein binding or activity determined using standard techniques.

In order to conduct such assay, the non-immobilized component (agent) is added to the coated surface containing the anchored component. After the reaction is complete, unreacted components are removed (e.g. by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g. using a labeled antibody specific for the immobilized component (the antibody, in turn, can be directly labeled or indirectly labeled with, e.g. a labeled anti-lg antibody).

Alternatively, cell free assays can be conducted in a liquid phase. In such an assay, the reaction products are separated from unreacted components, by any of a number of standard techniques, including but not limited to differential centrifugation, chromatography (gel filtration chromatography, ion-exchange chromatography) and/or electrophoresis. Such resins and chromatographic techniques are known to one skilled in the art. Further, fluorescence energy transfer may also be conveniently utilized, as described herein, to detect binding without further purification of the complex from solution.

The assays described herewith can be conducted in a heterogeneous or homogeneous format. Heterogeneous assays involve anchoring either the reagent or the binding partner onto a solid phase, and detecting complexes anchored on the solid phase at the end of the reaction. In homogeneous assays, the entire reaction is carried out in a liquid phase. In either approach, the order of addition of reactants can be varied to obtain different information about the agents being tested. For example, test agents that interfere with the interaction between the reagent and the binding partners, e.g. by competition, can be identified by conducting the reaction in the presence of the test substance. Alternatively, test agents that facilitates preformed complexes, can be tested by adding the test compound to the reaction mixture prior to complexes have been formed. The various formats are briefly described below.

In a heterogeneous assay system, either the reagent or the binding partner, is anchored onto a solid surface (e.g. a microtiter plate), while the non-anchored species is labeled, either directly or indirectly. The anchored species can be immobilized by non-covalent or covalent attachments. Alternatively, an immobilized antibody specific for the species to be anchored can be used to anchor the species to the solid surface.

In order to conduct such assay, the partner of the immobilized species is exposed to the coated surface with or without the agent. After the reaction is complete, unreacted components are removed (e.g. by washing) and any complexes formed will remain immobilized on the solid surface. Where the non-immobilized species is pre-labeled, the detection of label immobilized on the surface indicates that complexes were formed. Where the non-immobilized species is not pre-labeled, an indirect label can be used to detect complexes anchored on the surface, e.g. using a labeled antibody specific for the initially non-immobilized species (the antibody, in turn, can be directly labeled or indirectly labeled with, e.g., a labeled anti-lg antibody). Depending upon the order of addition of reaction components, agents that enable complex formation or that promote the stability of preformed complexes can be detected.

Alternatively, the reaction can be conducted in a liquid phase in the presence or absence of the agent, the reaction products separated from unreacted components, and complexes detected, e.g. using an immobilized antibody specific for one of the binding components to anchor any complexes formed in solution, and a labeled antibody specific for the other partner to detect anchored complexes. Again, depending upon the order of addition of reactants to the liquid phase, test compounds that enable complex or that promote the stability of preformed complexes can be identified.

In an alternate embodiment, a homogeneous assay can be used. For example, a preformed complex of the reagent and the interactive cellular or extracellular binding partner product is prepared in that either the target products or their binding partners are labeled, but the signal generated by the label is quenched due to complex formation. The addition of agent that favors the formation of the complex will result in the generation of a signal below the control value. In this way, agents that promote binding partner interaction can be identified.

In yet another aspect, the reagent can be used as “bait proteins” in a two-hybrid assay or three-hybrid assay, to identify other proteins, which bind to or interact with binding partners and are involved with the inflammation-enabling polypeptide activity. Such binding partners can be activators or inhibitors of signals or transcriptional control.

In another embodiment, the assay for selecting compounds which interact with the inflammation-enabling polypeptide can be a cell-based assay. Useful assays include assays in which a marker of inflammation is measured. The cell-based assay can include contacting a cell expressing the inflammation-enabling polypeptide with an agent and determining the ability of the test compound to modulate (e.g. stimulate or inhibit) the activity of the inflammation-enabling polypeptide, and/or determine the ability of the agent to modulate expression of the inflammation-enabling polypeptide or related proteins in the cell. Cell-based systems can be used to identify compounds that decrease the expression and/or activity and/or effect of the inflammation-enabling polypeptide. Such cells can be recombinant or non-recombinant, such as cell lines that express the inflammation-enabling gene. In some embodiments, the cells can be recombinant or non-recombinant cells which express a binding partner of the inflammation-enabling polypeptide. Exemplary systems include mammalian or yeast cells that express the inflammation-enabling polypeptide (for example from a recombinant nucleic acid). In utilizing such systems, cells are exposed to agents suspected of decreasing expression and/or activity of the inflammation-enabling polypeptide. After exposure, the cells are assayed, for example, for expression or activity of the inflammation-enabling polypeptide. A cell can be from a stable cell line or a primary culture obtained from an organism (for example an organism treated with the agent).

In addition to cell-based and in vitro assay systems, non-human organisms, e.g. transgenic non-human organisms or a model organism, can also be used. A transgenic organism is one in which a heterologous DNA sequence is chromosomally integrated into the germ cells of the animal. A transgenic organism will also have the transgene integrated into the chromosomes of its somatic cells. Organisms of any species, including, but not limited to: yeast, worms, flies, fish, reptiles, birds, mammals (e.g. mice, rats, rabbits, guinea pigs, pigs, micro-pigs, and goats), and non-human primates (e.g. baboons, monkeys, chimpanzees) may be used in the methods described herein.

A transgenic cell or animal used in the methods described herein can include a transgene that encodes the inflammation-enabling polypeptide, a corresponding fragment or a corresponding variant (such as the mutant or non-functional IEP described herein). The transgene can encode a protein that is normally exogenous to the transgenic cell or animal, including a human protein, e.g., a human inflammation-enabling polypeptide or one of its binding partner. The transgene can be linked to a heterologous or a native promoter. Methods of making transgenic cells and animals are known in the art.

In another assay format, the specific activity of the inflammation-enabling polypeptide, normalized to a standard unit, may be assayed in a cell-free system, a cell line, a cell population or animal model that has been exposed to the agent to be tested and compared to an unexposed control cell-free system, cell line, cell population or animal model. The specific activity of an screened compound can also be assessed using inflammation-enabling polypeptide-deficient systems (e.g. where at least one copy of the gene codes for a non-functional inflammation-enabling polypeptide).

In one embodiment, the measuring step includes (or consists in) measuring the test level of the parameter below a control level (usually associated with a lack of prevention, treatment and/or alleviation of symptoms associated with the inflammatory condition). In this embodiment, the presence of the measurement is indicative of the ability of the screened agent to prevent, treat and/or alleviate the symptoms associated with the inflammatory condition. On the other hand, the absence of the measurement is indicative of the lack of ability of the screened agent to prevent, treat and/or alleviate the symptoms associated with the inflammatory condition.

In other embodiments, once the measurement has been made, the value associated thereto can be extracted and compared to a control value. In screening application, the effect of the agent on the inflammation-enabling polypeptide's expression and/or activity is compared to a control value. In an embodiment, the control value is associated with a lack of prevention, treatment and/or alleviation of symptoms of the inflammatory condition and as such, agents useful in the prevention, treatment and/or alleviation of symptoms of the inflammatory condition are capable of decreasing the measured parameter below the control value. In this embodiment, agents which are not considered useful in the prevention, treatment and/or alleviation of symptoms of the inflammatory conditions will present a parameter which is either equal to or higher than the control value.

In another embodiment, the control value is associated with prevention, treatment and/or alleviation of symptoms of the inflammatory condition and as such the measured parameter associated agents useful in the prevention, treatment and/or alleviation of the inflammatory condition equal to or lower than the control value. In such embodiment, agents that are not useful in the prevention, treatment and/or alleviation of symptoms of the inflammatory condition show a test value that is higher than the control value.

In an embodiment, the comparison can be made by an individual. In another embodiment, the comparison can be made in a comparison module. Such comparison module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPUNPU), a physics processing unit (PPU), a digital signal processor and a network processor. The application is coupled to the processor and configured to determine the effect of the agent on the parameter of the based reagent with respect to the control value. An output of this comparison may be transmitted to a display device. The memory, accessible by the processor, receives and stores data, such as measured parameters of the reagent or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

Once the comparison between the parameter of the reagent and the control value is made, then it is possible to characterize the agent. This characterization is possible because, as shown herein, the expression and/or activity of the inflammation-enabling polypeptide is downregulated for agents capable of treating, preventing and/or alleviating the symptoms associated an inflammatory disorder.

In an embodiment, the characterization can be made by an individual. In another embodiment, the characterization can be made with a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to characterize the individual or the agent being screened. An output of this characterization may be transmitted to a display device. The memory, accessible by the processor, receives and stores data, such as measured parameters of the reagent or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

The screening methods described herein can be used to determine an agent's ability to prevent, treat or alleviate the symptoms of an inflammatory condition. The premise behind this screening method is that the inflammation-enabling polypeptide activity or expression is upregulated during inflammation. As such, by assessing if a downregulation of the inflammation-enabling polypeptide's activity or expression made by the agent, it can be linked to its ability to prevent, treat or alleviate the symptoms of an inflammatory disorder. In these methods, the control value may be the parameter of the reagent in the absence of the agent. In this particular embodiment, the parameter of the reagent can be measured prior to the combination of the agent with the reagent or in at least two replicates of the same reaction vessel where one of the screening system does not comprise the agent. The control value can also be the parameter of the reagent in the presence of a control agent that is known not to limit inflammation or prevent/treat/alleviate the symptoms of an inflammatory disorder. Such control agent may be, for example, a pharmaceutically inert excipient. The control value can also be the parameter of the reagent obtained from a reaction vessel comprising cells or tissues from a healthy subject that is not afflicted by inflammation. The control value can also be a pre-determined value associated with a lack of inflammation (or inflammatory disorder). The ability of the agent is determined based on the comparison of the value of the parameter of the reagent with respect to the control value.

The agent is characterized as being able to prevent, treat or alleviate the symptoms of an inflammatory disorder when the value of the parameter of the reagent is lower than the control value. On the other hand, the agent is characterized as lacking the ability to prevent, treat or alleviate the symptoms of an inflammatory disorder when the measurement of the parameter of the reagent is lower than or equal to the control value.

The present disclosure also provides screening systems for performing the characterizations and methods described herein. These systems comprise a reaction vessel for placing the agent and the reagent, a processor in a computer system, a memory accessible by the processor and an application coupled to the processor. The application or group of applications is(are) configured for receiving a test value of a parameter of the reagent in the presence of the agent; comparing the test value to a control value and/or characterizing the agent in function of this comparison.

The present disclosure also provides a software product embodied on a computer readable medium. This software product comprises instructions for characterizing the individual or the agent according to the methods described herein. The software product comprises a receiving module for receiving a test value of a parameter of the reagent in the presence of an agent; a comparison module receiving input from the measuring module for determining if the test value is lower than, equal to or higher than a control value; a characterization module receiving input from the comparison module for performing the characterization based on the comparison.

In an embodiment, an application found in the computer system of the system is used in the comparison module. A measuring module extracts/receives information from the reaction vessel with respect to the parameter of the reagent. The receiving module is coupled to a comparison module which receives the value(s) of the parameter of the reagent and determines if this value is lower than, equal to or higher than a control value. The comparison module can be coupled to a characterization module. In another embodiment, an application found in the computer system of the system is used in the characterization module. The comparison module is coupled to the characterization module which receives the comparison and performs the characterization based on this comparison. In a further embodiment, the receiving module, comparison module and characterization module are organized into a single discrete system. In another embodiment, each module is organized into different discrete system. In still a further embodiment, at least two modules are organized into a single discrete system.

### Non-Functional IEP and Heterozygote Animals for the Inflammation-Enabling Polypeptide for Validating Therapeutic Compounds

In some instances, it may be necessary to validate the results of an in vitro screening (from either a cell-free or a cell-based assay) by using a control (e.g., mutant or non-functional) IEP or an animal model. In other instances, it may also be necessary to perform the initial screening with the control non-functional IEP directly in an animal. For those circumstances, a control IEP as well as an heterozygote animal are herewith provided to provide an animal model of inflammation for determining if a particular agent is useful for the prevention, treatment or alleviations of symptoms associated with an inflammatory condition.

The control IEP polypeptide can be an isolated polypeptide. The control IEP is a fragment or a variant of the “wild-type” IEP polypeptide described herein. The control IEP has a reduced biological activity when compared to the wild-type IEP.

One of the IEP is the USP15 polypeptide. The wild-type USP15 has ubiquitin carboxyl-terminal hydrolase 15 activity. Amongst other things, the wild-type USP15 is capable of binding to and de-ubiquitinating the TRIM25 ligase. As used in the context of the present disclosure, a control or mutant USP15 polypeptide is derived from the wild-type USP15 polypeptide and can have some de-ubiquitinating activity, albeit to a lower level than the wild-type USP15 polypeptide. Wild-type USP15 is expressed in the mouse and the murine USP15 polypeptide can have the amino acid sequence of SEQ ID NO: 53. Wild-type USP15 is also expressed in humans and the human USP15 polypeptide can have the amino acid sequence of SEQ ID NO: 55 (isoform 1) or SEQ ID NO: 57 (isoform 2). In the context of the present disclosure, the control or mutant IEP corresponding to the wild-type USP15 can have one or more amino acid substitutions when compared to the wild-type USP15 polypeptide. For example, as shown herein, control USP15 polypeptides having an amino acid substitution (especially a leucine to arginine substitution) at position 749 of SEQ ID NO: 53 and 55 or at position 720 of SEQ ID NO: 57 have been shown to have reduced biological activity when compared to the wild-type USP15 polypeptide.

As such, in an embodiment, the control or mutant USP15 polypeptide can have the amino acid sequence of SEQ ID NO: 54, 56 or 58. In still another embodiment, the control USP15 can be a fragment of SEQ ID NO: 54, 56 or 58. As used in the context of the present disclosure, the term “fragment” refers to a polypeptide having at least one less amino acid residues that the corresponding polypeptide. The deletion can occur either at the N-, at the C-terminus or at both the N- and C-terminus of the polypeptide. In the context of the present disclosure, when the control USP15 is a fragment of the amino acid sequence of SEQ ID NO: 54, 56 or 58, it has at least at least 100, 200, 300, 400, 500, 600, 700, 800, 900 or more consecutive amino acid residues of the amino acid sequence of SEQ ID NO: 54, 56 or 58. In yet another embodiment, the control USP15 can be a variant of the amino acid sequence of SEQ ID NO: 54, 56 or 58. In the context of the present disclosure, a “variant” is referred to as an amino acid sequence having at least one or more amino acid substation, deletion or addition. In some embodiment, the variant of the amino acid sequence of SEQ ID NO: 54, 56 or 58 has at least one amino acid substitution. For example, the variant of the amino acid sequence of SEQ ID NO: 58 can include a substitution at position 269 (a cysteine to alanine substitution for example), 783 (a cysteine to alanine substitution for example) and/or at position 952 (a serine to leucine substitution for example). In still another example, the variant of the amino acid sequence of SEQ ID NO: 54 or 56, it can include a substitution at position 298 (a cysteine to alanine substitution for example) and/or 312 (a cysteine to alanine substitution for example).

As discussed herein, mouse bearing two copies of a gene coding for an non-functional or mutant inflammation-enabling polypeptide are not capable of inducing, mounting a complete inflammatory response when challenged with P. berghei. The animals, homozygote at the loci encompassing the gene coding for the inflammation-enabling polypeptide, are expected not to show a differential response to potential anti-inflammatory agents since they cannot induce an inflammatory response.

On the other hand, mouse bearing two copies of a gene coding for a functional inflammation-enabling polypeptide are capable of inducing, mounting and maintaining a complete and full inflammatory response when challenged with P. berghei. The animals, homozygote at the loci encompassing the gene coding for the inflammation-enabling polypeptide, are expected to show a very little differential response to potential anti-inflammatory agents because they mount a robust inflammatory response and died very rapidly.

Consequently, the present disclosure provides an animal heterozygote at the loci encompassing the gene coding for the inflammation-enabling polypeptide. As a first gene copy, a functional (wild-type) inflammation-enabling polypeptide is provided and allows the animal to mount an inflammatory response. Examplary wild-type IEPs include, but are not limited to, the wild-type USP15 polypeptide described herein (having, for example, the amino acid sequence of SEQ ID NO: 53, 55 or 57). As the second gene copy, a non functional (control or mutant) inflammation-enabling polypeptide is provided and limits the animal in mounting a robust inflammatory response. The non-functional copy can be, for example, encoding a mutated protein having lost its biological activity, a knock-out gene or a knocked-in gene. An exemplary wild-type IEPs include, but are not limited to, the control of mutant USP15 polypeptide described herein (having, for example, the amino acid sequence of SEQ ID NO: 54, 56 or 58). Consequently, the animal herewith provided is capable of mounting an inflammatory response, but not a robust one, and are capable of showing a differential response to potential anti-inflammatory agents. The present disclosure also provides the use of the heterozygous animals for assessing the usefulness of a screened agent for preventing, treating and/or alleviating the symptoms associated to an inflammatory condition. This is defined as a sensitized screen for inhibitors against a given target identified by the method described in the present disclosure.

In order to determine if an agent is capable of treating or alleviating the symptoms associated with an inflammatory condition, an inflammatory response is first induced (by administering a trigger) in the animal and then a screened agent is administered. On the other hand, to determine if an agent is capable of preventing the onset of an inflammatory condition, the screened agent is first administered and then a trigger is provided to the animal. The trigger (e.g. an infectious agent such as, for example, P. berghei), in the absence of an agent, is capable of inducing an inflammatory response in the animal.

Once the agent and the inflammatory trigger have been administered, then a parameter of an inflammatory response is measured. In embodiments where an infectious agent is administered as the inflammation trigger, and it is known that such trigger ultimately causes death in infected animals, the parameter may be the survival rate or the death rate. Although death is the end point of acute inflammation in the P. berghei infection model, other intermediate phenotypes precede death and are predictor of lethality; these include appearance of neurological symptoms such as fever, termors, lethargy, hind limb paralysis and coma. Other inflammation-associated parameters (surrogate markers) can also be measured such as, immune cells number and types, cytokine profiles, chemokine profiles, immunoglobulin profiles, edema, blood-brain-barrier permeability, etc.

In an embodiment, the measure can also include measuring a parameter in function of a control value. For example, when blood-brain-barrier permeability is the parameter that is measured, the measuring step can include measuring the blood-brain-barrier permeability above a control value associated with a non-inflammatory state. The presence of the measure (e.g. because the measure is higher than the control value) is indicative that the agent is useful. On the other hand, the absence of the measure (e.g. because the measure is below than the control value) is indicative that the agent lacks the utility.

Once measured, the test parameter (also referred to as the test level) can be compared to a control and the agent is characterized based on this comparison. Such control can be associated with the prevention, treatment and/or alleviation of the symptoms associated to the inflammatory condition or with the lack of prevention, treatment and/or alleviation of the symptoms associated with the inflammatory condition. The control can be obtained, for example, from animals that have not been treated with the agent or that have been treating with a mock agent that as been shown not to prevent, treat or alleviate the symptoms associated with the inflammatory condition (for example a pharmaceutically acceptable excipient).

For example, when the parameter that is assessed is the survival rate and the control that is used is associated with a lack of prevention, treatment and/or alleviation of the symptoms associated with the inflammatory condition, an agent is considered useful if the survival rate of the treated animals is higher than the survival rate of the non-treated/mock treated animals (e.g. control associated with a lack of prevention, treatment and/or alleviation of the symptoms associated with the inflammatory condition). On the other hand, the agent will not be considered useful (or will be considered as lacking the utility) if the survival rate of the treated animals is equal to or higher than the survival rate of the non-treated/mock treated animals (e.g. control associated with a lack of prevention, treatment and/or alleviation of the symptoms associated with the inflammatory condition).

In order to conduct use these animal models, it is envisaged that a copy of a single gene of an inflammation-enabling polypeptide (such as, for example, CCDC88B, FOXN1 or USP15) be modified to code for a non-functional polypeptide. However, it is also contemplated that a copy of at least two distinct genes each coding for a different inflammation-enabling polypeptide be modified in the animal (such as for example, a combination of CCDC88B and FOXN1, a combination of CCDC88B and USP15 or a combination of FOXN1 and USP15). It is also completed that a copy at least three distinct genes each coding for a different-inflammation-enabling polypeptide be modified in the animal (such as, for example, a combination of CCDC88B, FOXN1 and USP15). In some instances, it may be advisable to provide further genetic mutations associated with other loci implicated in inflammation in the animal. For example, it is possible to obtain an animal additionally bearing a copy of a non-functional gene coding for at least one of LYST, ZBTB7B, BPGM1, RASAL3, JAK3, THEMIS, IRGM1, IRF1 and IRF8, at least two of LYST, ZBTB7B, BPGM1, RASAL3, JAK3, THEMIS, IRGM1, IRF1 and IRF8, at least three of JAK3, THEMIS, IRGM1, IRF1 and IRF8, at least four of LYST, ZBTB7B, BPGM1, RASAL3, JAK3, THEMIS, IRGM1, IRF1 and IRF8 or all five JAK3, THEMIS, IRGM1, IRF1 and IRF8.

Methods for providing a non-functional gene copy include, but are not limited to, chemical mutagensis (e.g. ENU-directed mutagenesis) and transgenesis. The heterozygote animal can also be obtained by breeding an animal homozygote at the loci encompassing the gene coding for a non-functional inflammation-enabling polypeptide with an animal homozygote at the loci encompassing the gene coding for a functional inflammation-enabling polypeptide.

Various animals can be obtained and used in this method. In an embodiment, the animal is a non-human mammal, such as a mammal. In some embodiments, the animal is rodent (a mouse for example).

### Predictive Methods Using the Inflammation-Enabling Polypeptide Targets

The predictive methods described herein are designed to capture the relationship between the biological activity of the inflammation-enabling polypeptides and inflammatory conditions (such as, for example, neuroinflammation) to generate valuable information about the therapeutic agent that is being administered to an individual or the individual that is being characterized. As shown herein, the biological activity of the inflammation-enabling polypeptides is positively correlated with the ability of the individual to induce, mount and/or sustain an inflammatory response (such as, for example, neuroinflammation). When the inflammation-enabling polypeptide(s) is(are) expressed and functional in the individual (e.g. when the inflammation-enabling polypeptides show biological activity), it indicates that such individual is capable of developing or maintaining an inflammatory response (such as, for example, neuroinflammation). Alternatively, when the inflammation-enabling polypeptide(s) is(are) expressed at a lower level, are not expressed or are not functional in an individual (e.g. when the inflammation-enabling polypeptides show reduced or non-existent biological activity), it indicates that such individual is not capable of developing or maintaining a full and complete inflammatory response (such as, for example, neuroinflammation).

This correlation between the biological activity of the inflammation-enabling polypeptide and inflammation also provides a basis for determining if a therapeutic regimen is successful in the individual, either for preventing, treating and/or alleviating the symptoms associated to the inflammatory condition (such as, for example, neuroinflammation). If the biological activity of the inflammation-enabling polypeptide(s) is(are) reduced by the therapeutic regimen, it is assumed that the regimen is successful in preventing, treating and/or alleviating the symptoms associated with the inflammatory response (such as, for example, associated with neuroinflammation) in the individual. Alternatively, if the biological activity of the inflammation-enabling polypeptide(s) is(are) not reduced (e.g. remains the same or increases) upon the administration of the therapeutic regimen, it is assumed that the regimen is not successful for preventing, treating and/or alleviating the inflammatory condition (such as, for example, neuroinflammation) in the individual.

This correlation between the biological activity of the inflammation-enabling polypeptide and inflammation further provides a basis for assessing the risk of an individual of developing or being afflicted with an inflammatory condition (such as, for example, neuroinflammation). If the biological activity of the inflammation-enabling polypeptide(s) is(are) higher in the screened individual with respect to a control individual (for example a healthy individual known not to be at risk of developing or being afflicted with an inflammatory condition), then it is assumed that the screen individual is at risk of developing (predisposed) or being afflicted with an inflammatory condition. Alternatively, if the biological activity of the inflammation-enabling polypeptide(s) in the screened individual is(are) similar or lower than the biological activity observed in a control individual (for example a healthy individual known not to be at risk of developing or being afflicted with an inflammatory condition), then it is assumed that the screened individual is not at risk (predisposed) of developing or being afflicted with an inflammatory condition.

In these predictive applications, a biological sample of an individual is obtained and can placed in a reaction vessel. The biological sample comprises an analyte (also referred to as a reagent) associated with an inflammation-enabling polypeptide. In the assays, the reaction vessel can be any type of container that can accommodate the determination of the parameter of the analyte/reagent.

Once the biological sample has been provided, one of the parameters of the analyte/reagent can be measured. This measure may be made directly in the reaction vessel (by using a probe) or on a sample of such reaction vessel. As indicated above, in the screening section, the measure can be made either at the DNA level, the RNA level and/or the polypeptide level. As also indicated above in the screening section, the analyte/reagent can either be the inflammation-enabling polypeptide itself (corresponding transcript and/or gene) and/or for a polypeptide associated with the biological activity inflammation-enabling polypeptide (corresponding transcript and/or gene). The measure can concern a single inflammation-enabling polypeptide or a combination of inflammation-enabling polypeptides (for example two or three inflammation-enabling polypeptides).

In an embodiment, the measuring step can also comprise measuring the parameter in function of a control value. For example, when IEP's amount is the parameter that is measured, the measuring step can include measuring the IEP's amount below a control value associated with a non-inflammatory state. The presence of the measure (e.g. because the measure is lower than the control value) is indicative that the agent is useful in the treated individual. On the other hand, the absence of the measure (e.g. because the measure is higher than the control value) is indicative that the agent lacks the utility in the treated individual.

The methodology described in the “Screening methods for therapeutic agents” section above for measuring the reagent and obtaining a test level of the parameter of the biological activity of the IEP can be used in the predictive methods described herein.

Once the measure of the parameter has been made, a comparison to a control level is done. For example, if the method is for determining the usefulness of a therapeutic agent in an individual and the analyte/reagent that is being measure is the mRNA expression profile (identity and/or amount of transcripts of a plurality of genes whose expression is modulated by an inflammation-enabling polypeptide, for example IRF8 and/or USP15), the comparison step can comprise determining the identity of mRNA transcripts and/or the amount of each mRNA transcripts associated with a plurality of genes whose expression is modulated by an inflammation-enabling polypeptide. The mRNA profile obtained (either the genetic identity of the transcripts and/or the amount of the transcripts) is compared either to a healthy mRNA profile derived from a healthy individual (a control associated with a lack of an inflammatory condition) and/or to a disease mRNA profile derived from an afflicted and untreated individual (a control associated with the presence of an inflammatory condition and consequently the lack of prevention, treatment and/or alleviation of symptoms) to determine to which mRNA profile the obtained mRNA profile is more similar. If the obtained mRNA profile is more similar to the healthy mRNA profile than to the disease mRNA profile, then it can be assumed that the therapeutic agent is useful in the prevention, treatment and/or alleviation of symptoms associated to an inflammatory condition. Alternatively, if the obtained mRNA profile is more similar to the disease mRNA profile than to the healthy mRNA profile, then it can be assumed that the therapeutic agent is not useful in the prevention, treatment and/or alleviation of symptoms associated to an inflammatory condition.

In another example, if the method is for determining the predisposition or affliction of in an individual to the inflammatory condition and the analyte/reagent that is being measure is the biological activity of the inflammation-enabling polypeptide (for example USP15), the comparison step can comprise comparing the test level of biological activity in the screened individual identity to a control level of the inflammation-enabling polypeptide. The biological activity measured can be compared either to a healthy control level that can be derived from a healthy individual (a control associated with a lack of an inflammatory condition) and/or to a disease control level derived from an afflicted and untreated individual (a control associated with the presence of an inflammatory condition and consequently the lack of prevention, treatment and/or alleviation of symptoms) to determine to which control level the measured biological activity is more similar. If the measured test level is more similar (e.g. closer) to the healthy level than to the disease level, then it can be assumed that the individual is either not predisposed (or at least as predisposed as the healthy individual) or not afflicted with the inflammation-enabling polypeptide. Alternatively, if the measured is more similar (e.g. closer) to the disease control level than to the healthy control level, then it can be assumed that the individual is either predisposed or afflicted by the inflammatory condition.

In an embodiment, the comparison can be made by an individual. In another embodiment, the comparison can be made in a comparison module. Such comparison module may comprise a processor and a memory card to perform an application. The processor may access the memory to retrieve data. The processor may be any device that can perform operations on data. Examples are a central processing unit (CPU), a front-end processor, a microprocessor, a graphics processing unit (PPUNPU), a physics processing unit (PPU), a digital signal processor and a network processor. The application is coupled to the processor and configured to compare the test level to a control level. An output of this comparison may be transmitted to a display device. The memory, accessible by the processor, receives and stores data or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

Once the comparison is made, then it is possible to characterize the therapeutic agent's usefulness or the individual's predisposition. This characterization is possible because, as shown herein, the biological activity of at least one inflammation-enabling polypeptide is associated with the onset and maintenance of inflammation.

In an embodiment, the characterization can be made by an individual. In another embodiment, the characterization can be made with a processor and a memory card to perform an application. The processor may access the memory to retrieve data. The processor may be any device that can perform operations on data. Examples are a central processing unit (CPU), a front-end processor, a microprocessor, a graphics processing unit (PPU/VPU), a physics processing unit (PPU), a digital signal processor and a network processor. The application is coupled to the processor and configured to characterize the individual being tested. An output of this characterization may be transmitted to a display device. The memory, accessible by the processor, receives and stores data or any other information generated or used. The memory may be a main memory (such as a high speed Random Access Memory or RAM) or an auxiliary storage unit (such as a hard disk, a floppy disk or a magnetic tape drive). The memory may be any other type of memory (such as a Read-Only Memory or ROM) or optical storage media (such as a videodisc or a compact disc).

The predictive methods described herein are useful for determining if a therapeutic regimen that is being administered is useful or not in preventing, treating and/or alleviating the symptoms associated with an inflammatory condition. If a therapeutic regimen is useful, then it is assumed that the biological activity of at least one inflammation-enabling polypeptide will be reduced in the individual upon the administration of at least one dose of therapeutic agent. In some instances, it may be necessary to administer the therapeutic agent more than once to observe some therapeutic benefit(s). As such, it is possible to repeat the method in time to determine if the therapeutic agent (i) continues to provide therapeutic benefit or (ii) can provide therapeutic benefits when administered more than once. If warranted, it is also possible to perform this method before and after the intake of the therapeutic agent.

The predictive methods described herein are useful for determining if an individual is predisposed to/afflicted by an inflammatory condition. Such methods can be used in conjunction with other methods to provide a diagnosis of an inflammatory condition in the individual. If the individual is predisposed/afflicted, then it is assumed that the biological activity of at least one inflammation-enabling polypeptide will be higher than a control healthy individual. In some instances, it may be necessary to perform the method at more than once to determine if the predisposition further increases or the inflammatory condition continue to worsen.

The predictive methods presented herein can also be useful in classifying individuals already diagnosed with an inflammatory condition based on the level of activity of the inflammation-enabling polypeptides. The predictive methods presented herein can also be useful in determining the re-occurrence of an inflammatory condition in individuals previously diagnosed (and, optionally treated) with the condition.

The present disclosure also provides predictive systems for performing the characterizations and methods described herein. These systems comprise a reaction vessel for placing the biological sample, a processor in a computer system, a memory accessible by the processor and an application coupled to the processor. The application or group of applications is(are) configured for receiving a test level of parameter of the reagent; comparing the test level to a control level and/or characterizing the therapeutic agent/individual in function of this comparison.

The present disclosure also provides a software product embodied on a computer readable medium. This software product comprises instructions for characterizing the individual according to the methods described herein. The software product comprises a receiving module for receiving a test level from a parameter of a reagent in a biological sample; a comparison module receiving input from the measuring module for comparing the test level to a control level; a characterization module receiving input from the comparison module for performing the characterization based on the comparison.

In an embodiment, an application found in the computer system of the system is used in the comparison module. A measuring module extracts/receives information from the reaction vessel with respect to the test level. The receiving module is coupled to a comparison module which receives the value(s) of the test level and determines if this value is identical or different from the control level. The comparison module can be coupled to a characterization module.

In another embodiment, an application found in the computer system of the system is used in the characterization module. The comparison module is coupled to the characterization module which receives the comparison and performs the characterization based on this comparison.

In a further embodiment, the receiving module, comparison module and characterization module are organized into a single discrete system. In another embodiment, each module is organized into different discrete system. In still a further embodiment, at least two modules are organized into a single discrete system.

### Research Tools

The present disclosure also provides research tools based either on the altered (e.g. reduced) expression of inflammatory enabling polypeptides (IEP) or the expression of a mutant IEP.

One of the research tools that can be useful for the characterization of inflammation conditions, are mutant polypeptides of IEP. Mutant IEPs, when expressed in a subject, have the ability to prevent the onset and/or maintenance of an inflammatory condition in the subject. Mutant IEPs include truncated versions of IEP as well mutated versions of IEP having the ability to prevent the onset and/or maintenance of an inflammatory condition in the subject. A truncated version of an IEP is a polypeptide which is at least one amino acid shorter than the wild-type IEP. A mutated version of an IEP is a polypeptide which has at least one amino acid substitution or addition when compared to the wild-type IEP. A polypeptide or fragment thereof is “substantially homologous” or “substantially identical” to another if, when optimally aligned (with appropriate insertions and/or deletions) with the other polypeptide, there is nucleotide sequence identity in at least 60% of the nucleotide bases, usually at least 70%, more usually at least 80%, preferably at least 90%, and more preferably at least 95-98% of the amino acid residues. The length of homology or identity comparison, as described, may be over longer stretches, and in certain embodiments will often be over a stretch of at least 5 amino acids, at least 14 amino acids, at least 20 amino acids, more usually at least 24 amino acids, typically at least 28 amino acids, more typically at least 32 amino acids, and preferably at least 36 or more amino acids. In an embodiment, the mutant IEP is a truncated or mutated version of CCDC88B or USP15. One examplary mutant IEP is the polypeptides having the amino acid sequence shown in SEQ ID NO: 4, 54, 56 or 58 (as well as corresponding fragments or variants thereof).

Another research tool that can be used is a nucleic acid vector encoding the isolated IEP or mutant IEP described herein. As used herein, the term “vector” to expression vectors (derived, for example, from retroviruses, adenovirus, herpes or vaccinia viruses, bacterial or fungal plasmids) as well as integrative vectors (designed to, for example, specifically disrupt the appropriate expression of an IEP).

Cells (in some embodiments isolated cells) can also be used in the methods described herein. Some cells can be designed to be heterozygous for a gene encoding an inflammatory enabling polypeptide (IEP). In such cells, two different gene copy at the same loci are found. For example, the cell can bear a first gene copy encoding IEP and a second gene copy encoding a mutant of the IEP. Other cells can be designed to be homozygous for a gene encoding a mutant inflammatory enabling polypeptide (IEP). Combinations of heterozygous and homozygous cells are also contemplated. In some embodiments, the cells are going to be transgenic cell and can even bear the vectors described herein.

Animals (in some embodiments cells and tissues isolated therefrom) can also be used in the methods described herein. Some animals can be designed to be heterozygous for a gene encoding an inflammatory enabling polypeptide (IEP). In such animals, two different gene copy at the same loci are found. For example, the animal can bear a first gene copy encoding IEP and a second gene copy encoding a mutant of the IEP. Other animals can be designed to be homozygous for a gene encoding a mutant inflammatory enabling polypeptide (IEP). Combinations of heterozygous and homozygous animals are also contemplated. In some embodiments, the animals are going to be transgenic ones and can even bear the vectors described herein.

### Therapeutic Method

The present disclosure does hereby provide that the biological activity of the inflammation-enabling polypeptide(s) is increased with inflammation and that, conversely a reduction in the biological activity of the inflammation-enabling polypeptide(s) would be beneficial for preventing, treating and/or alleviating the symptoms associated to an inflammatory disorder. Consequently, it is expected that the reduction in expression of at least one (or two or three) inflammation-enabling polypeptide would be beneficial for reducing the level or length of a pathologic inflammation response. The present application thus provides a method for preventing, treating and/or alleviating the symptoms associated to an inflammatory conditions based on the inhibition of the biological activity of at least one IEP as well as the use of IEP's inhibitors for the prevention, treatment and/or alleviation of symptoms associated with an inflammatory condition.

The agents that can be administered for this purpose include, but are not limited to, small molecules, peptides, antibodies, nucleic acids, analogs thereof, multimers thereof, fragments thereof, derivatives thereof and combinations thereof.

In order to limit and even shut down the expression of the inflammation-enabling polypeptide, it is possible to use a nucletotide-based agent such as, an antisense nucleic acid or oligonucleotide wholly or partially complementary to, and can hybridize with, a target nucleic acids encoding the inflammation-enabling polypeptide (either DNA or RNA). For example, an antisense nucleic acid or oligonucleotide can be complementary to 5′ or 3′ untranslated regions, or can overlap the translation initiation codon (5′ untranslated and translated regions) of at least one nucleic acid molecule encoding for an inflammation-enabling polypeptide. As non-limiting examples, antisense oligonucleotides may be targeted to hybridize to the following regions: mRNA cap region; translation initiation site; translational termination site; transcription initiation site; transcription termination site; polyadenylation signal; 3′ untranslated region; 5′ untranslated region; 5′ coding region; mid coding region; 3′ coding region; DNA replication initiation and elongation sites. Preferably, the complementary oligonucleotide is designed to hybridize to the most unique 5′ sequence of a nucleic acid molecule encoding for an inflammation-enabling polypeptide, including any of about 15-35 nucleotides spanning the 5′ coding sequence.

In another embodiment, oligonucleotides can be constructed which will bind to duplex nucleic acid (i.e. DNA:DNA or DNA:RNA), to form a stable triple helix containing or triplex nucleic acid. Such triplex oligonucleotides can inhibit transcription and/or expression of a nucleic acid encoding an inflammation-enabling polypeptide. Triplex oligonucleotides are constructed using the base-pairing rules of triple helix formation.

In yet a further embodiment, oligonucleotides can be used in the present method. In the context of this application, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. In preferred embodiments, at least one of the phosphodiester bonds of the oligonucleotide has been substituted with a structure that functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the present disclosure. Oligonucleotides may also include species that include at least some modified base forms. Thus, purines and pyrimidines other than those normally found in nature may be so employed. Similarly, modifications on the furanosyl portions of the nucleotide subunits may also be affected, as long as the essential tenets of this disclosure are adhered to. Examples of such modifications are 2′-O-alkyl- and 2′-halogen-substituted nucleotides. Some non-limiting examples of modifications at the 2′ position of sugar moieties which are useful in the present disclosure include OH, SH, SCH₃, F, OCH₃, OCN, O(CH₂), NH₂ and O(CH₂)ₙCH₃, where n is from 1 to about 10. Such oligonucleotides are functionally interchangeable with natural oligonucleotides or synthesized oligonucleotides, which have one or more differences from the natural structure. All such analogs are comprehended herewith so long as they function effectively to hybridize with at least one nucleic acid molecule encoding an inflammation-enabling polypeptide to inhibit the function thereof.

Alternatively, expression vectors derived from retroviruses, adenovirus, herpes or vaccinia viruses or from various bacterial plasmids may be used for delivery of nucleotide sequences to the targeted organ, tissue or cell population. Methods which are well known to those skilled in the art can be used to construct recombinant vectors which will express nucleic acid sequence that is complementary to the nucleic acid sequence encoding an inflammation-enabling polypeptide.

RNA interference (RNAi) is a post-transcriptional gene silencing process that is induced by a miRNA or a dsRNA (a small interfering RNA; siRNA), and has been used to modulate gene expression. RNAi can be used in the therapeutic method describe herewith. Generally, RNAi is being performed by contacting cells with a double stranded siRNA ou a small hairpin RNA (shRNA). However, manipulation of RNA outside of cells is tedious due to the sensitivity of RNA to degradation. It is thus also encompassed herein a deoxyribonucleic acid (DNA) compositions encoding small interfering RNA (siRNA) molecules, or intermediate siRNA molecules (such as shRNA), comprising one strand of an siRNA be used. Accordingly, the present application provides an isolated DNA molecule, which includes an expressible template nucleotide sequence of at least about 16 nucleotides encoding an intermediate siRNA, which, when a component of an siRNA, mediates RNA interference (RNAi) of a target RNA. The present application further concerns the use of RNA interference (RNAi) to modulate the expression of nucleic acid molecules encoding the inflammation-enabling polypeptide in target cells. While the therapeutic applications are not limited to a particular mode of action, RNAi may involve degradation of messenger RNA (e.g. mRNA of genes of inflammation-enabling polypeptide) by an RNA induced silencing complex (RISC), preventing translation of the transcribed targeted mRNA. Alternatively, it may also involve methylation of genomic DNA, which shuts down transcription of a targeted gene. The suppression of gene expression caused by RNAi may be transient or it may be more stable, even permanent.

“Small interfering RNA” or siRNA can also be used in the present methods. It o refers to any nucleic acid molecule capable of mediating RNA interference “RNAi” or gene silencing. For example, siRNA can be double stranded RNA molecules from about 10 to about 30 nucleotides long that are named for their ability to specifically interfere with protein expression (e.g. the inflammation-enabling polypeptide expression). In one embodiment, siRNAs of the present disclosure are 12-28 nucleotides long, more preferably 15-25 nucleotides long, even more preferably 19-23 nucleotides long and most preferably 21-23 nucleotides long. Therefore preferred siRNA are 12, 13, 14, 15, 16, 17, 18, 19, 20, 21, 22, 23, 24, 25, 26, 27, 28 nucleotides in length. As used herein, siRNA molecules need not to be limited to those molecules containing only RNA, but further encompass chemically modified nucleotides and non-nucleotides. siRNA can be designed to decrease expression of inflammation-enabling polypeptide in a target cell by RNA interference. siRNAs can comprise a sense region and an antisense region wherein the antisense region comprises a sequence complementary to an mRNA sequence for a nucleic acid molecule encoding inflammation-enabling polypeptide and the sense region comprises a sequence complementary to the antisense sequence of the gene's mRNA. An siRNA molecule can be assembled from two nucleic acid fragments wherein one fragment comprises the sense region and the second fragment comprises the antisense region of siRNA molecule. The sense region and antisense region can also be covalently connected via a linker molecule. The linker molecule can be a polynucleotide linker or a non-polynucleotide linker.

A ribozyme (from ribonucleic acid enzyme, also called RNA enzyme or catalytic RNA) is an RNA molecule that catalyzes a chemical reaction. Some ribozymes may play an important role as therapeutic agents, as enzymes which target defined RNA sequences, as biosensors, and for applications in functional genomics and gene discovery. Ribozymes can be genetically engineered to specifically cleave a transcript of a gene from a nucleic acid molecule encoding inflammation-enabling polypeptide whose expression is upregulated with the disease.

The delivery of the gene or genetic material into the cell is the first step in gene therapy treatment of any disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells then administered to patients (ex vivo). Conventional viral based systems for the delivery of nucleic acids could include retroviral, lentiviral, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures.

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system.

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defective vector is propagated in human 293 cells that supply the deleted gene function in trans. Ad vectors can transduce multiple types of tissues in vivo, including non-dividing, differentiated cells such as those found in the liver, kidney and muscle tissues. Conventional Ad vectors have a large carrying capacity.

In many gene therapy applications, it is desirable that the gene therapy vector be delivered with a high degree of specificity to a particular tissue type, such as for example, the myeloid or lymphoid cells. A viral vector is typically modified to have specificity for a given cell type by expressing a ligand as a fusion protein with a viral coat protein on the viruses outer surface. The ligand is chosen to have affinity for a receptor known to be present on the cell type of interest.

Gene therapy vectors can be delivered in vivo by administration to an individual subject, typically by systemic administration (e.g. intravenous, intratumoral, intraperitoneal, intramuscular, subdermal, or intracranial infusion) or topical application. Alternatively, vectors can be delivered to cells ex vivo, such as cells explanted from an individual patient (e.g. lymphocytes, bone marrow aspirates, and tissue biopsy) or universal donor hematopoietic stem cells, followed by re-implantation of the cells into the subject, usually after selection for cells which have incorporated the vector.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft at an appropriate location (such as in the bone marrow). Methods for differentiating CD34+ cells in vitro into clinically important immune cell types using cytokines such as for example GM-CSF, IFN-γ and TNF-α are known.

Stem cells can be isolated for transduction and differentiation using known methods. For example, stem cells can be isolated from bone marrow cells by panning the bone marrow cells with antibodies which bind unwanted cells, such as CD4+ and CD8+(T cells), CD45+(panB cells), GR-1 (granulocytes), and lad (differentiated antigen presenting cells).

In another embodiment, the therapeutic agent can be an antibody (or a variant thereof) capable of limiting or even inhibiting the biological activity of the inflammation-enabling protein. Such antibodies are also known in the art as neutralizing antibodies.

Naturally occurring immunoglobulins have a common core structure in which two identical light chains (about 24 kD) and two identical heavy chains (about 55 or 70 kD) form a tetramer. The amino-terminal portion of each chain is known as the variable (V) region and can be distinguished from the more conserved constant (C) regions of the remainder of each chain. Within the variable region of the light chain is a C-terminal portion known as the J region. Within the variable region of the heavy chain, there is a D region in addition to the J region. Most of the amino acid sequence variation in immunoglobulins is confined to three separate locations in the V regions known as hypervariable regions or complementarity determining regions (CDRs) which are directly involved in antigen binding. Proceeding from the amino-terminus, these regions are designated CDR1, CDR2 and CDR3, respectively. The CDRs are held in place by more conserved framework regions (FRs). Proceeding from the amino-terminus, these regions are designated FR1, FR2, FR3, and FR4, respectively.

Antibody derivatives include, but are not limited to, chimeric and humanized antibodies. As used herein, the term “chimeric” antibodies refers to an antibody molecule derived from antibodies from two different species. A humanized antibody is a type of chimeric antibody. As used herein, the term “humanized antibody” refers to an immunoglobulin that comprises both a region derived from a human antibody or immunoglobulin and a region derived from a non-human antibody or immunoglobulin. The action of humanizing an antibody consists in substituting a portion of a non-human antibody with a corresponding portion of a human antibody. For example, a humanized antibody as used herein could comprise a non-human region variable region (such as a region derived from a murine antibody) capable of specifically recognizing the inflammation-enabling polypeptide and a human constant region derived from a human antibody. In another example, the humanized immunoglobulin can comprise a heavy chain and a light chain, wherein the light chain comprises a complementarity determining region derived from an antibody of non-human origin which binds (specifically) the inflammation-enabling polypeptide and a framework region derived from a light chain of human origin, and the heavy chain comprises a complementarity determining region derived from an antibody of non-human origin which binds (specifically) the inflammation-enabling polypeptide and a framework region derived from a heavy chain of human origin.

In an embodiment, the antibody can be a monoclonal antibody (e.g. derived from a single antibody-producing clone). In some embodiment, the present application also provides fragments of the monoclonal antibodies. As used herein, a “fragment” of an antibody (e.g. a monoclonal antibody) is a portion of an antibody that is capable of specifically recognizing the same epitope as the full version of the antibody. In the present patent application, antibody fragments are capable of specifically recognizing the inflammation-enabling polypeptide. Antibody fragments include, but are not limited to, the antibody light chain, single chain antibodies, Fv, Fab, Fab′ and F(ab′)₂ fragments. Such fragments can be produced by enzymatic cleavage or by recombinant techniques. For instance, papain or pepsin cleavage can be used to generate Fab or F(ab′)₂ fragments, respectively. Antibodies can also be produced in a variety of truncated forms using antibody genes in which one or more stop codons have been introduced upstream of the natural stop site. For example, a chimeric gene encoding the heavy chain of an F(ab′)₂ fragment can be designed to include DNA sequences encoding the CH1 domain and hinge region of the heavy chain. Antibody fragments can also be humanized. For example, a humanized light chain comprising a light chain CDR (i.e. one or more CDRs) of non-human origin and a human light chain framework region. In another example, a humanized immunoglobulin heavy chain can comprise a heavy chain CDR (i.e. one or more CDRs) of non-human origin and a human heavy chain framework region. The CDRs can be derived from a non-human immunoglobulin.

In other embodiment, a polyclonal antibody composition can be used. The polyclonal antibody composition can be used directly as it is generated by the method, or can be further processed prior to its use. For example, the polyclonal antibody composition can be further fragmented, humanized, linked to another agent, etc.

Administration is by any of the routes normally used for introducing the therapeutic agent into ultimate contact with blood or tissue cells. The nucleic acids molecules described herein can be administered in any suitable manner, preferably with the pharmaceutically acceptable carriers or excipients. The terms “pharmaceutically acceptable carrier”, “excipients” and “adjuvant” and “physiologically acceptable vehicle” and the like are to be understood as referring to an acceptable carrier or adjuvant that may be administered to a patient, together with a compound of this disclosure, and which does not destroy the pharmacological activity thereof. Further, as used herein “pharmaceutically acceptable carrier” or “pharmaceutical carrier” are known in the art and include, but are not limited to, 0.01-0.1 M and preferably 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

As used herein, “pharmaceutical composition” means therapeutically effective amounts (dose) of the agent together with pharmaceutically acceptable diluents, preservatives, solubilizers, emulsifiers, adjuvants and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g. Tris-HCl, acetate, phosphate), pH and ionic strength, additives such as albumin or gelatin to prevent absorption to surfaces, and detergents (e.g. Tween20™, Tween80™, Pluronic F68™, bile acid salts). The pharmaceutical composition can comprise pharmaceutically acceptable solubilizing agents (e.g. glycerol, polyethylene glycerol), anti-oxidants (e.g., ascorbic acid, sodium metabisulfite), preservatives (e.g. thimerosal, benzyl alcohol, parabens), bulking substances or tonicity modifiers (e.g. lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g. fatty acids, waxes, oils). Also comprehended by the disclosure are particulate compositions coated with polymers (e.g. poloxamers or poloxamines).

Suitable methods of administering the therapeutic agents are available and well known to those of skill in the art, and, although more than one route can be used to administer a particular composition, a particular route can often provide a more immediate and more effective reaction than another route. The preventive or therapeutic agents of the present disclosure may be administered, either orally or parenterally, systemically or locally. For example, intravenous injection such as drip infusion, intramuscular injection, intraperitoneal injection, subcutaneous injection, suppositories, intestinal lavage, oral enteric coated tablets, and the like can be selected, and the method of administration may be chosen, as appropriate, depending on the age and the conditions of the patient. The effective dosage is chosen from the range of 0.01 mg to 100 mg per kg of body weight per administration. Alternatively, the dosage in the range of 1 to 1000 mg, preferably 5 to 50 mg per patient may be chosen.

### Inflammation-Enabling Polypeptides

The inflammation-enabling polypeptide sequences (and nucleic acids associated thereto) encompass host polypeptides (also refer to as targets) which are herein shown to enable the induction and/or persistence of a pathological inflammatory response. These polypeptides are considered to be involved in any pathological inflammation response, regardless of the etiology of the disease. In an embodiment, the IEP include LYST, ZBTB7B, BPGM1, RASAL3, CCDC88B, FOXN1, TRIM25 and USP15. In yet another embodiment the IEP include LYST, ZBTB7B, BPGM1, RASAL3, CCDC88, FOXN1, USP15, TRIM25, THEMIS and IRF8. In another embodiment, the IEP include, LYST, ZBTB7B, BPGM1, RASAL3, CCDC88B, FOXN1, USP15, TRIM25, THEMIS, IRF1, IRGM1 and IRF8. In yet another embodiment, the IEF is USP15 and/or TRIM25. In still another embodiment, JAK3 is not considered to be an IEP. For nucleic acid molecules, this encompasses sequences that are identical or complementary to the coding sequences of the IEP, as well as sequence-conservative and function-conservative variants thereof. For IEP, this encompasses sequences that are identical to the polypeptide, as well as function-conservative variants thereof. Included are the alleles of naturally-occurring polymorphisms of the IEP-encoding genes which do not cause altered expression of their respective genes and alleles that do not cause altered protein levels, activity or stability.

Function-conservative variants are those in which a change in one or more nucleotides in a given codon position results in a polypeptide sequence in which a given amino acid residue in the polypeptide has been replaced by a conservative amino acid substitution. Function-conservative variants also include analogs of a given polypeptide and any polypeptides that have the ability to elicit antibodies specific to a designated polypeptide.

Sequence-conservative variants consists of variants in which a change of one or more nucleotides in a given codon position results in no alteration in the amino acid encoded at that position (e.g., silent mutation).

A nucleic acid or fragment thereof is “substantially homologous” or “substantially identical” to another if, when optimally aligned (with appropriate nucleotide insertions and/or deletions) with the other nucleic acid (or its complementary strand), there is nucleotide sequence identity in at least 60% of the nucleotide bases, usually at least 70%, more usually at least 80%, preferably at least 90%, and more preferably at least 95-98% of the nucleotide bases. Alternatively, substantial homology or substantial identity exists when a nucleic acid or fragment thereof will hybridize, under selective hybridization conditions, to another nucleic acid (or a complementary strand thereof). Selectivity of hybridization exists when hybridization which is substantially more selective than total lack of specificity occurs. Typically, selective hybridization will occur when there is at least about 55% sequence identity over a stretch of at least about nine or more nucleotides, preferably at least about 65%, more preferably at least about 75%, and most preferably at least about 90%. The length of homology or identity comparison, as described, may be over longer stretches, and in certain embodiments will often be over a stretch of at least 5 nucleotides, at least 14 nucleotides, at least 20 nucleotides, more usually at least 24 nucleotides, typically at least 28 nucleotides, more typically at least 32 nucleotides, and preferably at least 36 or more nucleotides.

A polypeptide or fragment thereof is “substantially homologous” or “substantially identical” to another if, when optimally aligned (with appropriate insertions and/or deletions) with the other polypeptide, there is nucleotide sequence identity in at least 60% of the nucleotide bases, usually at least 70%, more usually at least 80%, preferably at least 90%, and more preferably at least 95-98% of the amino acid residues. The length of homology or identity comparison, as described, may be over longer stretches, and in certain embodiments will often be over a stretch of at least 5 amino acids, at least 14 amino acids, at least 20 amino acids, more usually at least 24 amino acids, typically at least 28 amino acids, more typically at least 32 amino acids, and preferably at least 36 or more amino acids.

CCDC88B.

This IEP is also referred to as coiled-coil domain containing 88B (Homo sapiens Gene ID 283234). Its function has not been established yet, but it is a member of the hook-related protein family. Members of this family are characterized by an N-terminal potential microtubule binding domain, a central coiled-coiled and a C-terminal Hook-related domain. The encoded protein may be involved in linking organelles to microtubules. The amino acid mutant this protein is provided as SEQ ID NO: 4 (murine version).

LYST.

This IEP is a lysosomal-trafficking regulator (Homo sapiens GENE ID 1130). It regulates intracellular protein trafficking in endosomes, and may be involved in pigmentation. Mutations in this gene are associated with Chediak-Higashi syndrome, a lysosomal storage disorder.

ZBTB7B.

This IEP is also referred to as “zinc finger and BTB domain containing 7B” (Homo sapiens Gene ID 51043). It is a zinc finger-containing transcription factor that acts as a key regulator of lineage commitment of immature T-cell precursors. It is necessary and sufficient for commitment of CD4 lineage, while its absence causes CD8 commitment. It also functions as a transcriptional repressor of type I collagen genes.

BPGM1.

This IEP is a glycolytic enzyme. Bpgm1 is a tri-functional enzyme in the Rapoport-Luebering Shunt pathway that possesses synthase, mutase activity and catalyzes the transformation of 1,3 diphosphoglycerate to 2,3 biphosphoglycerate (2,3BPG). 2,3BPG is an allosteric regulator of hemoglobin and binds to unligated Hb. Bpgm1 is also part of glycolysis modulating the ratio of 1,3BPG and 3-phosphoglycerate.

RASAL3.

This IEP is a Ras GTPase-activating protein (RasGAP) (Mus musculus Gene ID 109747). It is expressed predominantly in hematopoietic cells, notably T cells, B cells, and NK cells. Possibly regulates Erk signaling. Rasal3 (RasGAP; negative regulator stimulating GTP hydrolysis from Ras-GTP into Ras-GDP) and Arhgef2 (RasGEF; positive regulator acting to stimulate conversion of RAS-GDP to RAS-GTP) could act together.

FOXN1.

This IEP is also referred to as forkhead box N1, WHN, RONU as well as FKHL20. FOXN1 is a winged-helix transcriptional regulator (Homo sapiens Gene ID 8456). FoxN1 mouse mutants show absence of thymus and are severely immuno-compromised. Known human FOXN1 mutations cause T-cell immunodeficiency, congenital alopecia and nail dystrophy.

USP15.

This IEP is also referred to as ubiquitin specific peptidase 15 (Homo sapiens Gene ID 9958). It is a member of the ubiquitin specific protease (USP) family of deubiquitinating enzymes. USP enzymes play critical roles in ubiquitin-dependent processes through polyubiquitin chain disassembly and hydrolysis of ubiquitin-substrate bonds. The encoded protein associates with the COP9 signalosome, and also plays a role in transforming growth factor beta signalling through deubiquitination of receptor-activated SMAD transcription factors. One of the enzymatic targets of USP15 is the TRIM25 ligase, which can be de-ubiquitinated (entirely or at least partially) by USP15. Alternatively spliced transcript variants encoding multiple isoforms (see for example, the amino acid sequence of SEQ ID NO: 55 (corresponding to the human isoform 1 of USP15) or 57 (corresponding to the human isoform 2 of USP15) have been observed for the gene encoding this IEP.

THEMIS.

This IEP is also known as thymocyte selection associated (Homo sapiens Gene ID 387357). This protein is known to play a regulatory role in both positive and negative T-cell selection during late thymocyte development. The protein functions through T-cell antigen receptor signaling, and seems necessary for proper lineage commitment and maturation of T-cells. Alternative splicing results of its corresponding gene in multiple transcript variants.

IRF8.

This IEP is also known as interferon regulatory factor 8 (Homo sapiens Gene ID 3394). This interferon consensus sequence-binding protein (ICSBP) is a transcription factor of the interferon (IFN) regulatory factor (IRF) family. Proteins of this family are composed of a conserved DNA-binding domain in the N-terminal region and a divergent C-terminal region that serves as the regulatory domain. The IRF family proteins bind to the IFN-stimulated response element (ISRE) and regulate expression of genes stimulated by type I IFNs, namely IFNα and IFNβ. IRF family proteins also control expression of IFNα and IFNβ-regulated genes that are induced by viral infection.

IRF1.

This IEP is also known as interferon regulatory factor 1 (Homo sapiens Gene ID 3659). IRF1 is a member of the interferon regulatory transcription factor (IRF) family. IRF1 serves as an activator of interferons alpha and beta transcription, and in mouse it has been shown to be required for double-stranded RNA induction of these genes. IRF1 also functions as a transcription activator of genes induced by interferons alpha, beta, and gamma. Further, IRF1 has been shown to play roles in regulating apoptosis and tumor-suppressoion. Finally, IRF1 physically interacts with and heterodimerizes with IRF8 in the transcriptional activation of many genes implicated in response to infections and to inflammation.

IRGM1.

This IEP is also known as immunity-related GTPase family, M (Homo sapiens Gene ID 345611). It is a member of the p47 immunity-related GTPase family. It is suggested to play a role in the innate immune response by regulating autophagy formation in response to intracellular pathogens. Polymorphisms that affect the normal expression of this gene are associated with a susceptibility to Crohn's disease and tuberculosis.

TRIM25.

This IEP is also known as tripartite motif containing 25 (Homo sapiens Gene ID 706). The protein encoded by this gene is a member of the tripartite motif (TRIM) family. The TRIM motif includes three zinc-binding domains, a RING, a B-box type 1 and a B-box type 2, and a coiled-coil region. The protein localizes to the cytoplasm. The presence of potential DNA-binding and dimerization-transactivation domains suggests that this protein may act as a transcription factor, similar to several other members of the TRIM family. TRIM25 is known to physically interact with USP15.

The present invention will be more readily understood by referring to the following examples which are given to illustrate the invention rather than to limit its scope.

### Example I—Genetic Screening in Enu-Induced Dominant Negative Mutations

The genetic screening was performed as presented in Bongfen et al. Briefly, a population of male G0 ENU-mutated mice was first generated. The mutant males were backcrossed for two-generations (G1 and G2) followed by breeding homozygosity in multiple G3 pedigrees. These mice were then infected with Plasmodium bergei to induce a cerebral malaria. Mice bearing mutations which prevented them from developing a full neuroinflammatory response and showing an unusual resistance to neuroinflammation (and ultimately survived the P. bergei challenge) were selected and their genome was sequenced to identify the genetic trait responsible for protecting the mice from succumbing to a P. bergei challenge.

As shown in Bongfen et al., the first phenodeviant pedigree characterized carries a mutation in Jak3 (Jak3^{W81R}), a cytosolic tyrosine kinase that interacts with the common γ_{c} chain of cytokine receptors, including IL-2, IL-4, IL-7, IL-9, IL-15 and IL-21. This recruitment is required for the engagement of STAT family members and the transcriptional activation of inflammatory pathways in NK, T and B cells. Jak3^{W81R} mutants show reduced numbers of NK cells, CD8+ T cells and B cells, and severely reduced production of the pro-inflammatory cytokine IFNγ by CD4+ T cells. Interestingly, genetic variants in JAK and STAT family members have been associated with inflammatory diseases (IBD, MS, RA, SLE). In addition, Jak3 is a known target for anti-inflammatory drugs, and a Jak3 inhibitor that is currently in clinical use for rheumatoid arthritis (RA) and Crohn's disease (CD) (e.g., tasocitinib; Pfizer) can blunt neuroinflammation and increase survival of Jak3−/+ heterozygotes infected with P. berghei. Therefore, the JAK3 inhibitor pharmacologically mimicks the effect of the genetic mutation obtained and characterized in Bongfen et al.

The first phenodeviant pedigree (number 48) was found to carry a mutation in Jak3 (Jak3W81R), a cytosolic tyrosine kinase that interacts with the common γ_{c} chain of type 1 and 2 cytokine receptors expressed on lymphocytes, which includes IL-2, IL-4, IL-7, IL-9, IL-15 and IL-21. Jak3 phosphorylation causes recruitment and phosphorylation of STAT family members for transcriptional activation of inflammatory pathways in T lymphocytes. Jak3W81R mutants show reduced numbers of CD8+ T cells and B cells, and severely reduced production of pro-inflammatory cytokines (IFNγ) by CD4+ T cells. Interestingly, genetic variants at JAK (JAK2 in inflammatory bowel diseases) and STAT family members (STAT3 in IBD and multiple sclerosis; STAT4 in rheumatoid arthritis, and Lupus erythematosus) have been found to be genetic risks in certain chronic inflammatory diseases in humans. These results confirm that the screen for mutations that protect against P. berghei infection-associated neuroinflammation can identify novel targets for anti-inflammatory drug discovery.

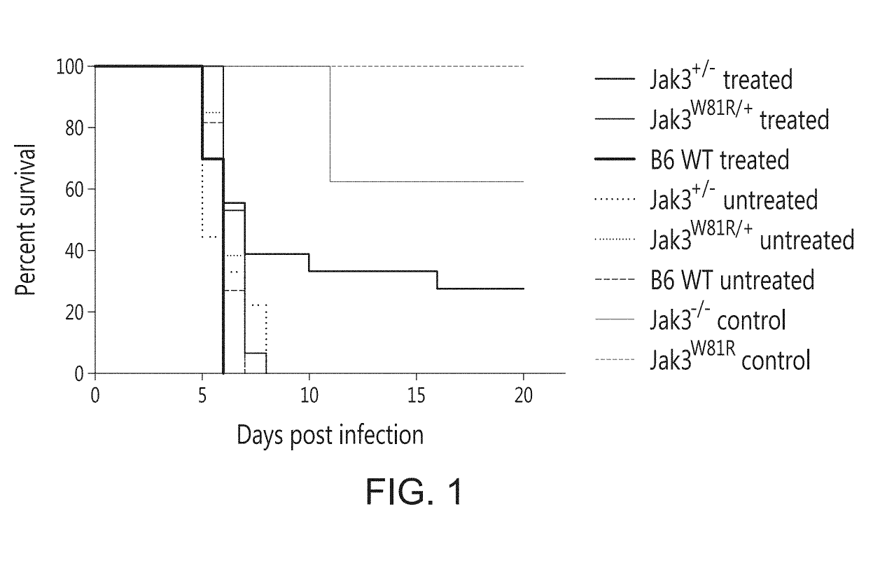

Mice were treated s.c. with 15 mg/kg/day of tasocitinib (a known JAK3 inhibitor) for 4 days. Mice infected on day 5 of treatment with 10⁶ PbA-pRBC, and treatment continued for 4 more days (total of 9 days of treatment). As shown on FIG. 1, treatment of Jak3+/− heterozygote mice with (starting 3 days before infection, and continuing for 7 days during infection with P. berghei) can blunt neuroinflammation and can significantly increase survival of P. berghei-infected Jak3^{+/-} heterozygotes (from 0% in untreated animals to 45% in treated animals). These results indicate that pharmacological modulation of a target discovered in the genetic screen can mimic the protective effect of a genetic lesion in the same gene. These results also indicate that the P. berghei infection model can be used to screen drug candidates for anti-inflammatory activity.

### Example II—Themis

A genetic screen has been performed as described in Example I and a further protective mutation was identified in Themis (123N), a protein associated with the T-cell receptor (TCR), which phosphorylation induces binding to Lck and Grb2 to stimulate the ERK1/ERK2 pathway. Themis is required for TCR activation and cytokine production by CD4+ and CD8+ lymphocytes in response to class I and class II MHC-dependent antigen presentation. In addition, inhibitors of the ERK pathway (PD184352, U0126) have been described and are available for testing in vivo and can be used for modulating the inflammatory response in vivo.

An heterozygote mouse strain has been produced.

### Example III—Foxn1

A genetic screen has been performed as described in Example I and a further protective mutation was identified in the winged-helix transcriptional regulator FoxN1. Two nude resistant mice show a splice-site mutation (A-to-C) at the exon 6 donor site. FoxN1 mouse mutants show absence of thymus and are severely immuno-compromised, while human FOXN1 mutations cause T-cell immunodeficiency, congenital alopecia and nail dystrophy.

An heterozygote mouse strain has been produced.

### Example IV—Ccdc88B

A genetic screen has been performed as described in Example I and a further protective mutation was identified in Ccdc88b, a gene expressed in the thymus, lymph nodes, and bone marrow. The Ccdc88b's mRNA expression in macrophages is increased upon engagement of innate immune receptors TLR4 (lipopolysaccharide) and TLR9 (CpG oligonucleotides). In addition, CCDC88B's expression is also increased in response to infection with Mycobacterium tuberculosis (lung), Plasmodium berghei (blood, liver cells), and Lesihmania (macrophages). The function, biochemical activity and mechanism of action of the CCDC88B protein in lymphoid cells and in myeloid cells remain unknown. Structurally, CCDC88B is characterized by an N-terminal potential microtubule binding domain, a central coiled-coiled domain, and a C-terminal Hook-related domain. These features plus additional observations suggest that CCDC88B may form scaffolds in T lymphocytes, possibly those required to assemble the T cell receptor signaling complex.

It was noted that mice bearing an homozygote mutation at CCDC88B (a mutation in the splice site in intron 22 that abrogate splicing of exon 22/23, and results in an out of frame transcript) show strongly reduced numbers of granulocytes in the spleen (neutropenia). More specifically, in the homozygous mutant, the T to C mutation in the donor splice site between exon 22 and exon 23 of CCDC88B results in the activation of an alternative donor splice site upstream of the mutation in exon 22. As a consequence, there is a five nucleotide deletion in exon 22 causing a frameshift in the mutant polypeptide sequence. Using the online Transeq Nucleotide to Protein Sequence Conversion tool by EBI, it is predicted that the frameshift leads to an early stop codon and thereby nonfunctional associated protein. The expected amino acid sequence of the mutant CCDC88B protein is provided in the sequence listing as SEQ ID NO: 4.

Stable cell lines expressing either CCDC88B or its mutant version were developed (e.g. in HEK293 EV cells). An heterozygote mouse strain has been produced. In addition, rabbit polyclonal sera specific either for the wild-type polypeptide or the mutant polypeptide have been prepared.

The expression of CCDC88B has been further characterized by in situ hybridization. Tissues were fixed in 4% formaldehyde and hybridized with ³⁵S-labeled cRNA antisense and sense probes overnight at 55° C. in 50% deionized formamide, 0.3 M NaCl, 20 mM Tris-HCl, pH 7.4, 5 mM EDTA, 10 nM NaPO₄, 10% dextran sulfate, 1×Denhardt's, 50 μg/ml total yeast RNA, and 50 to 80 000 cpm/μl ³⁵S-labeled cRNA probe. The tissues were subjected to stringent washing at 65° C. in 50% formamide, 2×SSC, and 10 mM DTT, followed by washing in PBS before treatment with 20 μg/ml RNAse A at 37° C. for 30 minutes. After washes in 2×SSC and 0.1×SSC for 10 minutes at 37° C., the slides were dehydrated, apposed to x-ray film for 3 and 6 days, then dipped in Kodak NTB nuclear track emulsion, and exposed for 18 days in light-tight boxes with desiccant at 4° C. Photographic development was undertaken with Kodak D-19. The slides are lightly counterstained with cresyl violet and analyzed under both light- and darkfield optics. In situ hybridization (ISH) was performed in 10-day old mice (p10) using the following probes:

Sense probe (SEQ ID NO: 5)-italics shows section
specific to T7 promoter
: GCGCTATAATACGACTCACTATAGGGAGATCCGAATCTTTGGACCTG
CCTTCT
Antisense probe (SEQ ID NO: 6)-italics shows
section specific for the SP6 promoter:
GCATTAATTTAGGTGACACTATAGAAGCGAAGCTAGCCGTATCCACTGC
TTCA

In these mice, Ccdc88b's mRNA expression was detected in the thymus (at a high-level), in the spleen and bone marrow (at a low-level), as well as in the ribs and hip bones (data not shown).

When ISH was performed in adult mice using the same approach, increased Ccdc88b's mRNA expression was found in the spleen, and bone marrow, as well as in the lymph node (data not shown). In the thymus, Ccdc88b's mRNA was observed both in the cortex (at a higher cell density) and in the medulla (data not show). In the spleen, Ccdc88b's mRNA expression was increased in the germinal centers close to lymphoid nodule around central artery (data not shown). In the lymph nodes, Ccdc88b's mRNA expression was observed in the medulla but not in lymphoid follicles (data not shown). Similar results were obtained by confocal microscopy using the rabbit polyclonal serum generated (data not shown).

To confirm the ISH results, qPCR was performed to assess the expression of Ccdc88b's mRNA levels. Real-time quantitative PCR (qPCR) amplifications were performed on the Roche LightCycler 480 system using 96-microwell plates in a total volume of 10 μL, containing 5 μL cDNA sample, 5 uL PerfeCTa SYBR Green SuperMix (Quanta BioSciences) and 150 nM each of forward and reverse primers. PCR amplifications were conducted in triplicate with one RT-control for each sample, using the temperature cycles: 10 min at 94° C., followed by 45 cycles of (15 s at 94° C. and 1 min at 60° C.). Ccdc88b cDNA of length 150 bp was amplified using 5′-GAT CTG GGG GCA CAG CGG TTG (SEQ ID NO: 7) and 5′-GCG TCT CAG CTG GGC CTT GGC (SEQ ID NO: 8), respectively. Gene expression was normalized to the reference gene HPRT, which was amplified as a 122 bp product using 5′-TCC AGC AGG TCA GCA AAG AAC (SEQ ID NO: 9) and 5′-GGA CTG ATT ATG GAC AGG ACT G (SEQ ID NO: 10) primers. Five samples of 10× dilutions were made to determine the amplification efficiencies of Hprt and Ccdc88b. Relative gene expression was quantified using the Pfaffl method as per the manufacturer's protocol, and normalized to uninfected C57BL/10 brain sample.

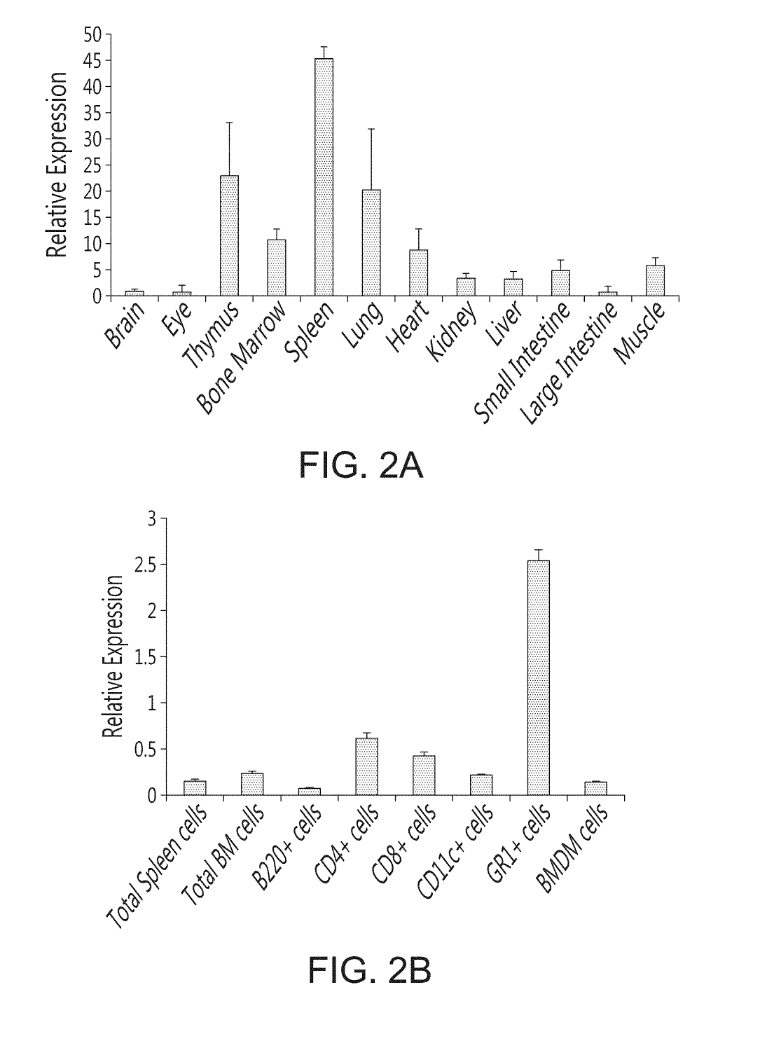

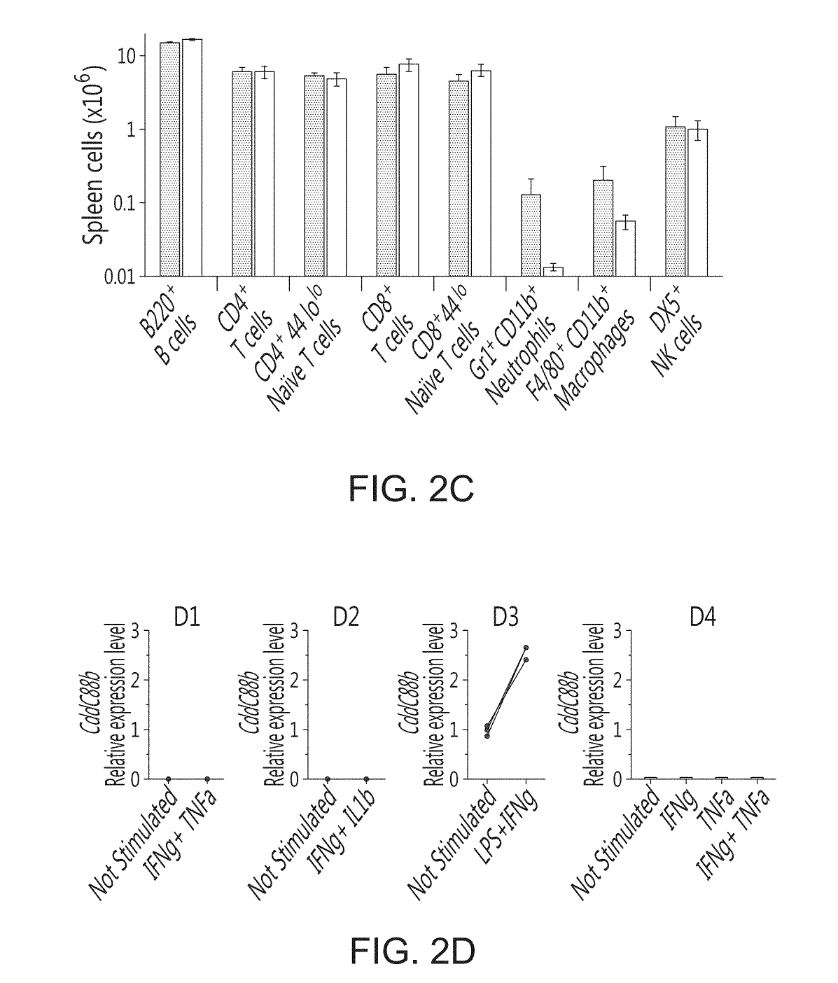

As shown on FIG. 2A, high Ccdc88b's mRNA expression was found in the spleen, the thymus, the bone marrow and the lungs. Since there was an increased expression of Ccdc88b's mRNA in organs involved in inflammation, expression of Ccdc88b's mRNA in various inflammatory cell types was determined. As shown in FIG. 2B, elevated expression of Ccdc88b's mRNA was found in GR1+ granulocytes, CD4+ and CD8+ T cells.

Immunophenotyping was performed on hemizygote mutant animals (CCDC88B^{+/-}). As shown on FIG. 2C, in the spleen, there was a significant decrease in Gr1+CD11b+ neutrophils in uninfected hemizyogte mice. Consequently, CCDC8*B^{+/-} animals had a severe neutropenia phenotype. It was also determined that, upon inflammatory challenge, no inflammatory cells were recruited to the brain (data not shown).

The expression of Ccdc88b's mRNA in human glial cells and blood brain barrier endothelial cells was characterized. All tissues samples taken from human epilepsy patients. Tissues were non-inflamed. Three different individuals were tested per cell types. As shown in FIG. 2D, the expression level of Ccdc88b's mRNA was determined in human astrocytes, microglia or blood brain barrier endothelial cells following 48 h stimulation by cytokines optionally in combination LPS. Expression of Ccdc88b's mRNA was increased in microglia following stimulation with IFNγ and LPS. No expression of Ccdc88b's mRNA was detected in astrocytes or in endothelial cells.

### Example V—Usp15

A genetic screen has been performed as described in Example I and a further protective mutation was identified in Usp15 (Ubiquitin carboxyl-terminal hydrolase 15), a deubiquitinating enzyme (DUB). The mutation (Usp15^{L749R}) is a non-conservative change at a highly conserved residue in the catalytic domain common to DUBs. USP15 has been shown to deubiquitinate receptor associated cytosolic SMADs to regulate downstream TGFβ signaling. Although the function of USP15 in inflammatory cells is unknown, addition/removal of ubiquitin from proteins is a common means of regulating immune signaling, and several genes associated with inflammatory diseases in humans either regulate ubiquitination (TNFAIP3), bind to ubiquitinated proteins (TNIP1, UBASH3A) or regulate enzymatic events in ubiquitination (UBE2L3). The USP15 protein appears to be expressed at least in the brain.

An heterozygote mouse strain has been produced.

### Example VI—Irf8

IRF8 is a member of the Interferon Regulatory Factor (IRF) family of transcriptional regulators that plays a central role in interferon signaling, response to infection and maturation of myeloid lineages, including dendritic cells (DC). It is composed of a helix-turn-helix DNA binding domain and a trans-activation domain also known as the IRF association domain. In myeloid and lymphoid cells, IRF8 regulates constitutive gene expression and also activates or suppresses pathogen responsive transcription programs following exposure of these cells to type I or type II interferon, lipopolysaccharides, and a range of microbial products. Heterodimerization of IRF8 with members of the IRF (IRF1, IRF4) or ETS (PU.1) families leads to DNA binding and transcriptional regulation of target genes containing ISRE (GAAAnnGAAA) (SEQ ID NO: 1) and EICE-type canonical motifs (GGAAnnGAAA) (SEQ ID NO: 2), respectively, in their promoters.

During hematopoiesis, IRF8 promotes differentiation of myeloid progenitors towards the mononuclear phagocyte lineages (monocytes, macrophages, DC) by acting as an antagonist of the polymorphonuclear granulocyte pathway. This is accomplished through positive regulation of pro-apoptotic signals (Cdkn2b, Nf1, Bax), and negative regulation of pro-survival signals (Bcl2, Bcl-XL) in CD11b+ myeloid precursors. Mice harboring either complete (null7) or severe (Irf8^{R294C} from BXH2 mice) loss of function mutations at Irf8 show a complete or partial absence of all classes of DCs, both CD11c+CD8α+ DCs and plasmacytoid DCs, and display a chronic myeloid leukemia-like phenotype dominated by expansion of Gr1+/CD11b+ granulocyte precursors. Additionally, IRF8 has been shown to be required for B lymphocytes lineage specification, commitment and differentiation, including expression of biochemical pathways that play a key role in the specialized functions of these antigen-presenting cells.

In the context of infection, IRF8 is required for the activation of anti-microbial defenses of resident myeloid cells, for propagation of pro-inflammatory signals and for amplification of the early immune response by these cells. IRF8 is essential for antigen presenting cell-mediated Th1 polarization of early immune responses, as it is necessary for expression of the IL12p40, IL12p35 and IL-18 genes in response to IFNγ. Consequently, Irf8-deficient mice display defective Th1 response (absence of antigen specific CD4+, IFNγ producing T cells), show enhanced Th17 response, and are susceptible to in vivo infection with many intracellular pathogens including tuberculosis and blood-stage malaria. Furthermore, genome-wide transcript profiling, chromatin immunoprecipitation experiments and individual gene studies show that IRF8 regulates several aspects of anti-microbial defenses in mononuclear phagocytes, including antigen recognition and processing, phagosome maturation, production of lysosomal enzymes and of other cytoplasmic microbicidal pathways. As a result, Irf8-deficient macrophages are extremely susceptible to ex vivo infection with a variety of intracellular pathogens.

IRF8 mutations in humans cause pathologies remarkably similar to those observed in Irf8 mutant mice, and affect myeloid cells in general and DCs in particular. In one patient, homozygosity for a transcriptionally inactive and DNA-binding incompetent IRF8 mutant variant (IRF8^{K108E}) was associated with severe and recurrent perinatal bacterial and fungal infections, with absence of blood monocytes and DCs in lymph nodes and bone marrow, and a lack of IL-12 and IFNγ production following stimulation of blood cells in vitro. A milder autosomal dominant form of IRF8-deficiency (IRF8^{T8}0A) was also recently discovered in two MSMD patients (Mendelian Susceptibility to Mycobacterial Disease) suffering from recurrent episodes of mycobacterial infections following perinatal vaccination with M. bovis BCG. These patients had a selective depletion of the CD11c+CD1c+DC subset, and impaired production of IL-12 by circulating peripheral blood cells. The IRF8^{T8}0A variant displays negative dominance and can suppress the trans-activation potential of wild type IRF8 for known transcriptional targets such as NOS2 and IL-12. Finally, recent results from genome wide association studies (GWAS) have pointed to a role of IRF8 in the complex genetic etiology of multiple human diseases with important inflammatory components. Strong and independently replicated associations have been detected between polymorphic variants within or near IRF8 in patients with systemic lupus erythematosus, Crohn's disease (CD) and multiple sclerosis (MS). In one study of MS patients, the susceptibility allele at IRF8 is associated with higher IRF8 mRNA expression.

An experimental model of cerebral malaria (CM) induced by infection with Plasmodium berghei ANKA (PbA) was used to investigate the role of IRF8 in pathological inflammation. In this model, adherence of PbA-infected erythrocytes to brain microvasculature leads to acute and rapidly fatal neuroinflammation, which symptoms include tremors, ataxia and seizures begining to appear between d5-d8 post-infection. IRF8-deficient BXH2 mice (Irf8^{R294C}) do not develop any neurological symptoms and are completely resistant to PbA-induced CM. Comparative transcript profiling studies in PbA-infected brains of wild-type C57BL/6 and Irf8-deficient BXH2 mice, together with IRF8 chromatin immunoprecipitation coupled to high-throughput DNA sequencing (ChIP-seq) have identified a list of key IRF8 targets whose expression is associated with acute CM-associated neuroinflammation, but is also found activated in lungs infected with M. tuberculosis. The role of several of these genes in CM-pathology has been validated in vivo in corresponding mouse mutants infected with P. berghei. These studies identify IRF8 as a key regulator of acute neuroinflammation during CM.

Mice.

C57BL/6J (B6), BXH2, 1112p40−/−, Irf1−/−, and Isg15−/− mutant mice were originally obtained from the Jackson Laboratory (Bar Harbor, Me.). Stat1−/− mutant mice were purchased from Taconic Farms (Germantown, N.Y.). Ifng−/− deficient mice were obtained from Dr. M. M. Stevenson (Montreal General Hospital Research Institute), Ifit1−/− mutant mice were obtained from Dr. M. Diamond (Washington University School of Medicine, St-Louis), Irgm11^{-/-} from Dr. J. D. MacMicking (Yale, New Haven, Conn.) and Nlrc4^{-/-} from Millenium Pharmaceuticals, Inc. and Dr. R. A. Flavell (Yale, New Haven, Conn.). All mice were kept under pathogen free conditions and were handled according to the guidelines and regulations of the Canadian Council on Animal Care.

Parasites and Infection.

P. berghei ANKA was obtained from the Malaria Reference and Research Reagent Resource Centre (MR4), and was stored frozen at −80° C. Prior to experimental infections, P. berghei ANKA was passaged in B6 mice until peripheral blood parasitemia levels reached 3-5%, at which point animals were euthanized by CO₂ inhalation, exsanguinated and an infectious stock was prepared. All experimental infections were done via intraperitoneal (i.p.) injection with 10⁶ parasitized red blood cells (pRBC). Blood parasitemia was monitored during infection by microscopic examination of thin-blood smears stained with Diff-Quick™ (Dade Behring, Newark, Del., USA). The appearance of neurological symptoms (shivering, tremors, ruffled fur, seizures) associated with cerebral malaria (CM) was monitored closely, and affected animals were immediately sacrificed. Survival curves were compared using Kaplan-Meier statistics.

Evans Blue Dye Extravasation Assay.

To monitor the integrity of the blood brain barrier during experimental CM, groups of control and PbA infected C57BL/6 and BXH2 mice were injected i.p. with 0.2 ml of 1% Evan's Blue dye (E2129; Sigma-Aldrich, Oakville, ON, Canada) in sterile phosphate-buffered saline (PBS) on d7 and d16 (BXH2 only) post-infection (n=3 mice/condition). The dye was allowed to circulate for 1 h, then the mice were sacrificed by CO₂ inhalation, perfused with PBS and the brains were dissected and photographed. To quantify dye accumulation in the brain, tissues were weighed and the dye was extracted 48 hours in 1 ml N,N-dimethylformamide, followed by measuring optical density at OD₆₂₀. A standard curve was prepared at the same time and linear regression was used to calculate the concentration of dye in each extracted sample solution. Uninfected mice were also injected with dye, perfused and dissected as controls.

Serum Cytokines.

Five male mice of each wild type B6, C3H/HeJ, and mutant BXH2 were infected i.p. with 10⁶ P. berghei ANKA pRBC, and at 6 days post-infection, they were sacrificed and serum was collected. Levels of circulating cytokines IFNγ, IL-2, IL-10, IL12p40, IL12p70, MCP-1 (CCL2), MIP-1β (CCL4), RANTES (CCL5), TNF-α, and VEGF were measured using an ELISA-based commercial reagent (Milliplex Assay™; Millipore).

Transcript Profiling.

Whole brains were dissected from B6 and BXH2 mice either prior to (d0) or 7 days (d7) post infection (n=3/condition). Total brain RNA was isolated using TRIzol™ reagent (Invitrogen, Burlington, Canada) according to the manufacturer's instructions, followed by further purification with RNeasy™ columns (Qiagen, Toronto, Canada) and hybridized to Illumina MouseWG-6 v2.0 microarrays (Genome Quebec Innovation Centre, Montreal, Canada). Unsupervised principal components analysis was done in R, using the lumi package to transform with vst (variance stabilizing tranformation) and to perform quartile normalization. For other analyses, microarray expression data was log 2 transformed, median normalized and analyzed using GeneSifter (Geospiza) software. Groups were compared using either a pairwise (t-test, >2-fold cutoff, Benjamini-Hochberg corrected p_{adj}-Values <0.05) or using a two factor ANOVA (>2-fold cutoff, Benjamini-Hochberg corrected p_{adj}-Values <0.05) to identify genes whose expression is modulated in a strain-dependent, infection-dependent and/or interactive fashion. Lists of genes that were differentially expressed were clustered according to fold change using Multi Experiment Viewer.

Chromatin immunoprecipitation (ChIP).

The J774 mouse macrophage cell line was grown to 80% confluence in complete Dulbecco's modified Eagle's medium (DMEM). The cells plated in 150 mm tissue culture-grade Petri dishes (Corning Inc., Corning, N.Y.) were treated with 400 U/ml IFNγ (Cell science, Canton, Mass.) and CpG DNA oligonucleotides (5′-TCCATGACGTTCCTGACGTT-3′) (SEQ ID NO: 3) for 3 h. Chromatin immunoprecipitations were performed as previously described with few modifications. Briefly, treated cells were crosslinked for 10 min at 20° C. with 1% formaldehyde in culture medium. Crosslink was stopped with ice-cold PBS containing 0.125M glycine for 5 min. Nuclei were prepared and chromatin was sonicated with a Branson Digital Sonifier (Branson Ultrasonics, Danbury, Conn.) to an average size of 250 bp. Sonicated chromatin was incubated overnight on a rotating platform at 4° C. with a mixture of 20p1 Protein A and 20 μl Protein G Dynabeads (Invitrogen, Carlsbad, Calif.) pre-bound with 6 μg of normal goat IgG (sc-2028) or IRF8 (sc-6058x) antibodies (Santa Cruz Biotechnologies, Santa Cruz, Calif.). Immune complexes were washed sequentially for 2 min at room temperature with 1 ml of the following buffers: Wash B (1% Triton X-100, 0.1% SDS, 150 mM NaCl, 2 mM EDTA, 20 mM Tris-HCl pH 8), Wash C (1% Triton X-100, 0.1% SDS, 500 mM NaCl, 2 mM EDTA, 20 mM Tris-HCl pH 8), Wash D (1% NP-40, 250 mM LiCl, 1 mM EDTA, 10 mM Tris-HCl pH 8), and TEN buffer (50 mM NaCl, 10 mM Tris-HCl pH 8, 1 mM EDTA). After decrosslinking, the DNA was purified with QIAquick™ PCR purification columns following manufacturer's procedure (Qiagen, Mississauga, Ca). IRF8 ChIP efficiency relative to the IgG control was assessed by qPCR using the Perfecta SYBR™ green PCR kit (Quanta Bioscience, Gaithersburg, Md.) for known IRF8 binding sites.

ChIP-Seq Preparation and Analysis.

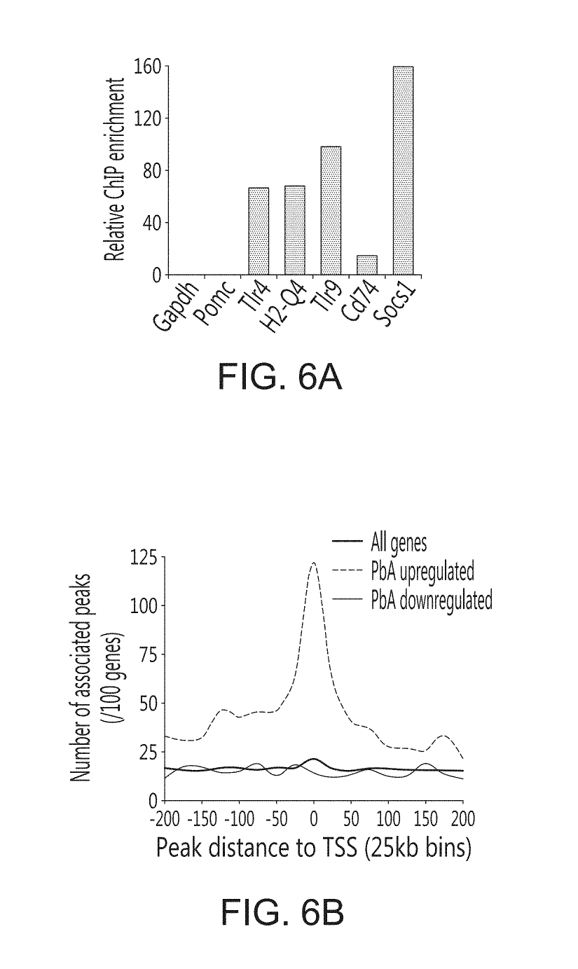

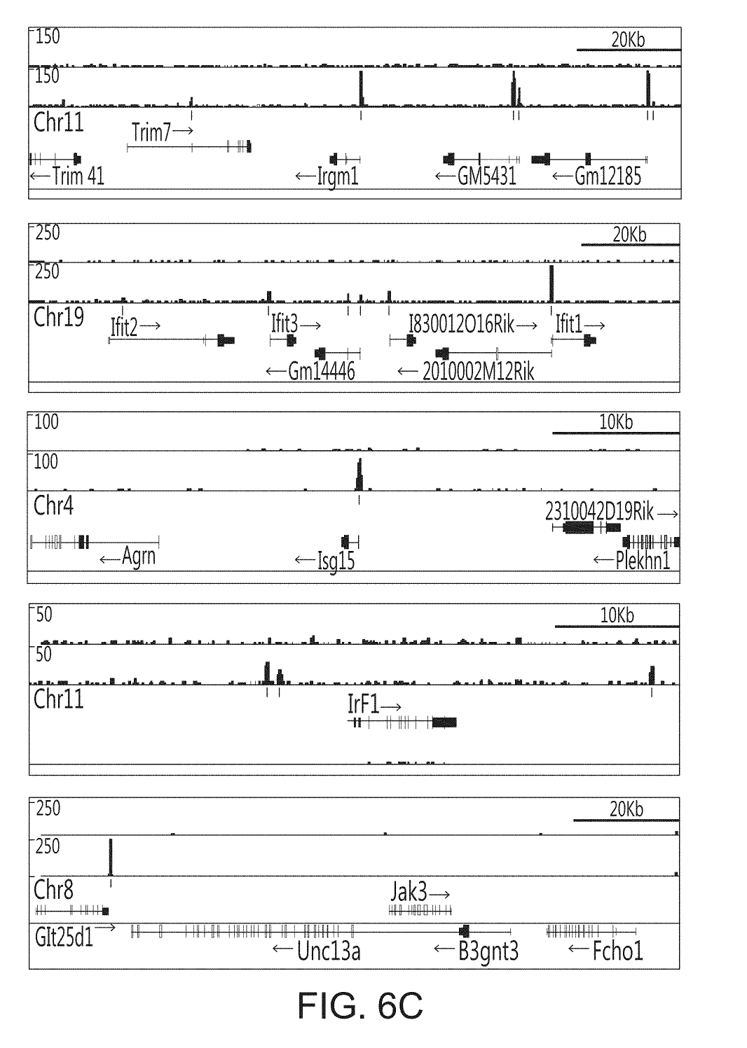

A total of 8 independent ChIPs were pooled for each condition (IRF8 and IgG). Libraries and flow cells were prepared by the IRCM Molecular Biology Core Facility following Illumina's recommendations (Illumina, San Diego, Calif.), with a size selection step targeting fragments between 250 and 500 bp. The ChIP libraries were sequenced on Illumina HiSeq 2000 sequencer. The sequencing yielded 86 and 79 million 50 bp sequence reads for IgG control and IRF8 samples, respectively. The reads were mapped to the mouse mm9 genome assembly using Bowtie with the following parameters: -t --solexa1.3-qual --sam --best mm9. The mapping efficiency was 91.7% for IgG and 91.9% for IRF8 samples. To identify IRF8 binding peaks, we used the MACS 1.4.1 peak finder with the following parameters: --bw 250 --mfold 7.30 --pvalue 1e-5 -g mm. This analysis yielded 11216 genomic regions bound by IRF8 with p-values under the threshold of 10⁻. The genes identified as affected by PbA infection in the expression profiling experiment were queried for the presence IRF8 binding peaks in a 20 kb interval around the gene transcription start site (TSS). This analysis was also performed for all the genes represented on the Illumina mouse WG-6 v2.0 array used in the microarray experiments, to assess the background association of IRF8 peaks with surrounding genes (FIG. 6).

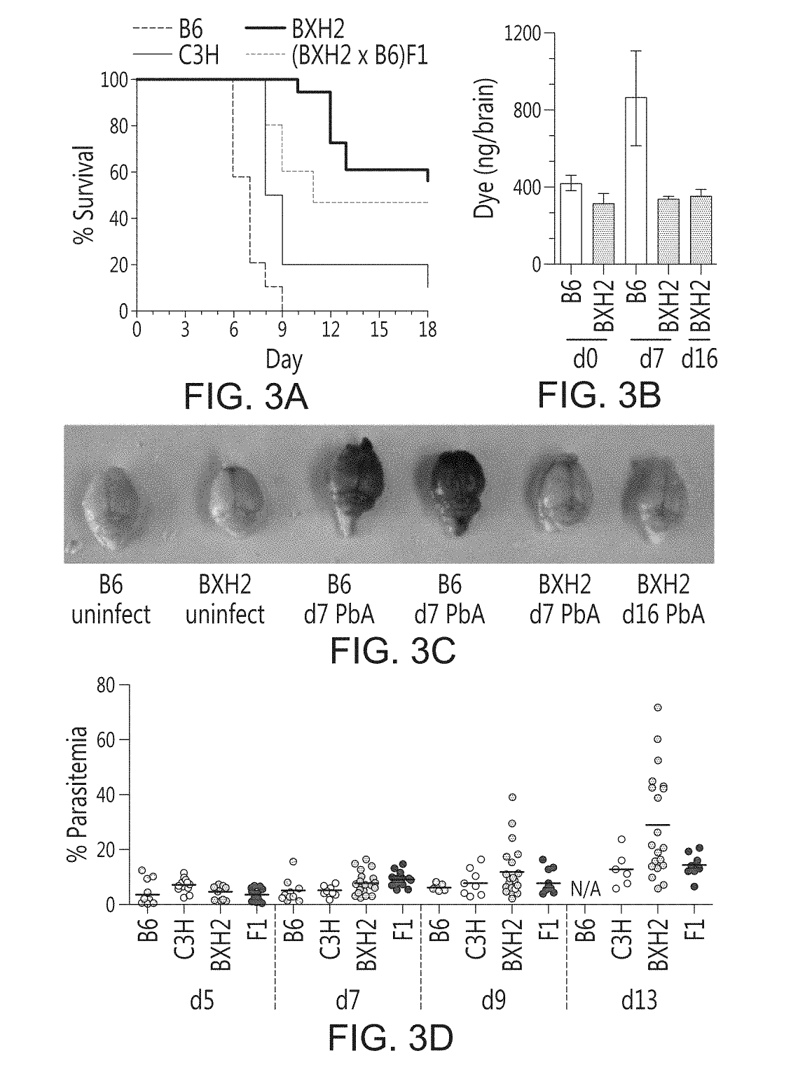

BXH2 is a recombinant inbred mouse strain derived from C57BL/6J (B6) and C3H/HeJ (C3H) parents that carries a severely hypomorphic allele at Irf8 (Irf8^{R294C}) and that causes a myeloid defect expressed as granulocytic hyperplasia, depletion of mononuclear phagocytes, and susceptibility to infections. To assess the contribution of Irf8 to pathological inflammation, BXH2 (n=18) and parental control mice B6 (n=19) and C3H mice (n=10) were infected with the murine agent of cerebral malaria (CM), P. berghei ANKA. Parasite replication in the blood, appearance of neurological symptoms of CM and overall survival were recorded in infected mice over 18 days (FIG. 3). While all B6 and 80% of C3H mice developed CM and succumbed by day 9, BXH2 mice were completely resistant to the CM phase, succumbing later to hyperanemia caused by uncontrolled blood-stage replication of the parasite (FIGS. 3A and D). [BXH2×B6]F1 mice (n=15) showed significant resistance to PbA induced CM when compared to susceptible B6 and C3H parental controls, with approximately 50% of the animals surviving past day 9 (p=0.03 versus C3H, p<0.0001 versus B6), suggesting that the CM-resistance trait of BXH2 is inherited in a co-dominant fashion. Additional phenotyping of a small group of [BXH2×B6]F2 mice (n=17) identified CM-resistance only in mice either homozygote or heterozygote for the Irf8^{R294C} allele, confirming that the protective effect is due to the Irf8^{R294C} mutation with minimal or no contribution of the mixed B6/C3H genetic background of BXH2. These data show that that partial or complete loss of IRF8 function protects mice against lethality in this CM-associated neuroinflammation model. They also confirm that the CM-protective effect of the Irf8^{R294C} mutation is inherited in a co-dominant fashion.

Previous reports have demonstrated that lethal CM in PbA-infected mice is associated with endothelial dysfunction, including loss of integrity of the blood brain barrier (BBB). Using the Evans Blue dye extravasation test (FIGS. 3B and C), it was noted that while PbA-infected B6 mice displayed obvious BBB permeability by d7, infected BXH2 mice retained integrity of the BBB, and excluded Evans Blue dye, both early (day 7) and late (day 16) during infection at levels comparable to uninfected mice (FIGS. 3B and C). Resistance to CM in BXH2 mice was not associated with decreased parasite burden (FIG. 3D), as surviving B6 and C3H controls, as well as BXH2 and [BXH2×B6]F1 showed similar circulating blood parasitemia at days 5, 7 and 9 post-infection (p>0.1). As the infection progressed, however, some of the BXH2 mice developed extremely high levels of blood parasitemia (between d12-21) in contrast to surviving controls and [BXH2×B6]F1s. This high parasitemia, rather than cerebral inflammation, was responsible for the observed mortality. These results demonstrate that, although loss of IRF8 activity is protective against cerebral malaria, it is required to control blood stage replication of PbA late in infection, and that partial IRF8 function in [BXH2×B6]F1 is sufficient to protect against high blood-stage replication.

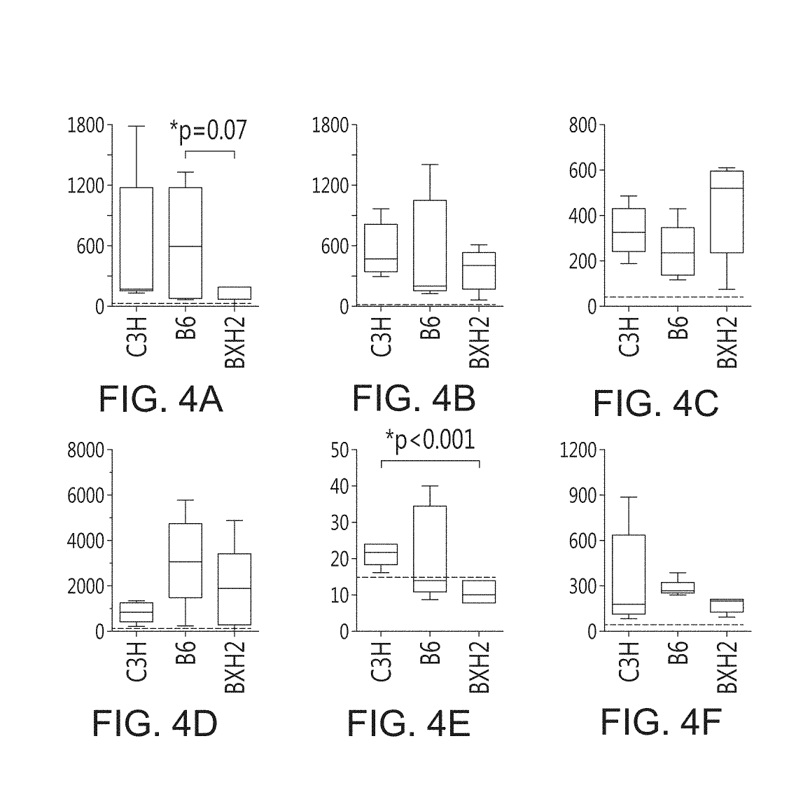

Although there was substantial variation between individual mice of the same group, serum cytokine analysis showed that 6 days post-infection, resistance to PbA in BXH2 mice was associated with reduced levels of TNF-α (compared to C3H controls; p<0.001), and IFNγ (compared to B6 controls; p=0.07), although these differences did not reach statistical significance for the other parental control. Average levels of IL-2, IL-12p70 and VEGF were below the assay limit of detection and no difference in serum levels were detected amongst the other tested cytokines (IL-10, CCL2 (MCP-1), CCL4 (MIP-1β) and CCL5 (RANTES))(FIG. 4).

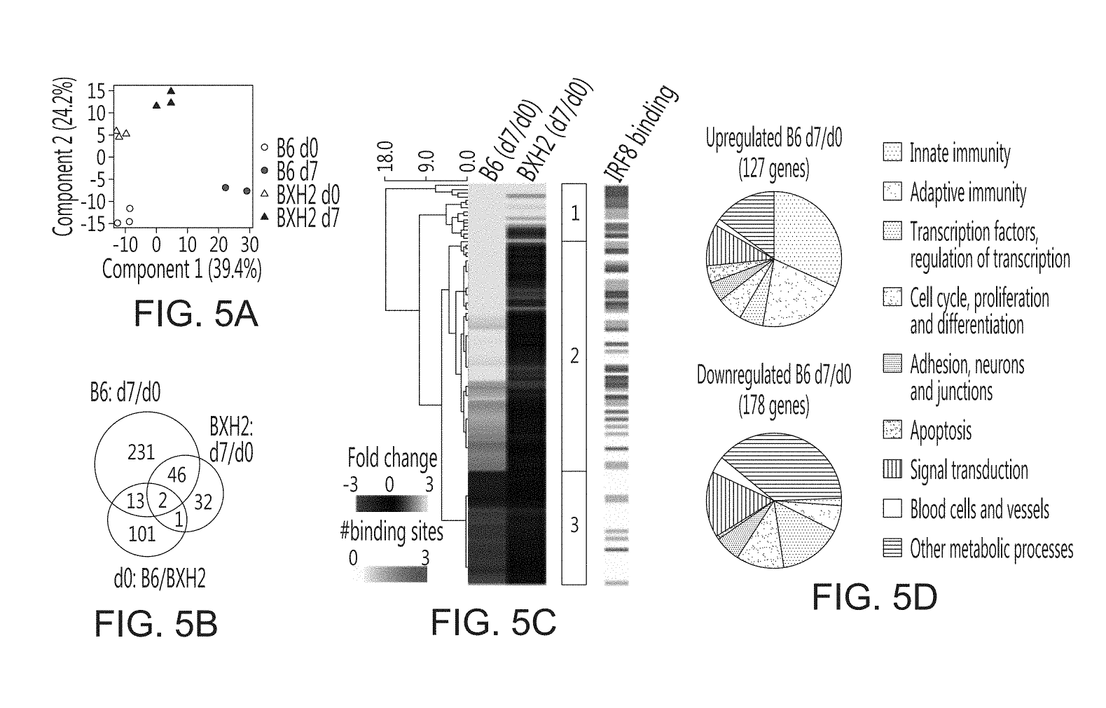

To gain further insight into the genes, proteins and pathways that play a key pathological role during neuroinflammation, and whose expression is regulated by IRF8, several experiments were conducted. First, transcript profiling was used to identify genes differentially regulated in the brains of BXH2 and B6 mice either prior to or during PbA-infection. Principal components analysis (PCA) clustered the samples along two axes: component 1, which explained 39.4% of the variance and was associated to infection status (infection component), and component 2, analogous to strain (genetic component), which explained 24.4% of the variance (FIG. 5A). PCA also indicates that PbA infection had a much stronger impact on transcriptional profiles in B6 mice than in BXH2, with the B6 d7 infected samples forming a remote out-group. In contrast, the BXH2 d7 cluster was only moderately shifted by infection and remained much closer to the BXH2 d0 group (compared to B6 d0 vs. B6 d7) indicating far more modest response by BXH2. Paired t-tests were used to assess the transcriptional response changes due to infection in both B6 and BXH2 mice in order to extract gene lists that include strong changes relevant to pathological neuroinflammation. As suggested by the PCA, B6 response to infection was robust, with 296 unique genes showing statistically significant differences in expression (d0 vs. d7; fold change >2, P_{adj}<0.0⁵). On the other hand, response to infection in BXH2 was more modest with 81 genes reaching statistical significance. More than half of the genes (n=48) regulated by infection in BXH2 were common to the B6 set and may correspond to IRF8-independent regulatory mechanisms. This analysis also identified a subset of 117 genes that show significant differences in expression in B6 vs. BXH2 mice prior to infection. Only ˜10% of these “genetically regulated” genes (n=16) were further significantly modulated by P. berghei infection (FIG. 4B). Importantly, this analysis also identified a subset of 231 genes that were uniquely regulated in B6 mice by infection.

To investigate the role of Irf8 in CM-associated neuroinflammation phenotype, a two-factor ANOVA was performed accounting for both differences in basal level of gene expression in the brain (B6 vs. BXH2 at day 0), and infection-induced transcriptional response to PbA (FIG. 50C). This analysis identified a total of 107 genes (123 probes; fold change >2, P_{adj}<0.05) that were strongly regulated by infection in an Irf8 dependent fashion (p_{adj}-interaction<0.05) (FIG. 50C, Table 1). Euclidean hierarchical clustering of this gene list identified three major categories of transcripts. Group 1 genes (n=15) were up-regulated by infection in both strains (up-regulation more pronounced in B6 than BXH2), group 2 genes were up-regulated by infection in B6 mice but not significantly induced in BXH2 (n=62) and group 3 genes (n=30) were downregulated by infection (stronger repression in B6 compared to BXH2). Using the online Database for Annotation and Integrated Discovery (DAVID) tool to examine the complete list of genes regulated by strain and by infection indicated substantial enrichment for immune response (4.4-fold enrichment above Illumina WG-6 v2.0 chip background, p_{adj}=3.3×10⁻¹²), antigen processing and presentation (11.0-fold enrichment, p_{adj}=1.4×10⁻⁹), defense response (3.8-fold enrichment, p_{adj}=1.7×10⁻⁸), chemotaxis (5.2-fold enrichment, p_{adj}=³.4×10⁻³⁾ and inflammatory response (3.6-fold enrichment, p_{adj}=3.7×10³). Up-regulated genes on these lists include potent pro-inflammatory chemoattractant chemokines that recruit myeloid and lymphoid cells to the site of infection and/or tissue injury such as Cxcl9, Cxcl10, Ccl4, and Ccl12, proteins in myeloid cells associated with phagocytosis of microbes (Fcgr4; low affinity IgG receptor) and maturation of phagosomes (small GTPases Igtp, Irgm1, Gbp2, Gbp3), IRF8's heterodimerization partner (Irf1), and early type I interferon response (Oasl2, Ifit3). Genes under these immune and inflammatory response categories were found to be expressed at a higher level in B6 than in BXH2 mice, consistent with the notion that resistance to CM-associated neuroinflammation in BXH2 is linked to reduced IRF8-dependent inflammatory and innate immune responses, with a strong involvement of the myeloid compartment.

TABLE 1
Fold change of transcripts differentially expressed in a strain and infection dependent
manner (2-way ANOVA, 2 > fold change cut-off, p_{adj}-interaction < 0.05).
Gene order is as seen in FIG. 5C; and genes indicated with a “†” indicate
an IRF8 binding site within 20 kb of the transcription start site. Fold change for
genes with an asterisk is reported as the average of two or more significant probes.

| Group 1 genes | | | Group 2 genes | | | Group 3 genes | | |
|---|---|---|---|---|---|---|---|---|
| GeneID | B6 | BXH2 | GeneID | B6 | BXH2 | GeneID | B6 | BXH2 |
| Igtp† | 17.29 | 6.56 | Xaf1† | 5.22 | 2.40 | H2afv | −1.44 | 1.06 |
| Cxcl10† | 14.90 | 3.18 | Ccl12*† | 5.67 | 1.62 | EG381438 | −1.36 | 1.07 |
| Rsad2† | 12.79 | 3.94 | Psmb9† | 6.09 | 1.16 | Prkag2 | −1.39 | −1.06 |
| Fcgr4† | 11.50 | 2.33 | Cdkn1a* | 5.17 | −1.36 | Tmcc1 | −1.42 | 1.00 |
| Cd274† | 10.38 | 4.03 | Serpina3k | 4.71 | −1.11 | Atp2c1 | −1.40 | 1.01 |
| Gbp3*† | 9.58 | 4.03 | Fosb | 4.19 | −1.34 | 1700123O20Rik | −1.35 | −1.00 |
| Irgm1† | 9.44 | 2.76 | Socs3† | 4.19 | 1.14 | Ccng2 | −1.33 | 1.00 |
| Isg15† | 7.61 | 3.99 | Ccl5† | 4.16 | 1.24 | Arl8b† | −1.25 | 1.01 |
| Oasl2† | 8.31 | 3.25 | Ms4a6d | 4.07 | 1.21 | Slc4a4 | −1.39 | −1.14 |
| Plac8 | 7.41 | 2.49 | Emp1 | 3.81 | 1.10 | Emid2 | −1.57 | −1.15 |
| Cd74*† | 6.66 | 2.97 | Ccl7*† | 3.77 | 1.15 | Hes5 | −2.68 | −1.54 |
| Irf1*† | 6.29 | 2.24 | C4b*† | 4.54 | 1.32 | Gga3 | −2.24 | −1.41 |
| Cxcl9† | 8.25 | 1.57 | H2-Ab1*† | 4.66 | 1.37 | D14Abb1e | −2.41 | −1.29 |
| Ccl4*† | 7.20 | 1.39 | Tap1† | 4.63 | 1.63 | Flt1 | −2.89 | −1.29 |
| Plin4 | 7.11 | −2.20 | Cd52† | 4.19 | 1.57 | Hcn3 | −2.01 | −1.31 |
| | | | Chi3l4† | 4.15 | 1.71 | Akap1 | −2.14 | −1.25 |
| | | | Tap2† | 3.39 | 1.40 | Zbtb44 | −2.05 | −1.26 |
| | | | Lyz1† | 3.62 | 1.36 | Zfp523† | −2.07 | −1.21 |
| | | | Nlrc5† | 3.61 | 1.94 | E330009J07Rik | −2.45 | −1.14 |
| | | | Fkbp5 | 3.07 | −1.44 | Pak1 | −2.11 | −1.12 |
| | | | Map3k6* | 2.85 | −1.13 | Ccm2† | −2.07 | −1.12 |
| | | | Atp5k | 2.72 | −1.09 | E2f6 | −2.00 | −1.08 |
| | | | Angptl4† | 2.71 | −1.08 | 6330407J23Rik | −2.04 | −1.05 |
| | | | Txnip*† | 2.60 | −1.09 | Fcrls | −2.15 | −1.03 |
| | | | AA467197 | 3.37 | 1.15 | Slco1c1 | −2.27 | −1.03 |
| | | | Slc10a6 | 3.23 | 1.01 | 2510022D24Rik | −1.90 | 1.01 |
| | | | Mt2 | 3.31 | −1.08 | Raf1 | −1.67 | 1.02 |
| | | | H2-L | 2.89 | 1.08 | Mettl17 | −2.27 | 1.08 |
| | | | Sult1a1 | 3.01 | 1.03 | Slc38a5 | −2.95 | 1.12 |
| | | | Slc15a3† | 3.04 | 1.15 | Tia1† | −3.07 | 1.03 |
| | | | Ch25h | 3.00 | 1.22 | | | |
| | | | Serping1* | 2.91 | 1.24 | | | |
| | | | Tagln2 | 2.81 | 1.28 | | | |
| | | | Fpr2† | 2.80 | 1.43 | | | |
| | | | Ifi47 | 3.01 | 1.60 | | | |
| | | | Oasl1 | 3.01 | 1.46 | | | |
| | | | Samhd1† | 3.10 | 1.50 | | | |
| | | | Icam1† | 3.10 | 1.36 | | | |
| | | | Psmb8† | 2.40 | 1.64 | | | |
| | | | Batf2† | 2.26 | 1.50 | | | |
| | | | Osmr | 2.39 | 1.23 | | | |
| | | | Ifi205† | 2.47 | 1.34 | | | |
| | | | Ubd† | 2.63 | 1.18 | | | |
| | | | Cyba† | 2.29 | 1.12 | | | |
| | | | Saa3 | 2.26 | 1.07 | | | |
| | | | Bcl2a1b† | 2.35 | 1.05 | | | |
| | | | H2-Eb1 | 2.41 | 1.05 | | | |
| | | | Bcl2a1d† | 2.48 | 1.05 | | | |
| | | | 2410039 | 2.41 | −1.09 | | | |
| | | | M03Rik | | | | | |
| | | | Mobp† | 2.37 | −1.10 | | | |
| | | | Pnpla2 | 2.28 | −1.13 | | | |
| | | | Gna13 | 2.28 | −1.06 | | | |
| | | | Cenpa | 2.20 | −1.32 | | | |
| | | | Synpo | 2.21 | −1.16 | | | |
| | | | Itgad* | 2.03 | −1.07 | | | |
| | | | Ugt1a6a† | 2.05 | −1.02 | | | |
| | | | Adamts9 | 2.12 | 1.02 | | | |
| | | | Phyhd1 | 2.19 | −1.03 | | | |
| | | | Fcgr3 | 2.14 | 1.09 | | | |
| | | | Ctsc† | 2.07 | 1.10 | | | |
| | | | Upp1*† | 2.11 | 1.14 | | | |
| | | | Arpc1b | 2.04 | 1.05 | | | |

Since total brain RNA was used, genes differentially regulated in response to infection in an Irf8-dependent fashion may represent direct transcriptional targets of IRF8 or may be secondary targets that correspond to markers of cell populations that are differentially recruited to the site of infection in B6 and BXH2 mice. To distinguish between genes that are directly or indirectly regulated by Irf8 in response to infection, we mapped genome-wide IRF8 binding sites. For this, chromatin immunoprecipitation was performed followed by high-throughput sequencing (ChIP-seq) on cultured macrophages treated with CpG and IFNγ. The resulting sequence reads were mapped to the mm mouse reference genome and IRF8 binding peaks were identified using MACS peaks finding algorithm. In order to validate ChIP-seq results, IRF8 recruitment was confirmed on several known target sites by independent ChIP-qPCR experiments (FIG. 6A). The list of IRF8-bound genes (identified as containing a IRF8 binding site within a 20 kb window from the transcriptional start site) was intersected with the list of genes differentially regulated by PbA in a strain, infection and putatively Irf8-dependent fashion (FIG. 5C). This intersection revealed a strong enrichment of IRF8 binding sites in genes up-regulated during infection, with IRF8 binding sites detected in 85% of Group 1 genes (13/15) and 50% of Group 2 genes (31/62) (Table 1). In contrast, differentially down-regulated genes did not show any enrichment with only 13% (4/30) of Group 3 genes associated with IRF8 peaks, lower than background peak association (21% of all genes represented on the Illumina array, FIG. 6B). These results strongly suggest that during neuroinflammation, IRF8 functions as a direct transcriptional activator to up-regulate expression of genes that play a key role in this pathological response to PbA infection.

The list of all genes whose expression is regulated in Irf8-competent B6 mice was additionally queried in response to PbA (infection regulated genes; pairwise comparison for B6 d7/d0), for the presence of IRF8 binding sites and in order to identify IRF8-bound genes associated with CM susceptibility and neuropathology. As depicted in FIG. 6B, this analysis showed very strong enrichment for IRF8 binding sites (p<0.0001, Fisher's Exact test) in the vicinity of genes up-regulated by infection, with 74% (92/125) of up-regulated genes bearing one or more IRF8 binding sites within 20 kb of the TSS (FIG. 6B with IRF8-binding profile examples provided in FIG. 6C). Genes showing down-regulation in response to infection did not show such any enrichment above background (FIG. 6B). The AMIGO gene ontology annotation tool was used to functionally examine the list of genes regulated by infection during neuroinflammation in B6 mice (FIG. 5D and Table 2). This list is dominated by genes involved in inflammatory and innate immune response, even more pronounced in the subset (74%) of IRF8-bound genes, which includes inflammatory cytokine and chemokines involved in chemotaxis of myeloid and lymphoid cell types to the sites of infection (Ccl4, Ccl5, Ccl7, Ccl12, Cxcl9, Cxcl10), early innate immune recognition and responses (Nlrc5, Ifi205), response to viral infections (Oasl2, Mx2, Oaslg), type I interferon responsive genes and pathways (Ifit2, Ifit3, Isg15, Rsad2), antigen capture (C1q, C4b, Fcerg1), phagosome maturation (Irgm1, Irgm2, Igtp, Gbp2, Gbp3), antigen processing (Tap1, Tap2) and Class I and Class II MHC-dependent antigen presentation in myeloid cells (B2m, H2-Abl, H2-D, H2-K, H2-L, H2-Q, H2-T22). Furthermore, other IRF family members implicated in early response to antigenic stimuli or danger signals (Irf1, Irf7, Irf9) were also induced (Table 2). These results support a key role for IRF8 as a transcriptional activator of pro-inflammatory genes and associated pathways that underlie the host-driven pathological neuroinflammation seen in PbA infection. These Irf8-regulated pro-inflammatory pathways appear linked primarily to the myeloid cellular compartment.

TABLE 2
Transcriptional response to P. berghei in CM-susceptible B6 mice, sorted according
to ontology category (AMIGO). Irf8 direct targets are significantly enriched in
the upregulated genes (indicated by bold text). Superscript letters refer to genes
where the human ortholog has been identified in GWAS studies for psoriasis (P),
rheumatoid arthritis (RH), celiac disease (C), Crohn's disease (CD), ulcerative
colitis (UC), diabetes (D), multiple sclerosis (MS), systemic lupus erythmatous
(SLE), irritable bowel disease (IBD), or where the human ortholog is found in the
MHC (MHC), which has been implicated in all of them to varying degrees.

| Ontology | Upregulated genes | Downregulated genes |
|---|---|---|
| Innate immunity | C1qb, C4b^{MHC}, Chi3l3, Chi3l4, Cyba, | C1qtnf4 |
| | Gvin1, Ifi205, Ifi47^{CD}, Ifit3, Map3k6, | |
| | Nlrc5, Oasl1, Oasl2, Pglyrp1, Saa3, | |
| | Samd9l, Serping1, Trim21 | |
| Response to | Bcl2a1b, Bst2, Eif2ak2, ler3^{MHC}, | Skiv2l^{MHC} |
| virus | Ifi27l2a, Ifit2, Ifitm1, Ifitm3, Isg15, | |
| | Ly6a, Mx2, Oas1g^{D}, Rsad2, Samhd1 | |
| Chemokines, | Ccl4, Ccl5, Ccl7^{UC}, Ccl12^{IBD}, Cxcl9, | Cxcl12 |
| cytokines, | Cxcl10, Osmr, Socs3^{MS, IBD, D} | |
| receptors | | |
| Response to | Angptl4, Ctsc, Fpr2, Mt2, Serpina3f, | Acvr2b, Dgkb, Dgkz, Dlgap1, Grm4, |
| stimulus, signal | | Lphn1, Mtss1l, Pacsin1^{MHC}, |
| transduction | | Prrt1^{MHC, IBD}, Psd2, Rasgef1a, |
| | | Rgs7bp, Tnrc6a, Unc13c |
| Adaptive | B2m, Cd274, Cd52, Cd74, Fcer1g, | Fclrs |
| Immunity, | Fcgr3^{SLE, IBD}, Fcgr4^{SLE}, H2-Ab1^{C, MHC}, | |
| antigen | H2-D1/L^{MHC}, H2-Eb1^{MHC, MS, RA}, H2- | |
| processing and | K1^{MHC}, H2-K2^{MHC}, H2-Q2^{MHC}, H2- | |
| presentation | Q7^{MHC}, H2-Qa1^{MHC}, H2-T22^{MHC}, | |
| | Psmb8^{MHC}, Psmb9^{MHC}, Tap1^{MHC}, | |
| | Tap2^{MHC} | |
| Transcription | Batf2, D14Ertd668e, Irf1^{UC}, Irf7^{SLE}, | 2210018M11Rik, Arid1a, Atxn7l3, |
| factor, regulation | Irf9, Stat1, Txnip | Atxn7l3b, Bcl11b, Carm1, E2f6, |
| of transcription | | Foxq1, Gtf3a, Hes5, Hist1h2bf, |
| | | Hopx, Jhdm1d, Klf7, Msl1, Myt1l, |
| | | Ncoa1, Nfix^{C}, Pbrm1, Prkcb, |
| | | Rbfox1, Rora, Tcf4, Usf2, Zbtb44, |
| | | Zbtb7a, Zfp523 |
| GTP signaling | Gbp2, Gbp3, Igtp, Irgm1, Irgm2 | Gdi1, Gna11^{MHC}, Gnao1, Rab14, |
| | | Rab5b, Rab6, Rhobtb2, Rnd2, |
| | | Sept3, Tbc1d17 |
| Cell cycle and | Arpc1b, Cdkn1a, Cenpa, Emp1, Gh, | Arl8b, Efna5, Elavl3, Gm16517, |
| proliferation, | Prl, Tagln2, Xdh | Itm2a, Ltbp4, Mau2, Mzt1, Nckap1, |
| cellular | | Nfib, Ntrk3, Ptn, Rnf167, Scrib, |
| differentiation | | Sema6d, Strbp, Thra, Tmod1, Tob1 |
| Adhesion | Icam1^{P, MS, IBD}, Itgad, Lgals3bp, Lgals9 | Bcan, Cd47, Celsr2, Ntm |
| Apoptosis | Bcl2a1d, Ifi27l1, Serpina3g, Tspo, | Ank2, Tia1 |
| | Xaf1 | |
| Protein kinase, | Cmpk2 | Akap1, Camkk2, Dusp8, Fjx1, |
| phosphotase | | Kalrn, Mark2, Pak3, Ppp1r35, |
| | | Ppp5c, Ptprd, Taok1 |
| Ubiquitination | Parp14, Rnf213, Trim25, Ubd^{MHC}, | Fbxo41, Usp11 |
| | Ube1l, Usp18 | |
| RNA processing, | | Brunol4, Eif5a, Mettl17, Pabpn1, |
| translation | | Rbfox1 |
| Transport | Slc15a3 | Abcf2, Apba2, Arf5, Cacng3, |
| | | Gabrb1, Gga3, Gria2, Hcn3, |
| | | Pea15a, Pltp, Scamp3⁷, Slc38a2, |
| | | Slc38a5, Slc38a9, Slc40a1, Slco1c1, |
| | | Syt4, Tmco3, Ugt8a |
| Blood cells and | Anxa2, Tgm2 | Alas2, Ccm2, Flt1, Hba-a1, Hbb-b1, |
| vessels | | Pak1, Ppap2b |
| Neuronal and | Mobp | Epn2, Gjc2, Kif3a, Klc1, Palm, |
| junctions | | Shank3, Spnb4 |
| Metabolic | Adamts9, Ch25h, Fkbp5^{MHC}, Lyz1, | 0610007P14Rik, 1190002N15Rik, |
| processes | Phyhd1, Pnpla2, Sult1a1, Ugt1a6a, | Acot7, Atp6v0d1, B4galt3, |
| | Upp1 | Cyp46a1, Glg1, Hsd3b2, Mbtps1, |
| | | Mgat4b, Mus81, Oxct1, Pcyt2, |
| | | Pde6d, Phldb1, Ppp1ca, Sdr39u1, |
| | | Smpd1 |
| Biological | 2410039M03Rik, 8430408G22Rik, | 1110012J17Rik, 2600009P04Rik, |
| processes or | Glipr2, Gm12250, Ms4a6d, Plac8, | 2900011O08Rik, 3110047P20Rik, |
| unannotated | Plin4 | 4930402H24Rik, 6330407J23Rik, |
| | | Al593442, Caln1, Cops7a, |
| | | D14Abble, D17Wsu92e, |
| | | E330009J07Rik, Fam126b, |
| | | Fam171b, Fam178a, Fam63b, |
| | | Fndc5, Gats, Jph4, Klhdc1, Lonrf2, |
| | | Orf61, Rtn1, Sgtb, Tmem63b, |
| | | Tspan3, Zfp385b, Zfp612 |

Involvement of the host-response pathways identified in Table 2 is not unique to cerebral malaria, so the list of genes regulated by infection in B6 brains during pathological neuroinflammation (d7/d0) was compared with the list of genes contributing to the protective response in the lungs of Mycobacterium tuberculosis infected B6 mice (d30/d0) (Table 3). B6 and BXH2 mice have opposite phenotypes in the two disease models B6 being susceptible to CM, but resistant to M. tuberculosis, while BXH2 is resistant to CM but succumbs extremely rapidly to disseminated mycobacterial disease when infected with M. tuberculosis. Strikingly, of the 123 genes up-regulated more than 2-fold during CM, 66 were also up-regulated >2-fold during M. tuberculosis infection (p<0.0001, Fisher's Exact Test). There was minimal overlap in the down-regulated genes (21 M. tuberculosis-regulated genes overlapping with the 170 PbA-regulated). Looking at the genes up-regulated by both infections, the overwhelming majority (80%) contained at least one IRF8 binding site, again highlighting IRF8 central role during inflammation and host response to infections (Table 3).

TABLE 3
List of genes contributing to the protective response in the lungs of Mycobacterium tuberculosis infected B6 mice (d30/d0).

| | | | | | #IRF8 |
|---|---|---|---|---|---|
| | PbA | Mtb | adj. p- | | binding |
| Gene ID | d7/d0 | 30/0 | value | Gene Name | peaks |
| Gh | 84.82 | | 1.1E−02 | Growth hormone (Gh), mRNA | 1 |
| Gbp2 | 25.38 | 16.53 | 2.0E−03 | Guanylate binding protein 2, mRNA (cDNA clone MGC: 41173 IMAGE: 1230883) | 1 |
| Prl | 22.77 | | 1.6E−02 | Prolactin (Prl), mRNA | |
| Igtp | 17.29 | 14.99 | 2.3E−03 | Interferon gamma induced GTPase (Igtp), mRNA | 3 |
| Cxcl10 | 14.90 | 92.96 | 1.1E−02 | Chemokine (C—X—C motif) ligand 10, mRNA (cDNA clone MGC: 41087 | 3 |
| | | | | IMAGE: 1446589) | |
| Ifit3 | 14.88 | 5.28 | 2.4E−03 | Interferon-induced protein with tetratricopeptide repeats 3, mRNA (cDNA clone | 3 |
| | | | | MGC: 6081 IMAGE: 3487345) | |
| Rsad2 | 12.79 | 3.93 | 5.3E−03 | Viperin (Vig1) | 4 |
| Fcgr4 | 11.50 | 13.19 | 3.1E−03 | Fc receptor, IgG, low affinity IV (Fcgr4), mRNA | 2 |
| Cd274 | 10.38 | 11.25 | 3.1E−03 | CD274 antigen (Cd274), mRNA | 2 |
| Irgm1 | 9.44 | 10.82 | 5.3E−03 | Immunity-related GTPase family M member 1 (Irgm1), mRNA | 1 |
| Gbp3 | 9.40 | 12.61 | 2.0E−03 | Guanylate binding protein 3, mRNA (cDNA clone MGC: 29218 IMAGE: 5036920) | 2 |
| Cxcl9 | 8.25 | 413.81 | 8.8E−03 | Chemokine (C—X—C motif) ligand 9, mRNA (cDNA clone MGC: 6179 IMAGE: | 1 |
| | | | | 3257716) | |
| Ifi27l2a | 7.91 | 2.22 | 5.3E−03 | Interferon stimulated gene 12 (Isg12) | |
| Usp18 | 7.68 | 3.63 | 5.3E−03 | Ubiquitin specific protease UBP43 | 3 |
| Isg15 | 7.61 | | 2.0E−03 | ISG15 ubiquitin-like modifier, mRNA (cDNA clone MGC: 18616 IMAGE: 3670747) | 1 |
| Plac8 | 7.41 | 4.20 | 7.6E−03 | C15 protein | |
| S3-12 | 7.11 | −4.02 | 1.9E−02 | Plasma membrane associated protein, S3-12 (S3-12), mRNA | |
| Cd74 | 6.51 | 2.25 | 2.5E−03 | CD74 antigen (invariant polypeptide of major histocompatibility complex, class II | 2 |
| | | | | antigen-associated), mRNA (cDNA clone MGC: 6 | |
| Irf1 | 6.49 | 3.53 | 2.5E−03 | Interferon regulatory factor 1, mRNA (cDNA clone MGC: 6190 IMAGE: 3600525) | 2 |
| Rnf213 | 6.38 | 2.06 | 2.4E−03 | D11Ertd759e | 2 |
| Lgals3bp | 6.36 | 3.86 | 6.5E−03 | Lectin, galactoside-binding, soluble, 3 binding protein (Lgals3bp), mRNA | |
| Psmb9 | 6.09 | 8.46 | 2.0E−03 | Proteasome (prosome, macropain) subunit, beta type 9 (large multifunctional | 5 |
| | | | | peptidase 2) (Psmb9), mRNA | |
| Ccl12 | 6.06 | 10.68 | 1.0E−02 | Chemokine (C-C motif) ligand 12, mRNA (cDNA clone MGC: 41146 IMAGE: 1548072) | 1 |
| Irgm2 | 5.79 | 7.88 | 2.7E−02 | Immunity-related GTPase family M member 2 (Irgm2), mRNA | 2 |
| Serpina3f | 5.63 | | 5.3E−03 | serine (or cysteine) peptidase inhibitor, clade A, member 3F | 1 |
| Oasl2 | 5.58 | 5.97 | 8.7E−03 | 2-5 oligoadenylate synthetase-like 2, mRNA (cDNA clone MGC: 6269 | 1 |
| | | | | IMAGE: 2646375) | |
| Ifitm3 | 5.57 | | 5.0E−03 | Interferon induced transmembrane protein 3 (Ifitm3), mRNA | 5 |
| Serpina3g | 5.49 | 24.45 | 2.5E−03 | serine (or cysteine) peptidase inhibitor, clade A, member 3G | 1 |
| Xaf1 | 5.22 | | 5.3E−03 | gene model 881, (NCBI) | 1 |
| Cdkn1a | 5.17 | | 4.5E−02 | Cyclin-dependent kinase inhibitor 1A (P21) (Cdkn1a), transcript variant 1, mRNA | |
| Ifitm1 | 4.84 | | 1.2E−02 | Interferon induced transmembrane protein 1 (Ifitm1), transcript variant 1, mRNA | 1 |
| Parp14 | 4.74 | 2.78 | 8.8E−03 | Poly (ADP-ribose) polymerase family, member 14, mRNA (cDNA clone | 2 |
| | | | | IMAGE: 5065398) | |
| Ccl4 | 4.71 | 4.51 | 2.7E−02 | Strain SJL/J small inducible cytokine A4 (ScyA4) | 4 |
| C4b | 4.64 | | 8.8E−03 | Complement component 4B (Childo blood group) (C4b), mRNA | 1 |
| Tap1 | 4.63 | | 6.9E−03 | Transporter 1, ATP-binding cassette, sub-family B (MDR/TAP), mRNA (cDNA clone | 5 |
| | | | | MGC: 6181 IMAGE: 3257734) | |
| Cd52 | 4.19 | 7.79 | 2.3E−02 | CD52 antigen, mRNA (cDNA clone MGC: 40993 IMAGE: 1396480) | 3 |
| Socs3 | 4.19 | 3.06 | 4.5E−02 | Suppressor of cytokine signaling 3 (Socs3), mRNA | 2 |
| H2-K1 | 4.17 | 7.92 | 3.0E−03 | MRNA similar to histocompatibility 2, D region locus 1 (cDNA clone MGC: 25703 | 1 |
| | | | | IMAGE: 3675316) | |
| Ccl5 | 4.16 | 23.91 | 2.4E−02 | Chemokine (C-C motif) ligand 5, mRNA (cDNA clone MGC: 35989 IMAGE: 4925413) | 4 |
| Chi3l4 | 4.15 | | 5.6E−03 | Chitinase 3-like 4 (Chi3l4), mRNA | 1 |
| Ms4a6d | 4.07 | 18.49 | 1.5E−02 | Membrane-spanning 4-domains, subfamily A, member 6D, mRNA (cDNA clone | |
| | | | | MGC: 25778 IMAGE: 4016611) | |
| Emp1 | 3.81 | | 3.2E−02 | Epithelial membrane protein 1 (Emp1), mRNA | |
| H2-Ab1 | 3.08 | 7.76 | 5.3E−03 | histocompatibility 2, class II antigen A, beta 1 | 1 |
| Mx2 | 3.75 | | 1.9E−02 | Myxovirus (influenza virus) resistance 2, mRNA (cDNA clone MGC: 5689 | 1 |
| | | | | IMAGE: 3591798) | |
| H2-D1 | 3.19 | 4.07 | 5.9E−03 | MHC class Ib antigen Qa-1 (H2-T23) | 4 |
| Irf7 | 3.65 | 8.19 | 2.0E−03 | Interferon regulatory factor 7 (Irf7), mRNA | 1 |
| Lyz1 | 3.62 | | 1.0E−02 | Lysozyme 1 (Lyz1), mRNA | 2 |
| Nlrc5 | 3.61 | 8.55 | 2.0E−03 | expressed sequence AI451557 (AI451557), mRNA. | 1 |
| Tap2 | 3.39 | 4.21 | 5.6E−03 | Transporter 2, ATP-binding cassette, sub-family B (MDR/TAP), mRNA (cDNA clone | 4 |
| | | | | MGC: 11732 IMAGE: 3968225) | |
| Txnip | 3.36 | | 4.1E−02 | Thioredoxin interacting protein, mRNA (cDNA clone MGC: 25534 IMAGE: 3591421) | 2 |
| Chi3l3 | 3.36 | | 2.6E−03 | Chitinase 3-like 3 (Chi3l3), mRNA | 1 |
| Eif2ak2 | 3.32 | 2.53 | 4.6E−03 | Eukaryotic translation initiation factor 2-alpha kinase 2, mRNA (cDNA clone | |
| | | | | MGC: 11397 IMAGE: 3964935) | |
| Mt2 | 3.31 | | 4.5E−02 | Metallothionein 2, mRNA (cDNA clone MGC: 19383 IMAGE: 2651471) | |
| Samdh9l | 3.30 | | 1.0E−02 | sterile alpha motif domain containing 9-like | 1 |
| Stat1 | 3.25 | 17.30 | 2.1E−02 | Signal transducer and activator of transcription 1, mRNA (cDNA clone MGC: 6411 | |
| | | | | IMAGE: 3587831) | |
| Oas1g | 3.22 | 5.59 | 2.5E−03 | 2-5 oligoadenylate synthetase 1G (Oas1g), mRNA | 1 |
| Ifit2 | 3.19 | 8.05 | 6.3E−03 | Interferon-induced protein with tetratricopeptide repeats 2 (Ifit2), mRNA | 1 |
| Glipr2 | 3.13 | 5.28 | 3.5E−02 | GLI pathogenesis-related 2, mRNA (cDNA clone MGC: 28417 IMAGE: 4037002) | 2 |
| Tgm2 | 3.11 | | 3.0E−02 | Transglutaminase 2, C polypeptide, mRNA (cDNA clone MGC: 6152 IMAGE: 3256943) | 2 |
| Icam1 | 3.10 | | 8.8E−03 | Intercellular adhesion molecule 1 (Icam1), mRNA | 2 |
| Psmb8 | 3.10 | 7.99 | 6.2E−03 | Proteasome (prosome, macropain) subunit, beta type 8 (large multifunctional | 5 |
| | | | | peptidase 7), mRNA (cDNA clone MGC: 6535 IMAGE: 265 | |
| Samhd1 | 3.10 | 3.93 | 5.9E−03 | SAM domain and HD domain, 1, mRNA (cDNA clone MGC: 14068 IMAGE: 4037046) | 3 |
| Fkbp5 | 3.07 | | 3.0E−02 | FK506 binding protein 5, mRNA (cDNA clone MGC: 18417 IMAGE: 4237766) | 1 |
| B2m | 3.04 | | 5.3E−03 | Beta-2 microglobulin mRNA, segment 1, clones pBRcB-(1-3). | 3 |
| Slc15a3 | 3.04 | 6.14 | 2.5E−02 | Solute carrier family 15, member 3 (Slc15a3), mRNA | 1 |
| Ifi47 | 3.01 | 8.99 | 6.2E−03 | Interferon gamma inducible protein 47, mRNA (cDNA clone MGC: 11403 | 3 |
| | | | | IMAGE: 2651113) | |
| Sult1a1 | 3.01 | | 1.0E−02 | Sulfotransferase family 1A, phenol-preferring, member 1 (Sult1a1), mRNA | |
| Ch25h | 3.00 | 5.86 | 1.2E−02 | Cholesterol 25-hydroxylase (Ch25h), mRNA | |
| 8430408G22Rik | 2.91 | | 3.5E−02 | RIKEN cDNA 8430408G22 gene (8430408G22Rik), mRNA | |
| Ccl7 | 2.91 | 5.76 | 2.6E−02 | Chemokine (C-C motif) ligand 7 (Ccl7), mRNA | 1 |
| Trim21 | 2.90 | 2.87 | 2.5E−03 | Tripartite motif-containing 21, mRNA (cDNA clone MGC: 6059 IMAGE: 3584654) | 3 |
| Serping1 | 2.88 | | 1.9E−02 | Serine (or cysteine) peptidase inhibitor, clade G, member 1, mRNA (cDNA clone | |
| | | | | MGC: 5908 IMAGE: 3485810) | |
| H2-K2 | 2.86 | | 9.0E−03 | LOC56628 | 1 |
| H2-T22 | 2.85 | 4.82 | 7.6E−03 | Histocompatibility 2, T region locus 10, mRNA (cDNA clone MGC: 25390 | 3 |
| | | | | IMAGE: 4165944) | |
| Map3k6 | 2.85 | | 3.3E−02 | Mitogen-activated protein kinase kinase kinase 6 (Map3k6), mRNA | |
| D14Ertd668e | 2.83 | | 6.0E−03 | DNA segment, Chr 14, ERATO Doi 668, expressed, mRNA (cDNA clone MGC: 29273 | 1 |
| | | | | IMAGE: 5067268) | |
| — | 2.81 | | 1.6E−02 | RIKEN cDNA 1200016E24 gene | |
| Tagln2 | 2.81 | | 2.0E−02 | Transgelin | |
| Fpr2 | 2.80 | 10.17 | 2.1E−02 | Formyl peptide receptor 2 (Fpr2), mRNA | 2 |
| Irf9 | 2.80 | 2.15 | 1.3E−02 | Interferon regulatory factor 9, mRNA (cDNA clone MGC: 13985 IMAGE: 3257714) | 1 |
| Pglyrp1 | 2.80 | 3.50 | 1.8E−02 | Peptidoglycan recognition protein 1, mRNA (cDNA clone MGC: 11430 | |
| | | | | IMAGE: 3969014) | |
| H2-Q2 | 2.78 | | 1.4E−02 | Histocompatibility 2, Q region locus 2 (H2-Q2), mRNA | 2 |
| Angptl4 | 2.71 | | 2.9E−02 | Angiopoietin-like 4, mRNA (cDNA clone MGC: 35885 IMAGE: 5137159) | 1 |
| Fcer1g | 2.71 | 5.57 | 2.1E−02 | Fc receptor, IgE, high affinity I, gamma polypeptide, mRNA (cDNA clone MGC: 36077 | 1 |
| | | | | IMAGE: 5065647) | |
| Ly6a | 2.68 | | 7.5E−03 | Lymphocyte antigen 6 complex, locus A, mRNA (cDNA clone MGC: 6188 | 1 |
| | | | | IMAGE: 3486025) | |
| Upp1 | 2.65 | 4.24 | 2.2E−02 | Uridine phosphorylase 1, mRNA (cDNA clone MGC: 41205 IMAGE: 5144694) | 1 |
| Ube1l | 2.64 | 2.60 | 1.1E−02 | Ubiquitin-activating enzyme E1-like, mRNA (cDNA clone IMAGE: 4013998) | 1 |
| Oasl1 | 2.63 | 3.93 | 2.2E−02 | Oligoadenylate synthetase-like protein-2 | |
| Ubd | 2.63 | 259.08 | 5.3E−03 | Ubiquitin D, mRNA (cDNA clone MGC: 41063 IMAGE: 1347593) | 1 |
| H2-L | 2.59 | | 2.7E−02 | H2-L | 1 |
| Tspo | 2.54 | | 1.6E−02 | Translocator protein, mRNA (cDNA clone MGC: 6086 IMAGE: 3493196) | 1 |
| H2-Q7 | 2.52 | | 8.8E−03 | Histocompatibility 2, Q region locus 7 (H2-Q7), mRNA | 2 |
| Trim25 | 2.51 | 2.40 | 2.2E−02 | tripartite motif-containing 25 | 1 |
| Anxa2 | 2.49 | | 3.3E−02 | Annexin A2, mRNA (cDNA clone MGC: 6547 IMAGE: 2655513) | 1 |
| Bcl2a1d | 2.48 | | 5.0E−03 | B-cell leukemia/lymphoma 2 related protein A1d, mRNA (cDNA clone MGC: 41219 | 1 |
| | | | | IMAGE: 1363928) | |
| Ifi205 | 2.47 | 8.65 | 2.5E−03 | (strain C57BI/6) mRNA sequence | 1 |
| 2410039M03Rik | 2.41 | | 4.6E−02 | 2410039M03Rik | |
| H2-Eb1 | 2.41 | 4.78 | 2.5E−03 | Histocompatibility 2, class II antigen E beta (H2-Eb1), mRNA | |
| Osmr | 2.39 | | 2.7E−02 | Oncostatin M receptor (Osmr), mRNA | |
| Mobp | 2.37 | | 4.0E−02 | Myelin-associated oligodendrocytic basic protein (Mobp), transcript variant 1, mRNA | 1 |
| Bcl2a1b | 2.35 | 2.63 | 1.3E−02 | B-cell leukemia/lymphoma 2 related protein A1a, mRNA (cDNA clone MGC: 41220 | 1 |
| | | | | IMAGE: 1226745) | |
| Bst2 | 2.33 | | 2.0E−03 | Bone marrow stromal cell antigen 2, mRNA (cDNA clone MGC: 28276 | 1 |
| | | | | IMAGE: 4009434) | |
| Cyba | 2.29 | 4.38 | 3.5E−02 | Cytochrome b-245, alpha polypeptide (Cyba), mRNA | 1 |
| Pnpla2 | 2.28 | | 2.9E−02 | Patatin-like phospholipase domain containing 2, mRNA (cDNA clone IMAGE: 4982482) | |
| Batf2 | 2.26 | | 4.6E−03 | Basic leucine zipper transcription factor, ATF-like 2, mRNA (cDNA clone MGC: 37488 | 1 |
| | | | | IMAGE: 4984403) | |
| Saa3 | 2.26 | 155.32 | 4.1E−02 | Serum amyloid A 3 (Saa3), mRNA | |
| Cmpk2 | 2.24 | 3.44 | 3.1E−03 | Cytidine monophosphate (UMP-CMP) kinase 2, mitochondrial (Cmpk2), nuclear gene | 3 |
| | | | | encoding mitochondrial protein, mRNA | |
| Itgad | 2.23 | | 4.1E−02 | integrin, alpha D | |
| Cenpa | 2.20 | 4.28 | 1.1E−02 | Centromere protein A, mRNA (cDNA clone MGC: 13888 IMAGE: 4018429) | |
| Phyhd1 | 2.19 | | 4.0E−02 | Phytanoyl-CoA dioxygenase domain containing 1, mRNA (cDNA clone MGC: 178922 | |
| | | | | IMAGE: 9053914) | |
| H2-T17 | 2.18 | | 1.4E−02 | H2-T17 | 3 |
| Gm12250 | 2.16 | | 3.0E−02 | LOC215405 | 2 |
| Xdh | 2.16 | | 1.3E−02 | Similar to hypothetical protein MGC37588, mRNA (cDNA clone MGC: 28125 | 4 |
| | | | | IMAGE: 3980327) | |
| Lgals9 | 2.15 | | 2.4E−03 | Lectin, galactose binding, soluble 9, mRNA (cDNA clone MGC: 5882 IMAGE: 3601419) | |
| Fcgr3 | 2.14 | 7.42 | 5.4E−03 | Fc gamma receptor III (Fcgr3) mRNA, Fcgr3-b allele | |
| Ier3 | 2.14 | | 4.8E−02 | Immediate early response 3 (Ier3), mRNA | 2 |
| Adamts9 | 2.12 | | 2.2E−02 | Adamts9 | |
| Ifi27l1 | 2.10 | | 6.2E−03 | Interferon, alpha-inducible protein 27 like 1, mRNA (cDNA clone MGC: 149996 | |
| | | | | IMAGE: 40091489) | |
| Ctsc | 2.07 | 5.59 | 8.8E−03 | Cathepsin C (Ctsc), mRNA | 1 |
| Gvin1 | 2.06 | | 1.6E−02 | GTPase, very large interferon inducible 1 (Gvin1), transcript variant B, mRNA | 1 |
| Ugt1a6a | 2.05 | | 7.6E−03 | UDP glucuronosyltransferase 1 family, polypeptide A6B, mRNA (cDNA clone | 2 |
| | | | | MGC: 36247 IMAGE: 5050488) | |
| Arpc1b | 2.04 | | 3.5E−02 | Actin related protein 2/3 complex, subunit 1B, mRNA (cDNA clone MGC: 8155 | |
| | | | | IMAGE: 3589768) | |
| C1qb | 2.02 | 13.98 | 2.5E−02 | Complement component 1, q subcomponent, beta polypeptide (C1qb), mRNA | 2 |
| Atxn7l3b | −2.00 | | 2.3E−02 | predicted gene, ENSMUSG00000074747 | |
| E2f6 | −2.00 | | 3.4E−02 | E2F transcription factor 6, mRNA (cDNA clone MGC: 46747 IMAGE: 5358554) | |
| Nckap1 | −2.00 | | 6.2E−03 | NCK-associated protein 1, mRNA (cDNA clone IMAGE: 3488144) | 1 |
| Pcyt2 | −2.00 | | 2.5E−02 | Phosphate cytidylyltransferase 2, ethanolamine, mRNA (cDNA clone MGC: 11578 | |
| | | | | IMAGE: 3707732) | |
| Tob1 | −2.00 | | 3.1E−02 | Transducer of ErbB-2.1 (Tob1), mRNA | |
| Abcf2 | −2.01 | | 4.6E−02 | ATP-binding cassette, sub-family F (GCN20), member 2 (Abcf2), nuclear gene | 1 |
| | | | | encoding mitochondrial protein, mRNA | |
| Hcn3 | −2.01 | | 7.4E−03 | Hyperpolarization-activated, cyclic nucleotide-gated K+ 3 (Hcn3), mRNA | |
| Mus81 | −2.01 | | 1.8E−02 | MUS81 endonuclease homolog (yeast), mRNA (cDNA clone MGC: 36246 | |
| | | | | IMAGE: 5038349) | |
| Ncoa1 | −2.01 | | 1.6E−02 | Nuclear receptor coactivator 1 (Ncoa1), mRNA | |
| Ntrk3 | −2.01 | | 3.0E−02 | Neurotrophic tyrosine kinase, receptor, type 3 (Ntrk3), transcript variant 1, mRNA | |
| Palm | −2.01 | | 1.3E−02 | Paralemmin, mRNA (cDNA clone MGC: 19169 IMAGE: 4223845) | |
| Pbrm1 | −2.01 | | 2.5E−02 | polybromo 1 | |
| Strbp | −2.01 | −2.17 | 3.3E−02 | RIKEN cDNA 6430510M02 gene (6430510M02Rik), mRNA. | |
| Arl8b | −2.02 | 2.23 | 1.1E−02 | ADP-ribosylation factor-like 8B (Arl8b), mRNA | 1 |
| D17Wsu92e | −2.02 | | 1.4E−02 | DNA segment, Chr 17, Wayne State University 92, expressed | 1 |
| Gria2 | −2.02 | | 1.5E−02 | Glutamate receptor, ionotropic, AMPA2 (alpha 2) (Gria2), transcript variant 2, mRNA | |
| Lonrf2 | −2.02 | | 2.9E−02 | LON peptidase N-terminal domain and ring finger 2, mRNA (cDNA clone MGC: 170754 | |
| | | | | IMAGE: 8862149) | |
| Pltp | −2.02 | −2.51 | 3.0E−02 | Phospholipid transfer protein, mRNA (cDNA clone MGC: 6006 IMAGE: 3491360) | 2 |
| Myt1l | −2.03 | | 6.1E−03 | Myelin transcription factor 1-like (Myt1l), transcript variant 2, mRNA | |
| Oxct1 | −2.03 | | 8.7E−03 | Scot mRNA for succinyl CoA transferase | 1 |
| Taok1 | −2.03 | | 2.8E−02 | TAO kinase 1, mRNA (cDNA clone IMAGE: 1380120) | |
| Zfp385b | −2.03 | | 4.6E−03 | Zinc finger protein 385B, mRNA (cDNA clone MGC: 169863 IMAGE: 8861258) | |
| 6330407J23Rik | −2.04 | | 2.9E−03 | RIKEN cDNA 6330407J23 gene (6330407J23Rik), mRNA | |
| Apba2 | −2.04 | | 2.5E−03 | X11 protein mRNA, 3 end | |
| Fam126b | −2.04 | | 1.1E−02 | Family with sequence similarity 126, member B, mRNA (cDNA clone MGC: 76460 | 1 |
| | | | | IMAGE: 30431671) | |
| Sdr39u1 | −2.04 | | 1.6E−02 | Short chain dehydrogenase/reductase family 39U, member 1 (Sdr39u1), mRNA | 1 |
| 1190002N15Rik | −2.05 | | 2.1E−02 | RIKEN cDNA 1190002N15 gene | |
| Atxn7l3 | −2.05 | | 2.3E−02 | ataxin 7-like 3 | |
| Cyp46a1 | −2.05 | | 2.0E−02 | Cytochrome P450, family 46, subfamily a, polypeptide 1, mRNA (cDNA clone | |
| | | | | MGC: 18311 IMAGE: 4195579) | |
| Gna11 | −2.05 | | 1.5E−02 | Guanine nucleotide binding protein, alpha 11, mRNA (cDNA clone MGC: 18562 | |
| | | | | IMAGE: 4206878) | |
| Ppp5c | −2.05 | | 1.9E−02 | Protein phosphatase 5, catalytic subunit, mRNA (cDNA clone MGC: 5847 | |
| | | | | IMAGE: 3590322) | |
| Ptn | −2.05 | | 1.7E−02 | Pleiotrophin (Ptn), mRNA | |
| Sept3 | −2.05 | | 2.6E−02 | Septin 3 (Sept3), mRNA | |
| Thra | −2.05 | −2.12 | 8.8E−03 | Thra | |
| Zbtb44 | −2.05 | | 6.2E−03 | BC038156 | |
| Hist1h2bf | −2.06 | | 1.9E−02 | Histone cluster 1, H2bf (Hist1h2bf), mRNA | |
| Ppp1r35 | −2.06 | | 3.0E−02 | RIKEN cDNA 2010007H12 gene, mRNA (cDNA clone MGC: 62925 IMAGE: 1429227) | |
| Ccm2 | −2.07 | | 1.6E−02 | Cerebral cavernous malformation 2 homolog (human) (Ccm2), mRNA | 2 |
| Glg1 | −2.07 | | 1.3E−02 | Golgi apparatus protein 1, mRNA (cDNA clone MGC: 29292 IMAGE: 4239405) | |
| Tmco3 | −2.07 | | 6.3E−03 | Transmembrane and coiled-coil domains 3, mRNA (cDNA clone IMAGE: 3967158) | 1 |
| Zfp523 | −2.07 | | 8.7E−03 | PREDICTED: Mus musculus similar to collagen, type VIII, alpha 2 (LOC100042764), | 1 |
| | | | | mRNA | |
| — | −2.08 | | 3.0E−02 | LOC385086 | |
| Carm1 | −2.08 | | 1.3E−02 | Coactivator-associated arginine methyltransferase 1, mRNA (cDNA clone MGC: 46828 | |
| | | | | IMAGE: 4935077) | |
| Gtrgeo22 | −2.08 | | 3.5E−02 | Gene trap ROSA b-geo 22 (Gtrgeo22), mRNA | 1 |
| Pacsin1 | −2.08 | | 3.8E−03 | Protein kinase C and casein kinase substrate in neurons 1, mRNA (cDNA clone | |
| | | | | MGC: 25285 IMAGE: 4527708) | |
| Pde6d | −2.08 | | 7.6E−03 | Phosphodiesterase 6D, cGMP-specific, rod, delta, mRNA (cDNA clone MGC: 11435 | |
| | | | | IMAGE: 3964336) | |
| Rnf167 | −2.08 | | 4.0E−02 | Ring finger protein 167, mRNA (cDNA clone MGC: 18686 IMAGE: 4241357) | 1 |
| Tmem63b | −2.08 | | 1.2E−02 | Transmembrane protein 63b, mRNA (cDNA clone IMAGE: 4160190) | |
| Nfix | −2.09 | −2.02 | 1.3E−02 | Nuclear factor I/X, mRNA (cDNA clone MGC: 5944 IMAGE: 3491917) | |
| Caln1 | −2.09 | | 3.1E−02 | Calneuron 1 (Caln1) | |
| Dgkb | −2.09 | | 8.7E−03 | Diacylglycerol kinase, beta, mRNA (cDNA clone MGC: 99855 IMAGE: 30649561) | |
| Mbtps1 | −2.09 | | 2.9E−02 | Membrane-bound transcription factor peptidase, site 1 (Mbtps1), mRNA | |
| Rora | −2.09 | | 3.0E−02 | RAR-related orphan receptor alpha, mRNA (cDNA clone MGC: 5892 IMAGE: 3592667) | |
| Scrib | −2.09 | | 1.7E−02 | Scribbled homolog (Drosophila), mRNA (cDNA clone IMAGE: 4459388) | |
| Shank3 | −2.09 | −3.49 | 3.8E−03 | SH3/ankyrin domain gene 3 (Shank3), mRNA | |
| Bcl11b | −2.10 | 5.31 | 8.8E−03 | B-cell leukemia/lymphoma 11B, mRNA (cDNA clone MGC: 27524 IMAGE: 4457123) | |
| Cops7a | −2.10 | | 1.3E−02 | COP9 (constitutive photomorphogenic) homolog, subunit 7a (Arabidopsis thaliana), | |
| | | | | mRNA (cDNA clone MGC: 5772 IMAGE: 3593979) | |
| Fam178a | −2.10 | | 1.4E−02 | family with sequence similarity 178, member A | |
| Foxq1 | −2.10 | | 8.8E−03 | Forkhead box Q1 (Foxq1), mRNA | |
| Rhobtb2 | −2.10 | | 1.6E−02 | Rho-related BTB domain containing 2 (Rhobtb2), mRNA | 1 |
| Zfp612 | −2.10 | | 8.8E−03 | Zinc finger protein 612, mRNA (cDNA clone IMAGE: 3586510) | |
| Eif5a | −2.11 | | 1.6E−02 | Eukaryotic translation initiation factor 5A, mRNA (cDNA clone MGC: 25474 | 1 |
| | | | | IMAGE: 4482804) | |
| Pak1 | −2.11 | 3.15 | 5.9E−03 | P21 (CDKN1A)-activated kinase 1 (Pak1), mRNA | |
| Tspan3 | −2.11 | | 3.6E−02 | Tspan-3 mRNA for tetraspanin | |
| 1110012J17Rik | −2.12 | −2.39 | 9.3E−03 | RIKEN cDNA 1110012J17 gene (1110012J17Rik), transcript variant 1, mRNA | |
| Dgkz | −2.12 | | 1.8E−02 | Diacylglycerol kinase zeta, mRNA (cDNA clone IMAGE: 2650291) | |
| Gnao1 | −2.12 | | 2.8E−02 | Guanine nucleotide binding protein, alpha O (Gnao1), transcript variant A, mRNA | |
| Hsd3b2 | −2.12 | | 2.7E−02 | Hydroxy-delta-5-steroid dehydrogenase, 3 beta- and steroid delta-isomerase 2 | |
| | | | | (Hsd3b2), mRNA | |
| Ntm | −2.13 | | 1.5E−02 | Neurotrimin, mRNA (cDNA clone MGC: 30504 IMAGE: 4480983) | |
| Slc38a9 | −2.13 | | 2.3E−02 | Solute carrier family 38, member 9 (Slc38a9), mRNA | 1 |
| Cd47 | −2.14 | | 1.6E−02 | CD47 antigen (Rh-related antigen, integrin-associated signal transducer), mRNA | 1 |
| | | | | (cDNA clone MGC: 13838 IMAGE: 4187965) | |
| Gats | −2.14 | | 2.2E−02 | Opposite strand transcription unit to Stag3 (Gats), mRNA | |
| Rab5b | −2.14 | | 1.3E−02 | RAB5B, member RAS oncogene family (Rab5b), transcript variant 2, mRNA | |
| Fam63b | −2.15 | | 6.5E−03 | MKIAA1164 protein | 1 |
| Fcrls | −2.15 | −2.29 | 7.4E−03 | IFGP2 | |
| Pea15a | −2.15 | | 3.9E−02 | Phosphoprotein enriched in astrocytes 15A, mRNA (cDNA clone MGC: 47406 | 1 |
| | | | | IMAGE: 4500957) | |
| Skiv2l | −2.15 | | 2.9E−02 | Superkiller viralicidic activity 2-like (S. cerevisiae), mRNA (cDNA clone | |
| | | | | IMAGE: 5007559) | |
| Usf2 | −2.15 | | 4.0E−02 | Upstream transcription factor 2, mRNA (cDNA clone IMAGE: 3969222) | 3 |
| Jph4 | −2.16 | | 1.6E−02 | Junctophilin 4 (Jph4), transcript variant b, mRNA | |
| Klhdc1 | −2.16 | | 7.6E−03 | Kelch domain containing 1, mRNA (cDNA clone MGC: 141301 IMAGE: 40057794) | |
| Ltbp4 | −2.16 | −4.99 | 1.5E−02 | Latent transforming growth factor beta binding protein 4 long splice variant (Ltbp4) | |
| | | | | mRNA, complete cds, alternatively splice | |
| Zbtb7a | −2.16 | | 2.5E−03 | Zinc finger and BTB domain containing 7a (Zbtb7a), mRNA | |
| Akap1 | −2.17 | | 3.6E−02 | A kinase (PRKA) anchor protein 1 (Akap1), nuclear gene encoding mitochondrial | 1 |
| | | | | protein, transcript variant 1, mRNA | |
| Gtf3a | −2.17 | | 7.6E−03 | General transcription factor III A, mRNA (cDNA clone MGC: 40923 IMAGE: 5374268) | |
| Mau2 | −2.17 | | 1.6E−02 | RIKEN cDNA 9130404D08 gene (9130404D08Rik), mRNA | |
| Rasgef1a | −2.17 | −2.06 | 6.6E−03 | RasGEF domain family, member 1A | |
| Slc40a1 | −2.17 | | 1.6E−02 | Solute carrier family 40 (iron-regulated transporter), member 1, mRNA (cDNA clone | |
| | | | | MGC: 6489 IMAGE: 2647365) | |
| Klf7 | −2.18 | | 2.3E−02 | Transcribed locus, strongly similar to NP_003700.1 Kruppel-like factor 7 (ubiquitous) | |
| | | | | [Homo sapiens] | |
| Camkk2 | −2.18 | | 2.6E−03 | Calcium/calmodulin-dependent protein kinase kinase 2, beta (Camkk2), mRNA | 1 |
| Fam171b | −2.18 | | 1.1E−02 | Family with sequence similarity 171, member B, mRNA (cDNA clone IMAGE: 4501762) | |
| Kalrn | −2.18 | | 6.3E−03 | 2210407G14Rik | |
| Scamp3 | −2.18 | | 7.6E−03 | CDC-like kinase 2, mRNA (cDNA clone MGC: 13872 IMAGE: 3995512) | |
| Mzt1 | −2.19 | | 3.0E−02 | RIKEN cDNA 2410129H14 gene, mRNA (cDNA clone MGC: 151382 | |
| | | | | IMAGE: 40126324) | |
| Phldb1 | −2.19 | | 2.5E−03 | Pleckstrin homology-like domain, family B, member 1 (Phldb1), mRNA | |
| Sgtb | −2.19 | | 2.0E−02 | Small glutamine-rich tetratricopeptide repeat (TPR)-containing, beta (Sgtb), mRNA | |
| Rnd2 | −2.20 | | 2.7E−02 | Rho family GTPase 2 (Rnd2), mRNA | 1 |
| Tcf4 | −2.20 | −2.12 | 7.6E−03 | Transcription factor 4, mRNA (cDNA clone MGC: 13998 IMAGE: 4014231) | |
| Celsr2 | −2.21 | | 2.0E−02 | Cadherin, EGF LAG seven-pass G-type receptor 2 (flamingo homolog, Drosophila), | |
| | | | | mRNA (cDNA clone IMAGE: 3488511) | |
| Ptprd | −2.21 | −2.54 | 1.4E−02 | protein tyrosine phosphatase, receptor type, D | |
| 3110047P20Rik | −2.22 | | 2.2E−02 | RIKEN cDNA 3110047P20 gene | |
| Dlgap1 | −2.22 | | 8.8E−03 | Discs, large (Drosophila) homolog-associated protein 1 (Dlgap1), transcript variant 2, | |
| | | | | mRNA | |
| Mgat4b | −2.22 | | 1.6E−02 | Mannoside acetylglucosaminyltransferase 4, isoenzyme B (Mgat4b), mRNA | 3 |
| Usp11 | −2.22 | | 3.1E−02 | Ubiquitin specific peptidase 11 (Usp11), mRNA | |
| Fbxo41 | −2.23 | | 9.8E−03 | F-box protein 41 (Fbxo41), mRNA | |
| B4galt3 | −2.24 | | 1.6E−02 | UDP-Gal:betaGlcNAc beta 1,4-galactosyltransferase, polypeptide 3, mRNA (cDNA | 1 |
| | | | | clone MGC: 11711 IMAGE: 3965561) | |
| Fjx1 | −2.24 | | 3.4E−02 | Four jointed box 1 (Drosophila) (Fjx1), mRNA | |
| Gga3 | −2.24 | | 6.1E−03 | Golgi associated, gamma adaptin ear containing, ARF binding protein 3, mRNA (cDNA | |
| | | | | clone IMAGE: 6477214) | |
| Sema6d | −2.24 | −2.23 | 1.4E−02 | Sema domain, transmembrane domain (TM), and cytoplasmic domain, (semaphorin) | |
| | | | | 6D (Sema6d), transcript variant 5, mRNA | |
| Smpd1 | −2.24 | | 2.9E−02 | Sphingomyelin phosphodiesterase 1, acid lysosomal, mRNA (cDNA clone MGC: 25355 | |
| | | | | IMAGE: 4482098) | |
| Rbfox1 | −2.25 | | 1.3E−02 | Hexaribonucleotide binding protein 1 (Hrnbp1) | 1 |
| Arid1a | −2.25 | | 7.6E−03 | AT rich interactive domain 1A (SWI-like) | |
| Bcan | −2.25 | | 4.6E−03 | Brevican (Bcan), transcript variant 1, mRNA | |
| Tmod1 | −2.25 | −2.04 | 3.1E−02 | Tropomodulin 1 (Tmod1), mRNA | |
| Rab14 | −2.26 | | 1.0E−02 | RAB14, member RAS oncogene family, mRNA (cDNA clone MGC: 36272 | 2 |
| | | | | IMAGE: 3980228) | |
| Brunol4 | −2.27 | | 1.1E−02 | BRUL4 (Brul4) | |
| Mettl17 | −2.27 | | 1.3E−02 | D14Ertd209e | |
| Slco1c1 | −2.27 | | 2.0E−02 | Solute carrier organic anion transporter family, member 1c1 (Slco1c1), mRNA | |
| Tbc1d17 | −2.27 | | 5.3E−03 | TBC1 domain family, member 17 | |
| Acot7 | −2.28 | | 3.7E−02 | BACH mRNA for acyl-CoA hydrolase, complete cds, isoform mBACHb | 1 |
| Efna5 | −2.28 | | 1.5E−02 | Ephrin A5 (Efna5), transcript variant 2, mRNA | |
| Ank2 | −2.29 | | 2.3E−02 | Ankyrin 2, brain (Ank2), transcript variant 3, mRNA | |
| C1qtnf4 | −2.29 | | 5.3E−03 | C1q and tumor necrosis factor related protein 4, mRNA (cDNA clone IMAGE: 3668760) | |
| Orf61 | −2.29 | | 9.0E−03 | open reading frame 61 | |
| AI593442 | −2.30 | | 1.5E−02 | expressed sequence AI593442 | |
| Jhdm1d | −2.30 | 2.03 | 2.2E−02 | jumonji C domain-containing histone demethylase 1 homolog D (S. cerevisiae) | 1 |
| Spnb4 | −2.30 | | 2.0E−02 | BetaIV-spectrin sigma1 | 2 |
| Acvr2b | −2.32 | | 6.3E−03 | Activin receptor IIB (Acvr2b), mRNA | |
| Elavl3 | −2.39 | | 5.3E−03 | RNA-binding protein mHuC-S | |
| Rab6 | −2.34 | | 1.2E−02 | RAB6, member RAS oncogene family, mRNA (cDNA clone IMAGE: 3491845) | |
| Pak3 | −2.35 | | 1.1E−02 | P21-activated kinase 3 (pak3 gene) | |
| Dusp8 | −2.38 | | 1.9E−02 | Dual specificity phosphatase 8 (Dusp8), mRNA | |
| Tnrc6a | −2.38 | | 1.1E−02 | Trinucleotide repeat containing 6a (Tnrc6a), mRNA | |
| Arl2bp | −2.39 | | 5.6E−03 | ADP-ribosylation factor-like 2 binding protein (Arl2bp), transcript variant 1, mRNA | |
| Mtss1l | −2.39 | | 2.9E−02 | Metastasis suppressor 1-like (Mtss1l), mRNA | 1 |
| Cacng3 | −2.40 | | 1.2E−02 | Calcium channel, voltage-dependent, gamma subunit 3 (Cacng3), mRNA | 1 |
| Ppp1ca | −2.40 | | 2.3E−02 | Protein phosphatase 1, catalytic subunit, alpha isoform, mRNA (cDNA clone | |
| | | | | MGC: 25955 IMAGE: 4239005) | |
| Rtn1 | −2.40 | | 8.8E−03 | Reticulon 1 (Rtn1), transcript variant 1, mRNA | |
| Gabrb1 | −2.41 | | 1.6E−02 | Gamma-aminobutyric acid (GABA-A) receptor, subunit beta 1 (Gabrb1), mRNA | |
| D14Abb1e | −2.41 | | 9.7E−03 | D14Abb1e | |
| Rgs7bp | −2.42 | | 1.5E−02 | Regulator of G-protein signalling 7 binding protein, mRNA (cDNA clone MGC: 143795 | |
| | | | | IMAGE: 40093423) | |
| Syt4 | −2.42 | | 6.6E−03 | Synaptotagmin IV (Syt4), mRNA | |
| Ugt8a | −2.44 | | 5.0E−02 | UDP galactosyltransferase 8A, mRNA (cDNA clone MGC: 18397 IMAGE: 4223057) | |
| E330009J07Rik | −2.45 | | 3.6E−02 | RIKEN cDNA E330009J07 gene (E330009J07Rik), mRNA | |
| Gjc2 | −2.45 | | 2.5E−03 | Gap junction protein, gamma 2 (Gjc2), transcript variant 1, mRNA | 1 |
| Kif3a | −2.46 | | 7.6E−03 | Kinesin family member 3A (Kif3a), mRNA | |
| Nfib | −2.48 | −5.75 | 6.2E−03 | Strain C57BL/6J nuclear factor I/B (Nfib) | |
| Unc13c | −2.48 | | 3.5E−03 | LOC235480 | |
| Atp6v0d1 | −2.49 | | 2.7E−02 | ATPase, H+ transporting, lysosomal V0 subunit D1, mRNA (cDNA clone MGC: 18332 | |
| | | | | IMAGE: 3662404) | |
| Prrt1 | −2.50 | | 1.7E−02 | Proline-rich transmembrane protein 1 (Prrt1), mRNA | 1 |
| 4930402H24Rik | −2.50 | −2.44 | 1.6E−02 | RIKEN cDNA 4930402H24 gene, mRNA (cDNA clone IMAGE: 5366525) | |
| Hes5 | −2.53 | | 1.1E−02 | Hairy and enhancer of split 5 (Drosophila) (Hes5), mRNA | |
| Mark2 | −2.59 | | 1.3E−02 | MAP/microtubule affinity-regulating kinase 2 (Mark2), transcript variant 1, mRNA | |
| Msl1 | −2.59 | | 8.8E−03 | Male-specific lethal 1 homolog (Drosophila) (Msl1), mRNA | |
| Gdi1 | −2.60 | | 3.1E−02 | Guanosine diphosphate (GDP) dissociation inhibitor 1, mRNA (cDNA clone | |
| | | | | MGC: 47005 IMAGE: 5249271) | |
| Slc38a2 | −2.64 | | 2.1E−02 | Solute carrier family 38, member 2, mRNA (cDNA clone IMAGE: 4164084) | 1 |
| Psd2 | −2.68 | | 1.3E−02 | Pleckstrin and Sec7 domain containing 2 (Psd2), mRNA | |
| Ppap2b | −2.69 | −2.65 | 1.5E−02 | Phosphatidic acid phosphatase type 2B (Ppap2b), mRNA | 1 |
| Grm4 | −2.75 | | 2.9E−02 | glutamate receptor, metabotropic 4 | |
| Epn2 | −2.79 | −2.06 | 2.1E−02 | Epsin 2, mRNA (cDNA clone MGC: 19376 IMAGE: 2647379) | |
| 2210018M11Rik | −2.83 | | 1.0E−02 | 2210018M11Rik | |
| Lphn1 | −2.85 | −2.17 | 1.6E−02 | latrophilin 1 | |
| Flt1 | −2.89 | −2.08 | 5.6E−03 | FMS-like tyrosine kinase 1, mRNA (cDNA clone MGC: 36074 IMAGE: 5368921) | |
| Slc38a5 | −2.95 | −3.45 | 2.3E−02 | Solute carrier family 38, member 5, mRNA (cDNA clone MGC: 173142 | |
| | | | | IMAGE: 40057282) | |
| Alas2 | −3.04 | −2.56 | 2.9E−02 | Aminolevulinic acid synthase 2, erythroid, mRNA (cDNA clone IMAGE: 5054102) | |
| Itm2a | −3.05 | | 5.9E−03 | Integral membrane protein 2A, mRNA (cDNA clone MGC: 18323 IMAGE: 3668557) | |
| Tia1 | −3.07 | | 1.5E−02 | cytotoxic granule-associated RNA binding protein 1 | 1 |
| Prkcb | −3.21 | 2.70 | 2.0E−02 | Protein kinase C, beta (Prkcb), mRNA | 1 |
| Cxcl12 | −4.18 | 2.58 | 1.4E−02 | Chemokine (C—X—C motif) ligand 12, mRNA (cDNA clone MGC: 6119 IMAGE: | |
| | | | | 3483088) | |
| Hbb-b1 | −23.47 | | 2.1E−02 | Hemoglobin, beta adult minor chain, mRNA (cDNA clone MGC: 40691 | |
| | | | | IMAGE: 3988455) | |
| Hba-a1 | −24.73 | | 2.9E−02 | Hemoglobin alpha, adult chain 1 (Hba-a1), mRNA | |

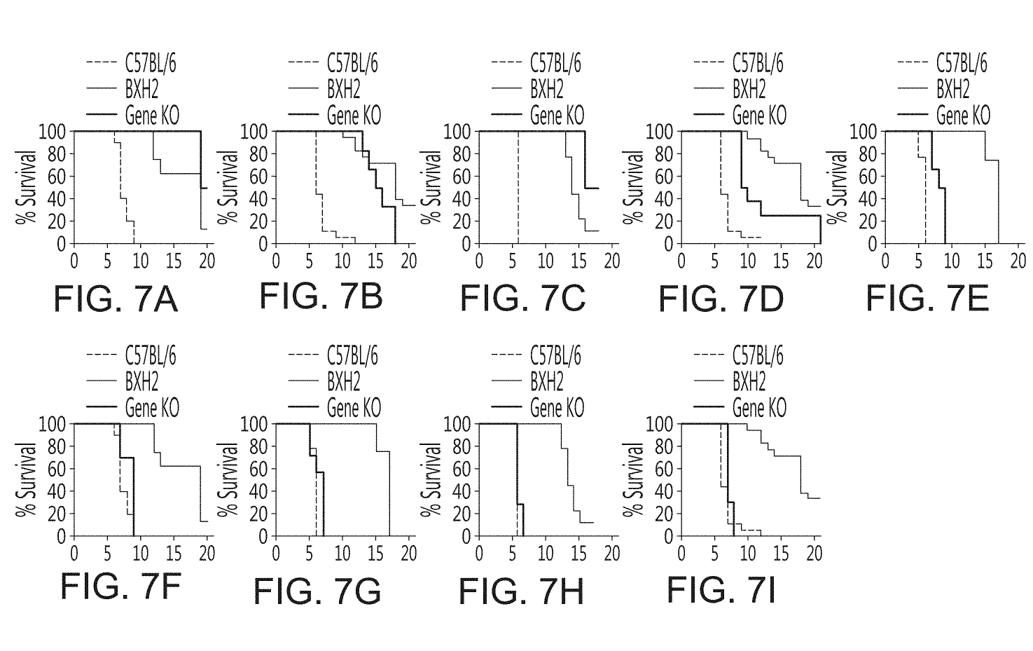

Finally, to validate the role of the identified IRF8 targets and associated pathways in innate immunity and pathological neuroinflammation, the susceptibility to PbA infection was phenotyped in mouse strains bearing null mutations at several of these loci. These included infection-regulated genes bearing IRF8 binding sites (Irf1, Ifit1, Isg15, Irgm1 and Nlrc4), and other genes known to play key roles in early innate immune response (Ifng, Jak3, Stat1, 1112p40). Results from these experiments (FIG. 7) show that IFNγ^{-/-}, JAK3^{-/-} and STAT1^{-/-} mutant mice were completely resistant to P. berghei infection and did not develop CM, highlighting key roles for these molecules in the progression or amplification of the pathological inflammatory response. Loss Irf1 and Irgm1 delayed appearance of neurological symptoms and prolonged survival of PbA infected mice. These results indicate that several of the transcriptional targets of IRF8 activated during PbA infection in lymphoid and myeloid cells play a critical role in neuroinflammation. On the other hand IL12p40^{-/-}, IFIT1^{-/-}, ISG15^{-/-} and NLRC4^{-/-} mutant mice remained susceptible to PbA-induced CM, suggesting that although these proteins may play important roles in neuroinflammation, their deletion is not sufficient to induce protection.

The demonstrated role of IRF8 in the ontogeny of myeloid cells, its known role in defense against infectious pathogens and the growing body of evidence from GWAS studies in humans linking IRF8 variants to chronic autoimmune inflammatory conditions such as multiple sclerosis, systemic lupus erythmatous and Crohn's disease prompted the investigation of a possible role for IRF8 in acute pathological inflammatory reactions. For this, a mouse model of acute encephalitis caused by infection with P. berghei (cerebral malaria) was used, which involves lethal neuroinflammation caused by recruitment of inflammatory mononuclear and polynuclear leukocytes, and ensuing loss of integrity of the blood brain barrier (BBB). It was found that the loss of Irf8 in BXH2 mice completely protects against this pathology, preventing the development of neurological symptoms and prolonging survival post infection. Interestingly, the protective effect was inherited in a co-dominant fashion as 50% of Irf8^{R294C/+} F1 heterozygotes survived through the cerebral phase when infected with PbA (FIG. 3A). These finding establish that IRF8 is critical to the development of acute lethal neuroinflammation associated with experimental cerebral malaria and further implicate Irf8 as a major regulator of this pathological response. Moreover, results from Irf8^{R294C/+} F1 heterozygotes indicate that Irf8 regulates key pro-inflammatory cells and pathways in a gene dosage dependent fashion.

In addition to its established role in ontogeny and function of myeloid cells, Irf8 is also required for certain aspects of B lymphocytes development and of T lymphocytes function (Th1, Th17 response). To identify the cell types and gene-dosage dependent pathways that are activated by IRF8 during neuroinflammation, brain transcripts profiles from PbA-infected B6 and BXH2 mice were compared to and extracted a list of genes that are induced by infection in an Irf8-dependent and independent fashion (2×2 ANOVA and pairwise analysis) (FIG. 5, Table 1). In parallel, ChIP-seq experiments we carried out to map genome-wide IRF8 binding sites. We compared these positions to the gene lists generated by transcript profiling and identified both IRF8-bound genes (Table 2), and IRF8 bound genes regulated in an Irf8-allele specific fashion (Table 1). There was substantial overlap between these gene sets, which were similarly dominated by markers and pathways characteristic of antigen-presenting cells (APC), including antigen processing and presentation, production of type I interferon, production of pro-inflammatory cytokines/chemokines and others. These combined analyses confirm that IRF8 plays a prominent role in the unique functions of APCs including antigen capture and microbial phagocytosis (C1q, C4b, Fcgr4, Fcgr1), cytoplasmic inflammasome platforms such as Nlrc5, and Ifi205; phagosome maturation including recruitment of key small GTPases (Irgm1, Irgm2, Igtp, Gbp2, Gbp3), endoplasmic reticulum membrane associated antigen transport (Tap1, Tap2), Class I and Class II MHC-dependent antigen presentation in APCs (B2m, H2-A, D, K, L, Q, T molecules). These gene lists also featured a number of inflammatory cytokine and chemokines involved in chemotaxis of myeloid and lymphoid cell types to the sites of infection (Ccl4, Ccl5, Ccl7, Ccl12, Cxcl9, Cxcl10). These findings are compatible with a simple functional model where myeloid cells (including APCs) are rapidly recruited in large numbers to the site of P. berghei infection and associated-tissue injury, namely capillaries of the blood brain barrier. This initiates a robust IRF8-dependent pro-inflammatory cascade. Local amplification of this response by recruited cells leads to excessive production of immunopathological soluble mediators such as IFNγ and TNFα by T lymphocytes and induces other transcription factors including Stat1 and other IRF family members (Irf1, Irf7, Irf9). Absence of IRF8 blunts this pathological response and allows mutant BXH2 mice to avoid developing neuroinflammation during CM, thus surviving the critical acute phase.

Although the severe depletion of dendritic cells and macrophages, along with a concomitant reduction in IL12 production and antigen-specific T-cell priming in BXH2 is likely to account for an important component of CM-resistance, it is proposed that even in the context of normal myeloid cell numbers, reduced IRF8-dependent transcriptional activation of APC-specific pathways is sufficient to significantly blunt inflammatory response and protect against acute pathological inflammation. This is based on three main observations. First, IRF8 behaves primarily as a transcriptional activator, not a repressor, in myeloid cells as can be seen by the enrichment of IRF8 binding sites in up-regulated genes only (FIG. 6B). Table 1 also highlights that for each gene regulated in a strain and infection specific way, IRF8 competent mice invariably show a higher magnitude fold change than BXH2, and the majority of these genes are up-regulated (Group 1 & 2) rather than downregulated (Group 3). Second, Irf8^{R294C/+} F1 heterozygotes show normal numbers of myeloid cells (DCs, macrophages) and lymphoid cells (data not shown), but still display significant resistance to P. berghei induced CM (FIG. 3A). Thirdly, the inactivation of several direct transcriptional targets of IRF8 (identified as bound and regulated by IRF8 during PbA infection) including the phagosome associated small GTPase Irgm1, the pro-inflammatory cytokines Cxcl9, and Cxcl10, the Cd11b receptor Icam1 and the transcriptional activator and IRF8 dimerization partner Irf1 have been shown to cause resistance to CM in corresponding deletion mouse mutants (FIG. 7). Finally, the inactivation of additional IRF8 targets, detected herein by ChIP-Seq, has been shown to protect against P. berghei induced CM, including Ifng, Jak3, Cd8, Cd14, Cd40, Hc, Fcgr2, Lta and Ltbr8. Together, these results highlight the role of IRF8 in regulating pro-inflammatory pathways in myeloid cells during CM-associated neuroinflammation.

Although IRF8-dependent activation of pro-inflammatory pathways in myeloid cells has detrimental and pathological consequences in PbA infection, it clearly plays a protective role in other types of infections including pulmonary tuberculosis. Indeed, using the same analysis and very stringent statistical parameters, a strong overlap was noted between the list of genes up-regulated in brains of B6 mice in response to PbA infection and up-regulated in lungs 30 days following aerosol infection with Mycobacterium tuberculosis (Table 3). Of the 123 genes up-regulated in P. berghei-infected brains, more than half (n=66) were also up-regulated in M. tuberculosis-infected lungs, and nearly 3/4 (90/123) of the P. berghei regulated genes harbored an IRF8 binding site. Amongst the 66 genes up-regulated during both M. tuberculosis infection and during CM, a striking 80% (n=53) display one or more IRF8 binding sites. Furthermore, inactivating mutations in several of these genes including Irf8, Irf1, and Irgm1 cause susceptibility to pulmonary TB, while conveying some degree of protection against CM. Mutations in Tap1 and B2m also cause susceptibility to TB, while their effects on CM susceptibility have yet to be tested. It is proposed that this set of 53 genes represents the core Irf8-dependent pro-inflammatory response pathways that play key roles in protection against TB, and pathological inflammation associated with CM.

Overall, the P. berghei infection model indicate that Irf8 and IRF8-regulated targets play a major role in pathological inflammation. Inactivating mutations or absence of Irf8 (or certain transcriptional targets) leads to complete protection against CM, and reduced Irf8 expression causes partial protection (in Irf8^{R294C/+} F1 heterozygotes). Such gene-dosage dependent effects also raise the possibility that even small changes in expression or activity of IRF8 may have phenotypic consequences, with increased Irf8 expression possibly associated with enhanced and/or chronic inflammation.

In agreement with this hypothesis, results from recent genome wide association studies (GWAS) have pointed to IRF8 as one of the genetic factors implicated in the complex genetic etiology of several human autoimmune and chronic inflammatory conditions. For example, a SNP near IRF8 was found associated with systemic lupus erythematosus, a disease where production of type I interferon is central to pathogenesis. In addition, SNPs in IRF8 are found within a risk haplotype detected using GWA studies and meta-analysis for inflammatory bowel disease (IBD) patients63, and in linkage disequilibrium mapping in certain groups of Crohn's disease patients (rs16940202). Finally, IRF8 is key risk factor in multiple sclerosis (MS), and its association with this disease has been validated in multiple GWA studies and meta-analyses. In MS, disease risk is associated with an expression SNP (rs17445836) which maps 61 kb downstream IRF8 and regulates gene expression with higher IRF8 mRNA levels associated with disease. These results not only support a role for IRF8 in human chronic inflammatory conditions but further suggest that, in agreement with our results in mice, even modest changes in expression or activity of Irf8 in the context of persistent microbial or autoimmune stimulus, may lead to chronic or pathological inflammation. In agreement with this proposal, it is noted that several IRF8 targets regulated during neuroinflammation in PbA-infected mice have also been detected as genetic risk factors in GWA studies of human chronic inflammatory conditions, including the MHC (type 1 diabetes, rheumatoid arthritis, lupus, MS, psoriasis), CCL7 (in IBD), IRF1 (IBD), IRF7 (Lupus) and ICAM1 (IBD) (Table 2). This highlights the role of IRF8 regulated pathways in pathological inflammation in humans.

The mouse model of acute neuroinflammation induced by P. berghei infection has proven valuable to identify novel genes, proteins and pathways involved in pathological inflammatory conditions. This model can help prioritize genes identified in human GWA studies for therapeutic development, including assessing activity of novel anti-inflammatory drug candidates for use in common human inflammatory conditions.

### Example VII—Usp15 and Trim25

Mice.

Inbred C57BL/6J (B6) and 129S1/SvImJ (129S1) mice were purchased from the Jackson laboratories (Bar Harbor, Me., USA). The protocol for ENU mutagenesis is described in Example I. Briefly, mutagenized G0 B6 males were bred to wild-type (WT) 129S1 to generate a G1 offspring, which were backcrossed to 129S1 females (G2 offspring). Two G2 females per pedigree were backcrossed to their G1 father, Corbin, to generate G3 mice for phenotyping. The Usp15^{L749R} mutation was backcrossed to B6 for 4 generations, and Usp15^{L749R} homozygotes were then intercrossed to generate a pure line. Genotyping with informative markers indicated that the genome of these mice was >90% of B6 origin. Trim25 mutant mice (Trim25⁻) were obtained from RIKEN (Japan; mutant stock RBRC 02844). Socs3^{fl/fl}-Socs1^{fl/fl} conditional knockout mice carrying or not a copy of the T-cell specific Lck-Cre driver transgene were obtained from Dr. S. Ilangumaran (Universite de Sherbrooke); Mavs^{-/-} mutant mice were a generous gift of Dr. C. Liang (Lady Davis Institute for Medical Research, Montreal); Irf3^{-/-} and Irf9^{-/-} mutant mice were obtained from Dr. K. Mossman (McMaster University). All mutants were maintained on C57BL/6J genetic background.

Parasites and Infections.

Plasmodium berghei ANKA (PbA) parasites were obtained from the Malaria Reference and Research Reagent Resource Center (MR4). Parasites were maintained as frozen stocks at −80° C. and passaged weekly in B6 mice. Blood parasitemia was determined on thin blood smears stained with Diff-Quick reagents. Seven to eight week-old mice were infected intravenously with 10⁶ parasitized red blood cells (pRBCs). Mice were monitored for appearance of neurological symptoms 3 times daily. Mice displaying severe cerebral symptoms were euthanized. Animals that survived the experimental cerebral phase (ECM; days 5-13) were considered ECM-resistant, and were euthanized on day 18 post-infection (experimental end-point).

Whole-Exome Sequencing.

Whole-exome sequencing was carried out on three ECM-resistant Corbin G3 mice. Exome capture was performed using a SureSelect Mouse All Exon™ kit (Agilent Technologies, USA) and parallel sequencing on an Illumina HiSeq2000™ (100 bp paired-end reads). Reads were aligned to the reference mouse genome assembly (NCBI37/mm9) with Burrows-Wheeler Alignment (BWA) tool and coverage was assessed with BEDTools. Variants were called using Samtools pileup and varFilter and were annotated using Annovar. This analysis identified a homozygous mutation in USP15 (L749R) that was associated with the ECM-resistance phenotype. The Usp15^{L749R} mutation was genotyped by PCR amplification from genomic DNA (5′-AATGAATGCCTTCAACAGTGG-3′ (SEQ ID NO: 11), 5′-ACAATGCCAACTTTCAGAAGC-3′ (SEQ ID NO: 12)) followed by DNA sequencing.

Plasmids.

A full-length mouse Usp15 cDNA (pFLCIII-Usp15-WT; Riken Fantom Clones) was modified to include a C-terminal hemagglutinin (HA) epitope tag, and restriction enzyme sites for Hindlll (CCACC) and Xhol (CTCGAG) to allow cloning into pcDNA3 expression plasmid. The resulting WT plasmid, pcDNA3-mUsp15-VVT-HA, was used as a template for the generation of mutants by site-directed mutagenesis, including Usp15^{L749R}. Plasmid encoding the full-length human WT USP15, pcDNA3-Xpress-His-USP15, was obtained from commercial sources (Addgene, plasmid ID: p5953), and was used as a template to produce mutants L720R, C269A, S923L, and C783A by site-directed mutagenesis. The pEF-TRIM25-FLAG plasmid was kindly provided by Dr. J. U. Jung (Harvard Medical School, University of Southern California Keck School of Medicine). The integrity of all plasmid constructs were verified by complete nucleotide sequencing of the corresponding cDNA inserts.

Antibodies.

Endogenous USP15 protein expression was monitored by immunoblotting using a rabbit polyclonal anti-USP15 antibody (Abcam, ab97533), while epitope tagged USP15 variants were detected using a mouse anti-HA (Covance, HA.11), or mouse anti-Xpress (LifeTechnologies, R910-25) monoclonal antibodies. Native TRIM25 was detected using a rabbit polyclonal anti-TRIM25 antiserum (Proteintech, 12573-1-AP), while epitope-tagged TRIM25 was detected using a mouse anti-FLAG (Sigma Aldrich, Clone M2) monoclonal antibody. Ubiquitinated TRIM25 was detected by immunoblotting with a mouse anti-Ubiquitin monoclonal antibody (Santa Cruz, P4D1; SC8017).

USP15 Protein Expression and Stability.

USP15 protein expression was monitored by immunoblotting in several tissues and cell types. Mouse tissues were homogenized in 50 mM Tris pH 7.5, 150 mM NaCl, 1% TritonX-100, and 0.1% sodium dodecyl sulfate (SDS), supplemented with protease and phosphatase inhibitors. Discrete immune cell populations were isolated from spleen and thymus by flow cytometry and cell sorting (FACSAriall) following staining with combinations of cell surface markers to obtain CD4 T cells (CD4⁺ CD8⁻), CD8 T cells (CD4⁻CD8⁺), NK cells (TCRb⁻CD49b⁺), B cells (TCRb⁻CD19⁺), and thymic double negative T cells (DN: CD4-CD8⁻), and thymic double positive T cells (DP: CD4⁺CD8⁺). For protein stability studies, HEK293 cells (ATCC-CRL-1573) were stably transfected with HA-tagged mouse Usp15 constructs using Lipofectamine 2000™ reagent (Life Technologies) followed by clonal selection and expansion in geneticin (G418, 500 μg/ml) (Invitrogen, CA, USA). Stably transfected cells were treated with cycloheximide (20 μg/ml) for 10, 15, 20, and 25 hours, followed by cell lysis in 50 mM Tris pH 7.5, 150 mM NaCl, 1% TritonX-100, and 0.1% sodium dodecyl sulfate. Equal amounts of protein (25 μg) were analyzed by immunoblotting.

Experimental Autoimmune Encephalomyelitis.

Experimental Autoimmune Encephalomyelitis (EAE) was induced. Briefly, 8-12 weeks female mice were treated with myelin oligodendrocyte glycoprotein (MOG; peptide sequence 35-55) emulsified in Complete Freund Adjuvent (CFA) (50 micrograms/mouse, s.c., at day 0) and pertussis toxin (PTX; 300 nanograms/mouse, i.p., at days 0 and 2). The mice were weighed and monitored daily for clinical signs of EAE, which were scored as follows: tail (0, no symptoms; 1, weak; 2 or hooked tail; 2, paralyzed); hind limb (0, no symptoms; 1, weak; 2, full paresis; 3, no movement); front limbs (0, no symptoms; 1, weak; 2, full paresis; 3, no movement). Each limb was scored individually and total scores were tabulated for each animal. For ethical reasons, severely impaired animals were euthanized. At pre-determined time intervals, spinal cords were dissected for histology and for extraction of RNA.

Cellular Immunophenotyping.

Five days post-infection with PbA, mice were ex-sanguinated and perfused with 20 mL PBS-containing 2 mM EDTA. Brains were harvested and homogenized in RPMI media-containing 0.5 mg/mL collagenase (Gibco LifeTechnologies), 0.01 mg/mL DNase I (Roche) and 2 mM EDTA. Infiltrating cells were separated on a 33.3% Percoll solution. Cells were stained for FACS analyses with the following antibodies; anti-CD45-APC-eFluor780 (clone 30-F11, eBioscience), anti-CD4-PerCP Cyanine5.5 (clone RM4-5, eBioscience), anti-CD8alpha-PE (clone 53-6.7, eBioscience), anti-TCRbeta-FITC (clone H57-597, eBioscience), anti-CD11b-APC (clone M1/70, eBioscience), anti-Ly6C-PE (clone HK1.4, eBioscience), and anti-Ly6G-FITC (clone 1A8, BioLegend). Leukocytes were gated as CD45^{hi} cells. Live cells were identified using Zombie Aqua Dye-V500 (BioLegend).

Splenocytes from naive and PbA-infected mice were analyzed by flow cytometry using markers of lymphoid cells (anti-CD45-APC-efluor780, anti-CD8-Bv421, anti-CD4-PE, anti-TCRI3-FITC), and myeloid cells (anti-CD45-APC-efluor780, anti-CD11b-APC, anti-Ly6G-FITC). In stimulation experiments, 4 million splenocytes per well were cultured with either anti-CD3 (5 μg/mL, eBioscience)/anti-CD28 (2 μg/mL, eBioscience), or with PMA (50 ng/mL) and ionomycin (500 ng/mL) for 4 hours, followed by assessment of intracellular staining for IFNγ by flow cytometry. Serum cytokines were also measured using a cytokine/chemokine 32-multiplex Luminex™ array (Eve Technologies, Calgary, Canada).

RNA Sequencing & Validation by qPCR.

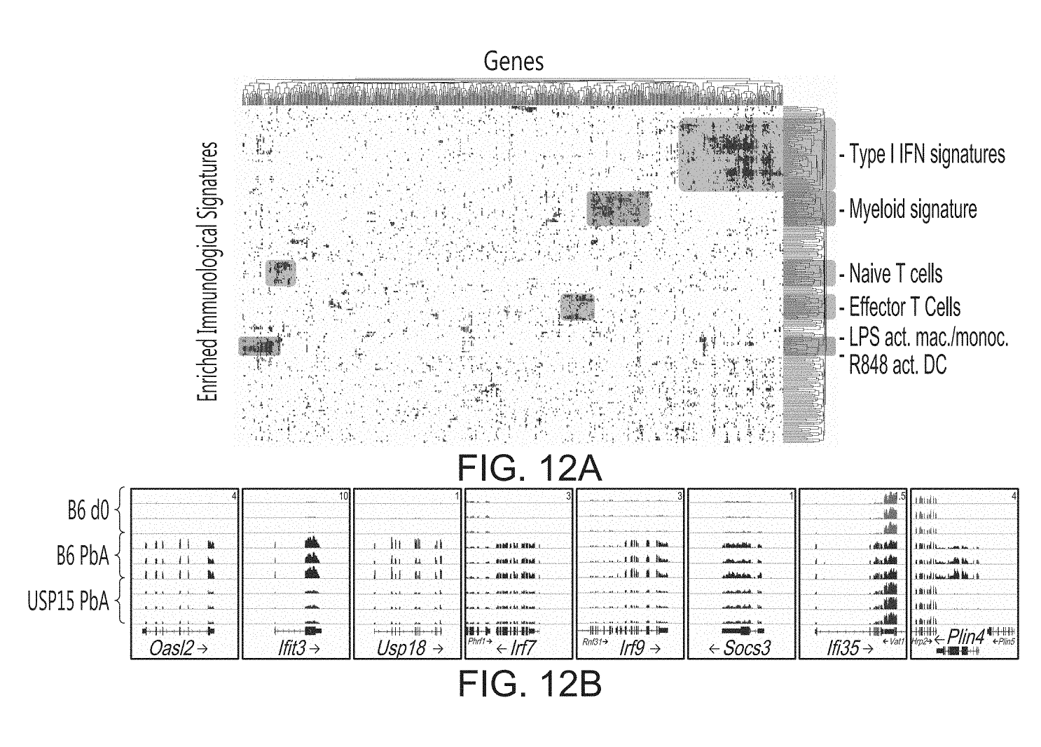

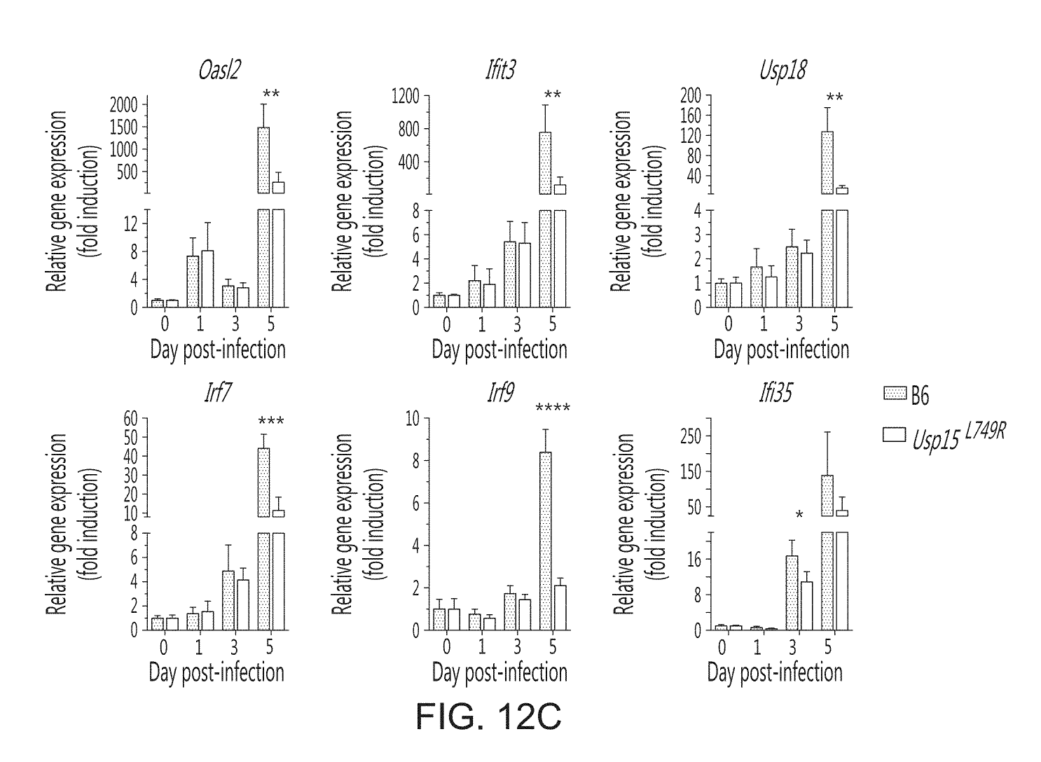

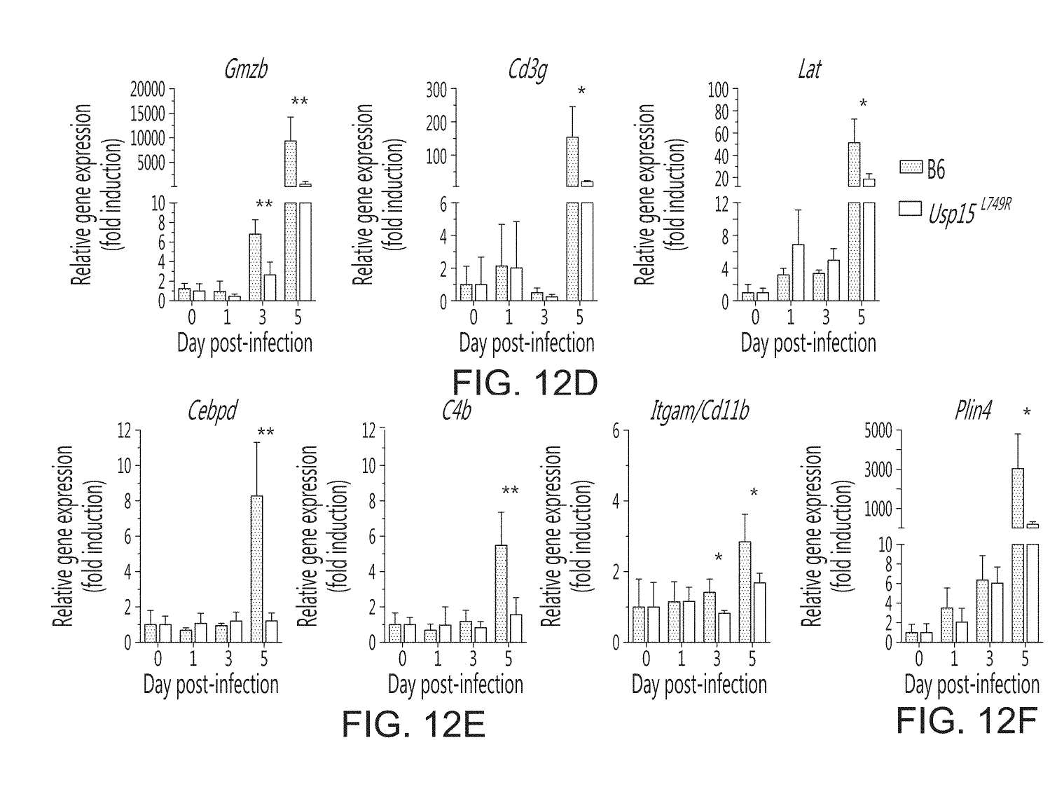

Perfused brains from PbA-infected mice (at day 5) and from spinal cords of mice undergoing EAE (at day 7) were harvested and frozen in liquid nitrogen. Total RNA was isolated using Trizol™-chloroform (Life Technologies), followed by DNase-digestion and purification on RNeasy columns (Qiagen). RNA integrity was assessed on a Bioanalyzer™ RNA pico chip, followed by rRNA depletion and library preparation using Illumina TruSeq™ Stranded Total RNA Library preparation kit. The RNA-seq libraries were sequenced on an Illumina HiSeq™ 2500 sequencer in paired-end 50 bp configuration, with an average of 103×10⁶ reads for PbA and 173×10⁶ reads EAE samples, with >80% successfully mapped to the mm9 reference genome using TopHat 2.0.9 and Bowtie 1.0.0 algorithm combination. Gene expression was quantified by counting the number of uniquely mapped reads with featureCounts tool using default parameters. Normalization and differential gene expression analysis was conducted independently for PbA and EAE datasets using the edgeR Bioconductor package. We retained genes that had a minimum expression level of 5 counts per million reads (CPM) in at least 3 of the 9 samples for PbA, and at least 2 out of 4 for EAE datasets. Genes with differential expression in Usp15^{L749R} were considered significant if fold change >11.51 and adjusted p value <0.05. For FIG. 12B preparation, RNA-seq sequence density profiles were normalized per 10 million reads using a succession of genomeCoverageBed and wigToBigWig tools and visualized in IGV genome browser. We proceeded to unbiased clustering of the 316 genes with significant dys-regulation in PbA and/or EAE (Table 4, FIG. 11B) using Pearson un-centered correlation with average distance metric within MeV software.

TABLE 4
List of dysregulated genes in Usp15^{L749R} mutant mice undergoing ECM
and EAE models neuroinflammatory diseases, related to FIG. 11B.

| Clustering | | PbA d5 (USP15/B6) | | | EAE d7 (USP15/B6) | | |
|---|---|---|---|---|---|---|---|
| order | Gene symbol | Fold change | Log2 FC | adj. p val. | Fold change | Log2 FC | adj. p val. |
| 1 | I830012O16Rik | 0.42 | −1.25 | 2.73E−07 | 0.62 | −0.68 | 5.90E−05 |
| 2 | Irf9 | 0.48 | −1.06 | 3.02E−11 | 0.66 | −0.60 | 6.39E−05 |
| 3 | Parp12 | 0.63 | −0.66 | 1.37E−04 | 0.80 | −0.32 | 1.76E−01 |
| 4 | Samd9l | 0.64 | −0.65 | 2.98E−03 | 0.80 | −0.32 | 5.74E−01 |
| 5 | Pglyrp1 | 0.64 | −0.65 | 3.60E−03 | 0.79 | −0.33 | 4.11E−01 |
| 6 | Tsc22d3 | 0.59 | −0.76 | 1.54E−08 | 0.71 | −0.50 | 1.76E−07 |
| 7 | Lgals3bp | 0.59 | −0.75 | 1.49E−09 | 0.71 | −0.49 | 6.59E−03 |
| 8 | Sult1a1 | 0.48 | −1.05 | 7.09E−05 | 0.62 | −0.70 | 3.69E−04 |
| 9 | Mt1 | 0.61 | −0.71 | 3.83E−08 | 0.72 | −0.48 | 6.11E−05 |
| 10 | Smim3 | 0.62 | −0.68 | 5.48E−04 | 0.75 | −0.41 | 1.41E−01 |
| 11 | Cd274 | 0.63 | −0.68 | 2.20E−05 | 0.76 | −0.40 | 8.23E−03 |
| 12 | Stat1 | 0.57 | −0.81 | 2.64E−08 | 0.72 | −0.48 | 8.66E−04 |
| 13 | Ncf1 | 0.66 | −0.59 | 3.54E−04 | 0.79 | −0.35 | 2.40E−01 |
| 14 | Sgk3 | 0.63 | −0.66 | 8.58E−04 | 0.75 | −0.41 | 1.22E−03 |
| 15 | Ifih1 | 0.61 | −0.72 | 1.12E−04 | 0.73 | −0.46 | 7.96E−02 |
| 16 | Apol8 | 0.65 | −0.63 | 1.19E−03 | 0.76 | −0.40 | 1.34E−01 |
| 17 | Mt2 | 0.50 | −0.99 | 4.77E−07 | 0.60 | −0.74 | 7.11E−15 |
| 18 | Lrrc33 | 0.67 | −0.59 | 7.86E−03 | 0.73 | −0.45 | 5.19E−02 |
| 19 | Ptprc | 0.60 | −0.74 | 2.90E−03 | 0.70 | −0.52 | 2.05E−01 |
| 20 | Ifit3 | 0.43 | −1.21 | 6.56E−07 | 0.55 | −0.86 | 3.25E−05 |
| 21 | Parp9 | 0.61 | −0.72 | 6.05E−04 | 0.70 | −0.51 | 1.45E−01 |
| 22 | C4a | 0.66 | −0.60 | 3.29E−03 | 0.74 | −0.44 | 4.84E−02 |
| 23 | Icosl | 0.65 | −0.62 | 4.32E−04 | 0.73 | −0.45 | 7.37E−02 |
| 24 | Fkbp5 | 0.51 | −0.97 | 4.19E−07 | 0.59 | −0.77 | 8.35E−12 |
| 25 | Trim34a | 0.55 | −0.87 | 2.60E−05 | 0.61 | −0.71 | 5.46E−04 |
| 26 | Slc25a37 | 0.62 | −0.70 | 1.46E−05 | 0.68 | −0.57 | 3.34E−06 |
| 27 | Plin4 | 0.24 | −2.08 | 1.41E−05 | 0.26 | −1.94 | 4.33E−51 |
| 28 | Fam107a | 0.47 | −1.08 | 1.23E−12 | 0.52 | −0.95 | 8.84E−25 |
| 29 | Ldb3 | 0.42 | −1.26 | 5.40E−10 | 0.45 | −1.14 | 8.29E−16 |
| 30 | Dao | 0.62 | −0.69 | 1.34E−04 | 0.65 | −0.62 | 1.60E−05 |
| 31 | Ifit1 | 0.49 | −1.02 | 3.36E−04 | 0.50 | −1.01 | 3.60E−08 |
| 32 | Rpph1 | 0.50 | −1.01 | 1.20E−21 | 0.50 | −0.99 | 9.59E−23 |
| 33 | Esd | 0.63 | −0.67 | 2.16E−08 | 0.64 | −0.65 | 1.40E−11 |
| 34 | Arrdc2 | 0.43 | −1.21 | 1.13E−05 | 0.42 | −1.25 | 1.19E−30 |
| 35 | 4930452B06Rik | 0.64 | −0.63 | 1.07E−03 | 0.64 | −0.64 | 1.09E−02 |
| 36 | C4b | 0.65 | −0.62 | 1.90E−03 | 0.63 | −0.67 | 5.67E−04 |
| 37 | Phyhd1 | 0.61 | −0.72 | 2.16E−05 | 0.59 | −0.76 | 8.47E−13 |
| 38 | Slc25a34 | 0.52 | −0.93 | 6.79E−08 | 0.50 | −0.99 | 1.00E−07 |
| 39 | Net1 | 0.67 | −0.58 | 2.58E−06 | 0.65 | −0.62 | 3.07E−07 |
| 40 | Fcgr3 | 0.66 | −0.59 | 8.69E−07 | 0.55 | −0.86 | 1.72E−02 |
| 41 | Samhd1 | 0.75 | −0.41 | 5.27E−03 | 0.66 | −0.59 | 2.38E−04 |
| 42 | Irgm1 | 0.61 | −0.71 | 1.82E−05 | 0.51 | −0.97 | 6.75E−08 |
| 43 | Lcn2 | 0.42 | −1.27 | 6.87E−02 | 0.30 | −1.74 | 8.10E−20 |
| 44 | Galnt15 | 0.45 | −1.17 | 5.06E−07 | 0.32 | −1.63 | 9.47E−15 |
| 45 | Txnip | 0.70 | −0.52 | 6.61E−03 | 0.61 | −0.72 | 1.24E−07 |
| 46 | Agxt2l1 | 0.47 | −1.08 | 3.14E−09 | 0.37 | −1.45 | 4.53E−48 |
| 47 | Entpd4 | 0.64 | −0.65 | 6.14E−07 | 0.55 | −0.87 | 9.90E−25 |
| 48 | B2m | 0.71 | −0.50 | 4.90E−08 | 0.63 | −0.67 | 2.02E−07 |
| 49 | Adipor2 | 0.70 | −0.52 | 1.82E−04 | 0.63 | −0.68 | 2.87E−13 |
| 50 | Trim30a | 0.54 | −0.89 | 1.62E−04 | 0.49 | −1.04 | 1.26E−04 |
| 51 | Apobec3 | 0.61 | −0.70 | 5.84E−04 | 0.57 | −0.81 | 1.77E−04 |
| 52 | Mertk | 0.69 | −0.54 | 2.34E−06 | 0.65 | −0.62 | 3.98E−08 |
| 53 | Oasl2 | 0.48 | −1.07 | 2.89E−07 | 0.40 | −1.33 | 2.88E−08 |
| 54 | Ucp2 | 0.65 | −0.63 | 1.89E−03 | 0.59 | −0.77 | 6.29E−11 |
| 55 | 6720456H20Rik | 0.62 | −0.68 | 6.82E−07 | 0.55 | −0.86 | 1.06E−19 |
| 56 | Slco4a1 | 0.70 | −0.50 | 7.68E−02 | 0.64 | −0.64 | 4.50E−03 |
| 57 | Map3k6 | 0.38 | −1.39 | 2.97E−05 | 0.19 | −2.36 | 4.84E−34 |
| 58 | Cebpd | 0.59 | −0.77 | 4.77E−07 | 0.40 | −1.32 | 4.97E−14 |
| 59 | Socs3 | 0.62 | −0.69 | 4.02E−03 | 0.43 | −1.21 | 6.58E−05 |
| 60 | H2-K1 | 0.74 | −0.44 | 2.60E−05 | 0.60 | −0.73 | 1.67E−11 |
| 61 | Irf1 | 0.69 | −0.54 | 7.57E−05 | 0.55 | −0.87 | 9.31E−03 |
| 62 | Klf15 | 0.76 | −0.40 | 5.27E−03 | 0.65 | −0.63 | 5.39E−08 |
| 63 | Uba7 | 0.79 | −0.33 | 1.33E−01 | 0.58 | −0.78 | 5.73E−03 |
| 64 | Apold1 | 0.74 | −0.44 | 1.40E−01 | 0.46 | −1.13 | 9.40E−11 |
| 65 | Ddit4 | 0.73 | −0.44 | 4.80E−02 | 0.47 | −1.09 | 5.63E−18 |
| 66 | Tap1 | 0.65 | −0.62 | 1.65E−03 | 0.40 | −1.32 | 2.14E−06 |
| 67 | Gbp6 | 0.65 | −0.62 | 1.67E−02 | 0.40 | −1.33 | 3.40E−04 |
| 68 | Gbp3 | 0.65 | −0.62 | 1.56E−03 | 0.41 | −1.29 | 1.61E−04 |
| 69 | Gbp7 | 0.67 | −0.58 | 1.08E−03 | 0.44 | −1.19 | 9.79E−05 |
| 70 | Parp14 | 0.72 | −0.48 | 2.93E−02 | 0.53 | −0.91 | 2.90E−03 |
| 71 | Ly6c2 | 0.75 | −0.42 | 4.66E−03 | 0.59 | −0.77 | 2.82E−03 |
| 72 | Nfkbia | 0.72 | −0.47 | 3.73E−02 | 0.53 | −0.92 | 8.10E−20 |
| 73 | Ccdc3 | 0.79 | −0.34 | 4.75E−02 | 0.63 | −0.67 | 2.66E−07 |
| 74 | Lrg1 | 0.69 | −0.54 | 3.25E−02 | 0.35 | −1.53 | 1.19E−07 |
| 75 | Il1r1 | 0.78 | −0.36 | 1.04E−01 | 0.51 | −0.96 | 2.18E−09 |
| 76 | Gm8979 | 0.66 | −0.60 | 3.38E−03 | 0.28 | −1.84 | 1.76E−13 |
| 77 | Zbtb16 | 0.80 | −0.32 | 1.31E−02 | 0.51 | −0.96 | 5.59E−08 |
| 78 | Tmem252 | 0.63 | −0.67 | 1.17E−02 | 0.18 | −2.47 | 1.68E−42 |
| 79 | Xdh | 0.69 | −0.54 | 9.25E−03 | 0.26 | −1.93 | 2.15E−42 |
| 80 | Nlrc5 | 0.68 | −0.56 | 1.07E−02 | 0.26 | −1.92 | 1.69E−12 |
| 81 | Plekhf1 | 0.77 | −0.38 | 2.02E−01 | 0.41 | −1.30 | 3.61E−20 |
| 82 | Vwf | 0.88 | −0.19 | 4.62E−01 | 0.66 | −0.61 | 1.36E−07 |
| 83 | Myo7a | 0.86 | −0.22 | 4.16E−01 | 0.61 | −0.72 | 9.37E−13 |
| 84 | Bub1b | 0.69 | −0.53 | 4.72E−02 | 0.30 | −1.76 | 1.19E−08 |
| 85 | Ptgs2 | 0.75 | −0.42 | 7.78E−03 | 0.38 | −1.40 | 2.30E−06 |
| 86 | Htr3a | 0.88 | −0.18 | 4.86E−01 | 0.66 | −0.60 | 2.23E−02 |
| 87 | Sgk1 | 0.64 | −0.64 | 8.07E−04 | 0.15 | −2.78 | 2.04E−234 |
| 88 | Igtp | 0.69 | −0.55 | 1.20E−02 | 0.20 | −2.35 | 7.16E−10 |
| 89 | Arl4d | 0.76 | −0.40 | 1.55E−01 | 0.31 | −1.69 | 1.31E−23 |
| 90 | Mgp | 0.81 | −0.30 | 2.53E−01 | 0.44 | −1.19 | 5.35E−12 |
| 91 | Arid5b | 0.88 | −0.18 | 1.79E−01 | 0.60 | −0.74 | 8.13E−17 |
| 92 | Gbp9 | 0.86 | −0.22 | 3.35E−01 | 0.48 | −1.05 | 6.90E−03 |
| 93 | Tgm2 | 0.91 | −0.14 | 4.90E−01 | 0.64 | −0.65 | 2.68E−08 |
| 94 | Ly6a | 0.83 | −0.27 | 4.58E−02 | 0.42 | −1.24 | 2.58E−18 |
| 95 | Acer2 | 0.88 | −0.19 | 5.61E−01 | 0.55 | −0.87 | 4.51E−08 |
| 96 | Cp | 0.88 | −0.18 | 5.17E−01 | 0.56 | −0.83 | 1.31E−14 |
| 97 | Ly6c1 | 0.87 | −0.19 | 8.64E−02 | 0.51 | −0.97 | 2.92E−19 |
| 98 | Iigp1 | 0.68 | −0.56 | 8.91E−03 | 0.14 | −2.88 | 3.39E−12 |
| 99 | Tob2 | 0.92 | −0.13 | 5.34E−01 | 0.63 | −0.67 | 7.81E−11 |
| 100 | Gbp5 | 0.84 | −0.26 | 9.95E−02 | 0.24 | −2.04 | 7.13E−09 |
| 101 | Osmr | 0.92 | −0.12 | 8.15E−01 | 0.54 | −0.88 | 1.43E−11 |
| 102 | Xlr3b | 0.91 | −0.13 | 9.00E−01 | 0.55 | −0.86 | 1.50E−05 |
| 103 | Gbp2 | 0.73 | −0.45 | 5.57E−03 | 0.17 | −2.59 | 9.29E−10 |
| 104 | Klf2 | 0.88 | −0.18 | 6.85E−01 | 0.50 | −0.99 | 1.50E−07 |
| 105 | Per2 | 0.92 | −0.12 | 4.68E−01 | 0.60 | −0.73 | 2.69E−11 |
| 106 | Csrnp1 | 0.93 | −0.11 | 7.49E−01 | 0.63 | −0.66 | 4.54E−04 |
| 107 | Grrp1 | 0.93 | −0.10 | 7.56E−01 | 0.49 | −1.02 | 1.49E−08 |
| 108 | Gbp4 | 0.85 | −0.23 | 4.36E−01 | 0.16 | −2.63 | 3.35E−10 |
| 109 | Tgtp2 | 0.81 | −0.30 | 1.13E−01 | 0.08 | −3.73 | 5.16E−26 |
| 110 | Extl1 | 0.97 | −0.05 | 7.86E−01 | 0.65 | −0.63 | 6.13E−08 |
| 111 | Ier3 | 1.01 | 0.01 | 9.80E−01 | 0.64 | −0.64 | 6.89E−03 |
| 112 | S100a9 | NE | NE | NE | 0.26 | −1.97 | 2.98E−04 |
| 113 | Hif3a | NE | NE | NE | 0.33 | −1.60 | 4.04E−23 |
| 114 | E030018B13Rik | NE | NE | NE | 0.47 | −1.09 | 2.83E−09 |
| 115 | Gm13152 | NE | NE | NE | 0.57 | −0.80 | 1.22E−04 |
| 116 | Pla2g3 | NE | NE | NE | 0.57 | −0.80 | 7.16E−10 |
| 117 | Xlr3c | NE | NE | NE | 0.61 | −0.70 | 1.26E−02 |
| 118 | Gm13275 | NE | NE | NE | 0.62 | −0.69 | 1.30E−03 |
| 119 | 4930564K09Rik | NE | NE | NE | 0.62 | −0.69 | 1.58E−03 |
| 120 | Znf41-ps | NE | NE | NE | 0.62 | −0.68 | 4.99E−04 |
| 121 | Paqr5 | NE | NE | NE | 0.63 | −0.66 | 1.67E−03 |
| 122 | 4931406H21Rik | NE | NE | NE | 0.66 | −0.59 | 1.43E−03 |
| 123 | Xlr3a | 1.06 | 0.09 | 9.38E−01 | 0.58 | −0.78 | 1.01E−03 |
| 124 | Hspa1a | 1.10 | 0.14 | 6.46E−01 | 0.56 | −0.83 | 3.37E−10 |
| 125 | Hspa1b | 1.11 | 0.15 | 6.06E−01 | 0.55 | −0.87 | 1.50E−10 |
| 126 | Trib1 | 1.14 | 0.19 | 6.19E−01 | 0.54 | −0.88 | 6.99E−07 |
| 127 | Per1 | 1.20 | 0.26 | 1.85E−01 | 0.55 | −0.87 | 6.95E−20 |
| 128 | Podxl | 1.23 | 0.30 | 5.47E−02 | 0.51 | −0.96 | 6.45E−21 |
| 129 | Pisd-ps2 | 1.32 | 0.40 | 2.25E−03 | 0.65 | −0.62 | 8.23E−07 |
| 130 | Hist1h1d | 0.63 | −0.67 | 6.56E−07 | 1.41 | 0.50 | 4.89E−03 |
| 131 | Hist1h2ae | 0.66 | −0.60 | 1.33E−10 | 1.32 | 0.40 | 7.12E−02 |
| 132 | Hist1h2ag | 0.64 | −0.63 | 4.05E−11 | 1.31 | 0.39 | 1.71E−01 |
| 133 | Hist1h2ab | 0.66 | −0.59 | 2.17E−09 | 1.28 | 0.36 | 1.36E−01 |
| 134 | Mki67 | 0.60 | −0.73 | 2.77E−03 | 1.30 | 0.38 | 8.97E−01 |
| 135 | Hist1h2ai | 0.67 | −0.59 | 1.95E−08 | 1.24 | 0.31 | 1.88E−01 |
| 136 | Hist1h2ac | 0.66 | −0.59 | 1.51E−09 | 1.26 | 0.33 | 2.71E−01 |
| 137 | Hist1h2bh | 0.66 | −0.59 | 4.34E−08 | 1.54 | 0.62 | 2.16E−02 |
| 138 | Hist1h2an | 0.68 | −0.56 | 2.02E−06 | 1.50 | 0.59 | 1.28E−02 |
| 139 | Hist1h2bf | 0.67 | −0.59 | 4.58E−08 | 1.44 | 0.52 | 1.01E−01 |
| 140 | Hist1h3g | 0.62 | −0.69 | 4.27E−08 | 1.17 | 0.22 | 8.49E−01 |
| 141 | Hist1h2bb | 0.63 | −0.66 | 3.29E−08 | 1.14 | 0.20 | 7.55E−01 |
| 142 | Hist1h3c | 0.53 | −0.93 | 8.38E−16 | 1.08 | 0.11 | 1.00E+00 |
| 143 | Icam1 | 0.66 | −0.61 | 4.13E−04 | 1.04 | 0.06 | 1.00E+00 |
| 144 | Hist1h3b | 0.59 | −0.75 | 2.18E−11 | 1.08 | 0.11 | 1.00E+00 |
| 145 | Hist2h3c2 | 0.66 | −0.60 | 5.67E−08 | 1.06 | 0.09 | 1.00E+00 |
| 146 | Hist1h3a | 0.56 | −0.83 | 5.06E−12 | 1.02 | 0.02 | 1.00E+00 |
| 147 | Hist1h3d | 0.64 | −0.64 | 2.88E−08 | 1.01 | 0.01 | 1.00E+00 |
| 148 | Hist2h3c1 | 0.66 | −0.59 | 4.90E−08 | 1.02 | 0.03 | 1.00E+00 |
| 149 | D7Ertd715e | 0.63 | −0.66 | 1.37E−04 | 1.02 | 0.03 | 1.00E+00 |
| 150 | Hist1h1a | 0.65 | −0.62 | 6.07E−07 | 0.99 | −0.01 | 1.00E+00 |
| 151 | Plac8 | 0.37 | −1.45 | 1.86E−05 | NE | NE | NE |
| 152 | Gzma | 0.37 | −1.44 | 9.16E−09 | NE | NE | NE |
| 153 | Usp18 | 0.39 | −1.35 | 4.80E−06 | NE | NE | NE |
| 154 | Ifi27l2a | 0.41 | −1.30 | 1.06E−08 | NE | NE | NE |
| 155 | Trim30d | 0.43 | −1.23 | 1.31E−07 | NE | NE | NE |
| 156 | Mx2 | 0.45 | −1.16 | 7.06E−04 | NE | NE | NE |
| 157 | Gzmb | 0.45 | −1.14 | 2.09E−03 | NE | NE | NE |
| 158 | Trim12a | 0.46 | −1.13 | 1.07E−07 | NE | NE | NE |
| 159 | Ddx60 | 0.47 | −1.09 | 5.44E−07 | NE | NE | NE |
| 160 | Cdkn1a | 0.47 | −1.09 | 1.93E−03 | NE | NE | NE |
| 161 | Rsad2 | 0.48 | −1.06 | 1.82E−04 | NE | NE | NE |
| 162 | Ifi204 | 0.49 | −1.04 | 7.21E−04 | NE | NE | NE |
| 163 | Cd52 | 0.49 | −1.04 | 1.86E−05 | NE | NE | NE |
| 164 | Mnda | 0.50 | −1.00 | 1.59E−02 | NE | NE | NE |
| 165 | Lilrb3 | 0.51 | −0.98 | 4.05E−04 | NE | NE | NE |
| 166 | Oasl1 | 0.51 | −0.97 | 1.23E−02 | NE | NE | NE |
| 167 | Ms4a6b | 0.51 | −0.97 | 3.67E−03 | NE | NE | NE |
| 168 | Rtp4 | 0.51 | −0.96 | 2.36E−06 | NE | NE | NE |
| 169 | Mx1 | 0.51 | −0.96 | 1.44E−02 | NE | NE | NE |
| 170 | Fcgr4 | 0.51 | −0.96 | 1.43E−03 | NE | NE | NE |
| 171 | Angptl4 | 0.52 | −0.95 | 1.31E−03 | NE | NE | NE |
| 172 | Top2a | 0.52 | −0.93 | 8.16E−04 | NE | NE | NE |
| 173 | 2010002M12Rik | 0.53 | −0.93 | 2.03E−05 | NE | NE | NE |
| 174 | Ccl12 | 0.53 | −0.93 | 2.76E−02 | NE | NE | NE |
| 175 | Cxcl10 | 0.53 | −0.92 | 3.84E−03 | NE | NE | NE |
| 176 | Slfn8 | 0.54 | −0.89 | 2.05E−03 | NE | NE | NE |
| 177 | Phf11b | 0.54 | −0.89 | 6.40E−04 | NE | NE | NE |
| 178 | Plekha4 | 0.54 | −0.88 | 4.00E−03 | NE | NE | NE |
| 179 | Kirrel2 | 0.55 | −0.87 | 1.54E−04 | NE | NE | NE |
| 180 | Epsti1 | 0.55 | −0.86 | 6.53E−06 | NE | NE | NE |
| 181 | Ch25h | 0.55 | −0.86 | 5.67E−05 | NE | NE | NE |
| 182 | Spn | 0.56 | −0.84 | 1.25E−05 | NE | NE | NE |
| 183 | Cybb | 0.56 | −0.83 | 8.69E−07 | NE | NE | NE |
| 184 | Ifi205 | 0.56 | −0.82 | 3.57E−02 | NE | NE | NE |
| 185 | Gm8989 | 0.57 | −0.81 | 4.19E−05 | NE | NE | NE |
| 186 | Oas1a | 0.57 | −0.81 | 2.73E−02 | NE | NE | NE |
| 187 | Hist1h1b | 0.57 | −0.80 | 1.00E−03 | NE | NE | NE |
| 188 | Zfp36 | 0.58 | −0.78 | 2.98E−03 | NE | NE | NE |
| 189 | Phf11d | 0.59 | −0.77 | 6.94E−03 | NE | NE | NE |
| 190 | Itgb7 | 0.60 | −0.75 | 1.55E−05 | NE | NE | NE |
| 191 | Bst2 | 0.60 | −0.74 | 4.99E−04 | NE | NE | NE |
| 192 | Isg15 | 0.60 | −0.73 | 3.96E−02 | NE | NE | NE |
| 193 | Maff | 0.61 | −0.72 | 3.18E−03 | NE | NE | NE |
| 194 | Cyp1b1 | 0.61 | −0.71 | 1.38E−02 | NE | NE | NE |
| 195 | Crybb1 | 0.61 | −0.71 | 5.63E−03 | NE | NE | NE |
| 196 | Ms4a6d | 0.62 | −0.70 | 1.14E−02 | NE | NE | NE |
| 197 | Hck | 0.62 | −0.69 | 1.61E−04 | NE | NE | NE |
| 198 | Slfn2 | 0.62 | −0.68 | 7.68E−06 | NE | NE | NE |
| 199 | Irf7 | 0.63 | −0.68 | 2.75E−04 | NE | NE | NE |
| 200 | Serpina3f | 0.63 | −0.67 | 2.31E−02 | NE | NE | NE |
| 201 | Parp10 | 0.63 | −0.66 | 1.91E−03 | NE | NE | NE |
| 202 | Hist1h3i | 0.64 | −0.65 | 1.24E−08 | 1.00 | 0.00 | 1.00E+00 |
| 203 | Dhx58 | 0.64 | −0.64 | 1.31E−02 | NE | NE | NE |
| 204 | Itgb2 | 0.64 | −0.64 | 5.96E−05 | NE | NE | NE |
| 205 | Nmi | 0.64 | −0.64 | 6.83E−04 | NE | NE | NE |
| 206 | Gm12250 | 0.64 | −0.63 | 3.80E−03 | NE | NE | NE |
| 207 | Psmb8 | 0.65 | −0.62 | 1.89E−03 | NE | NE | NE |
| 208 | Srgn | 0.65 | −0.62 | 1.49E−03 | NE | NE | NE |
| 209 | Serpina3g | 0.65 | −0.61 | 2.63E−02 | NE | NE | NE |
| 210 | AW112010 | 0.66 | −0.60 | 1.31E−02 | NE | NE | NE |
| 211 | Hmgb2 | 0.66 | −0.60 | 9.75E−04 | NE | NE | NE |
| 212 | Plaur | 0.66 | −0.59 | 4.96E−03 | NE | NE | NE |
| 213 | Gm7030 | 0.66 | −0.59 | 9.76E−04 | NE | NE | NE |
| 214 | Irf8 | 0.67 | −0.59 | 9.23E−04 | NE | NE | NE |
| 215 | AF251705 | 0.67 | −0.58 | 4.75E−04 | NE | NE | NE |
| 216 | Ltbp2 | 0.54 | −0.89 | 1.13E−05 | NE | NE | NE |
| 217 | Il20rb | 0.57 | −0.81 | 3.14E−05 | NE | NE | NE |
| 218 | Crtam | 0.57 | −0.81 | 1.80E−10 | NE | NE | NE |
| 219 | Ror1 | 0.57 | −0.80 | 5.44E−07 | NE | NE | NE |
| 220 | Mybpc3 | 0.65 | −0.63 | 3.22E−05 | NE | NE | NE |
| 221 | Fermt3 | 0.66 | −0.60 | 2.91E−03 | 0.92 | −0.13 | 1.00E+00 |
| 222 | Tor3a | 0.57 | −0.80 | 7.13E−07 | 0.87 | −0.20 | 8.75E−01 |
| 223 | Rac2 | 0.62 | −0.68 | 6.01E−04 | 0.89 | −0.17 | 7.92E−01 |
| 224 | Cmpk2 | 0.65 | −0.61 | 4.90E−08 | 0.91 | −0.14 | 8.28E−01 |
| 225 | Oas1b | 0.61 | −0.72 | 3.19E−04 | 0.89 | −0.17 | 6.37E−01 |
| 226 | Pyhin1 | 0.50 | −1.00 | 8.49E−04 | 0.85 | −0.24 | 8.25E−01 |
| 227 | Rorc | 0.58 | −0.78 | 5.91E−06 | 0.88 | −0.19 | 9.35E−01 |
| 228 | Ms4a6c | 0.62 | −0.70 | 5.10E−05 | 0.82 | −0.29 | 1.66E−01 |
| 229 | Rhoj | 0.67 | −0.59 | 1.16E−02 | 0.85 | −0.24 | 7.65E−01 |
| 230 | Trim21 | 0.60 | −0.73 | 5.90E−04 | 0.83 | −0.28 | 7.17E−01 |
| 231 | Pdk4 | 0.52 | −0.93 | 5.05E−06 | 0.79 | −0.33 | 1.44E−01 |
| 232 | Herc6 | 0.60 | −0.74 | 7.32E−06 | 0.83 | −0.27 | 6.61E−01 |
| 233 | Rbm3 | 0.58 | −0.78 | 3.09E−02 | 0.82 | −0.29 | 1.03E−02 |
| 234 | Zc3hav1 | 0.61 | −0.72 | 7.32E−06 | 0.85 | −0.24 | 6.88E−01 |
| 235 | Ddx58 | 0.60 | −0.73 | 3.04E−05 | 0.87 | −0.21 | 8.83E−01 |
| 236 | Gm4951 | 0.61 | −0.72 | 3.48E−04 | 0.86 | −0.21 | 5.77E−01 |
| 237 | Hist1h3f | 0.66 | −0.60 | 4.90E−08 | 0.89 | −0.17 | 8.44E−01 |
| 238 | Crlf2 | 0.63 | −0.67 | 2.89E−04 | 0.87 | −0.20 | 8.75E−01 |
| 239 | Hist1h3h | 0.63 | −0.67 | 4.90E−08 | 0.97 | −0.05 | 1.00E+00 |
| 240 | Naip2 | 0.62 | −0.70 | 1.25E−05 | 0.93 | −0.11 | 1.00E+00 |
| 241 | Hspb8 | 0.66 | −0.61 | 1.70E−04 | 0.95 | −0.08 | 1.00E+00 |
| 242 | Eif2ak2 | 0.60 | −0.73 | 7.47E−05 | 0.93 | −0.10 | 9.10E−01 |
| 243 | Car8 | 0.66 | −0.59 | 2.24E−08 | 0.94 | −0.08 | 1.00E+00 |
| 244 | Gdf10 | 0.66 | −0.59 | 1.68E−03 | 2.52 | 1.33 | 1.74E−11 |
| 245 | Atp1a4 | 1.13 | 0.18 | 4.19E−01 | 1.91 | 0.93 | 1.37E−02 |
| 246 | Aldoart1 | 1.15 | 0.20 | 2.86E−01 | 2.24 | 1.16 | 1.05E−03 |
| 247 | Gm10012 | 0.91 | −0.13 | 4.74E−01 | 1.84 | 0.88 | 8.81E−05 |
| 248 | Gm6548 | 0.88 | −0.18 | 1.69E−01 | 2.00 | 1.00 | 3.50E−03 |
| 249 | Hmgcs2 | 0.92 | −0.11 | 7.53E−01 | 1.56 | 0.64 | 6.15E−03 |
| 250 | Igfbp4 | 0.93 | −0.10 | 5.67E−01 | 1.53 | 0.61 | 4.73E−03 |
| 251 | Gm6402 | 0.93 | −0.10 | 7.26E−01 | 1.78 | 0.84 | 9.30E−03 |
| 252 | Gm12504 | 0.95 | −0.08 | 7.96E−01 | 1.99 | 0.99 | 4.03E−03 |
| 253 | Mid1 | 0.95 | −0.08 | 9.43E−01 | 1.81 | 0.86 | 2.23E−02 |
| 254 | Smarca5-ps | 0.95 | −0.07 | 8.32E−01 | 1.61 | 0.69 | 3.21E−02 |
| 255 | Gm6251 | 0.99 | −0.02 | 9.67E−01 | 1.84 | 0.88 | 1.13E−02 |
| 256 | Acta1 | 0.99 | −0.01 | 9.71E−01 | 2.56 | 1.36 | 7.95E−04 |
| 257 | Haus4 | NE | NE | NE | 1.53 | 0.61 | 5.03E−03 |
| 258 | Gm6194 | NE | NE | NE | 1.80 | 0.85 | 1.86E−03 |
| 259 | Calca | NE | NE | NE | 1.87 | 0.90 | 8.78E−13 |
| 260 | Caleb | NE | NE | NE | 1.93 | 0.95 | 1.26E−04 |
| 261 | Gm10413 | NE | NE | NE | 2.19 | 1.13 | 2.66E−07 |
| 262 | 4930555G01Rik | NE | NE | NE | 2.65 | 1.40 | 4.57E−30 |
| 263 | Gdpd3 | NE | NE | NE | 2.85 | 1.51 | 4.79E−16 |
| 264 | Gm5795 | NE | NE | NE | 3.15 | 1.66 | 3.95E−15 |
| 265 | Eps8l1 | NE | NE | NE | 3.56 | 1.83 | 3.55E−13 |
| 266 | Mmrn2 | 1.64 | 0.71 | 4.56E−06 | 1.62 | 0.70 | 2.48E−05 |
| 267 | Gm5796 | 2.76 | 1.47 | 1.25E−14 | 2.81 | 1.49 | 3.69E−36 |
| 268 | Beta-s | 2.73 | 1.45 | 1.17E−08 | 2.57 | 1.36 | 9.44E−03 |
| 269 | Gm3500 | 3.21 | 1.68 | 2.35E−18 | 3.02 | 1.59 | 3.13E−31 |
| 270 | Gm10409 | 2.59 | 1.37 | 2.97E−11 | 2.30 | 1.20 | 1.93E−18 |
| 271 | Gm3383 | 3.11 | 1.64 | 8.32E−19 | 2.70 | 1.43 | 1.45E−25 |
| 272 | Nlrp5-ps | 1.72 | 0.78 | 8.14E−03 | 1.59 | 0.67 | 1.07E−07 |
| 273 | Gm10406 | 3.20 | 1.68 | 1.11E−17 | 2.70 | 1.43 | 1.14E−20 |
| 274 | LOC100861615 | 3.12 | 1.64 | 3.66E−14 | 2.77 | 1.47 | 3.86E−21 |
| 275 | Bche | 1.55 | 0.64 | 8.08E−04 | 1.49 | 0.57 | 1.70E−04 |
| 276 | Serpinb1a | 1.72 | 0.78 | 1.28E−06 | 1.52 | 0.61 | 2.44E−08 |
| 277 | Sh3bp5 | 2.03 | 1.02 | 7.51E−15 | 1.77 | 0.82 | 1.03E−19 |
| 278 | Gm3264 | 3.37 | 1.75 | 5.48E−25 | 2.64 | 1.40 | 4.86E−17 |
| 279 | Gm3558 | 2.73 | 1.45 | 3.28E−14 | 2.26 | 1.18 | 3.80E−17 |
| 280 | Gjc2 | 1.57 | 0.65 | 1.02E−09 | 1.45 | 0.53 | 2.08E−09 |
| 281 | Rprl3 | 1632.08 | 10.67 | 0.00E+00 | 3550.60 | 11.79 | 0.00E+0 |
| 282 | Opalin | 1.44 | 0.53 | 6.52E−04 | 1.55 | 0.64 | 7.34E−11 |
| 283 | Rprl2 | 452.46 | 8.82 | 5.47E−146 | 3446.78 | 11.75 | 5.98E−148 |
| 284 | BC002163 | 1.33 | 0.42 | 1.13E−02 | 1.52 | 0.61 | 4.72E−09 |
| 285 | Hmcn1 | 1.24 | 0.31 | 2.71E−01 | 1.63 | 0.71 | 7.50E−14 |
| 286 | Scd4 | 1.20 | 0.26 | 3.42E−01 | 1.51 | 0.59 | 2.52E−02 |
| 287 | Olfml1 | 1.19 | 0.25 | 2.47E−01 | 1.61 | 0.69 | 2.83E−09 |
| 288 | Rps15a-ps6 | 1.25 | 0.33 | 2.48E−01 | 1.83 | 0.87 | 2.65E−05 |
| 289 | Kdr | 1.30 | 0.38 | 3.33E−02 | 1.98 | 0.99 | 5.68E−14 |
| 290 | Padi2 | 1.27 | 0.35 | 2.28E−02 | 1.60 | 0.68 | 2.08E−14 |
| 291 | Igf2 | 1.33 | 0.41 | 3.54E−02 | 1.65 | 0.73 | 4.81E−04 |
| 292 | Rgcc | 1.33 | 0.42 | 9.36E−04 | 1.65 | 0.72 | 1.42E−05 |
| 293 | Tnc | 1.59 | 0.67 | 1.24E−05 | 1.28 | 0.35 | 1.20E−01 |
| 294 | Gm20594 | 1.71 | 0.77 | 2.02E−02 | 1.32 | 0.40 | 1.38E−03 |
| 295 | Hddc3 | 2.90 | 1.54 | 1.06E−20 | 2.18 | 1.13 | 7.75E−16 |
| 296 | Gm3002 | 2.66 | 1.41 | 8.38E−16 | 1.96 | 0.97 | 5.64E−09 |
| 297 | Ide | 1.54 | 0.63 | 2.60E−05 | 1.34 | 0.42 | 9.93E−06 |
| 298 | Fabp7 | 2.65 | 1.41 | 9.83E−18 | 1.81 | 0.86 | 1.81E−11 |
| 299 | Gabra2 | 2.78 | 1.48 | 1.56E−19 | 1.95 | 0.96 | 2.84E−33 |
| 300 | Col3a1 | 1.73 | 0.79 | 1.50E−05 | 1.41 | 0.50 | 6.24E−03 |
| 301 | Ndn | 1.79 | 0.84 | 2.78E−18 | 1.24 | 0.31 | 6.22E−03 |
| 302 | Srsf4 | 1.65 | 0.72 | 1.16E−03 | 1.16 | 0.21 | 1.95E−01 |
| 303 | Txn2 | 1.56 | 0.64 | 6.94E−03 | 1.12 | 0.16 | 5.75E−01 |
| 304 | Arc | 1.84 | 0.88 | 3.60E−02 | 1.15 | 0.21 | 6.11E−01 |
| 305 | Nr4a1 | 2.46 | 1.30 | 1.26E−04 | 0.57 | −0.82 | 6.51E−06 |
| 306 | Mir5109 | 1.78 | 0.83 | 1.07E−02 | 0.83 | −0.26 | 8.24E−01 |
| 307 | Egr1 | 2.12 | 1.08 | 1.22E−05 | 0.85 | −0.24 | 7.14E−01 |
| 308 | Rasl11b | 2.06 | 1.04 | 1.72E−14 | 1.07 | 0.09 | 1.00E+00 |
| 309 | Atp10d | 2.74 | 1.46 | 9.97E−17 | 1.07 | 0.10 | 1.00E+00 |
| 310 | Lars2 | 1.74 | 0.80 | 4.32E−03 | 0.98 | −0.03 | 1.00E+00 |
| 311 | Bdnf | 1.51 | 0.59 | 2.21E−05 | 0.99 | −0.02 | 1.00E+00 |
| 312 | Efnb2 | 1.52 | 0.60 | 4.64E−05 | 0.99 | −0.02 | 1.00E+00 |
| 313 | Egr2 | 3.48 | 1.80 | 3.60E−03 | NE | NE | NE |
| 314 | Egr4 | 1.95 | 0.96 | 9.62E−05 | NE | NE | NE |
| 315 | Fosb | 1.67 | 0.74 | 2.83E−02 | NE | NE | NE |
| 316 | Cdhr1 | 4.23 | 2.08 | 3.70E−11 | NE | NE | NE |

Differences of expression between samples were validated by qPCR on independent biological samples at different time points in PbA-infected brains. Briefly, complimentary DNA (cDNA) was synthesized with M-MLV reverse transcriptase (Thermo Fisher Scientific). Quantitative polymerase chain reaction (qPCR) was performed on an Applied Biosystems instrument (Life Technologies) using PerfeCTa SYBR green Super Mix™+ROX reagent (Quanta Biosciences). qPCRs were performed using gene primer pairs listed in Table 5. Comparative quantification was calculated using the 2^{-ΔCt} method and target genes were expressed relative to the hypoxanthine phosphoribosyltransferase (Hprt) reference gene. Fold changes in gene expression of infected mice was expressed relative to those of naive mice.

TABLE 5
qPCR validation primers

| Gene | Orientation | Sequence (5′-3′) | SEQ ID NO: |
|---|---|---|---|
| C4b | sense | GATGAGGTTCGCCTGCTATT | 13 |
| C4b | antisense | GACTTGGGTGATCTTGGACTC | 14 |
| Cd3g | sense | TCTTCCTTGCTCTTGGTGTATATC | 15 |
| Cd3g | antisense | GAGATGGCTGTACTGGTCATATT | 16 |
| cEBPd | sense | TCGACTTCAGCGCCTACATTGA | 17 |
| cEBPd | antisense | CCGCTTTGTGGTTGCTGTTGAA | 18 |
| Gmzb | sense | CGGGAGTGTGAGTCCTACTTTA | 19 |
| Gmzb | antisense | GTGGAGGTGAACCATCCTTATATC | 20 |
| HPRT | sense | TCAGTCAACGGGGGACATAAA | 21 |
| HPRT | antisense | GGGGCTGTACTGCTTAACCAG | 22 |
| Ifi35 | sense | GATCCAGAAAGCCGAGATCAA | 23 |
| Ifi35 | antisense | CTGGAAGTGGATCTCAAGGATG | 24 |
| Ifit3 | sense | CTGAACTGCTCAGCCCACAC | 25 |
| Ifit3 | antisense | TGGACATACTTCCTTCCCTGA | 26 |
| Irf7 | sense | CGACTTCAGCACTTTCTTCCGAGA | 27 |
| Irf7 | antisense | AGATGGTGTAGTGTGGTGACCCTT | 28 |
| Ifr9 | sense | AAATGGGAGGACCAATGGCGTT | 29 |
| Ifr9 | antisense | ATAGATGAAGGTGAGCAGCAGCGA | 30 |
| Itgam (CD11b) | sense | GAAAGTAGCAAGGAGTGTGTTTG | 31 |
| Itgam (CD11b) | antisense | GGGTCTAAAGCCAGGTCATAAG | 32 |
| Lat | sense | GGATGAAGACGACTATCCCAAC | 33 |
| Lat | antisense | CCTCACTCTCAGGAACATTCAC | 34 |
| Oasl2 | sense | GGACCCGTTCCCCGACCTGT | 35 |
| Oasl2 | antisense | CGACCTCCCGGTTTCTCGCC | 36 |
| Plin4 | sense | CATCATGTCAGCTTCAGGAGAT | 37 |
| Plin4 | antisense | GGGTCTGTTGCTGTTTGTAAG | 38 |
| Trim25 | sense | AACTGAAGGCAGAGGTTGAG | 39 |
| Trim25 | antisense | CCCTTGGTAGATTCCCATTATCA | 40 |
| Usp18 | sense | CGTGCTTGAGAGGGTCATTTG | 41 |
| Usp18 | antisense | GGTCGGGAGTCCACAACTTC | 42 |

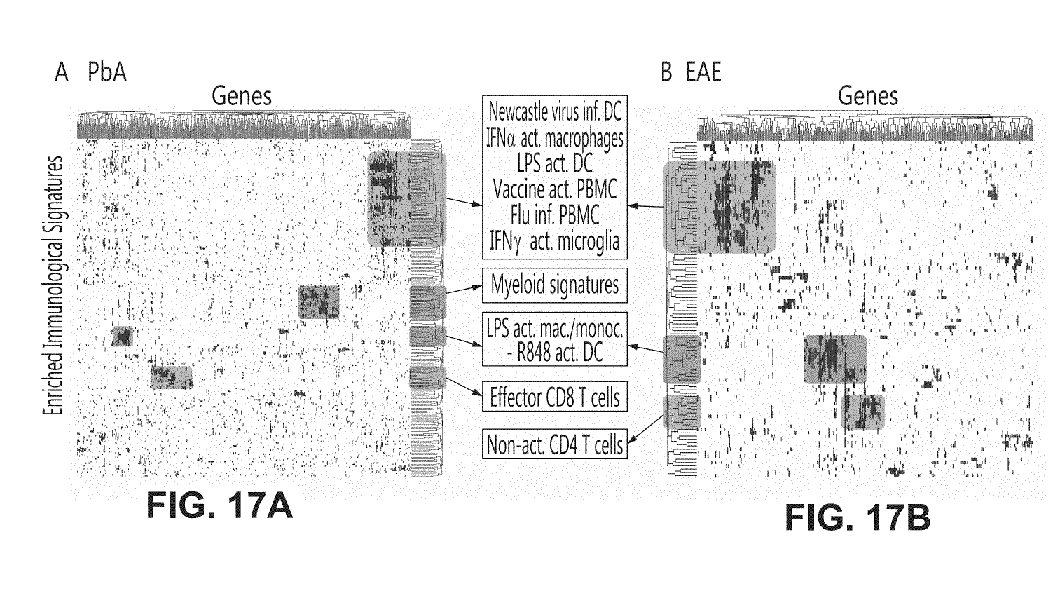

Identification of USP15-regulated pathways and cellular responses. Genes differentially expressed in a USP15-dependent fashion were identified in the RNA-sequencing datasets (fold change >11.51, adjusted p value <0.05) from brain and spinal cords obtained from control (B6), and from Usp15 mutant mice. Gene ontology enrichment analysis was performed using the DAVID bioinformatics resources. The USP15 differential gene expression profiles (brain, spinal cord) was also subjected to gene set enrichment analysis using GSEA and using MSigDB public immunologic gene signatures (cell specific, response to stimuli). Following identification of enriched immunological signatures in GSEA (FDR<0.01) (representative examples are shown in FIG. 11D), the leading-edge genes were identified for each of these signatures (genes that appear in the ranked list at or before the point at which the running sum reaches its maximum deviation from zero); the leading-edge genes are driving the enrichment signals during the statistical analysis. For PbA, 1347 genes were driving the enrichment of 235 immunological signatures, whereas in EAE, there was 849 genes for 102 signatures. In order to globally identify cellular responses and pathways regulated in situ by USP15 in both brain (in response to PbA infection) and in spinal cord (during EAE induction), we performed unbiased clustering (using Kendall's Tau distance metric with complete linkage) of the leading edge genes in the PbA and EAE datasets. For the clustering analyses, only genes implicated in at least 2% of the signatures were considered (PbA: 530 genes and EAE: 353 genes) (FIG. 17). Since the clustering analysis of individual datasets revealed the enrichment of common immunological functions, we have combined both leading datasets (PbA U EAE), kept genes associated in the enrichment of at least 5 signatures (627 genes for 264 signatures) and performed clustering. Results of the combined clustering analysis is shown in FIG. 12A.

A Mutation in the Catalytic Domain of USP15 Protects Mice Against Cerebral Malaria.

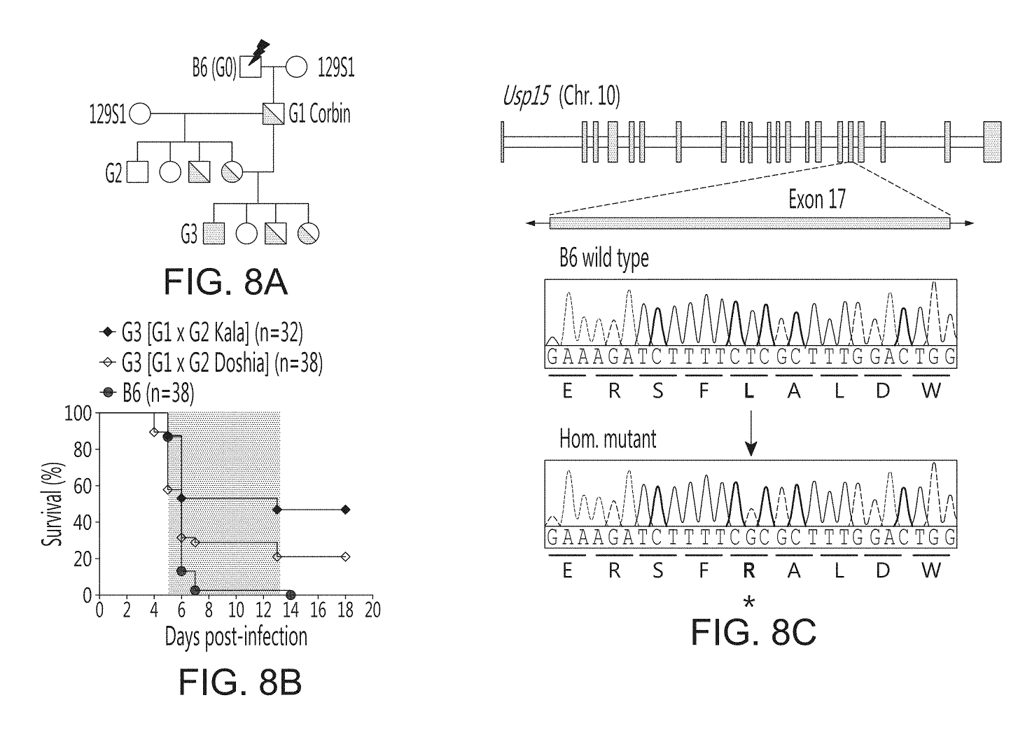

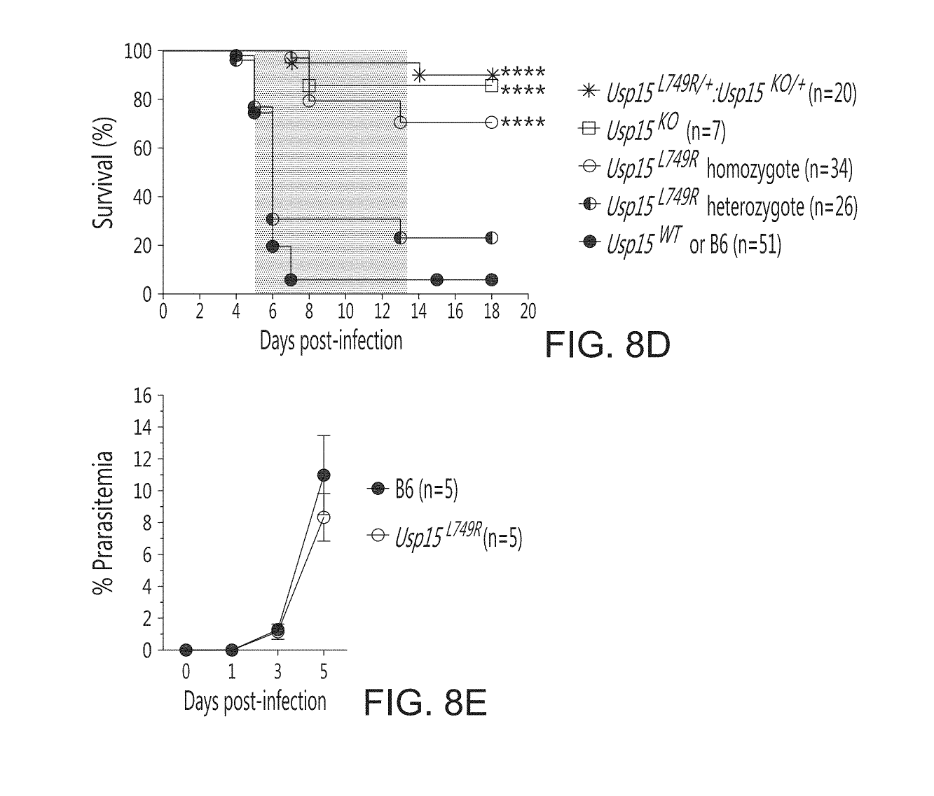

We used N-ethyl-N-nitrosourea (ENU) genome-wide mutagenesis to identify genes and pathways which when inactivated protect mice against lethal encephalitis in the experimental cerebral malaria (ECM) model of Plasmodium berghei ANKA (PbA) infection. Such ECM-protective mutations affect genes and pathways that are required for pathological neuroinflammation, and the corresponding proteins may represent valuable entry points to better understand the disease process and to suggest novel targets for drug discovery. In this screen (FIG. 8A), G3 pedigrees derived from mutagenized G0 males are infected with 10⁶ PbA-parasitized red blood cells (pRBCs) and monitored for appearance of neurological symptoms, and for overall survival. By day 5 post-infection, ECM-susceptible mice develop rapidly fatal encephalitis (coma, paralysis, tremors and seizures) while animals that survive beyond day 9 post-infection, without development of cerebral symptoms, are deemed ECM-resistant.

In pedigree Corbin (FIG. 8A), 40% of the G3 offspring produced by mating of Corbin (G1) to G2 females Doshia and Kala displayed ECM-resistance (28/70) (FIGS. 8A, 8B). To identify the ECM-protective mutation segregating in this pedigree, 3 ECM-resistant G3 offspring were characterized by whole-exome sequencing (WES). A single de novo homozygote sequence variant common to the 3 mice was identified as a T-to-G transversion (Chr 10: 122,562,078 bp, genome build NCBI37/mm9) in exon 17 of the Ubiquitin-specific protease 15 (Usp15) gene (FIG. 8C). The transversion causes a non-conservative leucine (L) to arginine (R) amino acid substitution at position 749 (L749R) in the carboxy terminal moiety of the protein (FIG. 8C). Leucine at position 749 of USP15 is invariant across vertebrates, hence its substitution to the large and positively charged arginine is likely to be detrimental to protein structure and function, and hence be pathological (FIG. 8A). Genotype-phenotype correlations in additional Corbin G3 mice validated that homozygosity for Usp15^{L749R} is ECM-protective (>80% survival); heterozygosity for Usp15^{L749R/+} conferred low but significant ECM-protection (˜30% survival), while Usp15^{+/+} mice were ECM susceptible (FIG. 8D). Continuous backcrossing of the Usp15^{L749R} mutation to a pure C57BL/6J genetic background confirmed that Usp15^{L749R} was ECM-protective (data not shown). We further observed that a null Usp15^{-/-} mutant was also protected against ECM (85% survival), validating the role of USP15 in neuroinflammation. In addition, we showed that Usp15^{L749R/+}:Usp15^{KO/+} double heterozygotes were also ECM-resistant (˜95% survival; FIG. 8D), suggesting that the protective effect of the L749R allele is caused by a loss of function inherited in an incompletely recessive fashion. Finally, we observed that the protective effect of Usp15^{L749R} against neuroinflammation was specific and independent of possible effects on blood-stage replication of the PbA parasite, as kinetics of blood parasitemia were identical in controls and in Usp15 mutant mice (FIG. 8E).

The USP15^{L749R} mutant variant shows reduced protein stability. USP15 is a member of the USP family of cysteine protease deubiquitination enzymes. It contains several structural motifs which include an N-terminal regulatory DUSP domain (domain in ubiquitin specific protease), two ubiquitin-like folds (UBL), a long C-terminal catalytic domain that includes a catalytic triad (C269, H862, D879) and Zn-coordinating cysteine residues (C419, C422, C780, C783). The role of L749 in structure or function of USP15 is unknown.

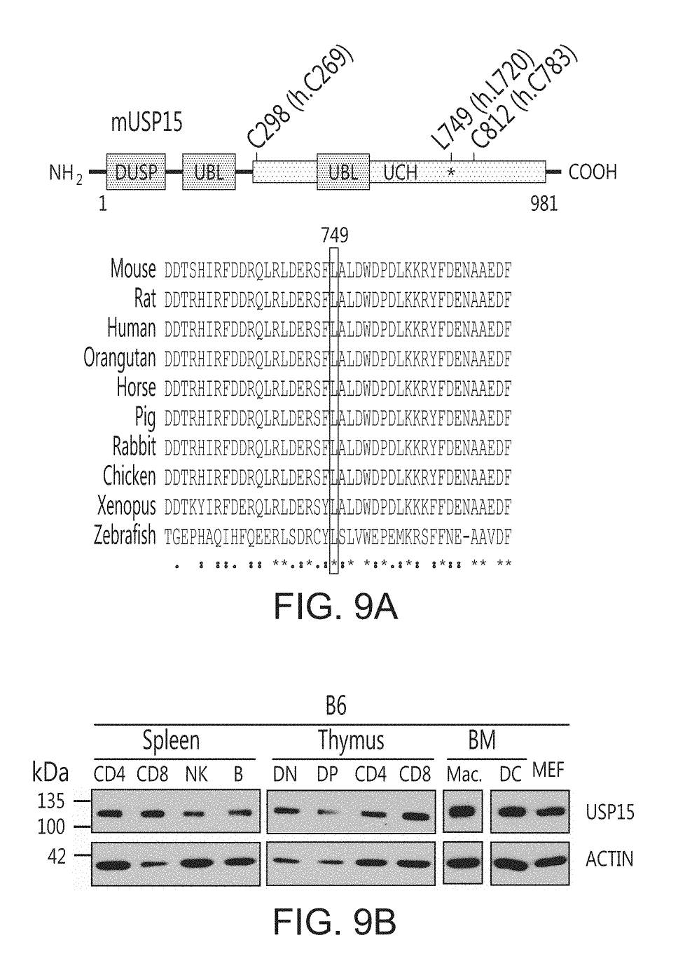

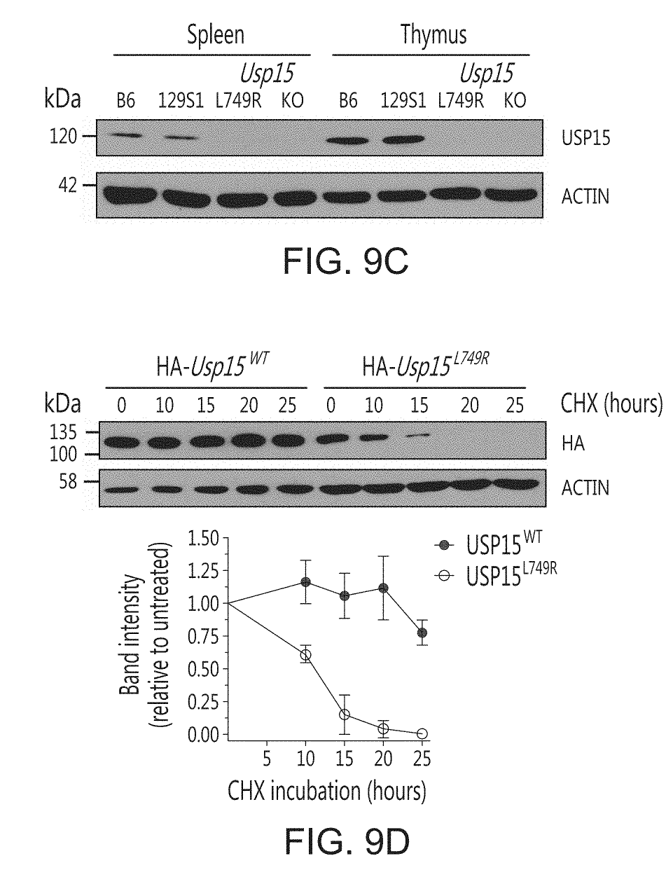

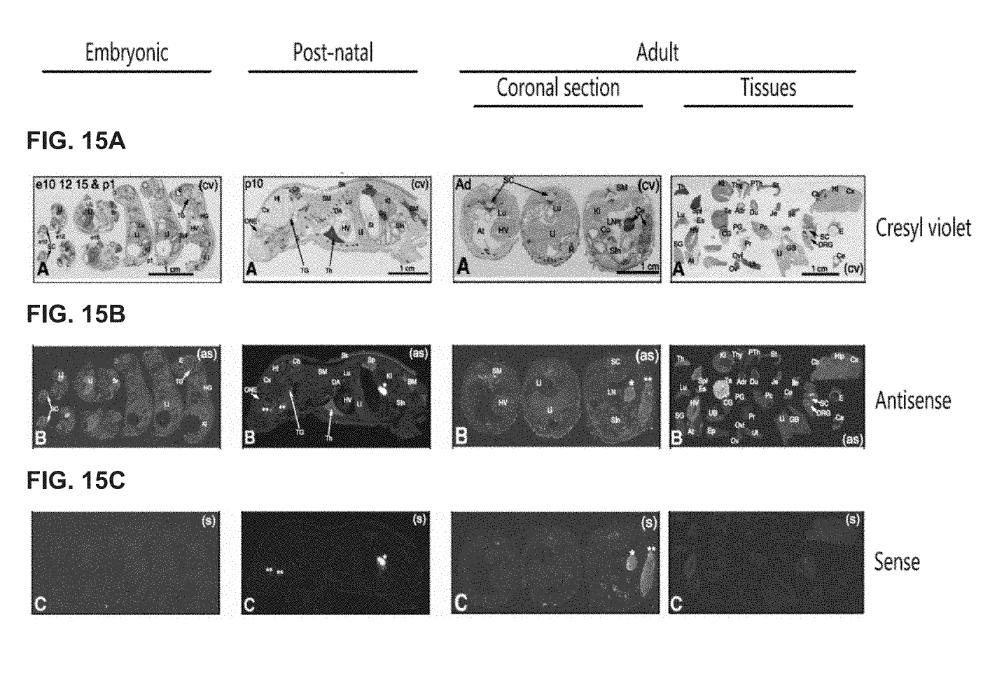

To gain insight into the role of USP15 in neuroinflammation, we investigated USP15 protein and mRNA expression in tissues and cell types that may play a role in ECM pathogenesis. In situ hybridization studies on whole embryonic, post-natal and adult mouse sections revealed low and ubiquitous Usp15 mRNA expression in most tissues and organs (FIG. 15). The USP15 protein was detected by immunoblotting as a 112-kilodalton species in spleen, and thymus of wild type B6 and 129S1 mice (FIG. 9C). In these organs, USP15 was ubiquitously expressed in the lymphoid and myeloid lineages including all singly and doubly positive CD3⁺ T cell subsets (CD4⁺, CD8⁺, CD4⁺/CD8⁺, CD4⁻/CD8⁻), NK cells, B cells, and in myeloid cells (macrophages, dendritic cells) and in primary embryo fibroblasts (FIG. 9B). The observed ubiquitous expression of Usp15 RNA and protein suggests that it may play a complex role during neuroinflammation, possibly implicating multiple organs, cell types and associated responses.

Importantly, USP15 protein could not be detected in the spleen and thymus of Usp15^{L749R} homozygote mutants (FIG. 9C), similar to Usp15^{-/-} mice used as controls (data not shown), suggesting reduced protein expression and/or stability of the L749R variant in vivo. To further investigate this possibility, cells stably expressing either HA-tagged USP15 (VVT) or USP15^{L749R} variants were treated with the protein synthesis inhibitor cycloheximide (CHX), and protein levels were monitored over time by immunoblotting (FIG. 9D). The USP15^{L749R} variant showed a reduced half-life (˜10 hours) compared to VVT (>25 hours), with no L749R variant protein detected after 20 hours of CHX incubation. These results strongly suggest that the L749R mutation behaves as a loss of function in USP15, phenotypically expressed as reduced protein stability. This is in agreement with genetic complementation studies (FIG. 8D) and the noted recessive mode of inheritance of Usp15^{L749R}.

Effect of Usp15^{L749R} on Microbial and Autoimmune Models of Neuroinflammation.

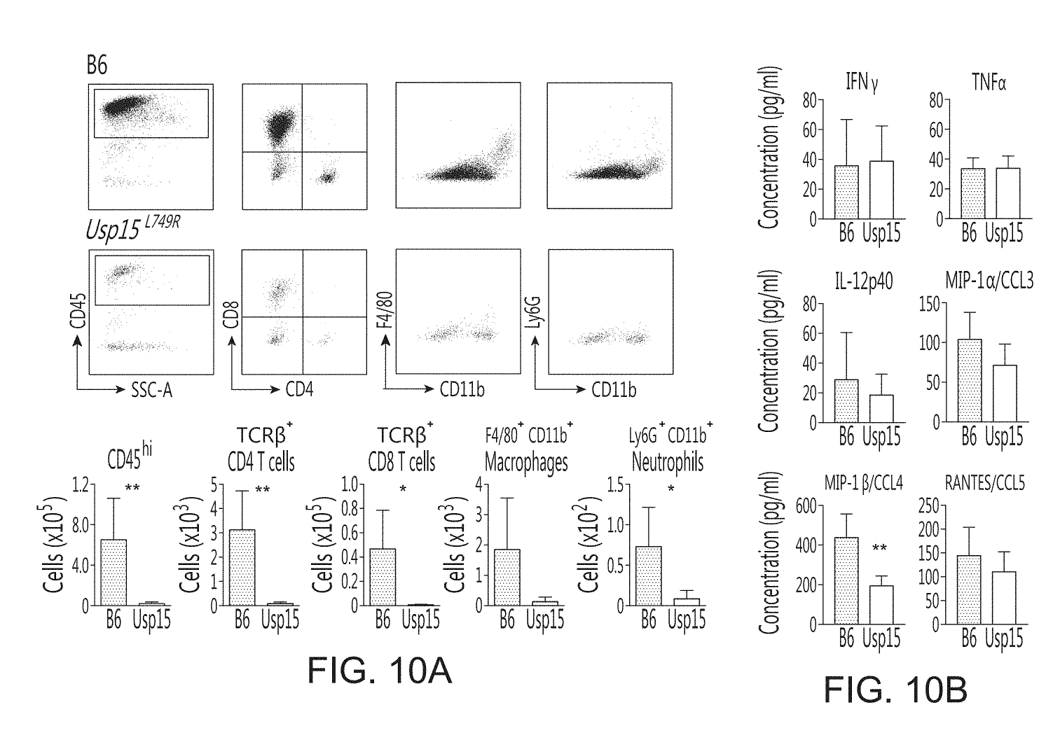

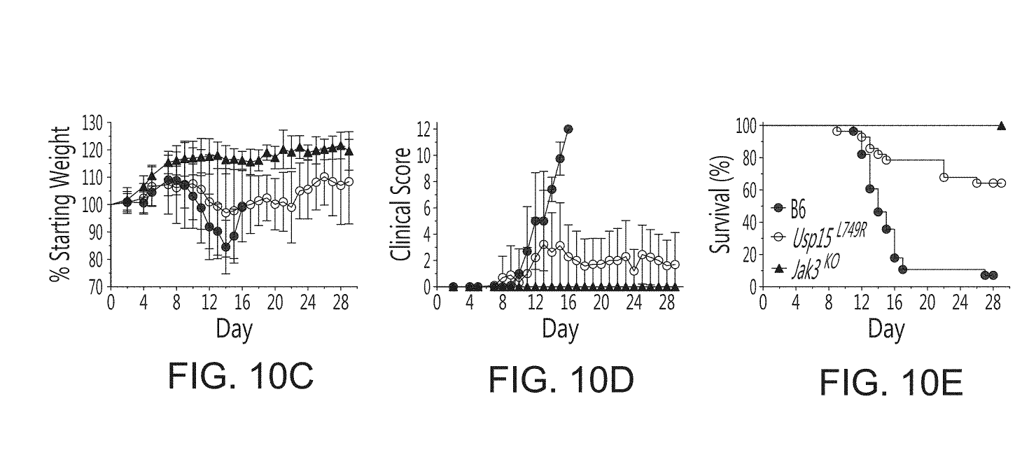

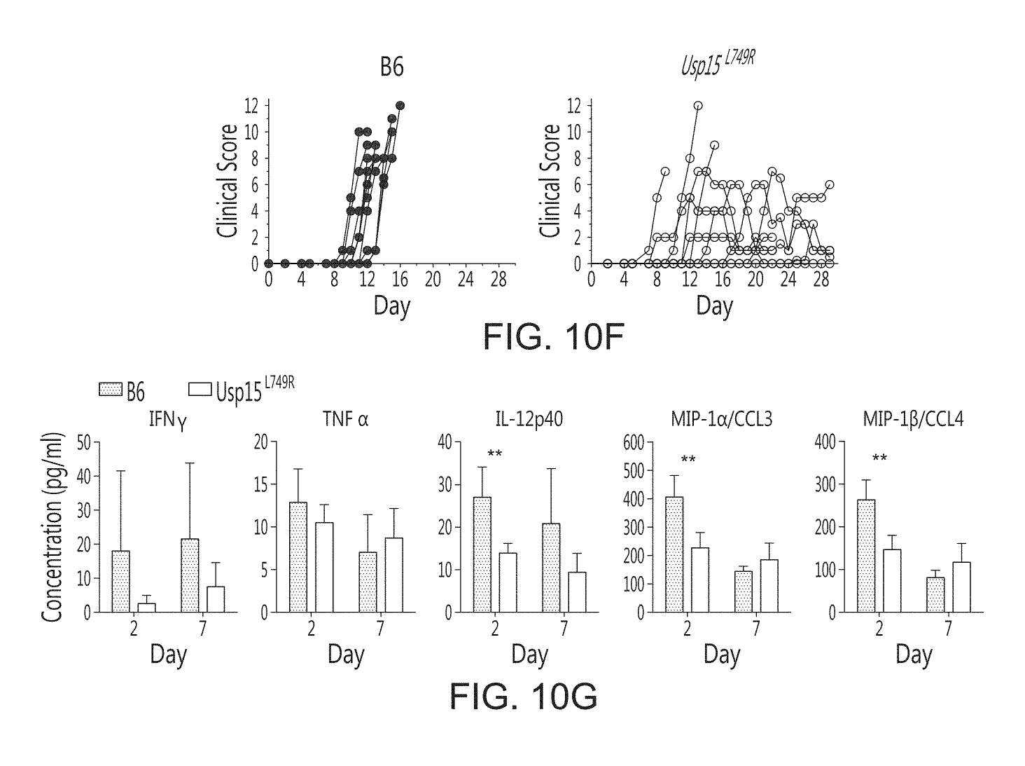

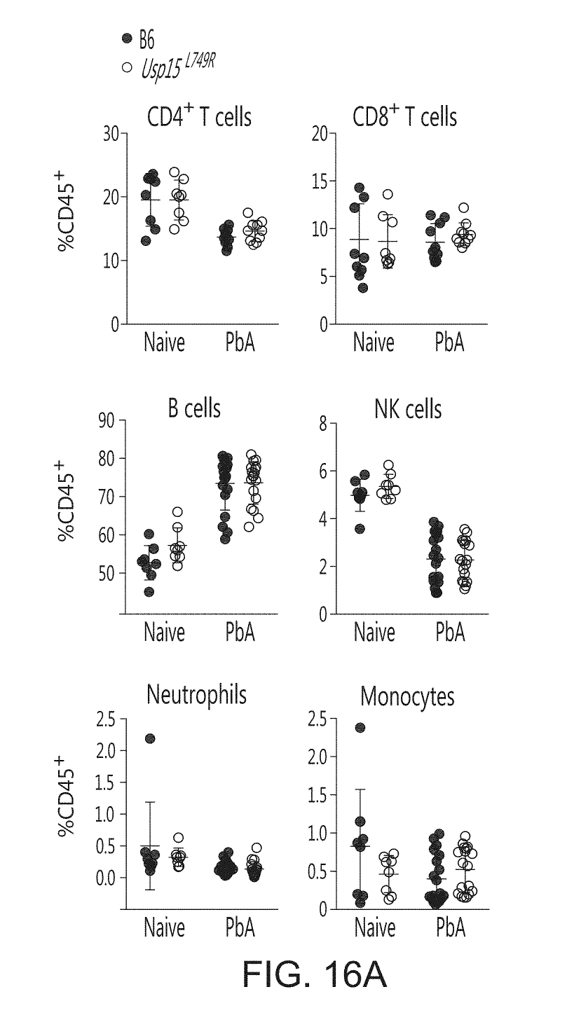

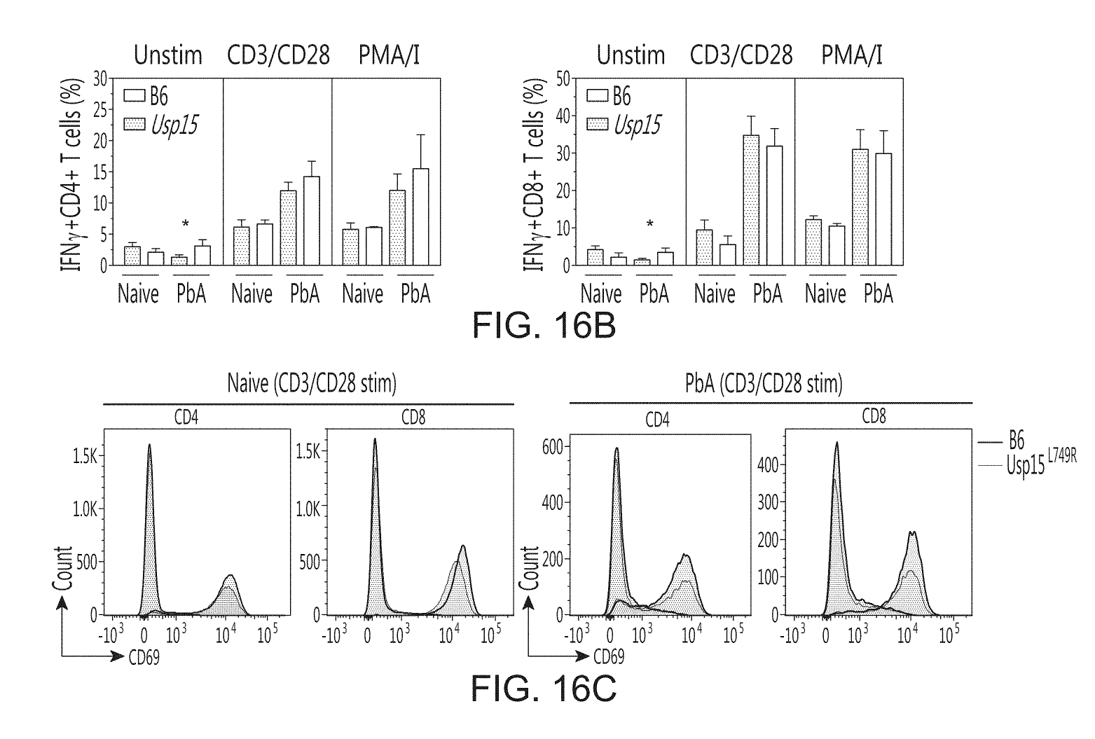

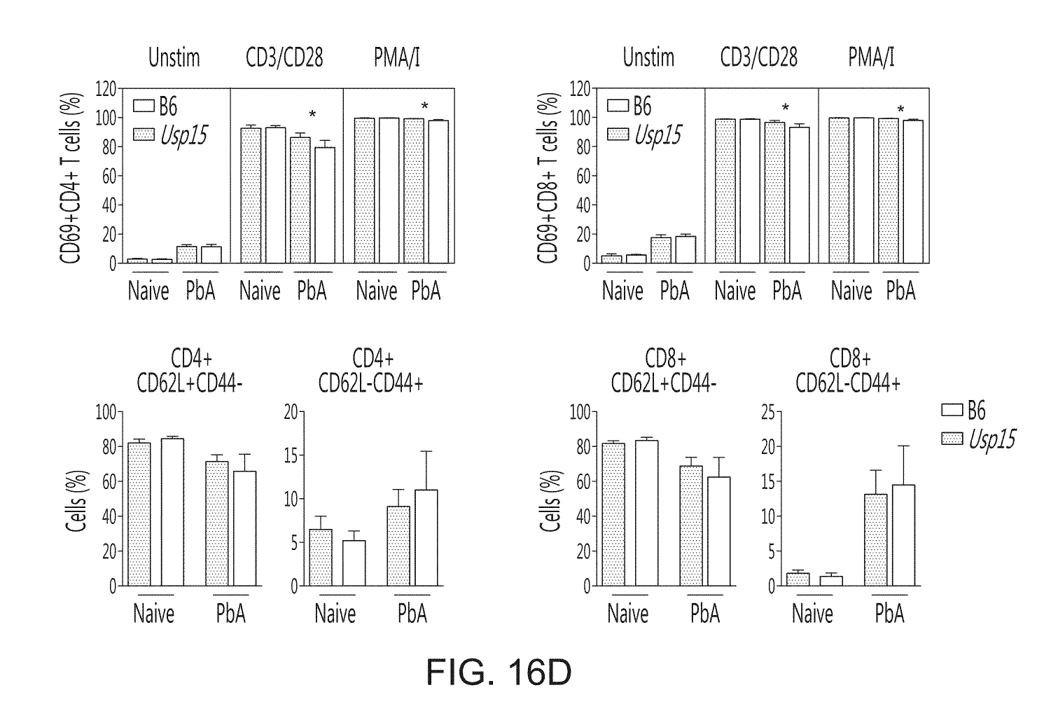

Neuroinflammation in the ECM model is associated with loss of integrity of the blood-brain barrier (BBB), driven in part by trapping of parasitized erythrocytes and ensuing pro-inflammatory immune responses in situ. We investigated the protective effect of the Usp15^{L749R} mutation on presence and activity of immune cells at day 5 post-PbA infection. We found reduced infiltration of CD45^{hi} leukocytes, T cells, and CD11c⁺Ly6C^{hi} monocytes in the brains of ECM-resistant Usp15^{L749R}-infected mice compared to wild type ECM-susceptible B6 controls (FIG. 10A). Analysis of major serum cytokines and chemokines in control and mutant infected mice at day 5, showed reduced levels of circulating pro-inflammatory chemoattractants MIP-1a/CCL3 and MIP-1β/CCL4, that are necessary for recruitment of immune cells in the mutants, while serum levels of major myeloid (IL-12p40) and lymphoid (IFNγ, TNFα) Th1 cytokines were similar in both groups (FIG. 10B, and data not shown). Additional immunophenotyping of myeloid and lymphoid cells at steady state and during PbA infection failed to reveal a major immune defect in the Usp15^{L749R} mutant animals, with respect to a) frequency of T cells, B cells, NK cells, neutrophils and monocytes subsets, b) in vitro cytokine production (IFNγ, TNFα), maturation (CD44+) and activation (CD69⁺) of CD4⁺ and CD8⁺ T cells upon TCR engagement or upon PMA/ionomycin stimulation, and c) activity of myeloid cells (IL-12p40 production) (FIG. 16), and ROS production (data not shown). Taken together, these results suggest that, ECM-resistance in the Usp15^{L749R} mutant is not caused by a dampened Th1 immune response.

We further investigated the effect of the Usp15^{L749R} mutation in a non-microbial model of neuroinflammation, experimental autoimmune encephalomyelitis (EAE) (FIG. 10C). In EAE, neuroinflammation and axonal damage is induced by autoimmune response to myelin oligodendrocyte glycoprotein (MOG) co-administered with pertussis toxin (PTX) that acts to disturb BBB integrity. We observed that Usp15^{L749R} mutant mice were resistant to EAE, compared to wild type B6 (susceptible) and Jak3^{-/-} mutants (resistant) that were used as controls. Resistance in Usp15^{L749R} mutants was expressed as absence of body weight loss (FIG. 10C), lower clinical scores (FIG. 10D) and reduced overall lethality (FIG. 10E). Examination of clinical scores for individual mice (FIG. 10F) further indicated that while B6 controls progress rapidly to fatal paralysis associated with infiltration of inflammatory cells in the spinal cord (data not shown), Usp15^{L749R} mutants display a much milder phenotype that resembles relapsing-remitting disease, with significant recovery and survival in ˜2/3 of the animals. Measurements of serum cytokines at different times following initiation of EAE consistently pointed to reduced levels of circulating MIP-1a/CCL3 and MIP-113/CCL4 in Usp15^{L749R} mutants very early during induction at day 2 (FIG. 10G).

Taken together, these results confirm the importance of USP15 in neuroinflammation and point to an effect of loss of USP15 function on early production of chemokines and cytokines known to play a key role in leukocyte recruitment to the site of injury.

Cellular Responses and Signaling Pathways Regulated by USP15 During Neuroinflammation.

We used global RNA sequencing (RNA-seq) to gain insight into the cells and pathways that are regulated in situ by USP15, and the inactivation of which may lead to protection against lethal neuroinflammation in Usp15 mutants. Such pathways would be detected as differentially expressed (VVT vs. Usp15^{L749R} mutant) in brain during PbA infection (ECM), and in spinal cord during EAE. We investigated RNA expression in brains at day 5 post-PbA infection, and in spinal cord at day 7 post-initiation of EAE, time points that precede appearance of clinical symptoms in either mouse groups. Dimension reduction analysis performed on normalized gene expression values shows clear clustering of the datasets for each experimental group with at least 3 principal components, linked to tissue origin, disease type and progression, and genotype (FIG. 11A). Differentially expressed genes were investigated in these datasets using a 1.5 fold cut-off, and an adjusted p value of <0.05. This analysis identified 173 genes which were downregulated in the brain of PbA infected Usp15^{L749R} mutant mice and 112 downregulated in the spinal cord of Usp15^{L749R} mutants undergoing ECM (FIG. 11B). This analysis identified sets of USP15-regulated transcripts that were specific for each tissue/condition (brain/ECM; spinal cord/EAE), but also revealed an overlapping set of 39 genes that were downregulated in a USP15-specific fashion in both the ECM and EAE datasets (complete listing in Table 4); together these down-regulated transcripts define the USP15-regulated transcripts. An analysis of gene ontology annotations (G0 term) associated with USP15-regulated genes (FIG. 11C) showed a very significant enrichment for immune responses-type functions (log₁₀ p<10³ to log₁₀ p<10²³), including “response to virus”.

In order to identify genes and associated cellular responses regulated by USP15 during neuroinflammation in the PbA and ECM datasets we performed gene set enrichment analyses [GSEA analysis]. This computational method performs pair-wise analysis of the experimental gene sets (i.e. B6 vs Usp15^{L749R}) using >1900 immune signatures associated with cell-specific and pathway-specific response. GSEA also provides a normalized enrichment score (NES) and a false-discovery rate (FDR) to evaluate the strength and significance of associations in the dataset. GSEA analysis identified a clear enrichment (FDR<0.01) for signatures associated with responses of different cell types to viral infections, response to virus vaccines in peripheral blood mononuclear cells, and response of different cell types to IFNa. FIG. 11D provides illustrative examples of this analysis, including several signatures found to be regulated by USP15 in both the ECM and EAE datasets (mRNA transcripts depleted in the Usp15^{L749R} mutants).

In GSEA, “leaders” are the genes that drive enrichment of a particular signature, thus genes that are low in USP15^{L749R} mice and located after the peak where the cumulative enrichment score reaches a maxima (green line; FIG. 11D). In leading edge analysis (LEA), these genes and intersecting signatures can be further clustered to examine their overall contribution to pathogenesis and the involvement of specific responses and associated cell types in the affected tissue. LEA of the ECM (FIG. 17A), EAE (FIG. 17B), and of the combined ECM/EAE datasets (FIGS. 12A, B) clearly identified the principal USP15-regulated “leaders” and associated signatures corresponding with the type I interferon response, which include genes such as Oasl1/2, Isg15, Ifi41, Ifit1/3, Irf7/9, Usp18, Mt1/2, Mx1 and several others. This was evidenced by the strength of the effect of USP15 on expression of drivers, the number of differentially expressed drivers, and the number of intersecting and biological signatures defined by these drivers. Moreover, 99% of the 125 genes driving the enrichment of IFN-related signatures (red cluster in FIG. 12A) were defined as type I IFN regulated genes in the Interferome database. Interestingly, several poorly annotated genes whose function in the immune system is unknown (e.g. Plin4) were also detected as strong drivers in this analysis. Other leaders and signatures were also detected in the dataset, albeit less strongly. They included cell-specific lymphoid and myeloid signatures as well as markers of activation of these cells, possibly reflecting infiltration of these cells at the site of tissue injury (FIG. 12A), in agreement with direct cellular infiltration data shown in FIG. 10A. Illustrative examples of USP15-regulated leaders are shown in FIG. 12B for the major classes.

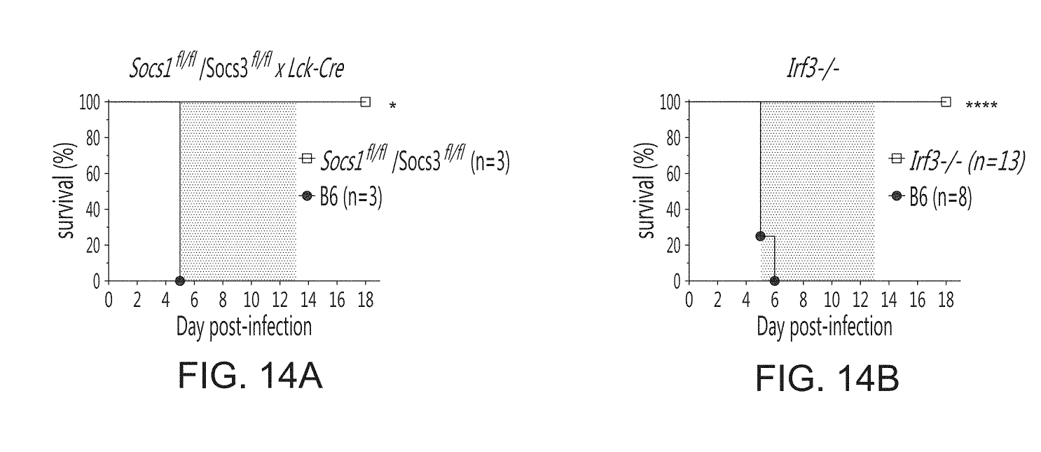

The effect of USP15 on differential gene expression was validated by qPCR, using RNA from PbA-infected brains at days 1, 3 and 5 post-infection (FIGS. 12C-F). This analysis confirmed the major effect of USP15 detected by GSEA and LEA on genes associated with type I interferon responses (Oasl2, Ifit3, Usp18, Irf7, Irf9, Ifi35; FIG. 12C), lymphoid cells (Gmzb, Cd3g, Lat; FIG. 12D), myeloid cells (Cebpd, C4b, Cd11b; FIG. 12E), and genes of unknown function (Plin4; FIG. 12F). These studies showed that the effect of USP15 genotype on gene expression occurred rapidly and was detectable as early as day 3 post-PbA infection. Finally, the relevance of USP15-regulated genes in the type I interferon pathway on susceptibility to neuroinflammation was investigated directly using the ECM model. In these experiments, we determined that mouse mutants bearing loss of function mutations in key LEA leaders such as Socs1/Socs3, Irf3 (FIG. 14) and Ifi35 (data not shown) display significant protection against neuroinflammation and are ECM-resistant.

Taken together, these data point to impaired USP15-dependent engagement of type I interferon responses as a key contributor to ECM and EAE resistance in the Usp15^{L749R} mutant.

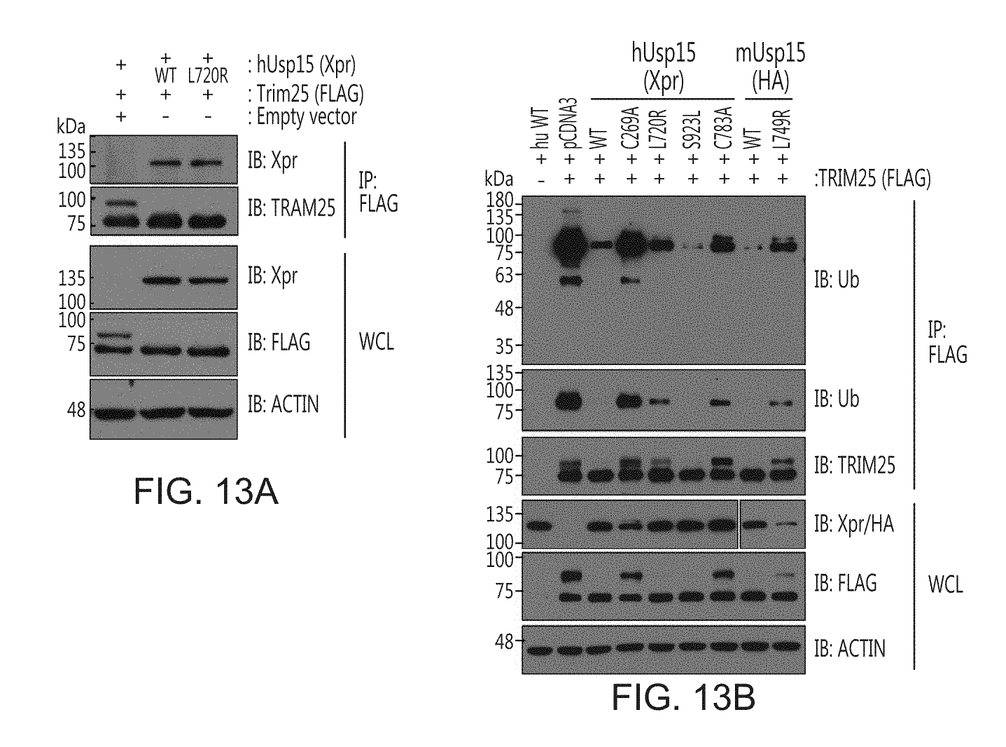

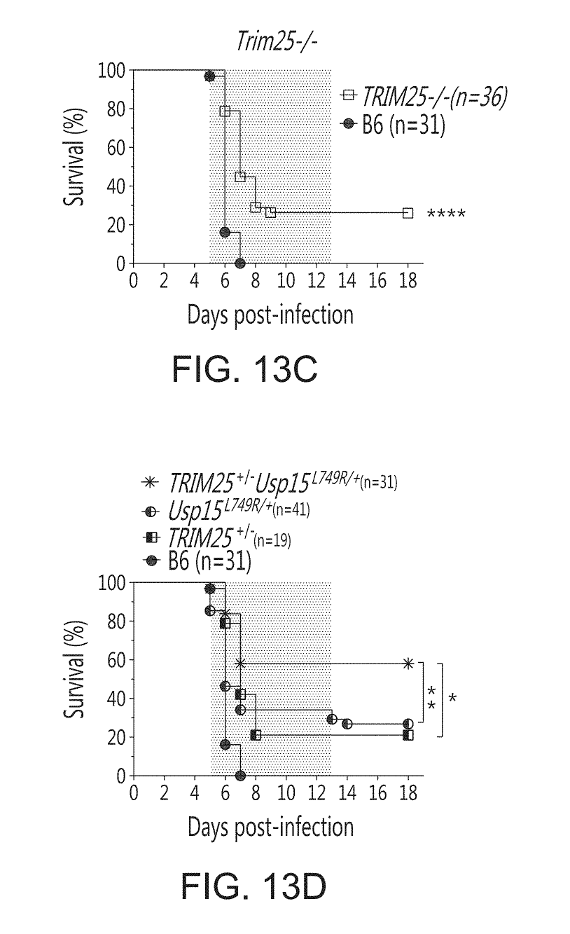

Interaction between USP15 and E3 ubiquitin ligase TRIM25 is required for pathogenesis in neuroinflammation. Recent studies have suggested that USP15 can deubiquitinate a number of proteins, including the E3-ubiquitin ligase, TRIM25, that plays a role in RIG-I signaling. To gain further insight into a potential role for the USP15/TRIM25 dyad in regulation of host response to neuroinflammation, we conducted several experiments. First, we co-transfected epitope tagged versions of wild-type USP15 and TRIM25 in HEK293T cells, and used co-immunoprecipitation and immunoblotting to demonstrate that the two proteins indeed physically interact when co-expressed in the same cells (FIG. 13A). These studies also established that the L749R mutation did not impair USP15/TRIM25 physical interaction, at least when tested in this system. Secondly, we observed that TRIM25 is a target for deubiquitination by USP15. This was demonstrated with anti-ubiquitin and anti-TRIM25 antibodies that allow detection of both the ubiquitinated and the un-ubiquitinated protein when used together (FIG. 13B; panels 1-3). Importantly, we observed a reduced deubiquitinase activity in the L749R variant (the corresponding human L720R isoform was tested), when compared to VVT, and to inactive catalytic mutants C269A and C783A that were used as positive and negative controls respectively (FIG. 13B). Thirdly, we determined that loss of function of TRIM25 in Trim25 mutant mice confers significant degree of ECM-resistance (FIG. 13C). Finally, we tested possible genetic interaction between Usp15 and Trim25 in neuroinflammation and ECM pathogenesis. We observed that introduction of one null Trim25 mutant allele on the background of heterozygosity for Usp15^{L749R} causes significant increase in ECM-resistance (as measured by survival) in Trim25^{-/-}:Usp15^{L749R/+} double heterozygotes (˜60%) compared to single heterozygote mice used as controls (FIG. 13D).

Genes whose expression are modulated in both Trim25 and Usp15^{L749R} are show in Table 6.

TABLE 6
Genes differentially expressed Trim25^{-/-} and
Usp15^{L749R}. Modulation is provided with respect to
the wild-type animals.

| Gene | Modulation | IFN-stimulated gene family? |
|---|---|---|
| Gzmb | Decrease | Yes |
| Gzma | Decrease | Yes |
| Fcgr4 | Decrease | Yes |
| Plaur | Decrease | Yes |
| Ms4a6d | Decrease | Yes |
| Cebpd | Decrease | Yes |
| Maff | Decrease | Yes |
| Socs3 | Decrease | Yes |
| Arrdc2 | Decrease | No |
| Mt1 | Decrease | Yes |
| Mt2 | Decrease | Yes |
| Cdkn1a | Decrease | Yes |
| Srgn | Decrease | No |
| Zfp36 | Decrease | Yes |
| Map3k6 | Decrease | No |
| Fkbp5 | Decrease | Yes |
| Itgb7 | Decrease | Yes |
| Rhoj | Decrease | No |
| Hmgb2 | Decrease | No |
| Ucp2 | Decrease | No |
| Entpd4 | Decrease | No |
| Rbm3 | Decrease | No |

Taken together, these results demonstrate physical and functional interaction between USP15 and TRIM25, and show that USP15^{L749R} impairs this functional interaction. Furthermore, the genetic interaction between Usp15 and Trim25 noted in complementation studies establishes that inactivation of the USP15/TRIM25 dyad is sufficient for ECM-protection. This highlights the role of USP15/TRIM25 in neuroinflammation.

We have used genome-wide ENU mutagenesis in mice to identify novel genes and pathways which when inactivated cause protection against neuroinflammation. We have used an accepted model of encephalitis and associated acute neuroinflammation caused by infection with Plasmodium berghei ANKA (experimental cerebral malaria). In this model, pathogenesis was driven in part by a) trapping of parasitized erythrocytes in microvasculature (brain, lung, spleen), b) tissue damage and early production of chemo-attractants, c) recruitment of myeloid and lymphoid pro-inflammatory cells leading to amplification of inflammatory response in situ, d) loss of BBB integrity, and e) appearance and rapid progression of neurological symptoms. Reverse and forward genetic studies in the ECM model have proven extremely useful to identify the cell types and proteins that are required for pathogenesis of neuroinflammation. These include genes that affect the number, the maturation and function of lymphoid cells (Cd8, Lck, Themis, Jak3, Stat1), and myeloid cells (Irf8, Irf1, Ccdc88b, Fcerg, Cd40), the production of soluble mediators of inflammation (Ifng, 11, complement components) and several others.

In the present example, we reported the identification of an ECM-protective mutation in the Usp15 gene (Usp15^{L749R}), a deubiquitinase member of the USP family. The causative nature of the mutation was validated by backcrossing the mutation on different genetic backgrounds, by genetic complementation testing using a Usp15 null allele, and by the demonstration that the USP15^{L749R} variant behaved as a loss of function mutation caused by impaired protein stability, linked to significantly reduced half-life of the protein and impaired enzymatic activity towards a known target. In PbA-infected animals, the ECM-protective effect of Usp15^{L749R} was associated with reduced infiltration of lymphoid and myeloid cells in the brain, reduced early production of pro-inflammatory chemokines, absence of neurological symptoms, and increased survival. The relevance of USP15 contribution to the pathogenesis of neuroinflammation was further demonstrated by the observation that Usp15^{L749R} mutant mice are also protected in another, non-microbial model of neuroinflammation, the experimental autoimmune encephalomyelitis (EAE) model.

What are the cellular and molecular pathways that are controlled by USP15 and that play a role in pathogenesis of acute neuroinflammation? Recent studies using loss of function (gene silencing) or gain of function (overexpression) approaches in cell-based model systems have implicated USP15 in multiple, seemingly unrelated biochemical pathways and cellular responses. USP15 deubiquitinase activity alone or in combination with other proteins has been associated with regulation of IκBα and activation of NF-κB, parkin-mediated mitochondrial ubiquitination and mitophagy, MAPK activity through stabilization of the E3 ligase BRAP/IMP, the Nrf2 pathway in anti-oxidant response, and histone (ubH2B) deubiquitination. USP15 has also been shown to regulate TGF-β signaling and associated transcriptional activation, with SMAD3, the E3 ubiquitin ligase SMURF2 and the type I TGF-β receptor being direct targets for USP15-dependent deubiquitination. Recently, USP15 has been implicated in regulation of certain aspects of the immune system. USP15 can negatively regulate Th1 responses in CD4⁺ T cells (anti-Listeria and anti-tumor activities) through active stabilization of the E3 ubiquitin ligase MDM2, and with concomitant degradation of NFATc2. Finally, recent studies have suggested that USP15 may contribute to regulation of type I interferon response. However, the role of USP15 in type I interferon response remains controversial, having been alternatively demonstrated to act as a strong activator (through deubiquitination of TRIM25) or a potent inhibitor of this response (through deubiquination of RIG-I).

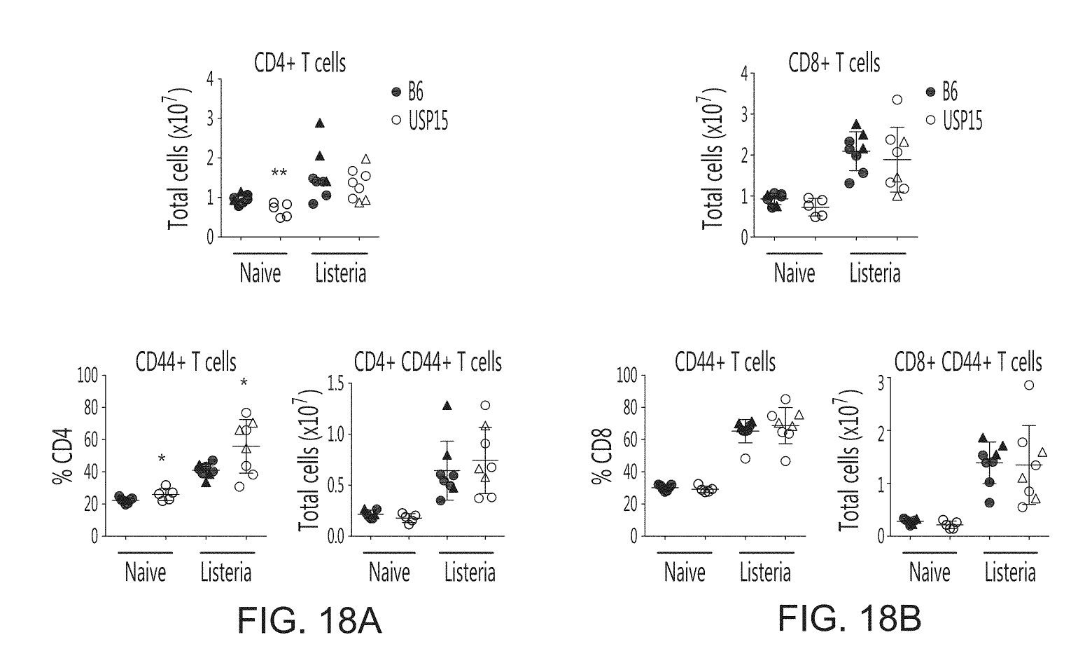

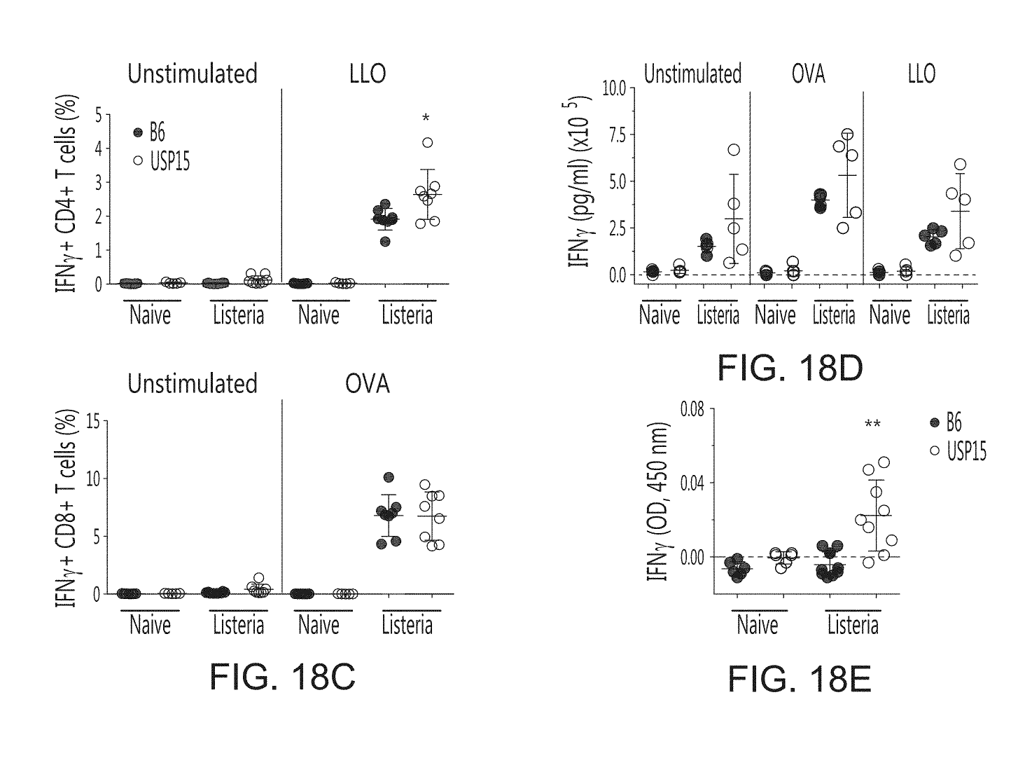

Cellular immunophenotyping of our Usp15^{L749R} mutant mice following L. monocytogenes infection suggest that in this model USP15 acted as a negative regulator of Th1 response. Indeed, L. monocytogenes-infected Usp15^{L749R} mutants showed increased maturation (fraction of CD4⁺/CD44⁺), and increased activation (IFNg production) of CD4⁺ T cells in response to listeriolysin (LLO) (FIG. 18), although this had no impact on bacterial survival and replication in target tissues (data not shown). However, parallel studies of PbA-infected mice failed to demonstrate an effect of USP15 on the fraction (%), maturation (CD44⁺), proliferation, and Th1 cytokines production (IFNg, TNFα, IL2) by CD4⁺ and by CD8⁺ T cells, and this in response to TCR engagement (dose-response) or to non-specific stimuli (FIG. 16). Therefore, ECM-resistance in our Usp15^{L749R} mutant did not appear to be linked to increased Th1 responses associated with loss of USP15 function. To the contrary, ECM protection has been previously associated with a dampening or inactivation (and not augmentation) of Th1 response in mouse strains bearing null alleles at loci such as Ifng, Stat1, Irf1, Irf8, Lck, Themis, and Jak3.

On the other hand, RNA sequencing datasets from brain (ECM model) and spinal cord (EAE model) showed a striking effect of the Usp15^{L749R} mutation on induction of type I interferon response. This differential induction was a) highly significant, b) detected as the dominant pathways both by G0 and GSEA/LEA analysis (response to virus, response to type I IFN, response to vaccine), and c) validated by RT-qPCR. These results establish for the first time that USP15 acted as an in vivo activator of type I interferon response during acute neuroinflammation and encephalitis. The E3 ubiquitin ligase TRIM25 ubiquitinates RIG-I and positively regulates RIG-I mediated production of IFNα and IFNb; ubiquitination of TRIM25 by LUBAC (HOIL-1L, HOIP) stimulated degradation of TRIM25 and suppresses RIG-I signaling. Here, we observed that USP15 physically interacted and deubiquitinated TRIM25. Importantly, we show that loss of TRIM25 function caused enhanced ECM resistance, and we further demonstrated genetic interaction between USP15 and TRIM25 expressed as robust ECM resistance in USP15^{L749/+}: TRIM25^{-/+} double heterozygotes (FIG. 13). These results confirm that USP15 acted as an activator of type I IFN response in vivo during neuroinflammation, and further demonstrated a critical role of the USP15/TRIM25 regulatory dyad in this activation.

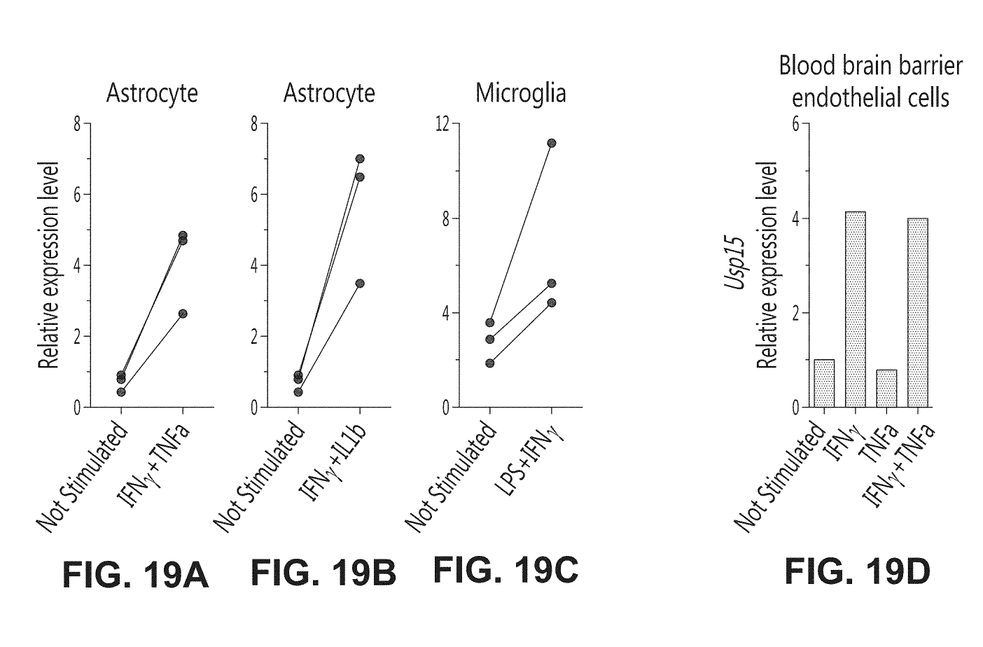

At present, we cannot determine with certainty if the USP15 effect on type I IFN response during neuroinflammation is driven primarily by differences in activity of infiltrating peripheral myeloid and lymphoid cells, or by differences in activity of resident cells (glia, astrocytes, endothelial cells of the BBB) in situ or both. On the one hand, kinetic analyses of RNA expression by qPCR (FIG. 12) showed that the differential expression of type I IFN response genes (Oasl2, Ifit3, Usp18, Irf7, Irf9) in wild type and Usp15^{L749R} mutant animals coincided with appearance of lymphoid (Gmzb, Lat, Cd3g) and myeloid cell markers (Cebpd, C4b, Cd11b) in brain, the extent of which is affected by the Usp15 genotype. On the other hand, studies in bone marrow radiation chimeras indicated that ECM-resistance in the Usp15^{L749R} mutant cannot be abrogated solely by transfer of hematopoietic cells from ECM-susceptible B6 controls, arguing against a major role of lymphoid and myeloid cells in phenotypic expression of the genetic effect at Usp15 (data not shown). In addition, we found that USP15 mRNA is expressed in human primary glial cells, astrocytes, and in endothelial cells of the BBB, where it was strongly induced following exposure to IFNg/TNFα, IFNg/IL1b, and IFNg/LPS combinations of pro-inflammatory stimuli (FIG. 19). Hence, without wishing to be bound to theory, it is tempting to speculate that USP15 contribution to pathogenesis of neuroinflammation is linked to regulatory activation of type I IFN response in situ by resident cells in the brain.

Our results suggested a critical dual but opposite role of type I IFN in the pathogenesis of cerebral malaria. Indeed, studies of the early liver stage disease in primary hepatocytes and in liver cell lines infected with P. berghei sporozoites (insect form) demonstrated strong induction of type I IFN within 36 to 48 hours of infection. This liver-stage response to PbA infection is defective in mice bearing null mutations at the type I IFN receptor (Ifnar1^{-/-}) or in proteins associated with nucleic acid sensing (Rig-I^{-/-}), induction (Mavs^{-/-}, Mda5^{-/-}) and amplification (Irf3⁻) of IFNa/IFNb production. This initial liver stage type I IFN response is protective, as Ifnar1^{-/-} mutant mice show increased liver infection load and increased blood stage parasitemia. Conversely, we demonstrated that engagement of type I IFN response in later stages of P. berghei infection (in response to blood-stage merozoites) was detrimental to the host, and drives pathogenesis of cerebral disease in the brain in situ. Indeed, we showed that the dampening of type I IFN response in Usp15^{L749R} and in Trim25⁻ mutant, and in markers of this pathway such as Irf3^{-/-}, Ifi35^{-/-}, and Socs1/Socs3^{-/-} mutants (FIGS. 13, 14, data not shown) protected against lethal ECM. These results demonstrated a critical dual role of type I IFN in malaria progression, being protective early (liver stage) and detrimental in late stages (cerebral malaria) of disease. They also strongly suggested that modulation of type I IFN response in neuroinflammation in general may be of therapeutic value.

### Example VIII—Lyst

A genetic screen has been performed as described in Example I and a further protective mutation (R1081*, e.g. a deletion) was identified in Lyst, a protein that regulates intracellular protein trafficking in endosomes.

An heterozygote mouse strain has been produced.

### Example IX—Zbtb7B

A genetic screen has been performed as described in Example I and a further protective mutation (R367Q) was identified in Zbtb7b, a transcription factor. The mutant Zbtb7p protein protected the mouse from a P. bergei challenge (data not shown).

An heterozygote mouse strain has been produced.

### Example X—Bpgm1

A genetic screen has been performed as described in Example I and a further protective mutation (L166P) was identified in Bpgm1. The protective mutation was identified by whole exome sequencing to be in the biphosphoglycerate mutase gene (Bpgm) in the form of a L166P mutation. The mutation is non-conservative and affects a highly conserved residue in the enzyme that is invariant from humans to fungi. The wild-type protein is a tri-functional enzyme in the Rapoport-Luebering Shunt pathway. It possesses synthase, mutase activity and that catalyzes the transformation of 1,3 diphosphoglycerate to 2,3 biphosphoglycerate (2,3BPG). 2,3BPG is an allosteric regulator of hemoglobin (Hb) and binds to unligated Hb. Bpgm1 is also part of glycolysis modulating the ratio of 1,3BPG and 3-phosphoglycerate. The mutation was backcrossed on two different genetic backgrounds, A/J and B6. Resistance to neuroinflammation caused by mutation in BPGM was detected on both genetic backgrounds validating the observation. The mutant Bpgm1p protein protected the mouse from a P. bergei challenge (data not shown).

An heterozygote mouse strain has been produced.

### REFERENCE

Bongfen S E, Rodrigue-Gervais I G, Berghout J, Torre S, Cingolani P, Wiltshire S A, Leiva-Torres G A, Letourneau L, Sladek R, Blanchette M, Lathrop M, Behr M A, Gruenheid S, Vidal S M, Saleh M, Gros P. An N-ethyl-N-nitrosourea (ENU)-induced dominant negative mutation in the JAK3 kinase protects against cerebral malaria. PLoS One. 2012; 7(2):e31012. Epub 2012 Feb. 21.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A method for assessing the ability of an agent to prevent, treat and/or alleviate the symptoms associated with an inflammatory condition in an individual, said method comprising:
a) combining the agent with an inflammatory enabling polypeptide selected from the group consisting of a USP15 polypeptide and a TRIM25 polypeptide;
b) measuring a biological activity of the inflammatory enabling polypeptide of step (a) to obtain a test level;
c) comparing the test level to a control level, wherein the control level is associated with the biological activity of the inflammatory enabling polypeptide observed during the onset or maintenance of the inflammatory condition; and
d) characterizing the agent as (i) useful for the prevention, treatment and/or alleviation of the symptoms associated with the inflammatory condition when the at least one biological activity associated with the test level is lower than the biological activity associated with the control level or (ii) lacking utility for the prevention, treatment and/or alleviation of the symptoms associated with the inflammatory condition when the at least one biological activity associated with the test level is equal to or higher than the biological activity associated with the control level.

2. The method of claim 1, wherein the biological activity of the USP15 polypeptide is a de-ubiquitinating activity.

3. The method of claim 2, further comprising, in step (a), combining the agent and the USP15 polypeptide with a ubiquinated TRIM25 polypeptide.

4. The method of claim 3, further comprising, in step (b), measuring the level of the ubiquinated TRIM25, the level of a partially ubiquinated TRIM25 or the level of a deubiquinated TRIM25 polypeptide to measure the biological activity of the USP15 polypeptide.

5. The method of claim 1, wherein step (a) is conducted or the control level of step (b) is obtained in vitro.

6. The method of claim 5, wherein step (a) is conducted in or the control level of step (b) is obtained from a cell.

7. The method of claim 6, wherein the cell bears one gene copy coding for a non-functional USP15 polypeptide.

8. The method of claim 1, wherein step (a) is conducted or the control of step (b) is obtained in vivo.

9. The method of claim 8, wherein step (a) is conducted in or the control level of step (b) is obtained from a non-human animal.

10. The method of claim 9, wherein the non-human animal bears one gene copy coding for a non-functional USP15 polypeptide.

11. The method of claim 1, wherein the inflammatory condition is neuroinflammation.

12. The method of claim 1, wherein step (b) comprises determining the test level of expression of at least one of the following genes Gzmb, Gzma, Fcgr4, Plaur, Ms4a6d, Cebpd, Maff, Socs3, Arrdc2, Mt1, Mt2, Cdknla, Srgn, Zfp36, Map3k6, Fkbp5, Itgb7, Rhoj, Hmgb2, Ucp2, Entpd4 or Rbm3 and step (c) comprises determining the control level of the corresponding genes.

13. The method of claim 12, wherein step (b) comprises measuring the test level of:
at least one of the following genes Gzmb, Gzma, Fcgr4, Plaur, Ms4a6d, Cebpd, Maff, Socs3, Mt1, Mt2, Cdknla, Zfp36, Fkbp5 or Itgb7; and
at least one of the following genes Arrdc2, Srgn, Map3k6, Rhoj, Hmgb2, Ucp2, Entpd4 or Rbm3.
